(19) United States
(12) Patent Application Publication
(10) Pub. No.: US 2017/0321256 A1
(43) Pub. Date: Nov. 9, 2017

(54) METHODS FOR DISTINGUISHING INFLAMMATORY BOWEL DISEASES USING MICROBIAL COMMUNITY SIGNATURES

(71) Applicants: Wolfgang F Fricke, Baltimore, MD (US); James Robert White, Baltimore, MD (US)

(72) Inventors: Wolfgang F Fricke, Baltimore, MD (US); James Robert White, Baltimore, MD (US)

(21) Appl. No.: 15/489,658

(22) Filed: Apr. 17, 2017

Related U.S. Application Data
(63) Continuation-in-part of application No. 15/028,253, filed on Apr. 8, 2016.
(60) Provisional application No. 62/323,257, filed on Apr. 15, 2016.
(60) Provisional application No. 61/888,288, filed on Oct. 8, 2013.

(51) Int. Cl. C12Q 1/68; G06F 19/20

(52) U.S. Cl. CPC C12Q 1/689; G06F 19/20; C12Q 2600/112; C12Q 2600/158

(57) ABSTRACT

The inventive subject matter relates broadly to novel techniques for identification and comparative analysis of sequence features in metagenomic whole-genome shotgun (WGS) sequence data associated with particular disease states in a subject. More particularly, the inventive subject matter relates to diagnostic methods for distinguishing between different types of inflammatory bowel disease in a subject based on the microbial community signature of the subject.

### CROSS REFERENCE TO RELATED APPLICATIONS

This application is a Continuation-In-Part which claims the benefit of U.S. Provisional Application No. 62/323,257, filed Apr. 15, 2016, and claims priority to U.S. patent application Ser. No. 15/028,253, filed Apr. 8, 2016, which claims priority from PCT/US2014/011321 filed Jan. 13, 2014, which claims the benefit of U.S. Provisional Application Ser. No. 61/888,288 filed Oct. 8, 2013, the disclosures of which are hereby incorporated by reference herein in their entirety.

### TECHNICAL FIELD

The inventive subject matter generally relates at least to the fields of molecular biology, molecular diagnostics, infectious disease, and medicine. The inventive subject matter relates broadly to novel techniques for identification and comparative analysis of sequence features in metagenomic whole-genome shotgun (WGS) sequence data associated with particular disease states in a subject. More particularly, the inventive subject matter relates to diagnostic methods for distinguishing between different types of inflammatory bowel disease in a subject based on the microbial community signature of the subject.

### BACKGROUND

The human body and its associated microbiota, the “human microbiome,” represent a complex superorganism with thousands of microbial species distributed biogeographically among niche body sites [1]. The fundamental role of the microbiome for human health is generally accepted and a growing body of literature reports associations between specific diseases or disorders and the microbiome [2-4]. However, in many cases it has remained difficult to correlate a defined microbiome characteristic with a specific disease, disorder, condition, or symptom thereof. Furthermore, recent studies have demonstrated significant diversity in microbial communities between healthy humans, most notably in the digestive tract [2,4-8]. Additionally, the host-microbiome relationship is highly dynamic; host-induced changes to diet or other perturbations, such as antibiotic treatment, can significantly alter microbial composition thereby potentially inducing secondary effects on human health [7,9-11]. Despite the known within- and between-subject taxonomic variation, Applicants have determined that by characterizing certain complex systems, specific microbiome signatures can be identified that are predictive of health and/or disease state(s) of the human host.

The rapid advancement of next-generation DNA sequencing technologies has allowed for deep sampling of human-associated microbial communities either in the form of whole genome shotgun sequencing of metagenomic DNA or multiplexed PCR amplicons of the 16S rRNA gene. The Human Microbiome Project (HMP) has already sequenced over ˜3.5 TB of microbial DNA from ˜750 human samples, revealing a vast phylogenetic diversity and functional capacity of commensals throughout the human population and across the human body [1,12,13]. There is extensive information to learn from the HMP data, including what constitutes a healthy or “normal” microbiome.

Assembly and taxonomic assignment of metagenomic data remain active areas of research for computational scientists, and although next-generation platforms produce tremendous amounts of sequence, many analysis protocols that were developed for larger sequence reads from Sanger or Roche/454 technologies are difficult to apply due to short read lengths. While there has been substantial effort to develop computational methods that assign single metagenomic reads and assembled contig fragments to specific taxonomic lineages or metabolic functions [14-20], identification of a group of specific nucleic acid sequences (a “signature”) that define a particular microbiome within samples as a way to associate sample composition with sample background has received less attention.

While recent applications of machine learning algorithms have demonstrated that metagenomic sample classification with some accuracy at some level is achievable [21-25], these studies were largely limited to analyses of targeted sequence data, i.e. products of polymerase chain reaction (PCR) amplifications of the universal bacterial marker gene, the small ribosomal RNA subunit or 16S rRNA. Though easy to obtain, the prior art 16S rRNA datasets suffer from several deficiencies, providing limited phylogenetic information which is typically heavily biased by primer design, amplicon region, and gene copy number variability. Moreover, although phylogenetically-related bacteria tend to show similar phenotypic presentations, 16S rRNA-based phylogenetic profiles and the composition of health-relevant functional microbiomes are not necessarily correlated. For example, similar functions could be performed by phylogenetically distinct organisms within the microbiome. According to the understanding prior to Applicants' work, in the extreme case, this could in theory result in a situation where two perfectly healthy human subjects have completely different, non-overlapping phylogenetic microbiome compositions. In contrast, WGS metagenomic datasets, which do not include a PCR amplification step, have fewer technical caveats relative to 16S rRNA surveys, which in turn provides novel opportunities to find distinctive features of different microbiomes and associations with phenotypic microbiota representations.

In this application, Applicants demonstrate that the elucidation of distinctive features of different microbiomes and phenotypic microbiota representations are useful for diagnosing and monitoring the state of a disease, disorder, or condition in a subject. The inventive subject matter provides solutions to this and other important problems associated with inflammatory bowel disease as an exemplary disease state.

### SUMMARY OF THE INVENTIVE SUBJECT MATTER

In one aspect, the inventive subject matter is directed to a method of diagnosing inflammatory bowel disease in a subject in need thereof, comprising the steps of:
(a) in a microbiome sample obtained from said subject, determining the presence of a nucleic acid oligomer that distinguishes subjects having inflammatory bowel disease from subjects not having inflammatory bowel disease;
(b) recording the presence and identity of said nucleic acid oligomer in an electronic format; and
(c) making and recording a diagnosis of inflammatory bowel disease based on the presence of said nucleic acid oligomer.

In another aspect, the inventive subject matter is directed to a method of differentially diagnosing Crohn's disease and ulcerative colitis in a subject in need thereof, comprising the steps of:
(a) in a microbiome sample obtained from said subject, determining the presence of a signature comprising a plurality of nucleic acid oligomers that distinguishes subjects having Crohn's Disease and subjects having ulcerative colitis;
(b) recording the presence and identity of said signature in an electronic format; and
(c) making a diagnosis of Crohn's disease or ulcerative colitis based on the presence of said signature.

In one aspect, the inventive subject matter is directed to methods for classifying patients with inflammatory bowel disease (IBD) from patients without IBD. In another aspect, the inventive subject matter is directed to methods for distinguishing between inflammatory bowel diseases in a subject, for example distinguishing between ulcerative colitis (UC) and Crohn's disease (CD) in a subject. The inventive subject matter is based on Applicants' discovery that the characterization and analysis of the particular microbiome associated with a subject, such as a human subject, can be used to classify the subject as having a disease, disorder, or condition such as having IBD or not, or having UC or having CD. The microbiome of a subject can thus be screened, and the health and/or medical condition of the subject can be determined.

Because microbiomes are comprised of bacterial populations that differ between individuals, even healthy subjects will have taxonomically different microbiomes. Therefore, a single sequence feature of the microbiome, considered alone, would generally not be expected to serve as a functional diagnostic. However, Applicants have further determined that a group of at least about 10 to larger groups of to 50 or more different sequence features can be screened in a microbiome sample from a subject, and statistically significant diagnoses about the relative health of the subject can be made using this information. In one aspect, some features of interest of the inventive subject matter include particular nucleic acid molecules produced by the population of bacteria that comprise a subject's microbiome. The inventive subject matter uses short nucleic acid oligomers, interchangeably termed “kmers” herein, to survey the entire composite metagenome of all individual microbial genomes obtained from a microbiome sample from a subject. Applicants have determined that by surveying the entire metagenome for the relative amount of pre-selected, relevant kmer sequences, also called significant features herein, one may determine the signature of the microbiome and classify the subject as having or not having a particular disease, disorder, or condition. Thus, the inventive subject matter takes advantage of Applicants' discovery that the composition and characteristics of a subject's microbiome is, in some cases, directly correlated with a particular disease, disorder, or condition.

In an aspect, the inventive subject matter is directed to a method of classifying a subject as having inflammatory bowel disease (IBD), comprising: (a) providing (i) a sample obtained from the subject, the sample comprising nucleic acid from the subject's microbiome, and (ii) a signature(s) that distinguishes subjects having IBD from a subject not having IBD; (b) determining in the sample the abundance of the features comprising the signature (“the dataset”); and (c) classifying the dataset from (b) as being either obtained from a subject having IBD or a subject not having IBD.

In certain aspects, the signature(s) that distinguishes a subject having IBD from a subject not having IBD is obtained by determining a set of kmers with statistically significant differential abundance between a microbiome sample from a subject having IBD and a microbiome sample from a subject not having IBD, and performing an algorithm to identify one or more groups of between 1 and 50 kmers from within the set of kmers that distinguish a subject having IBD from a subject not having IBD. In certain aspects, the kmers are oligomers comprising between 2 and 10 nucleotides, including 2, 3, 4, 5, 6, 7, 8, 9, or 10 nucleotides. In some aspects, the kmers are octamers comprising 8 nucleotides. In some aspects, the signature comprises at least one kmer selected from those listed in Tables 3, 7, and 10. In other aspects, the signature comprises at least one of the 21 kmers listed in Table 7.

In certain aspects, the data set is classified as being obtained from a subject having IBD or a subject not having IBD by performing a nearest neighbor analysis on the data set.

In another aspect, the inventive subject matter is directed to a method for recommending a subject for treatment, comprising: (a) providing (i) a sample obtained from the subject, the sample comprising nucleic acid from the subject's microbiome, and (ii) a signature that distinguishes subjects having IBD from a subject not having IBD; (b) determining in the sample the abundance of the features comprising the signature (“the dataset”); (c) classifying the dataset from (b) as being either obtained from a subject having IBD or a subject not having IBD; and (d) recommending the subject for a treatment when the dataset from (c) is classified as being from a subject with IBD.

In certain aspects, the signature(s) that distinguishes a subject having IBD from a subject not having IBD is obtained by determining a set of kmers with statistically significant differential abundance between a microbiome sample from a subject having IBD and a microbiome sample from a subject not having IBD, and performing an algorithm to identify one or more groups of between 1 and 50 kmers that distinguish a subject having IBD from a subject not having IBD. In certain aspects, the kmers are oligomers comprising between 2 and 10 nucleotides, including 2, 3, 4, 5, 6, 7, 8, 9, or 10 nucleotides. In some aspects, the kmers are octamers comprising 8 nucleotides. In some aspects, the signature comprises at least one kmer selected from those listed in Tables 3, 7, and 10. In other aspects, the signature comprises at least one of the 21 kmers listed in Table 7.

In certain aspects, the data set is classified as being obtained from a subject having IBD or a subject not having IBD by performing a nearest neighbor analysis on the data set.

In some aspects, the treatment is an endoscopic procedure. The endoscopic procedure maybe selected from colonoscopy, sigmoidoscopy, capsule endoscopy, and endoscopic ultrasound. In an aspect, the endoscopic test is a colonoscopy.

In another aspect, the inventive subject matter is directed to a method of classifying a subject as having ulcerative colitis (UC) or Crohn's disease (CD) comprising: (a) providing (i) a sample obtained from the subject, the sample comprising nucleic acid from the subject's microbiome, and (ii) a signature that distinguishes subjects having UC from a subject having CD; (b) determining in the sample the abundance of the features comprising the signature (“the dataset”); and (c) classifying the dataset from (b) as being either obtained from a subject having UC or a subject having CD.

In certain aspects, the signature(s) that distinguishes a subject having UC from a subject having CD is obtained by determining a set of kmers with statistically significant differential abundance between a microbiome sample from a subject having UC and a microbiome sample from a subject having CD, and performing an algorithm to identify one or more groups of between 1 and 50 kmers that distinguish a subject having UC from a subject having CD. In certain aspects, the kmers are oligomers comprising between 2 and 10 nucleotides, including 2, 3, 4, 5, 6, 7, 8, 9, or 10 nucleotides. In some aspects, the kmers are octamers comprising 8 nucleotides. In another aspect, the signature comprises one or more of those listed in Table 4. In another aspect, the signature comprises one or more of those listed in Table 5. In another aspect, the signature comprises one or more kmers listed in Table 3.

In a further aspect, the inventive subject matter is directed to a method of distinguishing between a subject having ulcerative colitis and a subject having Crohn's disease, comprising: (a) determining a set of kmers with statistically significant differential abundance between a microbiome sample from a subject having ulcerative colitis (UC) and a microbiome sample from a subject having Crohn's disease (CD); (b) performing an algorithm to identify one or more groups of between 1 and 50 kmers that distinguish a subject having UC from a subject having CD, wherein such groups are termed signatures; (c) obtaining a microbiome sample from a subject having an inflammatory bowel disease; (d) isolating and sequencing microbiome nucleic acid of the sample; (e) determining the abundance of nucleic acid in the sample corresponding to kmers comprising at least one of the signatures of (b) to produce a data set; (f) normalizing the data set; and (g) classifying the data set as being obtained from a subject having UC or a subject having CD, thereby distinguishing between a subject having UC and a subject having CD.

In certain aspects, the microbiome sample is a stool sample. In certain aspects, the nucleic acid is DNA, cDNA or RNA. In certain aspects, the signature is at least one signature selected from among the 1087 signatures of Table 4. In certain aspects, the signature is at least one signature selected from among the 17 signatures of Table 5.

In certain aspects, the data set is classified as being obtained from a subject having UC or a subject having CD by performing a nearest neighbor analysis on the data set.

The foregoing has outlined rather broadly the aspects and technical advantages of the present invention in order that the detailed description of the invention that follows may be better understood. Additional aspects and advantages of the invention will be described herein, which form the subject of the claims of the invention. It should be appreciated by those skilled in the art that any conception and specific aspect disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel aspects which are believed to be characteristic of the invention, both as to its organization and method of operation, together with further objects and advantages will be better understood from the following description. It is to be expressly understood, however, that any description, table, example, etc. is provided for the purpose of illustration and description only and is by no means intended to define the limits the invention.

### DETAILED DESCRIPTION OF THE INVENTION

### A. DEFINITIONS

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “drug” is as defined under 21 U.S.C. §321(g)(1), and means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals . . . .”

As used herein, the terms “patient” and “subject” refer to a mammal, for example, a human.

As used herein, the terms “treating” or “treatment” of any disease refers to reversing, alleviating, arresting, or ameliorating a disease or at least one of the clinical symptoms of a disease, reducing the risk of acquiring at least one of the clinical symptoms of a disease, inhibiting the progress of a disease or at least one of the clinical symptoms of the disease or reducing the risk of developing at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain aspects, “treating” or “treatment” refers to protecting against or delaying the onset of at least one or more symptoms of a disease in a patient.

### B. METHODS OF DIFFERENTIAL DIAGNOSIS OF INFLAMMATORY BOWEL DISEASES

The inventive subject matter is directed to a method of diagnosing inflammatory bowel disease in a subject in need thereof, comprising the steps of:
(a) in a microbiome sample obtained from said subject, determining the presence of a nucleic acid oligomer that distinguishes subjects having inflammatory bowel disease from subjects not having inflammatory bowel disease;
(b) recording the presence and identity of said nucleic acid oligomer in an electronic format; and
(c) making and recording a diagnosis of inflammatory bowel disease based on the presence of said nucleic acid oligomer.

In one aspect of the inventive subject matter, said microbiome sample is isolated from a stool sample.

In another aspect of the inventive subject matter, the presence of said nucleic acid oligomer is determined by whole-genome shotgun sequencing of total DNA isolated from said microbiome sample.

In a further aspect of the inventive subject matter, said nucleic acid oligomer comprises between 2 and 10 nucleotides.

In a preferred embodiment, said oligomer comprises an octamer.

In a more preferred embodiment, said octamer is selected from the group consisting of the octamers listed in Table 3 and 10.

In a most preferred embodiment, said octamer is selected from the group consisting of the octamers listed in Table 7.

In an alternate aspect of the inventive subject matter, the method comprises the additional step of reporting said diagnosis to a medical professional, to the subject or a representative of the subject, or to a combination thereof.

The inventive subject matter is further directed to a method of differentially diagnosing Crohn's disease and ulcerative colitis in a subject in need thereof, comprising the steps of:
(a) in a microbiome sample obtained from said subject, determining the presence of a signature comprising a plurality of nucleic acid oligomers that distinguishes subjects having Crohn's Disease and subjects having ulcerative colitis;
(b) recording the presence and identity of said signature in an electronic format; and
(c) making a diagnosis of Crohn's disease or ulcerative colitis based on the presence of said signature.

In one aspect of the inventive subject matter, said microbiome sample is isolated from a stool sample.

In another aspect of the inventive subject matter, the presence of each said nucleic acid oligomer is determined by whole-genome shotgun sequencing of total DNA isolated from said microbiome sample.

In a further aspect of the inventive subject matter each said nucleic acid oligomer comprises between 2 and 10 nucleotides.

In a preferred embodiment, each said nucleic acid oligomer comprises an octamer.

In an alternate aspect of the inventive subject matter, said signature is selected from the group consisting of the signatures listed in Table 4.

In a preferred embodiment, said signature is selected from the group consisting of the signatures listed in Table 5.

In yet another aspect of the inventive subject matter, said method comprises the additional step of reporting said diagnosis to a medical professional, to the subject or a representative of the subject, or to a combination thereof.

### C. METHOD TO IDENTIFY FEATURE SETS

In an aspect, the inventive subject matter provides methods to identify feature sets that can be used to classify an individual into one of the sets. Microbial communities associated with the human body are collectively summarized as the “human microbiome.” Differences in human microbiome compositions are generally believed to be associated with different body sites as well as with specific health and disease states. These complex communities of microbial organisms can be studied on the systems level using whole-genome shotgun (WGS) sequencing of total DNA isolated from microbiome samples (termed “metagenomics”). Using bioinformatic tools, microbiome-specific signatures can be identified and used to provide valuable information for basic research, forensics, and clinical diagnostics. However, which features of the microbiome provide the best signature to distinguish and classify microbiome samples remains yet to be determined, as many commonly used parameters in microbial ecology (e.g., 16S rRNA-based phylogenetic community compositions) show large variations across apparently related sample populations (e.g. healthy human stool samples).

Using a computationally efficient, but statistically rigorous, method developed by the inventors and described herein, predictive diagnostic markers (termed “features” herein) have been identified within metagenomic datasets that allow classification of samples as having particular signatures that correspond to selected gastrointestinal disease backgrounds. In particular, a method for identifying microbial community signatures that distinguish a priori-selected sample groups of interest has been developed. These signatures are based on sequence data compositions from metagenomic samples, i.e. total sequenced DNA isolates from entire microbiome samples. Classifiers based on these signatures were assessed for robust sensitivity and specificity using a cross-validation sub-sampling procedure in which random sets of samples were selected and subsequently re-classified individually using the remaining data. Additional testing could be performed using classifiers to assign samples that were not part of the original training sets. This approach was applied to data from the Human Microbiome Project as well as other gut microbiome datasets, including data from IBD patients, and IBD patients with Crohn's disease (CD) and/or ulcerative colitis (UC).

Using this method of statistical analysis, sets of features were identified that allow for patients with Crohn's disease to be distinguished from patients having ulcerative colitis at a statistically-significant rate of accuracy. The method was also applied to identify sets of features that allow for patients with IBD to be distinguished from patients without IBD. Thus, the method of statistical analysis has been applied to metagenomic datasets to create classifiers that accurately diagnose patients as having IBD, as well as distinguish between patients with ulcerative colitis and Crohn's disease. These feature sets can be used to assign additional samples to these two groups. The inventive subject matter may be used to (i) identify signatures characteristic for each of the two classes, (ii) assess the statistical significance associated with these signatures, and (iii) perform a classification of additional samples of unknown patient backgrounds to either of the two groups.

In particular, the inventive subject matter relates to the identification and comparative analysis of sequence-features in metagenomic whole-genome shotgun (WGS) sequence data derived from human clinical specimens. In combination with the statistical analysis method developed by the inventors, a group of sequence-features that correspond to particular human microbiomes, termed significant features, are identified, and together form microbiome signatures. Short nucleic acid sequences or oligomers (termed “kmers” herein) of generally 8 nucleotides are then identified that serve as classifiers for use in screening for signatures that will place a sample into a particular class (e.g., a patient with CD or UC, or a patient with IBD or without IBD). A set of terms that define specific relationships in the method are as follows:
i. A sample (e.g., a sample comprising nucleic acid from the microbiome of a patient) belongs to a particular class (e.g., a patient with CD or UC).
ii. A sample is described by a set of features (e.g., concentrations of particular short nucleic acid sequences (kmers) in the metagenomic sequence data as determined by counting the relative abundances of all possible kmers).
iii. Features that show different concentrations between samples are significant features (e.g., kmers that are significantly more often found in metagenomic sequence data from IBD compared to non-IBD patient samples or from CD compared to UC patient samples and vice versa).
iv. A signature is a set of significant features (e.g., a group of between one and 50 kmers with significantly different relative abundance in one patient class (e.g., IBD) compared to another patient class (e.g., non-IBD) or (e.g., CD) compared to another patient class (e.g., UC).
v. A signature is used to assign a sample to a particular class.

The general manner in which the statistical methodology detailed in the present application is performed can be summarized as follows:
(a) microbiome sequence data is obtained from two sample groups (e.g., subjects having IBD and subjects not having IBD or subjects having UC and subjects having CD);
(b) the abundance of oligomers (kmers) of a selected length (e.g., 2, 3, 4, 5, 6, 7, 8, 9, or 10 mers) in the two data sets is calculated;
(c) kmers with different relative abundances between the two data sets are identified;
(d) sets of kmers (e.g., between 1 and 50 different kmers per set) with different relative abundances between the two data sets are combined;
(e) the sensitivity, specificity, and p-values are determined for each set of kmers, with those showing the ability to distinguish between the two data sets being selected as candidate signatures; and
(f) the robustness of the candidate signatures is determined and scored using internal cross-validation by subsampling and reclassification and suitable signatures are selected as signatures for use in classifying a sample for inclusion in a particular class (e.g., as being isolated from a subject with IBD or a subject with UC or a subject with CD).

### Subjects

While the Examples provided herein are directed to the analysis of samples from human subjects, it will be apparent to one of ordinary skill in the art that the methods described herein can be conducted in conjunction with microbiome samples from a wide variety of animals, including, but not limited to, humans and non-human animals, e.g., a non-human primate, bird, horse, cow, goat, sheep, a companion animal, such as a dog, cat or rodent, or other mammal.

### Source of Microbiome Samples

Microbiome samples analyzed using the inventive methods are not limited in the source or location on the subject from which they are obtained or the means used to obtain them. Example sources include dental plaques, such as supragingival plaque, saliva, and stool. Example locations include all areas of the skin, including on and around the anus, vaginal, urethra, and the interior of the mouth. Additional example locations include the anterior nares, buccal mucosa, posterior fornix, and tongue dorsum. Exemplary techniques for obtaining samples are well known in the art, and include swabs.

### DNA Isolation and Sequencing

When the source of the microbiome sample is stool, extraction of total metagenomic DNA can be conducted using known techniques (Gajer et al., Sci Transl Med 4:132-152 (2012); Sellitto et al., PLoS One 7: e33387 (2012)), including community-approved standard operating procedures (SOPs) established and published by the Human Microbiome Project (http://hmpdacc.org/tools_protocols/tools_protocols.php). In particular, the MoBio UltraClean® Fecal DNA Isolation Kit (MO BIO Laboratories, Inc., Carlsbad, Calif.) can be used, which contains a humic acid inhibitor removal step.

Metagenomic DNA can be multiplexed and sequenced, for example, on a single channel of an Illumina HiSeq 2000 platform, following the manufacturer's recommendations as amended by the Genomics Resource Center at the Institute for Genome Sciences. For example, combining 100 samples on a single HiSeq 2000 channel will generate about 3 million 100 bp paired-end sequence reads per sample.

Working with stool samples provides advantages with respect to potential clinical integration as a high-throughput diagnostic tool: (i) sample amounts: typically 0.1 g of stool are sufficient to isolate genomic DNA for several rounds of sequencing; (ii) automation: robotic platforms and corresponding sample processing kits from several manufacturers (e.g. MoBio, Qiagen, Zymo) allow for automated processing of stool samples for DNA isolation. Together with automated workflow systems for sequencing library generation and sequencing on the Illumina HiSeq 2000 or MiSeq platform, fast, efficient and cost-effective sample processing can be achieved.

### Method of Statistical Analysis

A method of statistical analysis has been developed whereby metagenomic WGS sequence data of particular microbiomes is analyzed to identify particular signatures that can be used to distinguish samples as belonging to one of two (or more) defined classes. Short oligomers of 8 nucleotides (“kmers”), for example, are then identified that serve as features for use in screening for signatures to develop a classifier that will place a sample into a particular class (e.g., a patient with IBD, CD or UC). Kmers of other lengths, such as from 1 to 50 nucleotides in length, may also be used, and the lengths include each integer from 1 to 50.

The described procedure is comprised of three major phases: (1) significant feature selection, (2) signature selection and classifier development, and (3) classifier validation and testing.

### 1. Significant Feature Selection

The first phase of the analysis begins by obtaining a set of n metagenomic dataset samples S=[S₁, S₂, S₃, . . . , Sₙ] associated with a set of m mutually-exclusive classes where m≧2. The metagenomic datasets can be obtained from existing databases, such as the Human Microbiome Project (HMP) body site database or from the MetaHIT project [4] which contains WGS metagenomic samples from stool of European individuals.

### 1.1 Kmer Frequency Estimation

The number of times short DNA oligomers (kmers; e.g., oligonucleotide sequences of 8 nucleotides=an 8 mer) appear in each metagenomic sample S are counted using the Jellyfish program from the open source wgs-assembler software package (http://www.cbcb.umd.edu/software/jellyfish/). Overlapping kmers are counted on both DNA strands, i.e. in the original and reverse complement sequence of each read, resulting in sequences being counted twice. To reduce redundancy, only canonical kmers are stored, i.e., a specific kmer w and its reverse complement w_{rc} are not distinguished. The kmer that appears first in alphabetical order is recorded. Therefore, the feature space consisted of 32,896 canonical Kmers (4⁸(0.5*255/256+1/256)).

### 1.2 Identification of Differentially Abundant Kmers

Kmers are next assessed for differential mean abundance between the two classes, as defined a priori, using the Metastats program [26] (-b set to 5000 permutations). Due to the sizeable feature space of 32,896 kmers, accounting for multiple hypothesis tests is critical to assess false positives. This means that between any two groups, a certain number of kmers will show differential abundance by chance. Therefore, a p-value threshold is selected that controls the false discovery rate [27]. Given a p-value threshold, this results in a set of kmers, designated as corresponding to significant features, to be considered in the next phase. Lastly, kmer counts are normalized to relative abundances within each sample (i.e. given a value between 0 [0%] and 1 [100%]).

### 2. Signature Selection and Classifier Development

### 2.1 Normalization of Significant Features

To prevent biased weighting of significant features during the classification process, the distribution of each feature is centered to zero mean and scaled to unit variance. Specifically, given the distribution of values for a feature F across all datasets S_{F}=[S_{1F}, S_{2F}, S_{3F}, . . . , S_{nF}], the sample mean and variance of this vector, (μ_{F} and σ²_{F}, respectively), is computed and normalized by subtracting μ_{F} from each value in the feature vector and subsequently dividing by the square root of the variance:

$$norm ( {S}_{iF} ) = \frac{( {S}_{iF} - {μ}_{F} )}{\sqrt{{σ}_{F}^{2}}}$$

The associated scaling factors (μ_{F} and σ²_{F}) from this normalization are also applied to all downstream test sets.

### 2.2 Signature Selection for Classifier Development

For each class b, the data containing all samples and corresponding normalized values of significant features is processed according to the following algorithm. For each selection iteration i, a subset of Nᵢ significant features are first selected from the set of differentially abundant features determined in 1.2 (Nₘᵢₙ≦Nᵢ≦Nₘₐₓ). This set of Nᵢ features comprises a signature. Under this signature:
A. Using the nearest neighbor algorithm with a Euclidean distance metric, samples are assigned to either class b or simply not b. During this process each sample is assigned to the known class of its closest neighbor according to the signature. Specifically, given a sample Sₑ represented in the Nᵢ-dimensional feature space by a vector [Sₑ₁, . . . , S_{eNi}], Sₑ is assigned to the class of a different sample S_{f} that minimizes the following distance:

$$d ( {S}_{e} , {S}_{f} ) = \sqrt{{\sum }_{j = 1}^{{N}_{i}} {( {S}_{ej} - {S}_{fj} )}^{2}}$$

B. Next, examining the 2×2 outcome table of resulting assignments, the corresponding sensitivity, specificity, positive/negative predictive values, and accuracy values are computed and recorded. Fisher's exact test [28] is used to assess the significance of the classification accuracy relative to a randomized assignment.

At the end of this procedure, prediction results for each signature selection iteration i relative to class b are obtained. Signatures are ranked by overall classification accuracy.

The nearest neighbor algorithm is a mainstay of machine learning and has been successfully used for many classification problems ranging from secondary structures of proteins to face recognition. For high dimensional datasets, it is not possible to evaluate every unique combination of parameters, (e.g. [100 choose 8]=1.861×10¹¹) therefore selection of Nₘᵢₙ and Nₘₐₓ is important. For each application of the method of statistical analysis, broad searches are empirically performed to identify practical values for Nₘᵢₙ and Nₘₐₓ.

By performing this classification training separately for each class, the possibility that some signatures may distinguish a particular class from all others, but may not effectively distinguish all classes universally from one another, is allowed for. This flexibility leads to the application of multiple binary classifiers for class-specific assignment instead of a single universal classifier.

### 3. Classifier Validation and Testing

### 3.1 Cross-Validation of Classifiers

The robustness of top ranking classifiers is assessed using the following cross-validation (CV) procedure. For each repeated CV iteration, a random subset of samples is removed from the full dataset (e.g. 15%) and used for cross-validation consisting of re-classification of each removed sample individually on the basis of the remaining samples and the same nearest-neighbor algorithm as described above. The average sensitivity and specificity across all CV iterations are recorded for each classifier. Choices for iteration and size of validation set vary depending on the characteristics of the dataset.

### 3.2 Classifier Testing

To test the performance of a classifier on new samples, the significant features from the initial assessment are evaluated for these samples, i.e. the counts of those kmers that correspond to the significant features comprised in the signature are determined and normalized according to the scaling factors described in section 2.1. The sample is then assigned to either class b or not b using the nearest neighbor algorithm with a Euclidean distance metric as described in 2.2 A.

### C. Example Signatures

Another aspect of the inventive subject matter provides a signature comprising one or more feature selected from Tables 3, 7, and 10. For example, a signature may comprise about 1 feature and about 10 features, about 5 features to about 15 features, about 20 features to about 30 features, about 25 features to about 35 features, about 30 features to about 40 features, about 35 features to about 45 features, or about 40 features to about 50 features selected from Tables 3, 7, and 10. A signature may also comprise about 10, about 15, about 25, about 50, about 75, or about 100 features selected from Table 3. Alternatively, a signature may comprise about 250, about 500, about 1000, about 1500, about 2000, about 2500, about 3000, about 3500, about 4000, about 4500, or about 5000 features selected from Tables 3, 7, and 10. Preferably a signature comprises less than about 500 features, more preferably less than about 100 features.

In some aspects, a signature comprises between about 1 and about 50 features selected from Tables 3, 7, and 10. In an aspect, a signature is selected from Table 4. In another aspect, a signature is selected from Table 5. In another aspect, a signature comprises the features listed in Table 7.

In other aspects, a signature consists of the features listed in Table 7.

### D. PANEL OF PROBES

Another aspect of the inventive subject matter provides a panel comprising at least one probe. Each probe of the panel is capable of specifically hybridizing to a unique feature selected from Tables 3, 7, and 10 via Watson-Crick base pairing. A panel may comprise about 1 probe to about 10 probes, about 5 probes to about 15 probes, about 20 to about 30 probes, about 25 probes to about 35 probes, about 30 probes to about 40 probes, about 35 probes to about 45 probes, or about 40 probes to about 50 probes that each specifically hybridize to a unique feature selected from Table 3. A panel may also comprise about 10, about 15, about 25, about 50, about 75, or about 100 probes that each specifically hybridize to a unique feature selected from Tables 3, 7, and 10. Alternatively, a panel may comprise about 250, about 500, about 1000, about 1500, about 2000, about 2500, about 3000, about 3500, about 4000, about 4500, or about 5000 probes that each specifically hybridize to a unique feature selected from Tables 3, 7, and 10. Preferably a panel comprises less than about 500 probes, more preferably less than about 100 probes. In some aspects, a panel comprises between about 1 and about 50 probes that each specifically hybridize to a unique feature selected from Tables 3, 7, and 10. In an aspect, a panel comprises a plurality of probes that each specifically hybridize to a unique feature comprising a signature selected from Table 4. In another aspect, a panel comprises a plurality of probes that each specifically hybridize to a unique feature comprising a signature selected from Table 5. In another aspect, a panel comprises a plurality of probes that each specifically hybridize to a unique feature listed in Table 7.

A further aspect of the inventive subject matter is an array comprising at least one address. At least one address of the array has disposed thereon a probe that specifically hybridizes to a unique feature selected from Table 3, Table 7, or Table 10. In certain aspects, an array has disposed thereon a plurality of probes, wherein each probe specifically hybridizes to a unique feature selected from selected from Table 3, Table 7, or Table 10, and is located at a unique address of the array. Suitable panels of probes are described in the preceding paragraph.

Several substrates suitable for the construction of arrays are known in the art, and one skilled in the art will appreciate that other substrates may become available as the art progresses. The substrate may be a material that may be modified to contain discrete individual sites appropriate for the attachment or association of a probe and is amenable to at least one detection method. Non-limiting examples of substrate materials include glass, modified or functionalized glass, plastics (including acrylics, polystyrene and copolymers of styrene and other materials, polypropylene, polyethylene, polybutylene, polyurethanes, TeflonJ, etc.), nylon or nitrocellulose, polysaccharides, nylon, resins, silica or silica-based materials including silicon and modified silicon, carbon, metals, inorganic glasses and plastics. In an aspect, the substrate may allow optical detection without appreciably fluorescing.

A substrate may be planar, a substrate may be a well, e.g. a well of a 6, 24, 96, 384 or 1536 well plate, or alternatively, a substrate may be a bead. Additionally, the substrate may be the inner surface of a tube for flow-through sample analysis to minimize sample volume. Similarly, the substrate may be flexible, such as a flexible foam, including closed cell foams made of particular plastics.

A probe may be attached to the substrate in a wide variety of ways, as will be appreciated by those in the art. The probe may either be synthesized first, with subsequent attachment to the substrate, or may be directly synthesized on the substrate. The substrate and the probe may be derivatized with chemical functional groups for subsequent attachment of the two. For example, the substrate may be derivatized with a chemical functional group including, but not limited to, amino groups, carboxyl groups, oxo groups or thiol groups. Using these functional groups, the probe may be attached either directly or indirectly using linkers.

The probe may also be attached to the substrate non-covalently. For example, a biotinylated probe may be prepared, which may bind to surfaces covalently coated with streptavidin, resulting in attachment. Alternatively, a probe may be synthesized on the surface using techniques such as photopolymerization and photolithography. Additional methods of attaching probes to arrays and methods of synthesizing biomolecules on substrates are well known in the art.

A probe may be represented more than once on a given array. In other words, more than one address of an array may be comprised of the probe. In some aspects, two, three, or more than three addresses of the array may be comprised of the probe. In certain aspects, the array may comprise control probes and/or control addresses. The controls may be internal controls, positive controls, negative controls, or background controls.

### E. METHOD OF USE

In another aspect, the inventive subject matter provides a method for classifying a subject as having IBD or not. The method comprises (a) providing (i) a sample obtained from the subject, the sample comprising nucleic acid from the subject's microbiome, and (ii) a signature that distinguishes subjects having IBD from a subject not having IBD, (b) determining in the sample the abundance of the features comprising the signature (“the dataset”), and (c) classifying the dataset from (b) as being either obtained from a subject having IBD or a subject not having IBD, thereby classifying the subject as having IBD or not having IBD.

Another aspect of the inventive subject matter provides a method for classifying a subject as having UC or CD. In some aspects, the subject is diagnosed as having IBD. In other aspects, a diagnosis of IBD is suspected for the subject but not confirmed. The method comprises (a) providing (i) a sample obtained from the subject, the sample comprising nucleic acid from the subject's microbiome, and (ii) a signature that distinguishes subjects having UC from a subject having CD, (b) determining in the sample the abundance of the features comprising the signature (“the dataset”), and (c) classifying the dataset from (b) as being either obtained from a subject having UC or a subject having CD, thereby classifying the subject as having UC or CD.

Suitable sources of microbiome samples and methods for isolating nucleic acids from the microbiome samples are described above in Section B. Suitable signatures are described in Section C above, and further described in the Examples. The abundance of the features in the signatures can be determined by any method known in the art including, but not limited to, sequencing or hybridization to an array. Suitable arrays are described in Section D above.

A preferred nucleic acid sample may be a nucleic acid sample obtained from a suitable fecal sample. Fecal samples are commonly used in the art to sample gut microbiota. Methods for obtaining a fecal sample from a subject are known in the art and include, but are not limited to, rectal swab and stool collection. Suitable fecal samples may be freshly obtained or may have been stored under appropriate temperatures and conditions known in the art. Methods for extracting nucleic acids from a fecal sample are also well known in the art and described herein. The nucleic acids comprising the nucleic acid sample may or may not be amplified prior to being used in step (b) depending upon the type and sensitivity of the data acquisition component. When amplification is desired, nucleic acids may be amplified via polymerase chain reaction (PCR) from a nucleic acid sample. Methods for performing PCR are well known in the art. The nucleic acids comprising the nucleic acid sample may also be fluorescently or chemically labeled, fragmented, or otherwise modified prior to sequencing or hybridization to an array as is routinely performed in the art.

In some aspects, the abundance of the signature's features in the sample is determined by sequencing. In other aspects, the abundance of the signature's features in the sample is determined by using an array. The inventive subject matter is not limited to any particular sequencing or array platform. Suitable sequencing platforms are capable of single-molecule sequencing, ion semiconductor sequencing, pyrosequencing, sequencing by synthesis, sequencing by ligation, nanopore sequencing, or tunneling currents sequencing. Non-limiting examples of suitable sequencers include an Illumina sequencer, a MiSeq Desktop Sequencer, a NextSeq Sequencer, an Ion PGM™ sequencer, a MinION™ device, a GridION™ device, a Ion Proton™ device, and/or the like. Suitable array platforms are capable of generating array data that captures the intensity of each position on the array for use in producing a microbial nucleic acid feature. Data from the sequencers or array scanner may be used by a program to qualitatively or quantitatively determine the abundance of the features comprised in the signature. The abundance values may be normalized by methods known in the art, or as described in Section B.2.1. The sample is then assigned to either class b or not b using the nearest neighbor algorithm with a Euclidean distance metric as described in Section B.2.2 A.

Diagnosis of IBD is a difficult and lengthy process involving extensive testing and the systematic eliminate of other possible diagnoses. The lengthy nature of this process causes those subjects with IBD to suffer long waits for proper diagnosis and treatment. Thus, there is a need for method of rapidly classifying whether a subject has IBD. Such a method is presently disclosed. Classifying subjects as having IBD, CD, or UC allows the physician to take the most appropriate next steps in diagnosing and treating the subject. These steps may include prescribing or administering drugs, prescribing dietary and nutritional therapies, prescribing or preforming medical and surgical treatments, prescribing or performing additional diagnostic methods, enrolling patients in clinical trials, and other treatments.

Drugs that may be prescribed or administered for treatment, include, but are not limited to anti-inflammatory drugs (including, but not limited to aminosalicylates and corticosteroids), immune system suppressors, antibiotics, anti-diarrheal medications, and pain relievers. Dietary and nutritional therapies may include, but are not limited to, vitamin supplements, enteral nutrition, parenteral nutrition, probiotics, prebiotics, special diets (i.e., low-residue diet), and nutritional counseling. Additional diagnostic methods may include, but are not limited to, blood tests, genetic testing, stool tests, biopsy, radiology scans or diagnostic imaging, and endoscopy. Blood tests may include, but are not limited to, complete blood count, erythrocyte sedimentation rate (ESR), c-reactive protein (CRP), liver enzymes, electrolytes, vitamin B12, vitamin D, calprotectin, latoferrin, thiopurine methyltransferase (TMPT), perinuclear anti-neutirophil antibody (pANCA), anti-Saccharomyces cervisiae antibody (ASCA), anti-flagellin antibody (CBir1), and anti-OMPC antibody (OmpC). Radiology scans and diagnostic imaging may include, but are not limited to, barium enema, CT scan and CT enterography (CTE), leukocyte scintigraphy, magnetic resonance imaging (MRI) and MR enterography (MRE), small bowel follow-through and small bowel enteroclysis, ultrasounds, and X-rays. Endoscopic tests may include but are not limited to colonoscopies, sigmoidoscopy, capsule endoscopy (CE), and endoscopic ultrasound. A preferred endoscopy test for subject classified as having IBD is a colonoscopy.

Accordingly, the inventive subject matter provides a method for recommending a subject for a colonoscopy. The method comprises (a) providing (i) a sample obtained from the subject, the sample comprising nucleic acid from the subject's microbiome, and (ii) a signature that distinguishes subjects having IBD from a subject not having IBD, (b) determining in the sample the abundance of the features comprising the signature (“the dataset”), (c) classifying the dataset from (b) as being either obtained from a subject having IBD or a subject not having IBD, and (d) recommending the subject for a colonoscopy when the dataset from (c) is classified as being from a subject with IBD. The primary advantange such a method is that a patient can be accelerated to colonoscopy for confirmation of IBD by a specialist rather than endure long-term dietary evaluation by a general practitioner.

The following examples are included to demonstrate aspects of the invention. It should be appreciated by those of skill in the art that the techniques disclosed in the examples that follow represent techniques discovered by the inventors to function well in the practice of the invention. Those of skill in the art should, however, in light of the inventive subject matter as disclosed herein, appreciate that changes may be made in the specific aspects that are disclosed and still obtain a like or similar result without departing from the spirit and scope of the invention. Therefore, all matter set forth or shown in the accompanying drawings is to be interpreted as illustrative and not in a limiting sense.

### F. EXAMPLES

### Data Sets

Metagenomic data sets analyzed using the methods of statistical analysis described above include the following.

Human Microbiome Project (HMP) body site data. A total of 690 HMP WGS metagenomic samples from a six distinct body sites (anterior nares, buccal mucosa, posterior fornix, stool, supragingival plaque, and tongue dorsum) were analyzed. The minimum number of samples associated with a particular body site was 51. Given the large scale of the HMP WGS Illumina sequence datasets (over 15.3 terabytes of raw sequence), kmer counting was performed on random sequence subsets of each sample (˜20% of raw reads). This resulted in an average kmer frequency of 4,139 observations per kmer and sample.

MetaHIT data, healthy individuals, and inflammatory bowel disease (IBD) patients. Raw Illumina GA II read data was acquired from the MetaHIT project [4] which contained WGS metagenomic samples from stool of 124 European individuals. Of the 124 individuals, 21 had ulcerative colitis (UC) and four had Crohn's disease (CD). The remaining 99 were considered healthy.

Obese and lean twin gut microbiomes. The classifiers were additionally tested on WGS metagenomic datasets representing the gut microbiomes of 15 subjects who were either obese (BMI>30, n=9) or lean (BMI=18.5-24.9, n=6) [2]. This raw sequence data was generated using the Roche/454 platform, either with the FLX or Titanium generation of sequencers.

Japanese gut microbiomes. Nine gut microbiome samples were obtained from a study of Japanese infants and adults [5]. All subjects in this case were >1.5 years in age (mean: 26.6 yrs). Datasets consisted of assembled contigs and singleton reads generated using Sanger sequencing technology.

Healthy individual and Crohn's disease patient fecal microbiomes. Raw sequence data from two recent studies on inflammatory bowel disease was included. 1) Roche/454 Titanium reads corresponding to eight fecal samples from patients diagnosed with CD and four samples from healthy controls [29], which contained technical replicates, i.e. independently generated sequence data from the same sample, which were treated as independent samples in the analysis of this data. 2) Illumina MiSeq reads corresponding to four fecal samples from CD patients and seven samples from healthy controls [30].

### HMP Body Site Classifiers

A proof-of-concept study focused on assigning metagenomic samples from the Human Microbiome Project to their associated body sites. The analysis was restricted to 690 samples from six well-represented body regions (anterior nares, buccal mucosa, posterior fornix, supragingival mucosa, stool, tongue dorsum).

In the initial differential abundance detection of kmers (see section 1., above), a universal p-value cutoff (p<5e-04) was heuristically chosen such that the resulting expected feature false discovery rate (FDR) was below 0.08% for each pairwise comparison of body sites. Additionally, only kmers that were differentially abundant between a particular body site and all other sites were included. For example, if the kmer “ACGTTACG” was identified as differentially abundant in all pairwise comparisons involving buccal mucosa, it was designated as a significant feature to consider for buccal mucosa signature selection. Thus, significant feature sets differed by body site. After performing a broad scan of signature set sizes where Nₘᵢₙ=Nₘₐₓ (range 10-1000), Nₘᵢₙ=50 and Nₘₐₓ=100 was selected as the best balance between computational complexity and overall accuracy.

For each body site and its associated set of priority kmers, the feature selection algorithm (see section 2., above) was run for 10,000 iterations, and the 10 signatures with the best classification accuracy or 10 random classifiers if more showed 100% accuracy (e.g. for stool samples) were selected for cross-validation. The single best performing classifier was then chosen based on average sensitivity/specificity (SN/SP) results. In some cases, multiple classifiers had optimal performance (e.g. stool classifiers), and in these cases a single classifier was randomly selected to report.

Table 1 displays the cross-validation results of the top selected classifier for each body site. For each classifier, sensitivity and specificity values were calculated for each subsampling step of the cross-validation procedure. Fisher's p-values were determined to assess the statistical significance of the obtained accuracy of the classifier during cross-validation. All classifiers demonstrated high accuracy with a mean sensitivity of >97% and mean specificity of >99%. Furthermore, the corresponding range of p-values from Fisher's exact test showed that the classification results from cross-validation are significantly better than random.

TABLE 1
Cross-Validation Performance of Top-Ranking
Binary Classifiers for Each Body Site.

| | | | (MIN/MAX/ |
|---|---|---|---|
| | (MIN/MAX/ | (MIN/MAX/ | MEAN) |
| | MEAN) | MEAN) | FISHERS |
| | SN | SP | P-VALUE |
| ANTERIOR | 100.000/ | 100.000/ | 7.40e−21/ |
| NARES | 100.000/ | 100.000/ | 9.76e−11/ |
| | 100.000 | 100.000 | 9.97e−12 |
| BUCCAL | 83.333/ | 98.684/ | 1.70e−21/ |
| MUCOSA | 100.000/ | 100.000/ | 1.44e−14/ |
| | 97.219 | 99.738 | 3.00e−15 |
| POSTERIOR | 92.857/ | 100.000/ | 2.09e−15/ |
| FORNIX | 100.000/ | 100.000/ | 7.46e−06/ |
| | 99.286 | 100.000 | 7.81e−07 |
| STOOL | 100.000/ | 100.000/ | 6.70e−25/ |
| | 100.000/ | 100.000/ | 4.74e−19/ |
| | 100.000 | 100.000 | 7.67e−20 |
| SUPRA- | 100.000/ | 96.202/ | 6.32e−22/ |
| GINGIVAL | 100.000/ | 100.000/ | 1.21e−12/ |
| PLAQUE | 100.000 | 99.114 | 1.22e−13 |
| TONGUE | 94.736/ | 98.550/ | 6.34e−22/ |
| DORSUM | 100.000/ | 100.000/ | 8.60e−16/ |
| | 99.473 | 99.463 | 9.07e−17 |

The cross-validation procedure used a subsampling approach in which 15% of the samples were repeatedly selected as a validation set for re-classification using the remaining 85% of the data.

### Gut Microbiome Validation

Further investigation into how the body site classifiers would perform on datasets from metagenomic studies outside the HMP, including those employing different sequencing technologies, was undertaken. To do so, three datasets associated with previously published distal gut microbiome projects [2,4,5] were collected. The first dataset represented the gut microbiota of 85 healthy subjects from the MetaHIT project. Similarly to the HMP, the MetaHIT project utilized the Illumina sequencing platform. The second dataset was comprised of 15 samples generated by the Roche/454 sequencer from the study on obese and lean twins [2], while the third set of nine samples from the Japanese study [5] used Sanger sequencing and only provided assembled contigs and singleton reads. kmer counting was performed on all samples and they were assigned using each body site classifier from Table 1.

Table 2 displays the results of the HMP body site classifiers on the external gut microbiome datasets. For the stool classifier, the overall sensitivity was computed for each gut dataset, that is, the percentage of tested samples that were successfully assigned to stool. In contrast, for each of the other body site classifiers, the specificity was calculated, that is, the percentage of gut samples that were correctly classified as not belonging to that body site. It was observed that all classifiers performed perfectly on these test sets, either with 100% sensitivity or 100% specificity, respectively.

In spite of the highly variable characteristics of these datasets (e.g. different geographic sample origins, sequencing technologies, or read lengths, assembled vs. unassembled reads), the classifiers assigned all gut samples perfectly. This evidence supports the robustness of the microbial signatures to alterations in sequencing technology or data processing.

TABLE 2
HMP body-site classifier results on other gut microbiome datasets.

| | SENSITIVITY | SPECIFICITY | | | | |
|---|---|---|---|---|---|---|
| | CLASSIFIER: | | | | | |
| | DISTAL | ANTERIOR | BUCCAL | POSTERIOR | SUPRAGINGIVAL | TONGUE |
| DATASET | GUT | NARES | MUCOSA | FORNIX | PLAQUE | DORSUM |
| METAHIT GUT | 100% | 100% | 100% | 100% | 100% | 100% |
| MICROBIOME (n = 85) | | | | | | |
| TWIN GUT | 100% | 100% | 100% | 100% | 100% | 100% |
| MICROBIOMES (n = 15) | | | | | | |
| JAPANESE GUT | 100% | 100% | 100% | 100% | 100% | 100% |
| MICROBIOME (n = 9) | | | | | | |

MetaHIT data consists of raw illumina GA II sequences. The twin gut microbiomes consist of raw reads from 454/Roche FLX or Titanium platforms. The Japanese gut microbiome data consists of assembled contigs and singleton reads from a Sanger sequencing platform.
IBD vs. Non-IBD

The method of statistical analysis was applied to a setting of clinical importance. Clinicians often find it difficult to easily determine whether a patient has inflammatory bowel disease. Therefore, the method was performed to determine signatures that accurately distinguished patients with IBD from patients without IBD.

The training sets included shotgun metagenomic sequences the following: (i) 147 non-IBD control samples from Karlsson et al. (“Gut metagenome in European women with normal, impaired and diabetic glucose control.” Nature 498.7452 (2013): 99-103), (ii) 12 IBD/non-IBD control samples from Erickson et al. (“Integrated metagenomics/metaproteomics reveals human host-microbiota signatures of Crohn's disease.” PloS one 7.11 (2012): e49138), (iii) 43 IBD/non-IBD control samples from Gevers et al. (“The treatment-naive microbiome in new-onset Crohn's disease.” Cell host & microbe 15.3 (2014): 382-392), (iv) 342 IBD/non-IBD control samples from Lewis et al. (“Inflammation, Antibiotics, and Diet as Environmental Stressors of the Gut Microbiome in Pediatric Crohn's Disease.” Cell host & microbe 18.4 (2015): 489-500), (v) 11 IBD/non-IBD control samples from Morgan et al. (“Dysfunction of the intestinal microbiome in inflammatory bowel disease and treatment.” Genome Biol 13.9 (2012): R79), (vi) 124 non-IBD control samples from Qin et al. (“A human gut microbial gene catalogue established by metagenomic sequencing.” Nature 464.7285 (2010): 59-65), and (vii) 18 non-IBD control samples from Turnbaugh, ct al. (“A core gut microbiome in obese and lean twins.” Nature 457.7228 (2009): 480-484). In total, there were 389 and 308 IBD and non-IBD control samples, respectively.

Kmers (8 mers) with significant differential abundance between the two sample groups were determined with the Mann-Whitney test. After differential abundance analysis, a p-value threshold was selected to maintain a FDR <0.01%, resulting in a set of 18,680 significant features.

The signature selection algorithm was run on the 18,680 significant features for 800,000 iterations using signatures consisting of between 1 and 50 different 8 mers. Each sample was screened using the different signature iterations, and then classified as belonging to one of the two classes (i.e. IBD vs. non-IBD). This classification was performed with the nearest neighbor algorithm with a Euclidean distance metric (see section 2.2A above). Next, examining the 2×2 outcome table of the resulting assignments, the corresponding sensitivity (SN), specificity (SP), and positive/negative predictive values (PPV/NPV) were computed and recorded. Fisher's exact test was used to assess the significance of each classification relative to a randomized assignment. All 800,000 tested signatures, i.e. combinations of between 1 and 50 random 8 mers, were ranked based on 2×2 outcome tables.

Of these 800,000 iterations, a signature of 21 kmers was found that significantly distinguished IBD and non-IBD samples. This signature is provided in Table 7. The primary results of the signature is shown in Table 8.

The robustness of this top-performing classifier (=signature) was assessed using a re-sampling cross-validation procedure as described in section 3.1 above. This type of validation determines whether the accuracy of the classifiers is significantly reduced if only a subset of the original samples is used as a reference for the classification, i.e. it can help assess whether additional reference samples are needed to improve the classifier. For a particular classifier undergoing validation, a random subset of samples (20%) were removed from the full dataset and then individually re-classified. This process was repeated for 100 iterations for the classifier. The average SN, SP, PPV, NPV and Fisher's exact test p-values across all cross-validation iterations were recorded for the optimal signature (results shown in Table 9).

Crohn's Disease vs. Ulcerative Colitis

The method of statistical analysis was applied to a setting of clinical importance. Clinicians often find it difficult to easily distinguish between different forms of inflammatory bowel disease. Therefore, the method was performed to determine signatures that accurately distinguished patients with CD from patients with UC.

The training sets included the following: Crohn's Disease—the CD training set was comprised of metagenomic WGS data from stool samples from: 4 CD patients from the European MetaHIT study (Illumina HiSeq sequence data) [4] plus 12 CD patients (including several technical replicates) from two U.S. studies (Roche/454 sequence data, [29] and Illumina MiSeq data [30]), thus providing 16 CD samples; Ulcerative Colitis—the UC training set was comprised of gut samples from the MetaHIT project representing 21 patients with UC.

Kmers (8 mers) with significant differential abundance between the two sample groups were determined with the Metastats program [26]. After differential abundance analysis, a p-value threshold was selected to maintain a FDR <1%, resulting in a set of 5,287 significant features provided in Table 3.

The signature selection algorithm was run on the 5,287 significant features for 105,287 iterations using signatures consisting of between 1 and 50 different 8 mers. Each sample was screened using the different signature iterations, and then classified as belonging to one of the two classes (i.e. UC vs. CD). This classification was performed with the nearest neighbor algorithm with a Euclidean distance metric (see section 2.2A above). Next, examining the 2×2 outcome table of the resulting assignments, the corresponding sensitivity (SN), specificity (SP), and positive/negative predictive values (PPV/NPV) were computed and recorded. Fisher's exact test was used to assess the significance of each classification relative to a randomized assignment. All 105,287 tested signatures, i.e. combinations of between 1 and 50 random 8 mers, were ranked based on 2×2 outcome tables.

Of these 105,287 iterations, a total of 1,087 signatures were found that perfectly distinguished CD and UC samples. These signatures are provided in Table 4. Kmers listed in column A of Table 4 correspond to the kmers in Table 3, starting with position 0 (e.g. “AAAAAAAA” is number 0), running down a column, and then continuing to the next column to the right. Of the 5,287 significant 8-mers, 5,063 (95.8%) are included in at least one top-performing signature.

As Table 4 shows, using any of 1,087 signatures, the 37 samples were correctly classified as coming from the 16 patients with Crohn's disease or the 21 patients with ulcerative colitis, i.e. 100% specificity and sensitivity were achieved for this classification.

The robustness of the 1,087 top-performing classifiers (=signatures) was assessed using a re-sampling cross-validation procedure as described in section 3.1 above. This type of validation determines whether the accuracy of the classifiers is significantly reduced if only a subset of the original samples is used as a reference for the classification, i.e. it can help assess whether additional reference samples are needed to improve the classifier. For a particular classifier undergoing validation, a random subset of samples (20%) were removed from the full dataset and then individually re-classified. This process was repeated for 100 iterations for each classifier. The average SN, SP, PPV, NPV and Fisher's exact test p-values across all cross-validation iterations were recorded for each tested signature, i.e. for each set of 8 mers. Of all 1,087 top-performing classifiers, 17 also showed 100% mean SN, SP, PPV, and NPV values after cross-validation (Table 5). This indicates that the variation detected by these 17 signatures between samples from the CD and UC classes is stronger than variations between samples from the same class (i.e. CD or UC) and, thus, that classification of samples as UC or CD with these 17 signatures is most reliable. Classification with the remaining 1070 signatures on the other hand is hindered by the detection of signals resulting from variations between samples from the same class (i.e., CD or UC). The accuracy of these 1070 signatures is therefore expected to depend to a larger extent on the number of reference samples and to improve with larger numbers of reference samples.

To evaluate the importance of kmer length for sample classification, the signature identification method was also applied to kmer abundances of lengths of 2, 3, and 4 nucleotides and a training set of 21 UC and 12 CD samples (including the 4 CD patients from the MetaHIT and 8 CD patients from one of the U.S. studies [29]). The results, including mean SN, SP, PPV, NPV, and Fisher's exact test P-values, are shown in Table 6 and indicate that classification of CD and UC samples is possible using signatures of kmers combinations with length 2 nucleotides and longer, although at lower accuracy (i.e., SN, SP <100%) compared to kmers of length 8 nucleotides.

### Validation Using Random Sequence Data

To validate the statistical significance of the findings, the same type of kmer-based signature identification analysis was performed using computer-generated random kmer counts of the length 4 nucleotides. For this analysis, two groups with 100 samples each were compared. While the sensitivity of 73% of the top-performing signatures to distinguish these random sample data is relatively high, the specificity of 52% is low and the associated mean Fisher's exact test P-values of 0.25 are high, as would be expected. The results of this comparison are also shown in Table 6.

### REFERENCES

All patents and publications mentioned in this specification are indicative of the level of skill of those skilled in the art to which the inventive subject matter pertains. The following literature references are believed to useful to an understanding of the inventive subject matter in the context of its place in the relevant art. Citation here is not to be construed as an assertion or admission that any reference cited is material to patentability of the inventive subject matter. Applicants will properly disclose information material to patentability in an Information Disclosure Statement. Each of the following documents is hereby incorporated by reference in its entirety in this application.
1. Turnbaugh P J, Ley R E, Hamady M, Fraser-Liggett C M, Knight R, et al. (2007) The human microbiome project. Nature 449: 804-810.
2. Turnbaugh P J, Hamady M, Yatsunenko T, Cantarel B L, Duncan A, et al. (2009) A core gut microbiome in obese and lean twins. Nature 457: 480-484.
3. Turnbaugh P J, Ley R E, Mahowald M A, Magrini V, Mardis E R, et al. (2006) An obesity-associated gut microbiome with increased capacity for energy harvest. Nature 444: 1027-1031.
4. Qin J, Li R, Raes J, Arumugam M, Burgdorf K S, et al. (2010) A human gut microbial gene catalogue established by metagenomic sequencing. Nature 464: 59-65.
5. Kurokawa K, Itoh T, Kuwahara T, Oshima K, Toh H, et al. (2007) Comparative metagenomics revealed commonly enriched gene sets in human gut microbiomes. DNA Res 14: 169-181.
6. Ley R E, Backhed F, Turnbaugh P, Lozupone C A, Knight R D, et al. (2005) Obesity alters gut microbial ecology. Proceedings of the National Academy of Sciences of the United States of America 102: 11070-11075.
7. Ley R E, Turnbaugh P J, Klein S, Gordon J I (2006) Microbial ecology: human gut microbes associated with obesity. Nature 444: 1022-1023.
8. Turnbaugh P J, Quince C, Faith J J, McHardy A C, Yatsunenko T, et al. (2010) Organismal, genetic, and transcriptional variation in the deeply sequenced gut microbiomes of identical twins. Proc Natl Acad Sci USA 107: 7503-7508.
9. Dethlefsen L, Huse S, Sogin M L, Relman D A (2008) The Pervasive Effects of an Antibiotic on the Human Gut Microbiota, as Revealed by Deep 16S rRNA Sequencing. PLoS Biol 6: e280.
10. Turnbaugh P J, Backhed F, Fulton L, Gordon J I (2008) Diet-induced obesity is linked to marked but reversible alterations in the mouse distal gut microbiome. Cell Host Microbe 3: 213-223.
11. Turnbaugh P J, Ridaura V K, Faith J J, Rey F E, Knight R, et al. (2009) The effect of diet on the human gut microbiome: a metagenomic analysis in humanized gnotobiotic mice. Science translational medicine 1: 6ra14.
12. Proctor L M (2011) The Human Microbiome Project in 2011 and beyond. Cell Host Microbe 10: 287-291.
13. Hsiao W W, Fraser-Liggett C M (2009) Human Microbiome Project—paving the way to a better understanding of ourselves and our microbes. Drug Discov Today 14: 331-333.
14. Gerlach W, Stoye J (2011) Taxonomic classification of metagenomic shotgun sequences with CARMA3. Nucleic acids research 39: e91.
15. Huson D H, Auch A F, Qi J, Schuster S C (2007) MEGAN analysis of metagenomic data. Genome Res 17: 377-386.
16. Huson D H, Mitra S, Ruscheweyh H J, Weber N, Schuster S C (2011) Integrative analysis of environmental sequences using MEGAN4. Genome research 21: 1552-1560.
17. Krause L, Diaz N N, Goesmann A, Kelley S, Nattkemper T W, et al. (2008) Phylogenetic classification of short environmental DNA fragments. Nucleic Acids Res 36: 2230-2239.
18. Liu B, Gibbons T, Ghodsi M, Treangen T, Pop M (2011) Accurate and fast estimation of taxonomic profiles from metagenomic shotgun sequences. BMC Genomics 12 Suppl 2: S4.
19. McHardy A C, Martin H G, Tsirigos A, Hugenholtz P, Rigoutsos I (2007) Accurate phylogenetic classification of variable-length DNA fragments. Nat Methods 4: 63-72.
20. Patil K R, Haider P, Pope P B, Turnbaugh P J, Morrison M, et al. (2011) Taxonomic metagenome sequence assignment with structured output models. Nat Methods 8: 191-192.
21. Knights D, Parfrey L W, Zaneveld J, Lozupone C, Knight R (2011) Human-associated microbial signatures: examining their predictive value. Cell Host Microbe 10: 292-296.
22. Werner J J, Knights D, Garcia M L, Scalfone N B, Smith S, et al. (2011) Bacterial community structures are unique and resilient in full-scale bioenergy systems. Proceedings of the National Academy of Sciences of the United States of America 108: 4158-4163.
23. Knights D, Costello E K, Knight R (2011) Supervised classification of human microbiota. FEMS Microbiol Rev 35: 343-359.
24. Liu Z, Hsiao W, Cantarel B L, Drabek E F, Fraser-Liggett C (2011) Sparse distance-based learning for simultaneous multiclass classification and feature selection of metagenomic data. Bioinformatics 27: 3242-3249.
25. Segata N, Izard J, Waldron L, Gevers D, Miropolsky L, et al. (2011) Metagenomic biomarker discovery and explanation. Genome biology 12: R60.
26. White J R, Nagarajan N, Pop M (2009) Statistical methods for detecting differentially abundant features in clinical metagenomic samples. PLoS Comput Biol 5: e1000352.
27. Benjamini Y, Hochberg Y (1995) Controlling the False Discovery Rate—a Practical and Powerful Approach to Multiple Testing. Journal of the Royal Statistical Society Series B-Methodological 57: 289-300.
28. Zar J H (1999) Biostatistical analysis. Upper Saddle River, N.J.: Prentice Hall. 1 v. (various pagings) p.
29. Cantarel B L, Erickson A R, VerBerkmoes N C, Erickson B K, Carey P A, et al. (2011) Strategies for metagenomic-guided whole-community proteomics of complex microbial environments. PLoS One 6: e27173.
30. Morgan X C, Tickle T L, Sokol H, Gevers D, Devaney K L, et al. (2012) Dysfunction of the intestinal microbiome in inflammatory bowel disease and treatment. Genome Biol 13: R79.
31. Qin J, Li Y, Cal Z, Li S, Zhu J, et al. (2012) A metagenome-wide association study of gut microbiota in type 2 diabetes. Nature 490: 55-60.
32. Castellarin M, Warren R L, Freeman J D, Dreolini L, Krzywinski M, et al. (2012) Fusobacterium nucleatum infection is prevalent in human colorectal carcinoma. Genome Res 22: 299-306.

The inventive subject matter herein began with Applicants' essential observation that significant biases exist in the oligonucleotide compositions of metagenomic samples between different body sites and that some of these biases are remarkably stable across multiple individuals, in spite of an overall taxonomic variability of the microbiota, as determined by 16S rRNA phylogenetic analysis. Taking advantage of this information, robust signatures were discovered for accurately assigning metagenomic samples to different body sites or to a disease, disorder, condition, or symptom thereof such as the two inflammatory bowel diseases, Crohn's disease, and ulcerative colitis.

While the invention has been described with reference to certain particular aspects thereof, those skilled in the art will appreciate that various modifications may be made without departing from the spirit and scope of the invention. The scope of the appended claims is not to be limited to the specific aspects described.

The inventive subject matter being thus described, it will be obvious that the same may be modified or varied in many ways. Such modifications and variations are not to be regarded as a departure from the spirit and scope of the inventive subject matter, and all such modifications and variations are intended to be included within the scope of the following claims.

TABLE 3
Kmers.

| | |
|---|---|
| Kmr No. 1. | AAAAAAAA |
| Kmr No. 2. | AAAAAAAC |
| Kmr No. 3. | AAAAAAAG |
| Kmr No. 4. | AAAAAAAT |
| Kmr No. 5. | AAAAAACC |
| Kmr No. 6. | AAAAAACG |
| Kmr No. 7. | AAAAAAGC |
| Kmr No. 8. | AAAAACAC |
| Kmr No. 9. | AAAAACCC |
| Kmr No. 10. | AAAAACGC |
| Kmr No. 11. | AAAAAGCC |
| Kmr No. 12. | AAAAAGCG |
| Kmr No. 13. | AAAAAGGG |
| Kmr No. 14. | AAAACACC |
| Kmr No. 15. | AAAACACG |
| Kmr No. 16. | AAAACCCA |
| Kmr No. 17. | AAAACCCC |
| Kmr No. 18. | AAAACCCG |
| Kmr No. 19. | AAAACCTC |
| Kmr No. 20. | AAAACGAG |
| Kmr No. 21. | AAAACGCA |
| Kmr No. 22. | AAAACGCC |
| Kmr No. 23. | AAAACGCT |
| Kmr No. 24. | AAAACGGC |
| Kmr No. 25. | AAAACGTT |
| Kmr No. 26. | AAAACTCG |
| Kmr No. 27. | AAAACTGG |
| Kmr No. 28. | AAAAGCAC |
| Kmr No. 29. | AAAAGCCC |
| Kmr No. 30. | AAAAGCGC |
| Kmr No. 31. | AAAAGCGT |
| Kmr No. 32. | AAAAGCTC |
| Kmr No. 33. | AAAAGGCG |
| Kmr No. 34. | AAAAGGGC |
| Kmr No. 35. | AAAAGTGC |
| Kmr No. 36. | AAAATGCG |
| Kmr No. 37. | AAACACAC |
| Kmr No. 38. | AAACACCC |
| Kmr No. 39. | AAACACCG |
| Kmr No. 40. | AAACACGA |
| Kmr No. 41. | AAACACGC |
| Kmr No. 42. | AAACACGG |
| Kmr No. 43. | AAACAGGT |
| Kmr No. 44. | AAACAGTA |
| Kmr No. 45. | AAACAGTC |
| Kmr No. 46. | AAACAGTT |
| Kmr No. 47. | AAACATCG |
| Kmr No. 48. | AAACCAGT |
| Kmr No. 49. | AAACCATG |
| Kmr No. 50. | AAACCCAC |
| Kmr No. 51. | AAACCCCA |
| Kmr No. 52. | AAACCCCC |
| Kmr No. 53. | AAACCCCG |
| Kmr No. 54. | AAACCCCT |
| Kmr No. 55. | AAACCCGC |
| Kmr No. 56. | AAACCCTC |
| Kmr No. 57. | AAACCGAG |
| Kmr No. 58. | AAACCGGT |
| Kmr No. 59. | AAACCGTT |
| Kmr No. 60. | AAACCTCG |
| Kmr No. 61. | AAACGAGA |
| Kmr No. 62. | AAACGAGC |
| Kmr No. 63. | AAACGAGG |
| Kmr No. 64. | AAACGCCC |
| Kmr No. 65. | AAACGCTC |
| Kmr No. 66. | AAACGGCG |
| Kmr No. 67. | AAACGTCC |
| Kmr No. 68. | AAACGTCT |
| Kmr No. 69. | AAACGTGT |
| Kmr No. 70. | AAACGTTC |
| Kmr No. 71. | AAACGTTG |
| Kmr No. 72. | AAACTAGC |
| Kmr No. 73. | AAACTAGG |
| Kmr No. 74. | AAACTAGT |
| Kmr No. 75. | AAACTCGC |
| Kmr No. 76. | AAACTCGT |
| Kmr No. 77. | AAACTGCG |
| Kmr No. 78. | AAACTGCT |
| Kmr No. 79. | AAACTGGA |
| Kmr No. 80. | AAACTGGT |
| Kmr No. 81. | AAACTTCC |
| Kmr No. 82. | AAAGAACT |
| Kmr No. 83. | AAAGAATC |
| Kmr No. 84. | AAAGACCC |
| Kmr No. 85. | AAAGAGCG |
| Kmr No. 86. | AAAGAGGG |
| Kmr No. 87. | AAAGATTC |
| Kmr No. 88. | AAAGCACC |
| Kmr No. 89. | AAAGCCCA |
| Kmr No. 90. | AAAGCCCC |
| Kmr No. 91. | AAAGCCCG |
| Kmr No. 92. | AAAGCCCT |
| Kmr No. 93. | AAAGCCGC |
| Kmr No. 94. | AAAGCGAG |
| Kmr No. 95. | AAAGCGCA |
| Kmr No. 96. | AAAGCGCT |
| Kmr No. 97. | AAAGCGTC |
| Kmr No. 98. | AAAGCGTG |
| Kmr No. 99. | AAAGCTCA |
| Kmr No. 100. | AAAGCTCG |
| Kmr No. 101. | AAAGCTGC |
| Kmr No. 102. | AAAGCTTC |
| Kmr No. 103. | AAAGGCAC |
| Kmr No. 104. | AAAGGCGA |
| Kmr No. 105. | AAAGGCGC |
| Kmr No. 106. | AAAGGCTC |
| Kmr No. 107. | AAAGGGCA |
| Kmr No. 108. | AAAGGGCG |
| Kmr No. 109. | AAAGGGGG |
| Kmr No. 110. | AAAGGGTC |
| Kmr No. 111. | AAAGGGTG |
| Kmr No. 112. | AAAGGTCG |
| Kmr No. 113. | AAAGTAAC |
| Kmr No. 114. | AAAGTACT |
| Kmr No. 115. | AAAGTAGA |
| Kmr No. 116. | AAAGTCAC |
| Kmr No. 117. | AAAGTGAT |
| Kmr No. 118. | AAATACAG |
| Kmr No. 119. | AAATACCA |
| Kmr No. 120. | AAATACTG |
| Kmr No. 121. | AAATCAGT |
| Kmr No. 122. | AAATCCAG |
| Kmr No. 123. | AAATCCTG |
| Kmr No. 124. | AAATCGAG |
| Kmr No. 125. | AAATCTCG |
| Kmr No. 126. | AAATGATC |
| Kmr No. 127. | AAATGCGC |
| Kmr No. 128. | AAATGGTC |
| Kmr No. 129. | AACAAAAA |
| Kmr No. 130. | AACAACCC |
| Kmr No. 131. | AACAACTG |
| Kmr No. 132. | AACAATCG |
| Kmr No. 133. | AACACAAA |
| Kmr No. 134. | AACACACA |
| Kmr No. 135. | AACACACC |
| Kmr No. 136. | AACACACG |
| Kmr No. 137. | AACACCCA |
| Kmr No. 138. | AACACCCC |
| Kmr No. 139. | AACACCCG |
| Kmr No. 140. | AACACCCT |
| Kmr No. 141. | AACACCTC |
| Kmr No. 142. | AACACGCA |
| Kmr No. 143. | AACACGTA |
| Kmr No. 144. | AACACGTG |
| Kmr No. 145. | AACACGTT |
| Kmr No. 146. | AACACTCT |
| Kmr No. 147. | AACACTGG |
| Kmr No. 148. | AACACTTC |
| Kmr No. 149. | AACAGAAG |
| Kmr No. 150. | AACAGACG |
| Kmr No. 151. | AACAGATG |
| Kmr No. 152. | AACAGATT |
| Kmr No. 153. | AACAGCCA |
| Kmr No. 154. | AACAGCCG |
| Kmr No. 155. | AACAGGAA |
| Kmr No. 156. | AACAGGAC |
| Kmr No. 157. | AACAGGAT |
| Kmr No. 158. | AACAGGTA |
| Kmr No. 159. | AACAGGTT |
| Kmr No. 160. | AACAGTAA |
| Kmr No. 161. | AACAGTAC |
| Kmr No. 162. | AACAGTAG |
| Kmr No. 163. | AACAGTAT |
| Kmr No. 164. | AACAGTCA |
| Kmr No. 165. | AACAGTCC |
| Kmr No. 166. | AACAGTCG |
| Kmr No. 167. | AACAGTGA |
| Kmr No. 168. | AACAGTGG |
| Kmr No. 169. | AACAGTTA |
| Kmr No. 170. | AACAGTTC |
| Kmr No. 171. | AACATCCA |
| Kmr No. 172. | AACATGAC |
| Kmr No. 173. | AACATGAT |
| Kmr No. 174. | AACATGGT |
| Kmr No. 175. | AACATGTC |
| Kmr No. 176. | AACCAATC |
| Kmr No. 177. | AACCACAA |
| Kmr No. 178. | AACCACCC |
| Kmr No. 179. | AACCACTG |
| Kmr No. 180. | AACCAGTA |
| Kmr No. 181. | AACCAGTC |
| Kmr No. 182. | AACCAGTG |
| Kmr No. 183. | AACCAGTT |
| Kmr No. 184. | AACCATGA |
| Kmr No. 185. | AACCATGG |
| Kmr No. 186. | AACCATGT |
| Kmr No. 187. | AACCATTC |
| Kmr No. 188. | AACCCACA |
| Kmr No. 189. | AACCCACC |
| Kmr No. 190. | AACCCAGC |
| Kmr No. 191. | AACCCCAA |
| Kmr No. 192. | AACCCCAC |
| Kmr No. 193. | AACCCCAG |
| Kmr No. 194. | AACCCCCA |
| Kmr No. 195. | AACCCCCC |
| Kmr No. 196. | AACCCCCG |
| Kmr No. 197. | AACCCCCT |
| Kmr No. 198. | AACCCCGA |
| Kmr No. 199. | AACCCCGC |
| Kmr No. 200. | AACCCCTC |
| Kmr No. 201. | AACCCGCC |
| Kmr No. 202. | AACCCTCA |
| Kmr No. 203. | AACCCTCC |
| Kmr No. 204. | AACCCTCT |
| Kmr No. 205. | AACCGAGA |
| Kmr No. 206. | AACCGAGG |
| Kmr No. 207. | AACCGATC |
| Kmr No. 208. | AACCGCCA |
| Kmr No. 209. | AACCGCCT |
| Kmr No. 210. | AACCGGAA |
| Kmr No. 211. | AACCGGAC |
| Kmr No. 212. | AACCGGAG |
| Kmr No. 213. | AACCGGAT |
| Kmr No. 214. | AACCGGTC |
| Kmr No. 215. | AACCGGTG |
| Kmr No. 216. | AACCGGTT |
| Kmr No. 217. | AACCGTAC |
| Kmr No. 218. | AACCGTAG |
| Kmr No. 219. | AACCGTCA |
| Kmr No. 220. | AACCGTCT |
| Kmr No. 221. | AACCGTGG |
| Kmr No. 222. | AACCGTGT |
| Kmr No. 223. | AACCGTTA |
| Kmr No. 224. | AACCGTTC |
| Kmr No. 225. | AACCGTTG |
| Kmr No. 226. | AACCTAGT |
| Kmr No. 227. | AACCTCCT |
| Kmr No. 228. | AACCTCTC |
| Kmr No. 229. | AACCTGTA |
| Kmr No. 230. | AACCTGTC |
| Kmr No. 231. | AACGAATC |
| Kmr No. 232. | AACGACAA |
| Kmr No. 233. | AACGACCC |
| Kmr No. 234. | AACGAGAA |
| Kmr No. 235. | AACGAGAG |
| Kmr No. 236. | AACGAGAT |
| Kmr No. 237. | AACGAGGA |
| Kmr No. 238. | AACGAGGG |
| Kmr No. 239. | AACGAGGT |
| Kmr No. 240. | AACGATCA |
| Kmr No. 241. | AACGATCG |
| Kmr No. 242. | AACGATTC |
| Kmr No. 243. | AACGATTG |
| Kmr No. 244. | AACGCAAA |
| Kmr No. 245. | AACGCACA |
| Kmr No. 246. | AACGCAGA |
| Kmr No. 247. | AACGCATG |
| Kmr No. 248. | AACGCCCC |
| Kmr No. 249. | AACGCCCT |
| Kmr No. 250. | AACGCCTA |
| Kmr No. 251. | AACGCTCT |
| Kmr No. 252. | AACGCTGT |
| Kmr No. 253. | AACGCTTA |
| Kmr No. 254. | AACGGACC |
| Kmr No. 255. | AACGGATC |
| Kmr No. 256. | AACGGGGG |
| Kmr No. 257. | AACGGTAC |
| Kmr No. 258. | AACGGTCC |
| Kmr No. 259. | AACGGTTC |
| Kmr No. 260. | AACGTACC |
| Kmr No. 261. | AACGTACG |
| Kmr No. 262. | AACGTACT |
| Kmr No. 263. | AACGTATG |
| Kmr No. 264. | AACGTCAG |
| Kmr No. 265. | AACGTCCA |
| Kmr No. 266. | AACGTCCG |
| Kmr No. 267. | AACGTCTG |
| Kmr No. 268. | AACGTCTT |
| Kmr No. 269. | AACGTGGA |
| Kmr No. 270. | AACGTGTA |
| Kmr No. 271. | AACGTGTC |
| Kmr No. 272. | AACGTGTG |
| Kmr No. 273. | AACGTTAC |
| Kmr No. 274. | AACGTTAT |
| Kmr No. 275. | AACGTTCA |
| Kmr No. 276. | AACGTTCC |
| Kmr No. 277. | AACGTTCG |
| Kmr No. 278. | AACGTTCT |
| Kmr No. 279. | AACGTTGG |
| Kmr No. 280. | AACGTTTC |
| Kmr No. 281. | AACTAGCC |
| Kmr No. 282. | AACTAGCG |
| Kmr No. 283. | AACTAGCT |
| Kmr No. 284. | AACTAGGG |
| Kmr No. 285. | AACTAGGT |
| Kmr No. 286. | AACTAGTC |
| Kmr No. 287. | AACTCACA |
| Kmr No. 288. | AACTCCTC |
| Kmr No. 289. | AACTCGCA |
| Kmr No. 290. | AACTCTCG |
| Kmr No. 291. | AACTCTGC |
| Kmr No. 292. | AACTGATC |
| Kmr No. 293. | AACTGCCA |
| Kmr No. 294. | AACTGCCG |
| Kmr No. 295. | AACTGGAA |
| Kmr No. 296. | AACTGGAC |
| Kmr No. 297. | AACTGGAT |
| Kmr No. 298. | AACTGGCA |
| Kmr No. 299. | AACTGGGA |
| Kmr No. 300. | AACTGGTA |
| Kmr No. 301. | AACTGGTC |
| Kmr No. 302. | AACTGTAC |
| Kmr No. 303. | AACTGTCA |
| Kmr No. 304. | AACTGTCC |
| Kmr No. 305. | AACTGTTC |
| Kmr No. 306. | AACTTCCA |
| Kmr No. 307. | AACTTCCG |
| Kmr No. 308. | AAGAAATC |
| Kmr No. 309. | AAGAACAG |
| Kmr No. 310. | AAGAACCC |
| Kmr No. 311. | AAGAACGT |
| Kmr No. 312. | AAGAACTG |
| Kmr No. 313. | AAGAAGTG |
| Kmr No. 314. | AAGAATCA |
| Kmr No. 315. | AAGAATCC |
| Kmr No. 316. | AAGAATCG |
| Kmr No. 317. | AAGAATTC |
| Kmr No. 318. | AAGACAGT |
| Kmr No. 319. | AAGACATG |
| Kmr No. 320. | AAGACCCA |
| Kmr No. 321. | AAGACCCC |
| Kmr No. 322. | AAGACCCG |
| Kmr No. 323. | AAGACCCT |
| Kmr No. 324. | AAGACCTA |
| Kmr No. 325. | AAGACCTC |
| Kmr No. 326. | AAGACGGA |
| Kmr No. 327. | AAGACGGT |
| Kmr No. 328. | AAGACTGG |
| Kmr No. 329. | AAGAGAGC |
| Kmr No. 330. | AAGAGCAC |
| Kmr No. 331. | AAGAGCAT |
| Kmr No. 332. | AAGAGCCG |
| Kmr No. 333. | AAGAGCCT |
| Kmr No. 334. | AAGAGCGA |
| Kmr No. 335. | AAGAGCGC |
| Kmr No. 336. | AAGAGCGG |
| Kmr No. 337. | AAGAGCGT |
| Kmr No. 338. | AAGAGGGC |
| Kmr No. 339. | AAGAGGGG |
| Kmr No. 340. | AAGAGGGT |
| Kmr No. 341. | AAGAGGTC |
| Kmr No. 342. | AAGAGTGC |
| Kmr No. 343. | AAGATATC |
| Kmr No. 344. | AAGATTCC |
| Kmr No. 345. | AAGCAATC |
| Kmr No. 346. | AAGCACAA |
| Kmr No. 347. | AAGCACAC |
| Kmr No. 348. | AAGCACAT |
| Kmr No. 349. | AAGCACCC |
| Kmr No. 350. | AAGCACCT |
| Kmr No. 351. | AAGCACGA |
| Kmr No. 352. | AAGCACGT |
| Kmr No. 353. | AAGCACTC |
| Kmr No. 354. | AAGCCAGT |
| Kmr No. 355. | AAGCCCAC |
| Kmr No. 356. | AAGCCCAT |
| Kmr No. 357. | AAGCCCCC |
| Kmr No. 358. | AAGCCCCG |
| Kmr No. 359. | AAGCCCCT |
| Kmr No. 360. | AAGCCCGC |
| Kmr No. 361. | AAGCCCTA |
| Kmr No. 362. | AAGCCCTC |
| Kmr No. 363. | AAGCCCTT |
| Kmr No. 364. | AAGCCGCA |
| Kmr No. 365. | AAGCCTGC |
| Kmr No. 366. | AAGCGACA |
| Kmr No. 367. | AAGCGAGA |
| Kmr No. 368. | AAGCGAGG |
| Kmr No. 369. | AAGCGATC |
| Kmr No. 370. | AAGCGCAA |
| Kmr No. 371. | AAGCGCCC |
| Kmr No. 372. | AAGCGGCT |
| Kmr No. 373. | AAGCGGGG |
| Kmr No. 374. | AAGCGGGT |
| Kmr No. 375. | AAGCGTGA |
| Kmr No. 376. | AAGCTCTA |
| Kmr No. 377. | AAGCTCTC |
| Kmr No. 378. | AAGCTGCT |
| Kmr No. 379. | AAGCTTCC |
| Kmr No. 380. | AAGGAACC |
| Kmr No. 381. | AAGGAATC |
| Kmr No. 382. | AAGGAATT |
| Kmr No. 383. | AAGGACAT |
| Kmr No. 384. | AAGGACGA |
| Kmr No. 385. | AAGGACTA |
| Kmr No. 386. | AAGGAGCA |
| Kmr No. 387. | AAGGAGCG |
| Kmr No. 388. | AAGGAGGC |
| Kmr No. 389. | AAGGAGGG |
| Kmr No. 390. | AAGGAGGT |
| Kmr No. 391. | AAGGATTC |
| Kmr No. 392. | AAGGCAAA |
| Kmr No. 393. | AAGGCACA |
| Kmr No. 394. | AAGGCACT |
| Kmr No. 395. | AAGGCCCC |
| Kmr No. 396. | AAGGCGCA |
| Kmr No. 397. | AAGGCGGG |
| Kmr No. 398. | AAGGCTCA |
| Kmr No. 399. | AAGGCTCT |
| Kmr No. 400. | AAGGCTGA |
| Kmr No. 401. | AAGGCTGC |
| Kmr No. 402. | AAGGGCAA |
| Kmr No. 403. | AAGGGCAG |
| Kmr No. 404. | AAGGGCAT |
| Kmr No. 405. | AAGGGCGA |
| Kmr No. 406. | AAGGGCGG |
| Kmr No. 407. | AAGGGCGT |
| Kmr No. 408. | AAGGGCTA |
| Kmr No. 409. | AAGGGGCA |
| Kmr No. 410. | AAGGGGCG |
| Kmr No. 411. | AAGGGGGC |
| Kmr No. 412. | AAGGGGGG |
| Kmr No. 413. | AAGGGGGT |
| Kmr No. 414. | AAGGGGTC |
| Kmr No. 415. | AAGGGGTG |
| Kmr No. 416. | AAGGGTCA |
| Kmr No. 417. | AAGGGTCT |
| Kmr No. 418. | AAGGGTGA |
| Kmr No. 419. | AAGGGTGC |
| Kmr No. 420. | AAGGGTGG |
| Kmr No. 421. | AAGGGTGT |
| Kmr No. 422. | AAGGTCTA |
| Kmr No. 423. | AAGGTGCT |
| Kmr No. 424. | AAGGTGGG |
| Kmr No. 425. | AAGGTTCC |
| Kmr No. 426. | AAGTAACG |
| Kmr No. 427. | AAGTAACT |
| Kmr No. 428. | AAGTAATC |
| Kmr No. 429. | AAGTACAG |
| Kmr No. 430. | AAGTACCA |
| Kmr No. 431. | AAGTACCG |
| Kmr No. 432. | AAGTACGT |
| Kmr No. 433. | AAGTACTG |
| Kmr No. 434. | AAGTAGAA |
| Kmr No. 435. | AAGTAGAC |
| Kmr No. 436. | AAGTAGAT |
| Kmr No. 437. | AAGTATCC |
| Kmr No. 438. | AAGTCATG |
| Kmr No. 439. | AAGTCCCA |
| Kmr No. 440. | AAGTCTCG |
| Kmr No. 441. | AAGTGACA |
| Kmr No. 442. | AAGTGACG |
| Kmr No. 443. | AAGTGATC |
| Kmr No. 444. | AAGTGATT |
| Kmr No. 445. | AAGTGCCT |
| Kmr No. 446. | AAGTGGGG |
| Kmr No. 447. | AAGTGTGC |
| Kmr No. 448. | AAGTTCCA |
| Kmr No. 449. | AAGTTCCG |
| Kmr No. 450. | AATAACTG |
| Kmr No. 451. | AATAATAC |
| Kmr No. 452. | AATAATCC |
| Kmr No. 453. | AATACAGA |
| Kmr No. 454. | AATACAGG |
| Kmr No. 455. | AATACAGT |
| Kmr No. 456. | AATACCAG |
| Kmr No. 457. | AATACCAT |
| Kmr No. 458. | AATACCTG |
| Kmr No. 459. | AATACGAT |
| Kmr No. 460. | AATACGGA |
| Kmr No. 461. | AATACGGT |
| Kmr No. 462. | AATACGTC |
| Kmr No. 463. | AATACGTG |
| Kmr No. 464. | AATACTGG |
| Kmr No. 465. | AATAGAGC |
| Kmr No. 466. | AATAGGCG |
| Kmr No. 467. | AATCAATC |
| Kmr No. 468. | AATCAATG |
| Kmr No. 469. | AATCACAC |
| Kmr No. 470. | AATCACAG |
| Kmr No. 471. | AATCACAT |
| Kmr No. 472. | AATCACCA |
| Kmr No. 473. | AATCACCG |
| Kmr No. 474. | AATCACGT |
| Kmr No. 475. | AATCACTC |
| Kmr No. 476. | AATCACTG |
| Kmr No. 477. | AATCAGAC |
| Kmr No. 478. | AATCAGAT |
| Kmr No. 479. | AATCAGCC |
| Kmr No. 480. | AATCAGGA |
| Kmr No. 481. | AATCAGTC |
| Kmr No. 482. | AATCAGTG |
| Kmr No. 483. | AATCATGA |
| Kmr No. 484. | AATCATGC |
| Kmr No. 485. | AATCATGG |
| Kmr No. 486. | AATCATTG |
| Kmr No. 487. | AATCCAAC |
| Kmr No. 488. | AATCCAAG |
| Kmr No. 489. | AATCCAAT |
| Kmr No. 490. | AATCCACA |
| Kmr No. 491. | AATCCACT |
| Kmr No. 492. | AATCCAGA |
| Kmr No. 493. | AATCCAGG |
| Kmr No. 494. | AATCCAGT |
| Kmr No. 495. | AATCCATA |
| Kmr No. 496. | AATCCATC |
| Kmr No. 497. | AATCCATG |
| Kmr No. 498. | AATCCCAT |
| Kmr No. 499. | AATCCCTG |
| Kmr No. 500. | AATCCGAT |
| Kmr No. 501. | AATCCGGA |
| Kmr No. 502. | AATCCGGC |
| Kmr No. 503. | AATCCGGT |
| Kmr No. 504. | AATCCGTA |
| Kmr No. 505. | AATCCGTC |
| Kmr No. 506. | AATCCGTG |
| Kmr No. 507. | AATCCTGA |
| Kmr No. 508. | AATCCTGC |
| Kmr No. 509. | AATCCTGG |
| Kmr No. 510. | AATCCTGT |
| Kmr No. 511. | AATCCTTC |
| Kmr No. 512. | AATCGAAC |
| Kmr No. 513. | AATCGATA |
| Kmr No. 514. | AATCGATC |
| Kmr No. 515. | AATCGGAA |
| Kmr No. 516. | AATCGGAC |
| Kmr No. 517. | AATCGGAG |
| Kmr No. 518. | AATCGGAT |
| Kmr No. 519. | AATCGGTA |
| Kmr No. 520. | AATCGGTC |
| Kmr No. 521. | AATCGGTG |
| Kmr No. 522. | AATCGTAA |
| Kmr No. 523. | AATCGTAC |
| Kmr No. 524. | AATCGTAT |
| Kmr No. 525. | AATCGTCT |
| Kmr No. 526. | AATCGTTC |
| Kmr No. 527. | AATCTGAC |
| Kmr No. 528. | AATCTGAT |
| Kmr No. 529. | AATCTGGA |
| Kmr No. 530. | AATCTGGC |
| Kmr No. 531. | AATCTGGT |
| Kmr No. 532. | AATCTGTA |
| Kmr No. 533. | AATCTTCC |
| Kmr No. 534. | AATGAACG |
| Kmr No. 535. | AATGAATC |
| Kmr No. 536. | AATGACAG |
| Kmr No. 537. | AATGACCA |
| Kmr No. 538. | AATGACTG |
| Kmr No. 539. | AATGATCA |
| Kmr No. 540. | AATGATCC |
| Kmr No. 541. | AATGATCG |
| Kmr No. 542. | AATGATCT |
| Kmr No. 543. | AATGATTC |
| Kmr No. 544. | AATGATTG |
| Kmr No. 545. | AATGCATC |
| Kmr No. 546. | AATGCATG |
| Kmr No. 547. | AATGCCAG |
| Kmr No. 548. | AATGCCCT |
| Kmr No. 549. | AATGCCGG |
| Kmr No. 550. | AATGCGAT |
| Kmr No. 551. | AATGCGCA |
| Kmr No. 552. | AATGCGCT |
| Kmr No. 553. | AATGGAAG |
| Kmr No. 554. | AATGGACC |
| Kmr No. 555. | AATGGATC |
| Kmr No. 556. | AATGGATG |
| Kmr No. 557. | AATGGCAG |
| Kmr No. 558. | AATGGCTG |
| Kmr No. 559. | AATGGTCC |
| Kmr No. 560. | AATGGTTC |
| Kmr No. 561. | AATGTCGC |
| Kmr No. 562. | AATGTCTG |
| Kmr No. 563. | AATGTGAC |
| Kmr No. 564. | AATGTGAT |
| Kmr No. 565. | AATGTTCC |
| Kmr No. 566. | AATTACTG |
| Kmr No. 567. | AATTCACC |
| Kmr No. 568. | AATTCACG |
| Kmr No. 569. | AATTCACT |
| Kmr No. 570. | AATTCAGG |
| Kmr No. 571. | AATTCAGT |
| Kmr No. 572. | AATTCATC |
| Kmr No. 573. | AATTCATG |
| Kmr No. 574. | AATTCCAA |
| Kmr No. 575. | AATTCCAG |
| Kmr No. 576. | AATTCCCA |
| Kmr No. 577. | AATTCCGG |
| Kmr No. 578. | AATTCCGT |
| Kmr No. 579. | AATTCCTG |
| Kmr No. 580. | AATTCGAA |
| Kmr No. 581. | AATTCGTG |
| Kmr No. 582. | AATTCTGG |
| Kmr No. 583. | AATTCTTC |
| Kmr No. 584. | AATTGATC |
| Kmr No. 585. | AATTGCTG |
| Kmr No. 586. | AATTGGAC |
| Kmr No. 587. | AATTGGCC |
| Kmr No. 588. | AATTGGTC |
| Kmr No. 589. | AATTGGTG |
| Kmr No. 590. | AATTTCGC |
| Kmr No. 591. | ACAAAAAA |
| Kmr No. 592. | ACAAACAC |
| Kmr No. 593. | ACAAACCC |
| Kmr No. 594. | ACAACAAG |
| Kmr No. 595. | ACAACACA |
| Kmr No. 596. | ACAACATC |
| Kmr No. 597. | ACAACCCC |
| Kmr No. 598. | ACAACCCT |
| Kmr No. 599. | ACAACCTC |
| Kmr No. 600. | ACAACGAG |
| Kmr No. 601. | ACAACGCT |
| Kmr No. 602. | ACAACTGT |
| Kmr No. 603. | ACAAGAGC |
| Kmr No. 604. | ACAAGATG |
| Kmr No. 605. | ACAAGCAC |
| Kmr No. 606. | ACAAGCAG |
| Kmr No. 607. | ACAAGCCC |
| Kmr No. 608. | ACAAGCGA |
| Kmr No. 609. | ACAAGCGG |
| Kmr No. 610. | ACAAGCTG |
| Kmr No. 611. | ACAAGGAG |
| Kmr No. 612. | ACAAGGGC |
| Kmr No. 613. | ACAAGGGG |
| Kmr No. 614. | ACAAGGGT |
| Kmr No. 615. | ACAATCAC |
| Kmr No. 616. | ACAATCCG |
| Kmr No. 617. | ACAATCGG |
| Kmr No. 618. | ACAATCGT |
| Kmr No. 619. | ACAATTCC |
| Kmr No. 620. | ACACAAAA |
| Kmr No. 621. | ACACAAAC |
| Kmr No. 622. | ACACAACA |
| Kmr No. 623. | ACACAAGC |
| Kmr No. 624. | ACACACAA |
| Kmr No. 625. | ACACACAC |
| Kmr No. 626. | ACACACAG |
| Kmr No. 627. | ACACACAT |
| Kmr No. 628. | ACACACCA |
| Kmr No. 629. | ACACACCC |
| Kmr No. 630. | ACACACGA |
| Kmr No. 631. | ACACACGC |
| Kmr No. 632. | ACACACGG |
| Kmr No. 633. | ACACACTC |
| Kmr No. 634. | ACACAGAG |
| Kmr No. 635. | ACACAGCA |
| Kmr No. 636. | ACACAGCG |
| Kmr No. 637. | ACACATAG |
| Kmr No. 638. | ACACATCG |
| Kmr No. 639. | ACACCAAT |
| Kmr No. 640. | ACACCACA |
| Kmr No. 641. | ACACCAGC |
| Kmr No. 642. | ACACCCAC |
| Kmr No. 643. | ACACCCAG |
| Kmr No. 644. | ACACCCCA |
| Kmr No. 645. | ACACCCCC |
| Kmr No. 646. | ACACCCCG |
| Kmr No. 647. | ACACCCCT |
| Kmr No. 648. | ACACCCGA |
| Kmr No. 649. | ACACCCGC |
| Kmr No. 650. | ACACCCTA |
| Kmr No. 651. | ACACCCTC |
| Kmr No. 652. | ACACCCTG |
| Kmr No. 653. | ACACCGCT |
| Kmr No. 654. | ACACCGGA |
| Kmr No. 655. | ACACCTAT |
| Kmr No. 656. | ACACCTCA |
| Kmr No. 657. | ACACCTCC |
| Kmr No. 658. | ACACGAAA |
| Kmr No. 659. | ACACGACA |
| Kmr No. 660. | ACACGAGA |
| Kmr No. 661. | ACACGCAG |
| Kmr No. 662. | ACACGGAG |
| Kmr No. 663. | ACACGGCA |
| Kmr No. 664. | ACACGGGG |
| Kmr No. 665. | ACACGTAC |
| Kmr No. 666. | ACACGTAT |
| Kmr No. 667. | ACACGTTC |
| Kmr No. 668. | ACACGTTG |
| Kmr No. 669. | ACACTAAG |
| Kmr No. 670. | ACACTCAC |
| Kmr No. 671. | ACACTCAG |
| Kmr No. 672. | ACACTCGC |
| Kmr No. 673. | ACACTCGG |
| Kmr No. 674. | ACACTCTC |
| Kmr No. 675. | ACACTCTG |
| Kmr No. 676. | ACACTGAC |
| Kmr No. 677. | ACACTGAT |
| Kmr No. 678. | ACACTGCG |
| Kmr No. 679. | ACACTGGA |
| Kmr No. 680. | ACACTGGT |
| Kmr No. 681. | ACACTTCC |
| Kmr No. 682. | ACAGAAGA |
| Kmr No. 683. | ACAGAAGT |
| Kmr No. 684. | ACAGAATC |
| Kmr No. 685. | ACAGACCC |
| Kmr No. 686. | ACAGACGT |
| Kmr No. 687. | ACAGAGCA |
| Kmr No. 688. | ACAGAGCC |
| Kmr No. 689. | ACAGAGCG |
| Kmr No. 690. | ACAGAGTG |
| Kmr No. 691. | ACAGATTC |
| Kmr No. 692. | ACAGATTG |
| Kmr No. 693. | ACAGCAAG |
| Kmr No. 694. | ACAGCCAA |
| Kmr No. 695. | ACAGCCAT |
| Kmr No. 696. | ACAGCCCC |
| Kmr No. 697. | ACAGCCGT |
| Kmr No. 698. | ACAGCGAC |
| Kmr No. 699. | ACAGCGAG |
| Kmr No. 700. | ACAGCGCA |
| Kmr No. 701. | ACAGCGGC |
| Kmr No. 702. | ACAGCGGG |
| Kmr No. 703. | ACAGCGTA |
| Kmr No. 704. | ACAGGAAA |
| Kmr No. 705. | ACAGGAAC |
| Kmr No. 706. | ACAGGAAG |
| Kmr No. 707. | ACAGGAAT |
| Kmr No. 708. | ACAGGAGA |
| Kmr No. 709. | ACAGGCTC |
| Kmr No. 710. | ACAGGGGG |
| Kmr No. 711. | ACAGGGTC |
| Kmr No. 712. | ACAGGTAA |
| Kmr No. 713. | ACAGGTAC |
| Kmr No. 714. | ACAGGTAG |
| Kmr No. 715. | ACAGGTAT |
| Kmr No. 716. | ACAGGTTC |
| Kmr No. 717. | ACAGTAAC |
| Kmr No. 718. | ACAGTAAT |
| Kmr No. 719. | ACAGTACA |
| Kmr No. 720. | ACAGTACG |
| Kmr No. 721. | ACAGTACT |
| Kmr No. 722. | ACAGTAGA |
| Kmr No. 723. | ACAGTAGT |
| Kmr No. 724. | ACAGTCAA |
| Kmr No. 725. | ACAGTCAC |
| Kmr No. 726. | ACAGTCAG |
| Kmr No. 727. | ACAGTCAT |
| Kmr No. 728. | ACAGTCCA |
| Kmr No. 729. | ACAGTCTA |
| Kmr No. 730. | ACAGTGAA |
| Kmr No. 731. | ACAGTGAC |
| Kmr No. 732. | ACAGTGAT |
| Kmr No. 733. | ACAGTGGA |
| Kmr No. 734. | ACAGTGGT |
| Kmr No. 735. | ACAGTTAC |
| Kmr No. 736. | ACAGTTCC |
| Kmr No. 737. | ACAGTTGC |
| Kmr No. 738. | ACATAGAG |
| Kmr No. 739. | ACATAGCC |
| Kmr No. 740. | ACATAGCG |
| Kmr No. 741. | ACATAGGC |
| Kmr No. 742. | ACATAGGG |
| Kmr No. 743. | ACATAGTG |
| Kmr No. 744. | ACATCAAG |
| Kmr No. 745. | ACATCAGC |
| Kmr No. 746. | ACATCCAA |
| Kmr No. 747. | ACATCCAT |
| Kmr No. 748. | ACATCGCT |
| Kmr No. 749. | ACATGACT |
| Kmr No. 750. | ACATGCCA |
| Kmr No. 751. | ACATGGAA |
| Kmr No. 752. | ACATGTAC |
| Kmr No. 753. | ACATGTCC |
| Kmr No. 754. | ACATTGAG |
| Kmr No. 755. | ACATTGGA |
| Kmr No. 756. | ACCAAATC |
| Kmr No. 757. | ACCAAATG |
| Kmr No. 758. | ACCAAGTC |
| Kmr No. 759. | ACCAAGTG |
| Kmr No. 760. | ACCAATCA |
| Kmr No. 761. | ACCAATCC |
| Kmr No. 762. | ACCAATCG |
| Kmr No. 763. | ACCAATGA |
| Kmr No. 764. | ACCAATGC |
| Kmr No. 765. | ACCAATGG |
| Kmr No. 766. | ACCAATTC |
| Kmr No. 767. | ACCAATTG |
| Kmr No. 768. | ACCACAAA |
| Kmr No. 769. | ACCACAAC |
| Kmr No. 770. | ACCACAAG |
| Kmr No. 771. | ACCACACA |
| Kmr No. 772. | ACCACACC |
| Kmr No. 773. | ACCACCCC |
| Kmr No. 774. | ACCACCCG |
| Kmr No. 775. | ACCACCCT |
| Kmr No. 776. | ACCACCTC |
| Kmr No. 777. | ACCACGTA |
| Kmr No. 778. | ACCACTGA |
| Kmr No. 779. | ACCAGAAT |
| Kmr No. 780. | ACCAGCCT |
| Kmr No. 781. | ACCAGGTA |
| Kmr No. 782. | ACCAGTAA |
| Kmr No. 783. | ACCAGTAC |
| Kmr No. 784. | ACCAGTCA |
| Kmr No. 785. | ACCAGTGA |
| Kmr No. 786. | ACCAGTTA |
| Kmr No. 787. | ACCAGTTC |
| Kmr No. 788. | ACCATACG |
| Kmr No. 789. | ACCATTCC |
| Kmr No. 790. | ACCATTTC |
| Kmr No. 791. | ACCCACAA |
| Kmr No. 792. | ACCCACAC |
| Kmr No. 793. | ACCCACAG |
| Kmr No. 794. | ACCCACCA |
| Kmr No. 795. | ACCCACCC |
| Kmr No. 796. | ACCCACCG |
| Kmr No. 797. | ACCCACCT |
| Kmr No. 798. | ACCCACGA |
| Kmr No. 799. | ACCCACTC |
| Kmr No. 800. | ACCCAGAG |
| Kmr No. 801. | ACCCAGCA |
| Kmr No. 802. | ACCCAGCC |
| Kmr No. 803. | ACCCAGCG |
| Kmr No. 804. | ACCCCAAC |
| Kmr No. 805. | ACCCCAAG |
| Kmr No. 806. | ACCCCACA |
| Kmr No. 807. | ACCCCACC |
| Kmr No. 808. | ACCCCACT |
| Kmr No. 809. | ACCCCAGA |
| Kmr No. 810. | ACCCCAGC |
| Kmr No. 811. | ACCCCATC |
| Kmr No. 812. | ACCCCCAA |
| Kmr No. 813. | ACCCCCAC |
| Kmr No. 814. | ACCCCCAG |
| Kmr No. 815. | ACCCCCAT |
| Kmr No. 816. | ACCCCCCA |
| Kmr No. 817. | ACCCCCCC |
| Kmr No. 818. | ACCCCCCG |
| Kmr No. 819. | ACCCCCCT |
| Kmr No. 820. | ACCCCCGA |
| Kmr No. 821. | ACCCCCGC |
| Kmr No. 822. | ACCCCCGG |
| Kmr No. 823. | ACCCCCGT |
| Kmr No. 824. | ACCCCCTA |
| Kmr No. 825. | ACCCCCTC |
| Kmr No. 826. | ACCCCCTG |
| Kmr No. 827. | ACCCCGAC |
| Kmr No. 828. | ACCCCGCA |
| Kmr No. 829. | ACCCCGCC |
| Kmr No. 830. | ACCCCGCG |
| Kmr No. 831. | ACCCCGCT |
| Kmr No. 832. | ACCCCGGC |
| Kmr No. 833. | ACCCCGGG |
| Kmr No. 834. | ACCCCGTC |
| Kmr No. 835. | ACCCCGTG |
| Kmr No. 836. | ACCCCTAC |
| Kmr No. 837. | ACCCCTCA |
| Kmr No. 838. | ACCCCTCC |
| Kmr No. 839. | ACCCCTCG |
| Kmr No. 840. | ACCCCTCT |
| Kmr No. 841. | ACCCCTGC |
| Kmr No. 842. | ACCCGACA |
| Kmr No. 843. | ACCCGAGA |
| Kmr No. 844. | ACCCGCAC |
| Kmr No. 845. | ACCCGCAG |
| Kmr No. 846. | ACCCGCCA |
| Kmr No. 847. | ACCCGCCC |
| Kmr No. 848. | ACCCGCCG |
| Kmr No. 849. | ACCCGCCT |
| Kmr No. 850. | ACCCGCTC |
| Kmr No. 851. | ACCCGCTG |
| Kmr No. 852. | ACCCGGCG |
| Kmr No. 853. | ACCCGGGG |
| Kmr No. 854. | ACCCTAAG |
| Kmr No. 855. | ACCCTATG |
| Kmr No. 856. | ACCCTCAA |
| Kmr No. 857. | ACCCTCAC |
| Kmr No. 858. | ACCCTCAG |
| Kmr No. 859. | ACCCTCAT |
| Kmr No. 860. | ACCCTCCA |
| Kmr No. 861. | ACCCTCCC |
| Kmr No. 862. | ACCCTCCG |
| Kmr No. 863. | ACCCTCCT |
| Kmr No. 864. | ACCCTCGC |
| Kmr No. 865. | ACCCTCGG |
| Kmr No. 866. | ACCCTCTC |
| Kmr No. 867. | ACCCTCTG |
| Kmr No. 868. | ACCCTGAA |
| Kmr No. 869. | ACCCTGAC |
| Kmr No. 870. | ACCCTGAG |
| Kmr No. 871. | ACCCTGCA |
| Kmr No. 872. | ACCCTGCC |
| Kmr No. 873. | ACCCTGCT |
| Kmr No. 874. | ACCCTGTG |
| Kmr No. 875. | ACCCTTCT |
| Kmr No. 876. | ACCCTTGA |
| Kmr No. 877. | ACCCTTGC |
| Kmr No. 878. | ACCCTTTG |
| Kmr No. 879. | ACCGAGAA |
| Kmr No. 880. | ACCGAGAG |
| Kmr No. 881. | ACCGAGAT |
| Kmr No. 882. | ACCGAGGG |
| Kmr No. 883. | ACCGAGTG |
| Kmr No. 884. | ACCGATCA |
| Kmr No. 885. | ACCGATTC |
| Kmr No. 886. | ACCGATTG |
| Kmr No. 887. | ACCGCAAA |
| Kmr No. 888. | ACCGCAAG |
| Kmr No. 889. | ACCGCACA |
| Kmr No. 890. | ACCGCCAA |
| Kmr No. 891. | ACCGCCAT |
| Kmr No. 892. | ACCGCCCC |
| Kmr No. 893. | ACCGCCCT |
| Kmr No. 894. | ACCGCCTA |
| Kmr No. 895. | ACCGCTCT |
| Kmr No. 896. | ACCGCTGC |
| Kmr No. 897. | ACCGGAAC |
| Kmr No. 898. | ACCGGAAG |
| Kmr No. 899. | ACCGGAAT |
| Kmr No. 900. | ACCGGACC |
| Kmr No. 901. | ACCGGACT |
| Kmr No. 902. | ACCGGATA |
| Kmr No. 903. | ACCGGATC |
| Kmr No. 904. | ACCGGATG |
| Kmr No. 905. | ACCGGGGG |
| Kmr No. 906. | ACCGGTAC |
| Kmr No. 907. | ACCGGTCA |
| Kmr No. 908. | ACCGGTTC |
| Kmr No. 909. | ACCGGTTG |
| Kmr No. 910. | ACCGTACG |
| Kmr No. 911. | ACCGTACT |
| Kmr No. 912. | ACCGTATC |
| Kmr No. 913. | ACCGTATG |
| Kmr No. 914. | ACCGTCAT |
| Kmr No. 915. | ACCGTGTA |
| Kmr No. 916. | ACCGTTAC |
| Kmr No. 917. | ACCGTTCC |
| Kmr No. 918. | ACCTAGTG |
| Kmr No. 919. | ACCTATGC |
| Kmr No. 920. | ACCTCAAA |
| Kmr No. 921. | ACCTCACA |
| Kmr No. 922. | ACCTCAGC |
| Kmr No. 923. | ACCTCCCC |
| Kmr No. 924. | ACCTCCCG |
| Kmr No. 925. | ACCTCCGC |
| Kmr No. 926. | ACCTCCTC |
| Kmr No. 927. | ACCTCGTA |
| Kmr No. 928. | ACCTCTCA |
| Kmr No. 929. | ACCTCTCC |
| Kmr No. 930. | ACCTCTCG |
| Kmr No. 931. | ACCTCTGC |
| Kmr No. 932. | ACCTGAAT |
| Kmr No. 933. | ACCTGCAT |
| Kmr No. 934. | ACCTGGAA |
| Kmr No. 935. | ACCTGGTC |
| Kmr No. 936. | ACCTGTAC |
| Kmr No. 937. | ACCTGTAT |
| Kmr No. 938. | ACCTTCCA |
| Kmr No. 939. | ACCTTGGA |
| Kmr No. 940. | ACCTTGTG |
| Kmr No. 941. | ACGAAAAA |
| Kmr No. 942. | ACGAAAAG |
| Kmr No. 943. | ACGAAGAG |
| Kmr No. 944. | ACGAATCA |
| Kmr No. 945. | ACGAATCC |
| Kmr No. 946. | ACGAATCT |
| Kmr No. 947. | ACGAATGA |
| Kmr No. 948. | ACGAATTC |
| Kmr No. 949. | ACGACAAG |
| Kmr No. 950. | ACGACACA |
| Kmr No. 951. | ACGACACT |
| Kmr No. 952. | ACGACAGA |
| Kmr No. 953. | ACGACAGC |
| Kmr No. 954. | ACGACATA |
| Kmr No. 955. | ACGACCCC |
| Kmr No. 956. | ACGACCCT |
| Kmr No. 957. | ACGACTAG |
| Kmr No. 958. | ACGAGAAA |
| Kmr No. 959. | ACGAGAGA |
| Kmr No. 960. | ACGAGAGC |
| Kmr No. 961. | ACGAGAGG |
| Kmr No. 962. | ACGAGAGT |
| Kmr No. 963. | ACGAGATA |
| Kmr No. 964. | ACGAGGAG |
| Kmr No. 965. | ACGAGGCA |
| Kmr No. 966. | ACGAGGCT |
| Kmr No. 967. | ACGAGGGA |
| Kmr No. 968. | ACGAGGGG |
| Kmr No. 969. | ACGAGGTA |
| Kmr No. 970. | ACGATCAA |
| Kmr No. 971. | ACGATCCA |
| Kmr No. 972. | ACGATTAC |
| Kmr No. 973. | ACGATTCA |
| Kmr No. 974. | ACGATTGA |
| Kmr No. 975. | ACGATTGG |
| Kmr No. 976. | ACGCAAAA |
| Kmr No. 977. | ACGCAAAG |
| Kmr No. 978. | ACGCACAG |
| Kmr No. 979. | ACGCAGAA |
| Kmr No. 980. | ACGCAGAG |
| Kmr No. 981. | ACGCCAAT |
| Kmr No. 982. | ACGCCAGT |
| Kmr No. 983. | ACGCCCCC |
| Kmr No. 984. | ACGCCCCG |
| Kmr No. 985. | ACGCCCTC |
| Kmr No. 986. | ACGCCTAT |
| Kmr No. 987. | ACGCGGGG |
| Kmr No. 988. | ACGCGTAA |
| Kmr No. 989. | ACGCGTAC |
| Kmr No. 990. | ACGCTAAG |
| Kmr No. 991. | ACGCTCAG |
| Kmr No. 992. | ACGCTCCT |
| Kmr No. 993. | ACGCTCTC |
| Kmr No. 994. | ACGCTCTG |
| Kmr No. 995. | ACGCTGAG |
| Kmr No. 996. | ACGCTGCC |
| Kmr No. 997. | ACGCTGTA |
| Kmr No. 998. | ACGCTGTC |
| Kmr No. 999. | ACGCTGTG |
| Kmr No. 1000. | ACGCTTAT |
| Kmr No. 1001. | ACGGAACA |
| Kmr No. 1002. | ACGGAACC |
| Kmr No. 1003. | ACGGAAGT |
| Kmr No. 1004. | ACGGAATC |
| Kmr No. 1005. | ACGGACCA |
| Kmr No. 1006. | ACGGACCG |
| Kmr No. 1007. | ACGGAGAG |
| Kmr No. 1008. | ACGGAGCG |
| Kmr No. 1009. | ACGGAGCT |
| Kmr No. 1010. | ACGGAGGA |
| Kmr No. 1011. | ACGGAGGG |
| Kmr No. 1012. | ACGGATAC |
| Kmr No. 1013. | ACGGATCA |
| Kmr No. 1014. | ACGGATTC |
| Kmr No. 1015. | ACGGATTG |
| Kmr No. 1016. | ACGGCACT |
| Kmr No. 1017. | ACGGCAGA |
| Kmr No. 1018. | ACGGCGGG |
| Kmr No. 1019. | ACGGCTCT |
| Kmr No. 1020. | ACGGGGCA |
| Kmr No. 1021. | ACGGGGCG |
| Kmr No. 1022. | ACGGGGCT |
| Kmr No. 1023. | ACGGGGGC |
| Kmr No. 1024. | ACGGGGGG |
| Kmr No. 1025. | ACGGTAAT |
| Kmr No. 1026. | ACGGTACT |
| Kmr No. 1027. | ACGGTAGT |
| Kmr No. 1028. | ACGGTCCA |
| Kmr No. 1029. | ACGGTGGG |
| Kmr No. 1030. | ACGGTTAA |
| Kmr No. 1031. | ACGGTTCA |
| Kmr No. 1032. | ACGGTTCC |
| Kmr No. 1033. | ACGGTTGG |
| Kmr No. 1034. | ACGGTTTC |
| Kmr No. 1035. | ACGTAACG |
| Kmr No. 1036. | ACGTAAGA |
| Kmr No. 1037. | ACGTAATC |
| Kmr No. 1038. | ACGTACAG |
| Kmr No. 1039. | ACGTACAT |
| Kmr No. 1040. | ACGTACCA |
| Kmr No. 1041. | ACGTACCG |
| Kmr No. 1042. | ACGTACGA |
| Kmr No. 1043. | ACGTACGC |
| Kmr No. 1044. | ACGTACGG |
| Kmr No. 1045. | ACGTACGT |
| Kmr No. 1046. | ACGTACTC |
| Kmr No. 1047. | ACGTACTG |
| Kmr No. 1048. | ACGTAGAG |
| Kmr No. 1049. | ACGTAGGA |
| Kmr No. 1050. | ACGTAGTA |
| Kmr No. 1051. | ACGTATCC |
| Kmr No. 1052. | ACGTATCG |
| Kmr No. 1053. | ACGTATGA |
| Kmr No. 1054. | ACGTATGG |
| Kmr No. 1055. | ACGTATTA |
| Kmr No. 1056. | ACGTATTC |
| Kmr No. 1057. | ACGTCAAT |
| Kmr No. 1058. | ACGTCACT |
| Kmr No. 1059. | ACGTCAGA |
| Kmr No. 1060. | ACGTCAGG |
| Kmr No. 1061. | ACGTCAGT |
| Kmr No. 1062. | ACGTCCAA |
| Kmr No. 1063. | ACGTCCAT |
| Kmr No. 1064. | ACGTCCGA |
| Kmr No. 1065. | ACGTCCGG |
| Kmr No. 1066. | ACGTCTGA |
| Kmr No. 1067. | ACGTCTGG |
| Kmr No. 1068. | ACGTCTTG |
| Kmr No. 1069. | ACGTGCAC |
| Kmr No. 1070. | ACGTGCAT |
| Kmr No. 1071. | ACGTGCTA |
| Kmr No. 1072. | ACGTGGAA |
| Kmr No. 1073. | ACGTGGAT |
| Kmr No. 1074. | ACGTGTAC |
| Kmr No. 1075. | ACGTGTAG |
| Kmr No. 1076. | ACGTGTAT |
| Kmr No. 1077. | ACGTGTCA |
| Kmr No. 1078. | ACGTGTGA |
| Kmr No. 1079. | ACGTGTGG |
| Kmr No. 1080. | ACGTGTTG |
| Kmr No. 1081. | ACGTTACT |
| Kmr No. 1082. | ACGTTATG |
| Kmr No. 1083. | ACGTTCAA |
| Kmr No. 1084. | ACGTTCAT |
| Kmr No. 1085. | ACGTTCCA |
| Kmr No. 1086. | ACGTTCCG |
| Kmr No. 1087. | ACGTTCCT |
| Kmr No. 1088. | ACGTTCTA |
| Kmr No. 1089. | ACGTTGGA |
| Kmr No. 1090. | ACGTTTAA |
| Kmr No. 1091. | ACGTTTAG |
| Kmr No. 1092. | ACGTTTCC |
| Kmr No. 1093. | ACGTTTGA |
| Kmr No. 1094. | ACGTTTGG |
| Kmr No. 1095. | ACGTTTTA |
| Kmr No. 1096. | ACGTTTTG |
| Kmr No. 1097. | ACTAATCC |
| Kmr No. 1098. | ACTAATTC |
| Kmr No. 1099. | ACTACAAG |
| Kmr No. 1100. | ACTACACC |
| Kmr No. 1101. | ACTACCCC |
| Kmr No. 1102. | ACTACGGC |
| Kmr No. 1103. | ACTACGTG |
| Kmr No. 1104. | ACTAGGTA |
| Kmr No. 1105. | ACTAGTCA |
| Kmr No. 1106. | ACTAGTCC |
| Kmr No. 1107. | ACTAGTCG |
| Kmr No. 1108. | ACTAGTGG |
| Kmr No. 1109. | ACTAGTTC |
| Kmr No. 1110. | ACTATCTC |
| Kmr No. 1111. | ACTATGAG |
| Kmr No. 1112. | ACTATGCC |
| Kmr No. 1113. | ACTATGTC |
| Kmr No. 1114. | ACTATGTG |
| Kmr No. 1115. | ACTCACAC |
| Kmr No. 1116. | ACTCACAG |
| Kmr No. 1117. | ACTCATGA |
| Kmr No. 1118. | ACTCCCCC |
| Kmr No. 1119. | ACTCCCGC |
| Kmr No. 1120. | ACTCCGAT |
| Kmr No. 1121. | ACTCCGGA |
| Kmr No. 1122. | ACTCGCAG |
| Kmr No. 1123. | ACTCGCTG |
| Kmr No. 1124. | ACTCGGGG |
| Kmr No. 1125. | ACTCTCAC |
| Kmr No. 1126. | ACTCTCGA |
| Kmr No. 1127. | ACTCTCGC |
| Kmr No. 1128. | ACTCTCGG |
| Kmr No. 1129. | ACTCTCTC |
| Kmr No. 1130. | ACTCTGCC |
| Kmr No. 1131. | ACTCTGCG |
| Kmr No. 1132. | ACTCTGTG |
| Kmr No. 1133. | ACTGAACC |
| Kmr No. 1134. | ACTGAATA |
| Kmr No. 1135. | ACTGAATC |
| Kmr No. 1136. | ACTGACAA |
| Kmr No. 1137. | ACTGACAG |
| Kmr No. 1138. | ACTGACAT |
| Kmr No. 1139. | ACTGACCA |
| Kmr No. 1140. | ACTGACTC |
| Kmr No. 1141. | ACTGACTG |
| Kmr No. 1142. | ACTGAGAG |
| Kmr No. 1143. | ACTGAGGC |
| Kmr No. 1144. | ACTGAGGG |
| Kmr No. 1145. | ACTGATAT |
| Kmr No. 1146. | ACTGATCA |
| Kmr No. 1147. | ACTGATCC |
| Kmr No. 1148. | ACTGATCG |
| Kmr No. 1149. | ACTGATCT |
| Kmr No. 1150. | ACTGATTC |
| Kmr No. 1151. | ACTGATTG |
| Kmr No. 1152. | ACTGCAAG |
| Kmr No. 1153. | ACTGCCAT |
| Kmr No. 1154. | ACTGCCCC |
| Kmr No. 1155. | ACTGCGAC |
| Kmr No. 1156. | ACTGCGAG |
| Kmr No. 1157. | ACTGCGCA |
| Kmr No. 1158. | ACTGCGCC |
| Kmr No. 1159. | ACTGCGCT |
| Kmr No. 1160. | ACTGCGGG |
| Kmr No. 1161. | ACTGCTTG |
| Kmr No. 1162. | ACTGGAAA |
| Kmr No. 1163. | ACTGGAAC |
| Kmr No. 1164. | ACTGGAAG |
| Kmr No. 1165. | ACTGGAAT |
| Kmr No. 1166. | ACTGGACC |
| Kmr No. 1167. | ACTGGAGA |
| Kmr No. 1168. | ACTGGATA |
| Kmr No. 1169. | ACTGGATG |
| Kmr No. 1170. | ACTGGCAG |
| Kmr No. 1171. | ACTGGCAT |
| Kmr No. 1172. | ACTGGCCA |
| Kmr No. 1173. | ACTGGGAA |
| Kmr No. 1174. | ACTGGGAT |
| Kmr No. 1175. | ACTGGGGG |
| Kmr No. 1176. | ACTGGTAA |
| Kmr No. 1177. | ACTGGTAC |
| Kmr No. 1178. | ACTGGTAG |
| Kmr No. 1179. | ACTGGTAT |
| Kmr No. 1180. | ACTGGTCA |
| Kmr No. 1181. | ACTGGTCC |
| Kmr No. 1182. | ACTGGTCT |
| Kmr No. 1183. | ACTGGTTA |
| Kmr No. 1184. | ACTGGTTC |
| Kmr No. 1185. | ACTGTAAA |
| Kmr No. 1186. | ACTGTAAT |
| Kmr No. 1187. | ACTGTACC |
| Kmr No. 1188. | ACTGTACG |
| Kmr No. 1189. | ACTGTACT |
| Kmr No. 1190. | ACTGTAGA |
| Kmr No. 1191. | ACTGTATC |
| Kmr No. 1192. | ACTGTCAT |
| Kmr No. 1193. | ACTGTCCA |
| Kmr No. 1194. | ACTGTGAG |
| Kmr No. 1195. | ACTGTGAT |
| Kmr No. 1196. | ACTGTGCC |
| Kmr No. 1197. | ACTGTGGA |
| Kmr No. 1198. | ACTGTTAC |
| Kmr No. 1199. | ACTGTTCA |
| Kmr No. 1200. | ACTGTTCC |
| Kmr No. 1201. | ACTGTTCG |
| Kmr No. 1202. | ACTGTTCT |
| Kmr No. 1203. | ACTGTTTC |
| Kmr No. 1204. | ACTTCAGG |
| Kmr No. 1205. | ACTTCCAA |
| Kmr No. 1206. | ACTTCCAC |
| Kmr No. 1207. | ACTTCCAG |
| Kmr No. 1208. | ACTTCCAT |
| Kmr No. 1209. | ACTTCCCA |
| Kmr No. 1210. | ACTTCCGA |
| Kmr No. 1211. | ACTTCCGG |
| Kmr No. 1212. | ACTTCCTA |
| Kmr No. 1213. | ACTTCCTG |
| Kmr No. 1214. | ACTTCGAA |
| Kmr No. 1215. | ACTTCTTC |
| Kmr No. 1216. | ACTTGAGG |
| Kmr No. 1217. | ACTTGCCC |
| Kmr No. 1218. | ACTTGGAA |
| Kmr No. 1219. | ACTTGGAC |
| Kmr No. 1220. | ACTTGGAT |
| Kmr No. 1221. | ACTTGGTC |
| Kmr No. 1222. | ACTTTAAA |
| Kmr No. 1223. | ACTTTCCA |
| Kmr No. 1224. | ACTTTGAG |
| Kmr No. 1225. | ACTTTTCG |
| Kmr No. 1226. | AGAAACGC |
| Kmr No. 1227. | AGAAAGGC |
| Kmr No. 1228. | AGAACAGG |
| Kmr No. 1229. | AGAACCCC |
| Kmr No. 1230. | AGAACGAG |
| Kmr No. 1231. | AGAACGCA |
| Kmr No. 1232. | AGAACTGG |
| Kmr No. 1233. | AGAAGACG |
| Kmr No. 1234. | AGAAGGGC |
| Kmr No. 1235. | AGAAGTGA |
| Kmr No. 1236. | AGAATACG |
| Kmr No. 1237. | AGAATCAC |
| Kmr No. 1238. | AGAATCCA |
| Kmr No. 1239. | AGAATCCG |
| Kmr No. 1240. | AGAATCGG |
| Kmr No. 1241. | AGAATGCT |
| Kmr No. 1242. | AGAATTCC |
| Kmr No. 1243. | AGACAATC |
| Kmr No. 1244. | AGACACAA |
| Kmr No. 1245. | AGACACAC |
| Kmr No. 1246. | AGACACCC |
| Kmr No. 1247. | AGACACTC |
| Kmr No. 1248. | AGACATAG |
| Kmr No. 1249. | AGACCCAA |
| Kmr No. 1250. | AGACCCAC |
| Kmr No. 1251. | AGACCCAG |
| Kmr No. 1252. | AGACCCAT |
| Kmr No. 1253. | AGACCCCA |
| Kmr No. 1254. | AGACCCCC |
| Kmr No. 1255. | AGACCCCG |
| Kmr No. 1256. | AGACCCCT |
| Kmr No. 1257. | AGACCCGA |
| Kmr No. 1258. | AGACCCGC |
| Kmr No. 1259. | AGACCCTC |
| Kmr No. 1260. | AGACCCTG |
| Kmr No. 1261. | AGACCGCA |
| Kmr No. 1262. | AGACCGGA |
| Kmr No. 1263. | AGACCTAC |
| Kmr No. 1264. | AGACCTAT |
| Kmr No. 1265. | AGACCTCA |
| Kmr No. 1266. | AGACCTCC |
| Kmr No. 1267. | AGACCTGC |
| Kmr No. 1268. | AGACGAGG |
| Kmr No. 1269. | AGACGCAA |
| Kmr No. 1270. | AGACGCAG |
| Kmr No. 1271. | AGACGGAA |
| Kmr No. 1272. | AGACGGAT |
| Kmr No. 1273. | AGACGGGG |
| Kmr No. 1274. | AGACGTAC |
| Kmr No. 1275. | AGACGTTC |
| Kmr No. 1276. | AGACTGCG |
| Kmr No. 1277. | AGACTGGA |
| Kmr No. 1278. | AGACTTCC |
| Kmr No. 1279. | AGAGACCC |
| Kmr No. 1280. | AGAGACCG |
| Kmr No. 1281. | AGAGACCT |
| Kmr No. 1282. | AGAGACGC |
| Kmr No. 1283. | AGAGACGG |
| Kmr No. 1284. | AGAGACTC |
| Kmr No. 1285. | AGAGAGAC |
| Kmr No. 1286. | AGAGAGAG |
| Kmr No. 1287. | AGAGAGCA |
| Kmr No. 1288. | AGAGAGCC |
| Kmr No. 1289. | AGAGAGCG |
| Kmr No. 1290. | AGAGAGCT |
| Kmr No. 1291. | AGAGAGGC |
| Kmr No. 1292. | AGAGAGGG |
| Kmr No. 1293. | AGAGCAAA |
| Kmr No. 1294. | AGAGCAAC |
| Kmr No. 1295. | AGAGCAAG |
| Kmr No. 1296. | AGAGCACA |
| Kmr No. 1297. | AGAGCACC |
| Kmr No. 1298. | AGAGCACG |
| Kmr No. 1299. | AGAGCAGC |
| Kmr No. 1300. | AGAGCAGG |
| Kmr No. 1301. | AGAGCATA |
| Kmr No. 1302. | AGAGCATC |
| Kmr No. 1303. | AGAGCATG |
| Kmr No. 1304. | AGAGCCAC |
| Kmr No. 1305. | AGAGCCCC |
| Kmr No. 1306. | AGAGCCCG |
| Kmr No. 1307. | AGAGCCGA |
| Kmr No. 1308. | AGAGCCGC |
| Kmr No. 1309. | AGAGCCTC |
| Kmr No. 1310. | AGAGCCTG |
| Kmr No. 1311. | AGAGCGAA |
| Kmr No. 1312. | AGAGCGAC |
| Kmr No. 1313. | AGAGCGAG |
| Kmr No. 1314. | AGAGCGAT |
| Kmr No. 1315. | AGAGCGCA |
| Kmr No. 1316. | AGAGCGCC |
| Kmr No. 1317. | AGAGCGCG |
| Kmr No. 1318. | AGAGCGCT |
| Kmr No. 1319. | AGAGCGGA |
| Kmr No. 1320. | AGAGCGGC |
| Kmr No. 1321. | AGAGCGGG |
| Kmr No. 1322. | AGAGCGTA |
| Kmr No. 1323. | AGAGCGTC |
| Kmr No. 1324. | AGAGCGTG |
| Kmr No. 1325. | AGAGCTAC |
| Kmr No. 1326. | AGAGCTAT |
| Kmr No. 1327. | AGAGCTGC |
| Kmr No. 1328. | AGAGCTTG |
| Kmr No. 1329. | AGAGGAGG |
| Kmr No. 1330. | AGAGGCAC |
| Kmr No. 1331. | AGAGGCGA |
| Kmr No. 1332. | AGAGGCGC |
| Kmr No. 1333. | AGAGGCTC |
| Kmr No. 1334. | AGAGGCTG |
| Kmr No. 1335. | AGAGGGCA |
| Kmr No. 1336. | AGAGGGCG |
| Kmr No. 1337. | AGAGGGCT |
| Kmr No. 1338. | AGAGGGGC |
| Kmr No. 1339. | AGAGGGGG |
| Kmr No. 1340. | AGAGGGTC |
| Kmr No. 1341. | AGAGGGTG |
| Kmr No. 1342. | AGAGGTCA |
| Kmr No. 1343. | AGAGGTCG |
| Kmr No. 1344. | AGAGGTGC |
| Kmr No. 1345. | AGAGGTGG |
| Kmr No. 1346. | AGAGGTTG |
| Kmr No. 1347. | AGAGTCCC |
| Kmr No. 1348. | AGAGTCGC |
| Kmr No. 1349. | AGAGTCGG |
| Kmr No. 1350. | AGAGTCTC |
| Kmr No. 1351. | AGAGTGAG |
| Kmr No. 1352. | AGAGTGCA |
| Kmr No. 1353. | AGAGTGCC |
| Kmr No. 1354. | AGAGTGCG |
| Kmr No. 1355. | AGAGTGCT |
| Kmr No. 1356. | AGAGTGGC |
| Kmr No. 1357. | AGAGTGGG |
| Kmr No. 1358. | AGAGTGTC |
| Kmr No. 1359. | AGAGTGTG |
| Kmr No. 1360. | AGATACGC |
| Kmr No. 1361. | AGATACGG |
| Kmr No. 1362. | AGATAGAG |
| Kmr No. 1363. | AGATAGCG |
| Kmr No. 1364. | AGATAGGC |
| Kmr No. 1365. | AGATATCA |
| Kmr No. 1366. | AGATATCC |
| Kmr No. 1367. | AGATATCT |
| Kmr No. 1368. | AGATCACT |
| Kmr No. 1369. | AGATCCCC |
| Kmr No. 1370. | AGATCCGA |
| Kmr No. 1371. | AGATGCCC |
| Kmr No. 1372. | AGATGGAG |
| Kmr No. 1373. | AGATGGGC |
| Kmr No. 1374. | AGATGGGG |
| Kmr No. 1375. | AGATTCAC |
| Kmr No. 1376. | AGATTCCC |
| Kmr No. 1377. | AGATTCCG |
| Kmr No. 1378. | AGATTGGA |
| Kmr No. 1379. | AGATTTCC |
| Kmr No. 1380. | AGCAAAGC |
| Kmr No. 1381. | AGCAAGCA |
| Kmr No. 1382. | AGCAAGCC |
| Kmr No. 1383. | AGCAAGCG |
| Kmr No. 1384. | AGCAAGCT |
| Kmr No. 1385. | AGCAAGGA |
| Kmr No. 1386. | AGCAAGGC |
| Kmr No. 1387. | AGCAAGGG |
| Kmr No. 1388. | AGCAATCA |
| Kmr No. 1389. | AGCAATCC |
| Kmr No. 1390. | AGCAATCG |
| Kmr No. 1391. | AGCAATTG |
| Kmr No. 1392. | AGCACAAA |
| Kmr No. 1393. | AGCACAAC |
| Kmr No. 1394. | AGCACAAG |
| Kmr No. 1395. | AGCACACA |
| Kmr No. 1396. | AGCACACC |
| Kmr No. 1397. | AGCACAGC |
| Kmr No. 1398. | AGCACATA |
| Kmr No. 1399. | AGCACATC |
| Kmr No. 1400. | AGCACCCA |
| Kmr No. 1401. | AGCACCCC |
| Kmr No. 1402. | AGCACCCG |
| Kmr No. 1403. | AGCACCCT |
| Kmr No. 1404. | AGCACCTC |
| Kmr No. 1405. | AGCACGCA |
| Kmr No. 1406. | AGCACGCT |
| Kmr No. 1407. | AGCACGGG |
| Kmr No. 1408. | AGCACGTA |
| Kmr No. 1409. | AGCACTCC |
| Kmr No. 1410. | AGCAGAGC |
| Kmr No. 1411. | AGCAGCCC |
| Kmr No. 1412. | AGCAGCCT |
| Kmr No. 1413. | AGCAGGCT |
| Kmr No. 1414. | AGCAGGGC |
| Kmr No. 1415. | AGCAGGGG |
| Kmr No. 1416. | AGCAGTAG |
| Kmr No. 1417. | AGCAGTCA |
| Kmr No. 1418. | AGCAGTGA |
| Kmr No. 1419. | AGCATAGC |
| Kmr No. 1420. | AGCATCTC |
| Kmr No. 1421. | AGCATTCC |
| Kmr No. 1422. | AGCCACAC |
| Kmr No. 1423. | AGCCACCC |
| Kmr No. 1424. | AGCCCACA |
| Kmr No. 1425. | AGCCCACC |
| Kmr No. 1426. | AGCCCAGC |
| Kmr No. 1427. | AGCCCATC |
| Kmr No. 1428. | AGCCCCAC |
| Kmr No. 1429. | AGCCCCCA |
| Kmr No. 1430. | AGCCCCCC |
| Kmr No. 1431. | AGCCCCCG |
| Kmr No. 1432. | AGCCCCCT |
| Kmr No. 1433. | AGCCCCGA |
| Kmr No. 1434. | AGCCCCGC |
| Kmr No. 1435. | AGCCCCGG |
| Kmr No. 1436. | AGCCCCTA |
| Kmr No. 1437. | AGCCCCTC |
| Kmr No. 1438. | AGCCCGCA |
| Kmr No. 1439. | AGCCCGCC |
| Kmr No. 1440. | AGCCCTAC |
| Kmr No. 1441. | AGCCCTCA |
| Kmr No. 1442. | AGCCCTCC |
| Kmr No. 1443. | AGCCCTGC |
| Kmr No. 1444. | AGCCCTTG |
| Kmr No. 1445. | AGCCGAGA |
| Kmr No. 1446. | AGCCGCAA |
| Kmr No. 1447. | AGCCGCAC |
| Kmr No. 1448. | AGCCGCAG |
| Kmr No. 1449. | AGCCGCCC |
| Kmr No. 1450. | AGCCGCTG |
| Kmr No. 1451. | AGCCGGGG |
| Kmr No. 1452. | AGCCTAAC |
| Kmr No. 1453. | AGCCTAAG |
| Kmr No. 1454. | AGCCTCAA |
| Kmr No. 1455. | AGCCTCAC |
| Kmr No. 1456. | AGCCTCCT |
| Kmr No. 1457. | AGCCTCTC |
| Kmr No. 1458. | AGCCTGAG |
| Kmr No. 1459. | AGCCTGCA |
| Kmr No. 1460. | AGCCTGCC |
| Kmr No. 1461. | AGCCTGTG |
| Kmr No. 1462. | AGCCTTGA |
| Kmr No. 1463. | AGCCTTGC |
| Kmr No. 1464. | AGCGAAAA |
| Kmr No. 1465. | AGCGACAA |
| Kmr No. 1466. | AGCGACAC |
| Kmr No. 1467. | AGCGACAG |
| Kmr No. 1468. | AGCGACAT |
| Kmr No. 1469. | AGCGACCT |
| Kmr No. 1470. | AGCGAGAA |
| Kmr No. 1471. | AGCGAGAG |
| Kmr No. 1472. | AGCGAGAT |
| Kmr No. 1473. | AGCGAGGA |
| Kmr No. 1474. | AGCGAGGG |
| Kmr No. 1475. | AGCGAGTA |
| Kmr No. 1476. | AGCGAGTG |
| Kmr No. 1477. | AGCGATCA |
| Kmr No. 1478. | AGCGATCG |
| Kmr No. 1479. | AGCGCAAA |
| Kmr No. 1480. | AGCGCAAG |
| Kmr No. 1481. | AGCGCACA |
| Kmr No. 1482. | AGCGCACT |
| Kmr No. 1483. | AGCGCAGA |
| Kmr No. 1484. | AGCGCAGC |
| Kmr No. 1485. | AGCGCATA |
| Kmr No. 1486. | AGCGCCCC |
| Kmr No. 1487. | AGCGCCCT |
| Kmr No. 1488. | AGCGCTAT |
| Kmr No. 1489. | AGCGCTGC |
| Kmr No. 1490. | AGCGGAGC |
| Kmr No. 1491. | AGCGGAGG |
| Kmr No. 1492. | AGCGGCAA |
| Kmr No. 1493. | AGCGGCAG |
| Kmr No. 1494. | AGCGGCCC |
| Kmr No. 1495. | AGCGGCGG |
| Kmr No. 1496. | AGCGGCTA |
| Kmr No. 1497. | AGCGGCTG |
| Kmr No. 1498. | AGCGGGAC |
| Kmr No. 1499. | AGCGGGAG |
| Kmr No. 1500. | AGCGGGAT |
| Kmr No. 1501. | AGCGGGCA |
| Kmr No. 1502. | AGCGGGCG |
| Kmr No. 1503. | AGCGGGGC |
| Kmr No. 1504. | AGCGGGGG |
| Kmr No. 1505. | AGCGGGTC |
| Kmr No. 1506. | AGCGGGTG |
| Kmr No. 1507. | AGCGGTAT |
| Kmr No. 1508. | AGCGGTGC |
| Kmr No. 1509. | AGCGTAAG |
| Kmr No. 1510. | AGCGTACG |
| Kmr No. 1511. | AGCGTAGA |
| Kmr No. 1512. | AGCGTATC |
| Kmr No. 1513. | AGCGTCAT |
| Kmr No. 1514. | AGCGTCCC |
| Kmr No. 1515. | AGCGTCCT |
| Kmr No. 1516. | AGCGTGAA |
| Kmr No. 1517. | AGCGTGAG |
| Kmr No. 1518. | AGCGTGCA |
| Kmr No. 1519. | AGCGTGGG |
| Kmr No. 1520. | AGCTACGC |
| Kmr No. 1521. | AGCTATGC |
| Kmr No. 1522. | AGCTCAAA |
| Kmr No. 1523. | AGCTCACA |
| Kmr No. 1524. | AGCTCCAC |
| Kmr No. 1525. | AGCTCCCC |
| Kmr No. 1526. | AGCTCCCG |
| Kmr No. 1527. | AGCTCCGC |
| Kmr No. 1528. | AGCTCTAC |
| Kmr No. 1529. | AGCTCTAT |
| Kmr No. 1530. | AGCTCTCA |
| Kmr No. 1531. | AGCTCTCC |
| Kmr No. 1532. | AGCTCTCG |
| Kmr No. 1533. | AGCTCTGC |
| Kmr No. 1534. | AGCTGAAC |
| Kmr No. 1535. | AGCTGACC |
| Kmr No. 1536. | AGCTGACT |
| Kmr No. 1537. | AGCTGAGC |
| Kmr No. 1538. | AGCTGATG |
| Kmr No. 1539. | AGCTGCAC |
| Kmr No. 1540. | AGCTGCCC |
| Kmr No. 1541. | AGCTGCGG |
| Kmr No. 1542. | AGCTGCTG |
| Kmr No. 1543. | AGCTGGAG |
| Kmr No. 1544. | AGCTGGGG |
| Kmr No. 1545. | AGCTGTCG |
| Kmr No. 1546. | AGCTGTGC |
| Kmr No. 1547. | AGCTTAGG |
| Kmr No. 1548. | AGCTTATC |
| Kmr No. 1549. | AGCTTCCA |
| Kmr No. 1550. | AGCTTGAA |
| Kmr No. 1551. | AGCTTGTA |
| Kmr No. 1552. | AGCTTTGC |
| Kmr No. 1553. | AGCTTTTC |
| Kmr No. 1554. | AGGAACAT |
| Kmr No. 1555. | AGGAACCA |
| Kmr No. 1556. | AGGAACCG |
| Kmr No. 1557. | AGGAACGA |
| Kmr No. 1558. | AGGAAGTA |
| Kmr No. 1559. | AGGAAGTG |
| Kmr No. 1560. | AGGAATCA |
| Kmr No. 1561. | AGGAATCC |
| Kmr No. 1562. | AGGAATCG |
| Kmr No. 1563. | AGGAATGA |
| Kmr No. 1564. | AGGAATGC |
| Kmr No. 1565. | AGGAATTA |
| Kmr No. 1566. | AGGAATTC |
| Kmr No. 1567. | AGGAATTG |
| Kmr No. 1568. | AGGACAAC |
| Kmr No. 1569. | AGGACAAG |
| Kmr No. 1570. | AGGACACA |
| Kmr No. 1571. | AGGACATC |
| Kmr No. 1572. | AGGACCAA |
| Kmr No. 1573. | AGGACCCC |
| Kmr No. 1574. | AGGACGAA |
| Kmr No. 1575. | AGGACGCA |
| Kmr No. 1576. | AGGACTAC |
| Kmr No. 1577. | AGGACTGA |
| Kmr No. 1578. | AGGAGAGC |
| Kmr No. 1579. | AGGAGCAC |
| Kmr No. 1580. | AGGAGCAG |
| Kmr No. 1581. | AGGAGCGA |
| Kmr No. 1582. | AGGAGCGG |
| Kmr No. 1583. | AGGAGGAC |
| Kmr No. 1584. | AGGAGGAG |
| Kmr No. 1585. | AGGAGGGC |
| Kmr No. 1586. | AGGAGGGG |
| Kmr No. 1587. | AGGAGGTC |
| Kmr No. 1588. | AGGAGGTG |
| Kmr No. 1589. | AGGAGTGC |
| Kmr No. 1590. | AGGATCAA |
| Kmr No. 1591. | AGGATTCC |
| Kmr No. 1592. | AGGATTCG |
| Kmr No. 1593. | AGGCAAAC |
| Kmr No. 1594. | AGGCAAAG |
| Kmr No. 1595. | AGGCAAGG |
| Kmr No. 1596. | AGGCAATC |
| Kmr No. 1597. | AGGCACAA |
| Kmr No. 1598. | AGGCACAG |
| Kmr No. 1599. | AGGCACCC |
| Kmr No. 1600. | AGGCACGA |
| Kmr No. 1601. | AGGCACTC |
| Kmr No. 1602. | AGGCAGAC |
| Kmr No. 1603. | AGGCAGAG |
| Kmr No. 1604. | AGGCAGCG |
| Kmr No. 1605. | AGGCAGGC |
| Kmr No. 1606. | AGGCATAG |
| Kmr No. 1607. | AGGCCAAT |
| Kmr No. 1608. | AGGCCCCC |
| Kmr No. 1609. | AGGCGAAA |
| Kmr No. 1610. | AGGCGACA |
| Kmr No. 1611. | AGGCGCAA |
| Kmr No. 1612. | AGGCGGAG |
| Kmr No. 1613. | AGGCGGGA |
| Kmr No. 1614. | AGGCGGGC |
| Kmr No. 1615. | AGGCGGGG |
| Kmr No. 1616. | AGGCGTAA |
| Kmr No. 1617. | AGGCGTAG |
| Kmr No. 1618. | AGGCTAAG |
| Kmr No. 1619. | AGGCTCAA |
| Kmr No. 1620. | AGGCTCAC |
| Kmr No. 1621. | AGGCTCAG |
| Kmr No. 1622. | AGGCTCTC |
| Kmr No. 1623. | AGGCTCTG |
| Kmr No. 1624. | AGGCTGAC |
| Kmr No. 1625. | AGGCTGAG |
| Kmr No. 1626. | AGGCTGCA |
| Kmr No. 1627. | AGGCTGCG |
| Kmr No. 1628. | AGGCTGGG |
| Kmr No. 1629. | AGGCTGTG |
| Kmr No. 1630. | AGGCTTAC |
| Kmr No. 1631. | AGGCTTGC |
| Kmr No. 1632. | AGGCTTTC |
| Kmr No. 1633. | AGGGACAC |
| Kmr No. 1634. | AGGGACTC |
| Kmr No. 1635. | AGGGAGAG |
| Kmr No. 1636. | AGGGAGCG |
| Kmr No. 1637. | AGGGAGGG |
| Kmr No. 1638. | AGGGCAAA |
| Kmr No. 1639. | AGGGCAAC |
| Kmr No. 1640. | AGGGCAAG |
| Kmr No. 1641. | AGGGCACA |
| Kmr No. 1642. | AGGGCACT |
| Kmr No. 1643. | AGGGCAGA |
| Kmr No. 1644. | AGGGCAGC |
| Kmr No. 1645. | AGGGCAGG |
| Kmr No. 1646. | AGGGCATA |
| Kmr No. 1647. | AGGGCATC |
| Kmr No. 1648. | AGGGCCCC |
| Kmr No. 1649. | AGGGCGAA |
| Kmr No. 1650. | AGGGCGAC |
| Kmr No. 1651. | AGGGCGAG |
| Kmr No. 1652. | AGGGCGCA |
| Kmr No. 1653. | AGGGCGGA |
| Kmr No. 1654. | AGGGCGGC |
| Kmr No. 1655. | AGGGCGGG |
| Kmr No. 1656. | AGGGCGTA |
| Kmr No. 1657. | AGGGCGTG |
| Kmr No. 1658. | AGGGCTAC |
| Kmr No. 1659. | AGGGCTAT |
| Kmr No. 1660. | AGGGCTGC |
| Kmr No. 1661. | AGGGCTTG |
| Kmr No. 1662. | AGGGGAGA |
| Kmr No. 1663. | AGGGGAGG |
| Kmr No. 1664. | AGGGGCAA |
| Kmr No. 1665. | AGGGGCAC |
| Kmr No. 1666. | AGGGGCAG |
| Kmr No. 1667. | AGGGGCCC |
| Kmr No. 1668. | AGGGGCGA |
| Kmr No. 1669. | AGGGGCGC |
| Kmr No. 1670. | AGGGGCGG |
| Kmr No. 1671. | AGGGGCTG |
| Kmr No. 1672. | AGGGGGAC |
| Kmr No. 1673. | AGGGGGAG |
| Kmr No. 1674. | AGGGGGCA |
| Kmr No. 1675. | AGGGGGCC |
| Kmr No. 1676. | AGGGGGCG |
| Kmr No. 1677. | AGGGGGGA |
| Kmr No. 1678. | AGGGGGGC |
| Kmr No. 1679. | AGGGGGGG |
| Kmr No. 1680. | AGGGGGTA |
| Kmr No. 1681. | AGGGGGTC |
| Kmr No. 1682. | AGGGGGTG |
| Kmr No. 1683. | AGGGGTAG |
| Kmr No. 1684. | AGGGGTCA |
| Kmr No. 1685. | AGGGGTCG |
| Kmr No. 1686. | AGGGGTGA |
| Kmr No. 1687. | AGGGGTGC |
| Kmr No. 1688. | AGGGGTGG |
| Kmr No. 1689. | AGGGGTTG |
| Kmr No. 1690. | AGGGTAAG |
| Kmr No. 1691. | AGGGTACC |
| Kmr No. 1692. | AGGGTAGC |
| Kmr No. 1693. | AGGGTCAA |
| Kmr No. 1694. | AGGGTCAG |
| Kmr No. 1695. | AGGGTCCC |
| Kmr No. 1696. | AGGGTCGG |
| Kmr No. 1697. | AGGGTCTC |
| Kmr No. 1698. | AGGGTCTG |
| Kmr No. 1699. | AGGGTGAA |
| Kmr No. 1700. | AGGGTGAC |
| Kmr No. 1701. | AGGGTGAG |
| Kmr No. 1702. | AGGGTGAT |
| Kmr No. 1703. | AGGGTGCA |
| Kmr No. 1704. | AGGGTGCG |
| Kmr No. 1705. | AGGGTGGG |
| Kmr No. 1706. | AGGGTGTA |
| Kmr No. 1707. | AGGGTGTC |
| Kmr No. 1708. | AGGGTGTG |
| Kmr No. 1709. | AGGTATTA |
| Kmr No. 1710. | AGGTCAGC |
| Kmr No. 1711. | AGGTCCGA |
| Kmr No. 1712. | AGGTCGCA |
| Kmr No. 1713. | AGGTCTAT |
| Kmr No. 1714. | AGGTCTGC |
| Kmr No. 1715. | AGGTGCAG |
| Kmr No. 1716. | AGGTGCTG |
| Kmr No. 1717. | AGGTGGAG |
| Kmr No. 1718. | AGGTGGCA |
| Kmr No. 1719. | AGGTGGGC |
| Kmr No. 1720. | AGGTGGGG |
| Kmr No. 1721. | AGGTGTGC |
| Kmr No. 1722. | AGGTTCCA |
| Kmr No. 1723. | AGGTTCCG |
| Kmr No. 1724. | AGGTTGAC |
| Kmr No. 1725. | AGGTTTCC |
| Kmr No. 1726. | AGGTTTGC |
| Kmr No. 1727. | AGTAAAAG |
| Kmr No. 1728. | AGTAAAGA |
| Kmr No. 1729. | AGTAACAG |
| Kmr No. 1730. | AGTAACGA |
| Kmr No. 1731. | AGTAACGG |
| Kmr No. 1732. | AGTAACTG |
| Kmr No. 1733. | AGTAATCC |
| Kmr No. 1734. | AGTAATCG |
| Kmr No. 1735. | AGTAATGC |
| Kmr No. 1736. | AGTAATGG |
| Kmr No. 1737. | AGTAATTC |
| Kmr No. 1738. | AGTACACC |
| Kmr No. 1739. | AGTACAGA |
| Kmr No. 1740. | AGTACAGG |
| Kmr No. 1741. | AGTACCCC |
| Kmr No. 1742. | AGTACCTG |
| Kmr No. 1743. | AGTACGGA |
| Kmr No. 1744. | AGTACGTG |
| Kmr No. 1745. | AGTACTGA |
| Kmr No. 1746. | AGTACTGG |
| Kmr No. 1747. | AGTAGAAC |
| Kmr No. 1748. | AGTAGAAG |
| Kmr No. 1749. | AGTAGACG |
| Kmr No. 1750. | AGTAGAGC |
| Kmr No. 1751. | AGTAGGAA |
| Kmr No. 1752. | AGTAGGGG |
| Kmr No. 1753. | AGTATAGC |
| Kmr No. 1754. | AGTATCCA |
| Kmr No. 1755. | AGTATCCG |
| Kmr No. 1756. | AGTATGTG |
| Kmr No. 1757. | AGTCAATC |
| Kmr No. 1758. | AGTCACAA |
| Kmr No. 1759. | AGTCACAT |
| Kmr No. 1760. | AGTCACTG |
| Kmr No. 1761. | AGTCAGAT |
| Kmr No. 1762. | AGTCAGGA |
| Kmr No. 1763. | AGTCAGTA |
| Kmr No. 1764. | AGTCAGTC |
| Kmr No. 1765. | AGTCAGTG |
| Kmr No. 1766. | AGTCATGA |
| Kmr No. 1767. | AGTCATGG |
| Kmr No. 1768. | AGTCATTG |
| Kmr No. 1769. | AGTCCAAT |
| Kmr No. 1770. | AGTCCAGC |
| Kmr No. 1771. | AGTCCCAT |
| Kmr No. 1772. | AGTCCCCC |
| Kmr No. 1773. | AGTCCCGC |
| Kmr No. 1774. | AGTCCGAT |
| Kmr No. 1775. | AGTCCGGA |
| Kmr No. 1776. | AGTCGCCC |
| Kmr No. 1777. | AGTCGGGG |
| Kmr No. 1778. | AGTCTCGA |
| Kmr No. 1779. | AGTCTCGC |
| Kmr No. 1780. | AGTCTCGG |
| Kmr No. 1781. | AGTCTGAT |
| Kmr No. 1782. | AGTCTGCG |
| Kmr No. 1783. | AGTGAACA |
| Kmr No. 1784. | AGTGAACC |
| Kmr No. 1785. | AGTGAATC |
| Kmr No. 1786. | AGTGACAG |
| Kmr No. 1787. | AGTGACCA |
| Kmr No. 1788. | AGTGACGG |
| Kmr No. 1789. | AGTGACTG |
| Kmr No. 1790. | AGTGAGAG |
| Kmr No. 1791. | AGTGAGCC |
| Kmr No. 1792. | AGTGAGCG |
| Kmr No. 1793. | AGTGAGGG |
| Kmr No. 1794. | AGTGATCA |
| Kmr No. 1795. | AGTGATCC |
| Kmr No. 1796. | AGTGATCG |
| Kmr No. 1797. | AGTGATGA |
| Kmr No. 1798. | AGTGATTC |
| Kmr No. 1799. | AGTGATTG |
| Kmr No. 1800. | AGTGCAAA |
| Kmr No. 1801. | AGTGCAAG |
| Kmr No. 1802. | AGTGCAGC |
| Kmr No. 1803. | AGTGCATC |
| Kmr No. 1804. | AGTGCCAC |
| Kmr No. 1805. | AGTGCCCC |
| Kmr No. 1806. | AGTGCCGC |
| Kmr No. 1807. | AGTGCCTC |
| Kmr No. 1808. | AGTGCGCC |
| Kmr No. 1809. | AGTGCGGG |
| Kmr No. 1810. | AGTGCTTG |
| Kmr No. 1811. | AGTGGAAG |
| Kmr No. 1812. | AGTGGATG |
| Kmr No. 1813. | AGTGGGCA |
| Kmr No. 1814. | AGTGGGGC |
| Kmr No. 1815. | AGTGGGGG |
| Kmr No. 1816. | AGTGGGTG |
| Kmr No. 1817. | AGTGTCCA |
| Kmr No. 1818. | AGTGTCGC |
| Kmr No. 1819. | AGTGTGAG |
| Kmr No. 1820. | AGTGTGCA |
| Kmr No. 1821. | AGTGTGCC |
| Kmr No. 1822. | AGTGTGCG |
| Kmr No. 1823. | AGTGTGGA |
| Kmr No. 1824. | AGTGTGTC |
| Kmr No. 1825. | AGTGTGTG |
| Kmr No. 1826. | AGTGTTCC |
| Kmr No. 1827. | AGTTAACT |
| Kmr No. 1828. | AGTTACCA |
| Kmr No. 1829. | AGTTATCC |
| Kmr No. 1830. | AGTTATGG |
| Kmr No. 1831. | AGTTCCAG |
| Kmr No. 1832. | AGTTCCAT |
| Kmr No. 1833. | AGTTCCGA |
| Kmr No. 1834. | AGTTCCGG |
| Kmr No. 1835. | AGTTCTTC |
| Kmr No. 1836. | AGTTGAGG |
| Kmr No. 1837. | AGTTGCCC |
| Kmr No. 1838. | AGTTTGAG |
| Kmr No. 1839. | ATAAACAG |
| Kmr No. 1840. | ATAAAGTA |
| Kmr No. 1841. | ATAAATAC |
| Kmr No. 1842. | ATAACCAG |
| Kmr No. 1843. | ATAACGTC |
| Kmr No. 1844. | ATAACTGG |
| Kmr No. 1845. | ATAAGCTC |
| Kmr No. 1846. | ATAAGGGC |
| Kmr No. 1847. | ATAAGTGC |
| Kmr No. 1848. | ATAATCCA |
| Kmr No. 1849. | ATAATCTG |
| Kmr No. 1850. | ATAATTCC |
| Kmr No. 1851. | ATACAAAG |
| Kmr No. 1852. | ATACAGAA |
| Kmr No. 1853. | ATACAGAT |
| Kmr No. 1854. | ATACAGGA |
| Kmr No. 1855. | ATACAGTA |
| Kmr No. 1856. | ATACAGTC |
| Kmr No. 1857. | ATACAGTG |
| Kmr No. 1858. | ATACATGG |
| Kmr No. 1859. | ATACCAGG |
| Kmr No. 1860. | ATACCCCC |
| Kmr No. 1861. | ATACCCTC |
| Kmr No. 1862. | ATACCGAG |
| Kmr No. 1863. | ATACCGGA |
| Kmr No. 1864. | ATACGAAC |
| Kmr No. 1865. | ATACGATC |
| Kmr No. 1866. | ATACGCTC |
| Kmr No. 1867. | ATACGGAA |
| Kmr No. 1868. | ATACGGAC |
| Kmr No. 1869. | ATACGGAT |
| Kmr No. 1870. | ATACGGTC |
| Kmr No. 1871. | ATACGGTG |
| Kmr No. 1872. | ATACGTCC |
| Kmr No. 1873. | ATACGTGA |
| Kmr No. 1874. | ATACGTGG |
| Kmr No. 1875. | ATACTCGA |
| Kmr No. 1876. | ATACTCGC |
| Kmr No. 1877. | ATACTCGG |
| Kmr No. 1878. | ATACTGGA |
| Kmr No. 1879. | ATACTTCC |
| Kmr No. 1880. | ATAGACCC |
| Kmr No. 1881. | ATAGACGC |
| Kmr No. 1882. | ATAGAGCA |
| Kmr No. 1883. | ATAGAGCG |
| Kmr No. 1884. | ATAGCACA |
| Kmr No. 1885. | ATAGCGCA |
| Kmr No. 1886. | ATAGCGGG |
| Kmr No. 1887. | ATAGCTCA |
| Kmr No. 1888. | ATAGGCGA |
| Kmr No. 1889. | ATAGGCTC |
| Kmr No. 1890. | ATAGGGCG |
| Kmr No. 1891. | ATAGGGGG |
| Kmr No. 1892. | ATAGGGTG |
| Kmr No. 1893. | ATAGGTCG |
| Kmr No. 1894. | ATAGGTGC |
| Kmr No. 1895. | ATAGGTGG |
| Kmr No. 1896. | ATAGTCGA |
| Kmr No. 1897. | ATAGTCGC |
| Kmr No. 1898. | ATATCATG |
| Kmr No. 1899. | ATATCCAA |
| Kmr No. 1900. | ATATCCAT |
| Kmr No. 1901. | ATATCCGG |
| Kmr No. 1902. | ATATCGAT |
| Kmr No. 1903. | ATATCGGA |
| Kmr No. 1904. | ATATCTCG |
| Kmr No. 1905. | ATATCTGG |
| Kmr No. 1906. | ATATGATC |
| Kmr No. 1907. | ATATGCTC |
| Kmr No. 1908. | ATATGGAA |
| Kmr No. 1909. | ATATGGAT |
| Kmr No. 1910. | ATATGGGA |
| Kmr No. 1911. | ATATGGTC |
| Kmr No. 1912. | ATATGTCG |
| Kmr No. 1913. | ATATTCCA |
| Kmr No. 1914. | ATATTGGA |
| Kmr No. 1915. | ATCAAACG |
| Kmr No. 1916. | ATCAAAGA |
| Kmr No. 1917. | ATCAAATC |
| Kmr No. 1918. | ATCAATAC |
| Kmr No. 1919. | ATCAATCA |
| Kmr No. 1920. | ATCAATCC |
| Kmr No. 1921. | ATCAATCG |
| Kmr No. 1922. | ATCAATGA |
| Kmr No. 1923. | ATCAATGG |
| Kmr No. 1924. | ATCAATTC |
| Kmr No. 1925. | ATCAATTG |
| Kmr No. 1926. | ATCACAAT |
| Kmr No. 1927. | ATCACACG |
| Kmr No. 1928. | ATCACAGG |
| Kmr No. 1929. | ATCACATC |
| Kmr No. 1930. | ATCACCCC |
| Kmr No. 1931. | ATCACCTC |
| Kmr No. 1932. | ATCACGTA |
| Kmr No. 1933. | ATCACTCC |
| Kmr No. 1934. | ATCACTGA |
| Kmr No. 1935. | ATCACTGC |
| Kmr No. 1936. | ATCACTGG |
| Kmr No. 1937. | ATCACTTC |
| Kmr No. 1938. | ATCAGACA |
| Kmr No. 1939. | ATCAGACG |
| Kmr No. 1940. | ATCAGCCA |
| Kmr No. 1941. | ATCAGGAA |
| Kmr No. 1942. | ATCAGGAT |
| Kmr No. 1943. | ATCAGGGA |
| Kmr No. 1944. | ATCAGGTA |
| Kmr No. 1945. | ATCAGTAC |
| Kmr No. 1946. | ATCAGTCA |
| Kmr No. 1947. | ATCAGTCC |
| Kmr No. 1948. | ATCAGTCG |
| Kmr No. 1949. | ATCAGTGA |
| Kmr No. 1950. | ATCAGTGG |
| Kmr No. 1951. | ATCAGTTA |
| Kmr No. 1952. | ATCATAAG |
| Kmr No. 1953. | ATCATACG |
| Kmr No. 1954. | ATCATATC |
| Kmr No. 1955. | ATCATCCA |
| Kmr No. 1956. | ATCATGAA |
| Kmr No. 1957. | ATCATGAT |
| Kmr No. 1958. | ATCATGCA |
| Kmr No. 1959. | ATCATGGA |
| Kmr No. 1960. | ATCATTAC |
| Kmr No. 1961. | ATCATTCA |
| Kmr No. 1962. | ATCATTCC |
| Kmr No. 1963. | ATCATTCG |
| Kmr No. 1964. | ATCATTGA |
| Kmr No. 1965. | ATCATTGG |
| Kmr No. 1966. | ATCATTTC |
| Kmr No. 1967. | ATCCAACA |
| Kmr No. 1968. | ATCCAACC |
| Kmr No. 1969. | ATCCAACG |
| Kmr No. 1970. | ATCCAAGA |
| Kmr No. 1971. | ATCCAAGC |
| Kmr No. 1972. | ATCCAAGG |
| Kmr No. 1973. | ATCCAATC |
| Kmr No. 1974. | ATCCAATG |
| Kmr No. 1975. | ATCCACAG |
| Kmr No. 1976. | ATCCACAT |
| Kmr No. 1977. | ATCCACTG |
| Kmr No. 1978. | ATCCAGAT |
| Kmr No. 1979. | ATCCAGTA |
| Kmr No. 1980. | ATCCAGTC |
| Kmr No. 1981. | ATCCAGTG |
| Kmr No. 1982. | ATCCATAA |
| Kmr No. 1983. | ATCCATAC |
| Kmr No. 1984. | ATCCATCA |
| Kmr No. 1985. | ATCCATCC |
| Kmr No. 1986. | ATCCATGA |
| Kmr No. 1987. | ATCCATGC |
| Kmr No. 1988. | ATCCATTA |
| Kmr No. 1989. | ATCCATTG |
| Kmr No. 1990. | ATCCCATA |
| Kmr No. 1991. | ATCCCATG |
| Kmr No. 1992. | ATCCCCCC |
| Kmr No. 1993. | ATCCCCCG |
| Kmr No. 1994. | ATCCCCGA |
| Kmr No. 1995. | ATCCCCGC |
| Kmr No. 1996. | ATCCCGCA |
| Kmr No. 1997. | ATCCCGCC |
| Kmr No. 1998. | ATCCCTCG |
| Kmr No. 1999. | ATCCCTGA |
| Kmr No. 2000. | ATCCGAAC |
| Kmr No. 2001. | ATCCGAAG |
| Kmr No. 2002. | ATCCGATC |
| Kmr No. 2003. | ATCCGATG |
| Kmr No. 2004. | ATCCGGAA |
| Kmr No. 2005. | ATCCGGAC |
| Kmr No. 2006. | ATCCGGAG |
| Kmr No. 2007. | ATCCGGAT |
| Kmr No. 2008. | ATCCGGTA |
| Kmr No. 2009. | ATCCGGTC |
| Kmr No. 2010. | ATCCGGTG |
| Kmr No. 2011. | ATCCGTAC |
| Kmr No. 2012. | ATCCGTCA |
| Kmr No. 2013. | ATCCGTGA |
| Kmr No. 2014. | ATCCGTGG |
| Kmr No. 2015. | ATCCTACG |
| Kmr No. 2016. | ATCCTCAC |
| Kmr No. 2017. | ATCCTGAA |
| Kmr No. 2018. | ATCCTGTA |
| Kmr No. 2019. | ATCCTGTC |
| Kmr No. 2020. | ATCCTTCC |
| Kmr No. 2021. | ATCCTTGC |
| Kmr No. 2022. | ATCGAACA |
| Kmr No. 2023. | ATCGAATC |
| Kmr No. 2024. | ATCGAGAG |
| Kmr No. 2025. | ATCGATAC |
| Kmr No. 2026. | ATCGATCA |
| Kmr No. 2027. | ATCGATCC |
| Kmr No. 2028. | ATCGATCG |
| Kmr No. 2029. | ATCGATTA |
| Kmr No. 2030. | ATCGGAAC |
| Kmr No. 2031. | ATCGGAAG |
| Kmr No. 2032. | ATCGGAAT |
| Kmr No. 2033. | ATCGGACA |
| Kmr No. 2034. | ATCGGACC |
| Kmr No. 2035. | ATCGGACG |
| Kmr No. 2036. | ATCGGAGA |
| Kmr No. 2037. | ATCGGAGC |
| Kmr No. 2038. | ATCGGAGG |
| Kmr No. 2039. | ATCGGATA |
| Kmr No. 2040. | ATCGGATC |
| Kmr No. 2041. | ATCGGATG |
| Kmr No. 2042. | ATCGGGGG |
| Kmr No. 2043. | ATCGGTAC |
| Kmr No. 2044. | ATCGGTCC |
| Kmr No. 2045. | ATCGGTTC |
| Kmr No. 2046. | ATCGTACG |
| Kmr No. 2047. | ATCGTATC |
| Kmr No. 2048. | ATCGTTAC |
| Kmr No. 2049. | ATCGTTCC |
| Kmr No. 2050. | ATCTCCCA |
| Kmr No. 2051. | ATCTCCGC |
| Kmr No. 2052. | ATCTCCTC |
| Kmr No. 2053. | ATCTCTCG |
| Kmr No. 2054. | ATCTGAAA |
| Kmr No. 2055. | ATCTGAAC |
| Kmr No. 2056. | ATCTGAAT |
| Kmr No. 2057. | ATCTGACG |
| Kmr No. 2058. | ATCTGATC |
| Kmr No. 2059. | ATCTGGAA |
| Kmr No. 2060. | ATCTGGTA |
| Kmr No. 2061. | ATCTGTAA |
| Kmr No. 2062. | ATCTGTAC |
| Kmr No. 2063. | ATCTGTCA |
| Kmr No. 2064. | ATCTGTTC |
| Kmr No. 2065. | ATCTTCCA |
| Kmr No. 2066. | ATCTTCCG |
| Kmr No. 2067. | ATCTTGGA |
| Kmr No. 2068. | ATCTTTCC |
| Kmr No. 2069. | ATCTTTGG |
| Kmr No. 2070. | ATGAACAG |
| Kmr No. 2071. | ATGAACCA |
| Kmr No. 2072. | ATGAACCG |
| Kmr No. 2073. | ATGAATCA |
| Kmr No. 2074. | ATGAATCC |
| Kmr No. 2075. | ATGAATCG |
| Kmr No. 2076. | ATGAATGA |
| Kmr No. 2077. | ATGAATGG |
| Kmr No. 2078. | ATGAATTC |
| Kmr No. 2079. | ATGACCCC |
| Kmr No. 2080. | ATGACGGA |
| Kmr No. 2081. | ATGACTAG |
| Kmr No. 2082. | ATGACTGA |
| Kmr No. 2083. | ATGACTGG |
| Kmr No. 2084. | ATGACTTC |
| Kmr No. 2085. | ATGAGAGC |
| Kmr No. 2086. | ATGAGGGC |
| Kmr No. 2087. | ATGAGGGG |
| Kmr No. 2088. | ATGAGTGA |
| Kmr No. 2089. | ATGATATC |
| Kmr No. 2090. | ATGATCAG |
| Kmr No. 2091. | ATGATCCG |
| Kmr No. 2092. | ATGATCGG |
| Kmr No. 2093. | ATGATGGA |
| Kmr No. 2094. | ATGATTCA |
| Kmr No. 2095. | ATGATTCG |
| Kmr No. 2096. | ATGATTGC |
| Kmr No. 2097. | ATGATTGG |
| Kmr No. 2098. | ATGCACAC |
| Kmr No. 2099. | ATGCACCC |
| Kmr No. 2100. | ATGCACTC |
| Kmr No. 2101. | ATGCATAC |
| Kmr No. 2102. | ATGCATCC |
| Kmr No. 2103. | ATGCATGG |
| Kmr No. 2104. | ATGCATTA |
| Kmr No. 2105. | ATGCATTG |
| Kmr No. 2106. | ATGCCCAC |
| Kmr No. 2107. | ATGCCCCC |
| Kmr No. 2108. | ATGCCCCG |
| Kmr No. 2109. | ATGCCCTC |
| Kmr No. 2110. | ATGCGAAT |
| Kmr No. 2111. | ATGCGGAA |
| Kmr No. 2112. | ATGCGGGG |
| Kmr No. 2113. | ATGCGTGA |
| Kmr No. 2114. | ATGCTCAA |
| Kmr No. 2115. | ATGCTCTC |
| Kmr No. 2116. | ATGCTGCC |
| Kmr No. 2117. | ATGCTTCC |
| Kmr No. 2118. | ATGGAAAC |
| Kmr No. 2119. | ATGGAAAT |
| Kmr No. 2120. | ATGGAACA |
| Kmr No. 2121. | ATGGAACC |
| Kmr No. 2122. | ATGGAAGA |
| Kmr No. 2123. | ATGGAAGC |
| Kmr No. 2124. | ATGGAAGG |
| Kmr No. 2125. | ATGGACCA |
| Kmr No. 2126. | ATGGACCG |
| Kmr No. 2127. | ATGGAGGG |
| Kmr No. 2128. | ATGGATAC |
| Kmr No. 2129. | ATGGATCC |
| Kmr No. 2130. | ATGGATCG |
| Kmr No. 2131. | ATGGATGA |
| Kmr No. 2132. | ATGGATGC |
| Kmr No. 2133. | ATGGATGG |
| Kmr No. 2134. | ATGGATTC |
| Kmr No. 2135. | ATGGCGGG |
| Kmr No. 2136. | ATGGGAAC |
| Kmr No. 2137. | ATGGGAAG |
| Kmr No. 2138. | ATGGGAAT |
| Kmr No. 2139. | ATGGGACC |
| Kmr No. 2140. | ATGGGAGC |
| Kmr No. 2141. | ATGGGATC |
| Kmr No. 2142. | ATGGGCAG |
| Kmr No. 2143. | ATGGGCTA |
| Kmr No. 2144. | ATGGGGGG |
| Kmr No. 2145. | ATGGGTCA |
| Kmr No. 2146. | ATGGTCCA |
| Kmr No. 2147. | ATGGTCCG |
| Kmr No. 2148. | ATGGTGGG |
| Kmr No. 2149. | ATGGTTCC |
| Kmr No. 2150. | ATGGTTTC |
| Kmr No. 2151. | ATGTACAT |
| Kmr No. 2152. | ATGTACTG |
| Kmr No. 2153. | ATGTAGGA |
| Kmr No. 2154. | ATGTATCC |
| Kmr No. 2155. | ATGTCAAG |
| Kmr No. 2156. | ATGTCCAA |
| Kmr No. 2157. | ATGTCCTC |
| Kmr No. 2158. | ATGTCGGG |
| Kmr No. 2159. | ATGTCTGC |
| Kmr No. 2160. | ATGTGACA |
| Kmr No. 2161. | ATGTGAGC |
| Kmr No. 2162. | ATGTGATC |
| Kmr No. 2163. | ATGTGGAA |
| Kmr No. 2164. | ATGTGGGC |
| Kmr No. 2165. | ATGTGGGG |
| Kmr No. 2166. | ATGTGTGC |
| Kmr No. 2167. | ATGTTCCA |
| Kmr No. 2168. | ATGTTCCG |
| Kmr No. 2169. | ATGTTGGA |
| Kmr No. 2170. | ATGTTGTC |
| Kmr No. 2171. | ATTAAAAG |
| Kmr No. 2172. | ATTAAAGA |
| Kmr No. 2173. | ATTAAAGG |
| Kmr No. 2174. | ATTAATCC |
| Kmr No. 2175. | ATTAATCG |
| Kmr No. 2176. | ATTAATGA |
| Kmr No. 2177. | ATTAATGC |
| Kmr No. 2178. | ATTAATGG |
| Kmr No. 2179. | ATTACAGA |
| Kmr No. 2180. | ATTACATG |
| Kmr No. 2181. | ATTACCAG |
| Kmr No. 2182. | ATTACCTG |
| Kmr No. 2183. | ATTACGTG |
| Kmr No. 2184. | ATTACTGA |
| Kmr No. 2185. | ATTACTGG |
| Kmr No. 2186. | ATTAGAAG |
| Kmr No. 2187. | ATTAGCGG |
| Kmr No. 2188. | ATTAGTGG |
| Kmr No. 2189. | ATTATCTG |
| Kmr No. 2190. | ATTATGGA |
| Kmr No. 2191. | ATTATGGG |
| Kmr No. 2192. | ATTCAATC |
| Kmr No. 2193. | ATTCACCG |
| Kmr No. 2194. | ATTCACTG |
| Kmr No. 2195. | ATTCAGGA |
| Kmr No. 2196. | ATTCAGTC |
| Kmr No. 2197. | ATTCAGTG |
| Kmr No. 2198. | ATTCATCC |
| Kmr No. 2199. | ATTCATGA |
| Kmr No. 2200. | ATTCATGG |
| Kmr No. 2201. | ATTCATTC |
| Kmr No. 2202. | ATTCCAAA |
| Kmr No. 2203. | ATTCCAAC |
| Kmr No. 2204. | ATTCCAAT |
| Kmr No. 2205. | ATTCCACA |
| Kmr No. 2206. | ATTCCACC |
| Kmr No. 2207. | ATTCCACG |
| Kmr No. 2208. | ATTCCAGA |
| Kmr No. 2209. | ATTCCAGG |
| Kmr No. 2210. | ATTCCATA |
| Kmr No. 2211. | ATTCCATC |
| Kmr No. 2212. | ATTCCATG |
| Kmr No. 2213. | ATTCCCAG |
| Kmr No. 2214. | ATTCCGGA |
| Kmr No. 2215. | ATTCCGGC |
| Kmr No. 2216. | ATTCCGTA |
| Kmr No. 2217. | ATTCCTAC |
| Kmr No. 2218. | ATTCCTGA |
| Kmr No. 2219. | ATTCCTGC |
| Kmr No. 2220. | ATTCCTGG |
| Kmr No. 2221. | ATTCCTTC |
| Kmr No. 2222. | ATTCGAAA |
| Kmr No. 2223. | ATTCGAAC |
| Kmr No. 2224. | ATTCGAAG |
| Kmr No. 2225. | ATTCGGAA |
| Kmr No. 2226. | ATTCGGAC |
| Kmr No. 2227. | ATTCGTAC |
| Kmr No. 2228. | ATTCGTCA |
| Kmr No. 2229. | ATTCGTGA |
| Kmr No. 2230. | ATTCGTGG |
| Kmr No. 2231. | ATTCGTTC |
| Kmr No. 2232. | ATTCTAGG |
| Kmr No. 2233. | ATTCTTCC |
| Kmr No. 2234. | ATTCTTTA |
| Kmr No. 2235. | ATTGAAGA |
| Kmr No. 2236. | ATTGAATC |
| Kmr No. 2237. | ATTGACCA |
| Kmr No. 2238. | ATTGACGG |
| Kmr No. 2239. | ATTGACTG |
| Kmr No. 2240. | ATTGATCA |
| Kmr No. 2241. | ATTGATCC |
| Kmr No. 2242. | ATTGATCG |
| Kmr No. 2243. | ATTGATTC |
| Kmr No. 2244. | ATTGATTG |
| Kmr No. 2245. | ATTGCAGG |
| Kmr No. 2246. | ATTGCATG |
| Kmr No. 2247. | ATTGCCTG |
| Kmr No. 2248. | ATTGCTGG |
| Kmr No. 2249. | ATTGCTTC |
| Kmr No. 2250. | ATTGGAAC |
| Kmr No. 2251. | ATTGGAAG |
| Kmr No. 2252. | ATTGGACA |
| Kmr No. 2253. | ATTGGACC |
| Kmr No. 2254. | ATTGGACG |
| Kmr No. 2255. | ATTGGAGC |
| Kmr No. 2256. | ATTGGATC |
| Kmr No. 2257. | ATTGGATG |
| Kmr No. 2258. | ATTGGCCA |
| Kmr No. 2259. | ATTGGCGG |
| Kmr No. 2260. | ATTGGCTG |
| Kmr No. 2261. | ATTGGTCC |
| Kmr No. 2262. | ATTGGTCG |
| Kmr No. 2263. | ATTGGTGA |
| Kmr No. 2264. | ATTGGTGC |
| Kmr No. 2265. | ATTGGTGG |
| Kmr No. 2266. | ATTGGTTC |
| Kmr No. 2267. | ATTGTGAC |
| Kmr No. 2268. | ATTGTGGA |
| Kmr No. 2269. | ATTGTTCC |
| Kmr No. 2270. | ATTTAAAG |
| Kmr No. 2271. | ATTTACCA |
| Kmr No. 2272. | ATTTATGG |
| Kmr No. 2273. | ATTTCATG |
| Kmr No. 2274. | ATTTCCAA |
| Kmr No. 2275. | ATTTCCAC |
| Kmr No. 2276. | ATTTCCAG |
| Kmr No. 2277. | ATTTCCCA |
| Kmr No. 2278. | ATTTCCGG |
| Kmr No. 2279. | ATTTCCTG |
| Kmr No. 2280. | ATTTGATC |
| Kmr No. 2281. | ATTTGGAA |
| Kmr No. 2282. | ATTTGGAC |
| Kmr No. 2283. | ATTTGGTC |
| Kmr No. 2284. | ATTTGGTG |
| Kmr No. 2285. | ATTTTCGC |
| Kmr No. 2286. | ATTTTGCC |
| Kmr No. 2287. | ATTTTGCG |
| Kmr No. 2288. | CAAAAAAA |
| Kmr No. 2289. | CAAAAAAC |
| Kmr No. 2290. | CAAAAAGC |
| Kmr No. 2291. | CAAAAAGG |
| Kmr No. 2292. | CAAAACCC |
| Kmr No. 2293. | CAAAACGC |
| Kmr No. 2294. | CAAAAGGC |
| Kmr No. 2295. | CAAAATGC |
| Kmr No. 2296. | CAAACACC |
| Kmr No. 2297. | CAAACACG |
| Kmr No. 2298. | CAAACCCC |
| Kmr No. 2299. | CAAACCCG |
| Kmr No. 2300. | CAAACCTC |
| Kmr No. 2301. | CAAACGAG |
| Kmr No. 2302. | CAAACGTC |
| Kmr No. 2303. | CAAACTGC |
| Kmr No. 2304. | CAAAGACC |
| Kmr No. 2305. | CAAAGAGC |
| Kmr No. 2306. | CAAAGAGG |
| Kmr No. 2307. | CAAAGCCC |
| Kmr No. 2308. | CAAAGCGC |
| Kmr No. 2309. | CAAAGCTC |
| Kmr No. 2310. | CAAAGCTG |
| Kmr No. 2311. | CAAAGGCG |
| Kmr No. 2312. | CAAAGGGC |
| Kmr No. 2313. | CAAAGGTC |
| Kmr No. 2314. | CAAATCCA |
| Kmr No. 2315. | CAACAAGC |
| Kmr No. 2316. | CAACAAGG |
| Kmr No. 2317. | CAACACAA |
| Kmr No. 2318. | CAACACAC |
| Kmr No. 2319. | CAACACCC |
| Kmr No. 2320. | CAACAGGA |
| Kmr No. 2321. | CAACATAG |
| Kmr No. 2322. | CAACATCC |
| Kmr No. 2323. | CAACCACA |
| Kmr No. 2324. | CAACCCCA |
| Kmr No. 2325. | CAACCCCC |
| Kmr No. 2326. | CAACCCCG |
| Kmr No. 2327. | CAACCCTC |
| Kmr No. 2328. | CAACCCTG |
| Kmr No. 2329. | CAACCGTA |
| Kmr No. 2330. | CAACCGTC |
| Kmr No. 2331. | CAACCTCC |
| Kmr No. 2332. | CAACGAGA |
| Kmr No. 2333. | CAACGCCC |
| Kmr No. 2334. | CAACTATG |
| Kmr No. 2335. | CAACTCTG |
| Kmr No. 2336. | CAACTGCC |
| Kmr No. 2337. | CAAGACCC |
| Kmr No. 2338. | CAAGAGCC |
| Kmr No. 2339. | CAAGAGCG |
| Kmr No. 2340. | CAAGAGGG |
| Kmr No. 2341. | CAAGATGC |
| Kmr No. 2342. | CAAGCACA |
| Kmr No. 2343. | CAAGCCCC |
| Kmr No. 2344. | CAAGCCTG |
| Kmr No. 2345. | CAAGCGGG |
| Kmr No. 2346. | CAAGCTCC |
| Kmr No. 2347. | CAAGCTGA |
| Kmr No. 2348. | CAAGGACA |
| Kmr No. 2349. | CAAGGAGA |
| Kmr No. 2350. | CAAGGAGC |
| Kmr No. 2351. | CAAGGAGG |
| Kmr No. 2352. | CAAGGCAG |
| Kmr No. 2353. | CAAGGCGG |
| Kmr No. 2354. | CAAGGCTC |
| Kmr No. 2355. | CAAGGCTG |
| Kmr No. 2356. | CAAGGGAC |
| Kmr No. 2357. | CAAGGGCA |
| Kmr No. 2358. | CAAGGGGC |
| Kmr No. 2359. | CAAGGGGG |
| Kmr No. 2360. | CAAGGGTC |
| Kmr No. 2361. | CAAGGGTG |
| Kmr No. 2362. | CAATAAGC |
| Kmr No. 2363. | CAATAATC |
| Kmr No. 2364. | CAATCACA |
| Kmr No. 2365. | CAATCACC |
| Kmr No. 2366. | CAATCAGA |
| Kmr No. 2367. | CAATCATG |
| Kmr No. 2368. | CAATCCAG |
| Kmr No. 2369. | CAATCCCA |
| Kmr No. 2370. | CAATCCGG |
| Kmr No. 2371. | CAATCCTG |
| Kmr No. 2372. | CAATCGGA |
| Kmr No. 2373. | CAATCGTA |
| Kmr No. 2374. | CAATCGTC |
| Kmr No. 2375. | CAATCTGA |
| Kmr No. 2376. | CAATGAGC |
| Kmr No. 2377. | CAATGATC |
| Kmr No. 2378. | CAATGTGC |
| Kmr No. 2379. | CAATTCCG |
| Kmr No. 2380. | CAATTGGA |
| Kmr No. 2381. | CAATTGGC |
| Kmr No. 2382. | CAATTGTC |
| Kmr No. 2383. | CACAAAAA |
| Kmr No. 2384. | CACAAACA |
| Kmr No. 2385. | CACAAACC |
| Kmr No. 2386. | CACAAACG |
| Kmr No. 2387. | CACAAAGC |
| Kmr No. 2388. | CACAAAGG |
| Kmr No. 2389. | CACAACAA |
| Kmr No. 2390. | CACAACAC |
| Kmr No. 2391. | CACAACCC |
| Kmr No. 2392. | CACAACGA |
| Kmr No. 2393. | CACAACGC |
| Kmr No. 2394. | CACAAGAC |
| Kmr No. 2395. | CACAAGCA |
| Kmr No. 2396. | CACAAGCG |
| Kmr No. 2397. | CACAAGGA |
| Kmr No. 2398. | CACAAGGC |
| Kmr No. 2399. | CACAAGGG |
| Kmr No. 2400. | CACACAAA |
| Kmr No. 2401. | CACACAAC |
| Kmr No. 2402. | CACACACA |
| Kmr No. 2403. | CACACACC |
| Kmr No. 2404. | CACACACG |
| Kmr No. 2405. | CACACAGC |
| Kmr No. 2406. | CACACCCA |
| Kmr No. 2407. | CACACCCC |
| Kmr No. 2408. | CACACCCG |
| Kmr No. 2409. | CACACCTA |
| Kmr No. 2410. | CACACCTC |
| Kmr No. 2411. | CACACGGG |
| Kmr No. 2412. | CACACTCA |
| Kmr No. 2413. | CACACTCC |
| Kmr No. 2414. | CACACTCG |
| Kmr No. 2415. | CACAGAGC |
| Kmr No. 2416. | CACAGCAA |
| Kmr No. 2417. | CACAGCAC |
| Kmr No. 2418. | CACAGCCC |
| Kmr No. 2419. | CACAGCGA |
| Kmr No. 2420. | CACAGCGC |
| Kmr No. 2421. | CACAGCGG |
| Kmr No. 2422. | CACAGCTC |
| Kmr No. 2423. | CACAGGAA |
| Kmr No. 2424. | CACAGGGC |
| Kmr No. 2425. | CACAGTGC |
| Kmr No. 2426. | CACATAGA |
| Kmr No. 2427. | CACATAGC |
| Kmr No. 2428. | CACATTGC |
| Kmr No. 2429. | CACCAATC |
| Kmr No. 2430. | CACCAATG |
| Kmr No. 2431. | CACCACAA |
| Kmr No. 2432. | CACCACAC |
| Kmr No. 2433. | CACCACCC |
| Kmr No. 2434. | CACCAGCC |
| Kmr No. 2435. | CACCCACA |
| Kmr No. 2436. | CACCCACC |
| Kmr No. 2437. | CACCCAGA |
| Kmr No. 2438. | CACCCAGC |
| Kmr No. 2439. | CACCCCAA |
| Kmr No. 2440. | CACCCCAC |
| Kmr No. 2441. | CACCCCAG |
| Kmr No. 2442. | CACCCCCA |
| Kmr No. 2443. | CACCCCCC |
| Kmr No. 2444. | CACCCCCG |
| Kmr No. 2445. | CACCCCGC |
| Kmr No. 2446. | CACCCCGG |
| Kmr No. 2447. | CACCCCTA |
| Kmr No. 2448. | CACCCCTC |
| Kmr No. 2449. | CACCCCTG |
| Kmr No. 2450. | CACCCGAC |
| Kmr No. 2451. | CACCCGCA |
| Kmr No. 2452. | CACCCGCC |
| Kmr No. 2453. | CACCCGGC |
| Kmr No. 2454. | CACCCGGG |
| Kmr No. 2455. | CACCCTAC |
| Kmr No. 2456. | CACCCTCA |
| Kmr No. 2457. | CACCCTCC |
| Kmr No. 2458. | CACCCTGA |
| Kmr No. 2459. | CACCCTGC |
| Kmr No. 2460. | CACCCTTC |
| Kmr No. 2461. | CACCGCCC |
| Kmr No. 2462. | CACCGCTC |
| Kmr No. 2463. | CACCGCTG |
| Kmr No. 2464. | CACCGGAA |
| Kmr No. 2465. | CACCGGGG |
| Kmr No. 2466. | CACCTATC |
| Kmr No. 2467. | CACCTATG |
| Kmr No. 2468. | CACCTCAA |
| Kmr No. 2469. | CACCTCAC |
| Kmr No. 2470. | CACCTCAG |
| Kmr No. 2471. | CACCTCCC |
| Kmr No. 2472. | CACCTCCG |
| Kmr No. 2473. | CACCTCTA |
| Kmr No. 2474. | CACCTCTC |
| Kmr No. 2475. | CACCTGCC |
| Kmr No. 2476. | CACCTTGC |
| Kmr No. 2477. | CACGAAAA |
| Kmr No. 2478. | CACGAAAG |
| Kmr No. 2479. | CACGAATC |
| Kmr No. 2480. | CACGACAA |
| Kmr No. 2481. | CACGACAG |
| Kmr No. 2482. | CACGACCC |
| Kmr No. 2483. | CACGAGAA |
| Kmr No. 2484. | CACGAGAG |
| Kmr No. 2485. | CACGAGGG |
| Kmr No. 2486. | CACGCCCC |
| Kmr No. 2487. | CACGCTCC |
| Kmr No. 2488. | CACGGAGA |
| Kmr No. 2489. | CACGGAGC |
| Kmr No. 2490. | CACGGAGG |
| Kmr No. 2491. | CACGGCAG |
| Kmr No. 2492. | CACGGGGA |
| Kmr No. 2493. | CACGGGGC |
| Kmr No. 2494. | CACGGGGG |
| Kmr No. 2495. | CACGGTTC |
| Kmr No. 2496. | CACGTAAA |
| Kmr No. 2497. | CACGTAAC |
| Kmr No. 2498. | CACGTACA |
| Kmr No. 2499. | CACGTACC |
| Kmr No. 2500. | CACGTACG |
| Kmr No. 2501. | CACGTATC |
| Kmr No. 2502. | CACGTATG |
| Kmr No. 2503. | CACGTGAA |
| Kmr No. 2504. | CACGTGAC |
| Kmr No. 2505. | CACGTTCC |
| Kmr No. 2506. | CACGTTTA |
| Kmr No. 2507. | CACTATGC |
| Kmr No. 2508. | CACTCACA |
| Kmr No. 2509. | CACTCACG |
| Kmr No. 2510. | CACTCAGC |
| Kmr No. 2511. | CACTCCAC |
| Kmr No. 2512. | CACTCCTC |
| Kmr No. 2513. | CACTCGCA |
| Kmr No. 2514. | CACTCGGA |
| Kmr No. 2515. | CACTCGGG |
| Kmr No. 2516. | CACTCTAC |
| Kmr No. 2517. | CACTCTCA |
| Kmr No. 2518. | CACTCTCC |
| Kmr No. 2519. | CACTCTCG |
| Kmr No. 2520. | CACTCTGC |
| Kmr No. 2521. | CACTGAAG |
| Kmr No. 2522. | CACTGACA |
| Kmr No. 2523. | CACTGAGG |
| Kmr No. 2524. | CACTGATA |
| Kmr No. 2525. | CACTGATC |
| Kmr No. 2526. | CACTGCCC |
| Kmr No. 2527. | CACTGGAA |
| Kmr No. 2528. | CACTGGGG |
| Kmr No. 2529. | CACTGGTA |
| Kmr No. 2530. | CACTGTAA |
| Kmr No. 2531. | CACTGTAG |
| Kmr No. 2532. | CACTGTTA |
| Kmr No. 2533. | CACTGTTC |
| Kmr No. 2534. | CACTTCAG |
| Kmr No. 2535. | CACTTCCA |
| Kmr No. 2536. | CACTTCCG |
| Kmr No. 2537. | CACTTGAG |
| Kmr No. 2538. | CACTTGGA |
| Kmr No. 2539. | CAGAACGA |
| Kmr No. 2540. | CAGAATCC |
| Kmr No. 2541. | CAGAATGC |
| Kmr No. 2542. | CAGACACA |
| Kmr No. 2543. | CAGACACC |
| Kmr No. 2544. | CAGACCCA |
| Kmr No. 2545. | CAGACCCC |
| Kmr No. 2546. | CAGACCCG |
| Kmr No. 2547. | CAGACCTC |
| Kmr No. 2548. | CAGAGACC |
| Kmr No. 2549. | CAGAGAGC |
| Kmr No. 2550. | CAGAGAGG |
| Kmr No. 2551. | CAGAGCAA |
| Kmr No. 2552. | CAGAGCAC |
| Kmr No. 2553. | CAGAGCCA |
| Kmr No. 2554. | CAGAGCCG |
| Kmr No. 2555. | CAGAGCGA |
| Kmr No. 2556. | CAGAGCGC |
| Kmr No. 2557. | CAGAGCGG |
| Kmr No. 2558. | CAGAGCTA |
| Kmr No. 2559. | CAGAGGGC |
| Kmr No. 2560. | CAGAGGGG |
| Kmr No. 2561. | CAGAGGTC |
| Kmr No. 2562. | CAGAGTCG |
| Kmr No. 2563. | CAGAGTGC |
| Kmr No. 2564. | CAGAGTGG |
| Kmr No. 2565. | CAGATAAC |
| Kmr No. 2566. | CAGATAGC |
| Kmr No. 2567. | CAGATATC |
| Kmr No. 2568. | CAGATTAC |
| Kmr No. 2569. | CAGATTCA |
| Kmr No. 2570. | CAGATTCC |
| Kmr No. 2571. | CAGATTGA |
| Kmr No. 2572. | CAGCAAGC |
| Kmr No. 2573. | CAGCAAGG |
| Kmr No. 2574. | CAGCAATC |
| Kmr No. 2575. | CAGCACAA |
| Kmr No. 2576. | CAGCACAC |
| Kmr No. 2577. | CAGCACCC |
| Kmr No. 2578. | CAGCACTC |
| Kmr No. 2579. | CAGCAGCC |
| Kmr No. 2580. | CAGCAGGG |
| Kmr No. 2581. | CAGCCCAC |
| Kmr No. 2582. | CAGCCCCC |
| Kmr No. 2583. | CAGCCCCG |
| Kmr No. 2584. | CAGCCCGC |
| Kmr No. 2585. | CAGCCCTC |
| Kmr No. 2586. | CAGCCGCA |
| Kmr No. 2587. | CAGCCTCA |
| Kmr No. 2588. | CAGCCTCC |
| Kmr No. 2589. | CAGCCTGC |
| Kmr No. 2590. | CAGCGAAA |
| Kmr No. 2591. | CAGCGACA |
| Kmr No. 2592. | CAGCGAGA |
| Kmr No. 2593. | CAGCGAGG |
| Kmr No. 2594. | CAGCGATA |
| Kmr No. 2595. | CAGCGCAA |
| Kmr No. 2596. | CAGCGCAG |
| Kmr No. 2597. | CAGCGCCC |
| Kmr No. 2598. | CAGCGCCG |
| Kmr No. 2599. | CAGCGCTA |
| Kmr No. 2600. | CAGCGCTG |
| Kmr No. 2601. | CAGCGGCA |
| Kmr No. 2602. | CAGCGGCG |
| Kmr No. 2603. | CAGCGGGA |
| Kmr No. 2604. | CAGCGGGC |
| Kmr No. 2605. | CAGCGGGG |
| Kmr No. 2606. | CAGCGGTA |
| Kmr No. 2607. | CAGCGTAG |
| Kmr No. 2608. | CAGCGTCC |
| Kmr No. 2609. | CAGCTACG |
| Kmr No. 2610. | CAGCTCTA |
| Kmr No. 2611. | CAGCTCTC |
| Kmr No. 2612. | CAGCTTAG |
| Kmr No. 2613. | CAGCTTGC |
| Kmr No. 2614. | CAGGAAAC |
| Kmr No. 2615. | CAGGAACA |
| Kmr No. 2616. | CAGGAACC |
| Kmr No. 2617. | CAGGAACG |
| Kmr No. 2618. | CAGGAAGA |
| Kmr No. 2619. | CAGGAAGC |
| Kmr No. 2620. | CAGGAATC |
| Kmr No. 2621. | CAGGAATG |
| Kmr No. 2622. | CAGGAGCG |
| Kmr No. 2623. | CAGGAGGG |
| Kmr No. 2624. | CAGGATTA |
| Kmr No. 2625. | CAGGATTC |
| Kmr No. 2626. | CAGGCACA |
| Kmr No. 2627. | CAGGCGGG |
| Kmr No. 2628. | CAGGGCAA |
| Kmr No. 2629. | CAGGGCTA |
| Kmr No. 2630. | CAGGGGCA |
| Kmr No. 2631. | CAGGGGCG |
| Kmr No. 2632. | CAGGGGGC |
| Kmr No. 2633. | CAGGGGGG |
| Kmr No. 2634. | CAGGGGTC |
| Kmr No. 2635. | CAGGGTGC |
| Kmr No. 2636. | CAGGTAAA |
| Kmr No. 2637. | CAGGTAAC |
| Kmr No. 2638. | CAGGTACA |
| Kmr No. 2639. | CAGGTACG |
| Kmr No. 2640. | CAGGTGGG |
| Kmr No. 2641. | CAGGTTCC |
| Kmr No. 2642. | CAGTAACA |
| Kmr No. 2643. | CAGTAACG |
| Kmr No. 2644. | CAGTAATA |
| Kmr No. 2645. | CAGTAATC |
| Kmr No. 2646. | CAGTAATG |
| Kmr No. 2647. | CAGTACAA |
| Kmr No. 2648. | CAGTACAG |
| Kmr No. 2649. | CAGTACCA |
| Kmr No. 2650. | CAGTACCG |
| Kmr No. 2651. | CAGTAGAA |
| Kmr No. 2652. | CAGTAGAC |
| Kmr No. 2653. | CAGTAGGA |
| Kmr No. 2654. | CAGTAGGC |
| Kmr No. 2655. | CAGTATCC |
| Kmr No. 2656. | CAGTCAAA |
| Kmr No. 2657. | CAGTCACA |
| Kmr No. 2658. | CAGTCACC |
| Kmr No. 2659. | CAGTCAGA |
| Kmr No. 2660. | CAGTCATA |
| Kmr No. 2661. | CAGTCATG |
| Kmr No. 2662. | CAGTCCAA |
| Kmr No. 2663. | CAGTCTCG |
| Kmr No. 2664. | CAGTGAAA |
| Kmr No. 2665. | CAGTGAAC |
| Kmr No. 2666. | CAGTGAAG |
| Kmr No. 2667. | CAGTGACA |
| Kmr No. 2668. | CAGTGACG |
| Kmr No. 2669. | CAGTGATA |
| Kmr No. 2670. | CAGTGATC |
| Kmr No. 2671. | CAGTGATG |
| Kmr No. 2672. | CAGTGCCC |
| Kmr No. 2673. | CAGTGGAA |
| Kmr No. 2674. | CAGTGGTA |
| Kmr No. 2675. | CAGTGTAC |
| Kmr No. 2676. | CAGTGTGA |
| Kmr No. 2677. | CAGTTACC |
| Kmr No. 2678. | CAGTTACG |
| Kmr No. 2679. | CAGTTATC |
| Kmr No. 2680. | CAGTTATG |
| Kmr No. 2681. | CAGTTCAC |
| Kmr No. 2682. | CAGTTCCA |
| Kmr No. 2683. | CAGTTCCG |
| Kmr No. 2684. | CAGTTGTA |
| Kmr No. 2685. | CATAAATA |
| Kmr No. 2686. | CATAACCA |
| Kmr No. 2687. | CATAACCG |
| Kmr No. 2688. | CATAAGGA |
| Kmr No. 2689. | CATAATCC |
| Kmr No. 2690. | CATACGGA |
| Kmr No. 2691. | CATACTCG |
| Kmr No. 2692. | CATAGACG |
| Kmr No. 2693. | CATAGAGC |
| Kmr No. 2694. | CATAGCAC |
| Kmr No. 2695. | CATAGCCC |
| Kmr No. 2696. | CATAGCGC |
| Kmr No. 2697. | CATAGCGG |
| Kmr No. 2698. | CATAGCTC |
| Kmr No. 2699. | CATAGGGG |
| Kmr No. 2700. | CATAGGTC |
| Kmr No. 2701. | CATAGTCG |
| Kmr No. 2702. | CATAGTGC |
| Kmr No. 2703. | CATATCCA |
| Kmr No. 2704. | CATATCGC |
| Kmr No. 2705. | CATCAAGG |
| Kmr No. 2706. | CATCAATC |
| Kmr No. 2707. | CATCACCC |
| Kmr No. 2708. | CATCAGAC |
| Kmr No. 2709. | CATCAGCA |
| Kmr No. 2710. | CATCCAAA |
| Kmr No. 2711. | CATCCAAC |
| Kmr No. 2712. | CATCCAAG |
| Kmr No. 2713. | CATCCATA |
| Kmr No. 2714. | CATCCCCC |
| Kmr No. 2715. | CATCCCCG |
| Kmr No. 2716. | CATCCCGC |
| Kmr No. 2717. | CATCCGGA |
| Kmr No. 2718. | CATCCTGC |
| Kmr No. 2719. | CATCGGAA |
| Kmr No. 2720. | CATCGGGG |
| Kmr No. 2721. | CATCTGAC |
| Kmr No. 2722. | CATGAATC |
| Kmr No. 2723. | CATGACTA |
| Kmr No. 2724. | CATGATTC |
| Kmr No. 2725. | CATGCAAG |
| Kmr No. 2726. | CATGGAAA |
| Kmr No. 2727. | CATGGAAC |
| Kmr No. 2728. | CATGGAAG |
| Kmr No. 2729. | CATGGATA |
| Kmr No. 2730. | CATGGGAA |
| Kmr No. 2731. | CATGGTTC |
| Kmr No. 2732. | CATGTACA |
| Kmr No. 2733. | CATGTACC |
| Kmr No. 2734. | CATGTAGC |
| Kmr No. 2735. | CATGTCAC |
| Kmr No. 2736. | CATGTTCC |
| Kmr No. 2737. | CATTAAAG |
| Kmr No. 2738. | CATTAATC |
| Kmr No. 2739. | CATTAATG |
| Kmr No. 2740. | CATTACAG |
| Kmr No. 2741. | CATTATGG |
| Kmr No. 2742. | CATTCCAA |
| Kmr No. 2743. | CATTCCCA |
| Kmr No. 2744. | CATTCGAA |
| Kmr No. 2745. | CATTCGTA |
| Kmr No. 2746. | CATTCTGC |
| Kmr No. 2747. | CATTGAGC |
| Kmr No. 2748. | CATTGAGG |
| Kmr No. 2749. | CATTGATC |
| Kmr No. 2750. | CATTGGAA |
| Kmr No. 2751. | CATTGGAC |
| Kmr No. 2752. | CATTGGTC |
| Kmr No. 2753. | CATTTCCA |
| Kmr No. 2754. | CATTTGGA |
| Kmr No. 2755. | CATTTTGC |
| Kmr No. 2756. | CCAAAAAA |
| Kmr No. 2757. | CCAAACAC |
| Kmr No. 2758. | CCAAACCC |
| Kmr No. 2759. | CCAAATAC |
| Kmr No. 2760. | CCAAATCC |
| Kmr No. 2761. | CCAAATGC |
| Kmr No. 2762. | CCAACCCC |
| Kmr No. 2763. | CCAACCTC |
| Kmr No. 2764. | CCAAGGGG |
| Kmr No. 2765. | CCAAGTAC |
| Kmr No. 2766. | CCAAGTCC |
| Kmr No. 2767. | CCAAGTGA |
| Kmr No. 2768. | CCAATCAA |
| Kmr No. 2769. | CCAATCAC |
| Kmr No. 2770. | CCAATCAG |
| Kmr No. 2771. | CCAATCCG |
| Kmr No. 2772. | CCAATCGG |
| Kmr No. 2773. | CCAATGAC |
| Kmr No. 2774. | CCAATGCA |
| Kmr No. 2775. | CCAATGGA |
| Kmr No. 2776. | CCAATTCC |
| Kmr No. 2777. | CCAATTGA |
| Kmr No. 2778. | CCAATTGC |
| Kmr No. 2779. | CCAATTGG |
| Kmr No. 2780. | CCACAAAA |
| Kmr No. 2781. | CCACAAAC |
| Kmr No. 2782. | CCACAAAG |
| Kmr No. 2783. | CCACAACA |
| Kmr No. 2784. | CCACAACC |
| Kmr No. 2785. | CCACAAGA |
| Kmr No. 2786. | CCACAAGC |
| Kmr No. 2787. | CCACAAGG |
| Kmr No. 2788. | CCACAATC |
| Kmr No. 2789. | CCACACAC |
| Kmr No. 2790. | CCACACCC |
| Kmr No. 2791. | CCACACTC |
| Kmr No. 2792. | CCACAGAG |
| Kmr No. 2793. | CCACAGCG |
| Kmr No. 2794. | CCACCCAC |
| Kmr No. 2795. | CCACCCCA |
| Kmr No. 2796. | CCACCCCC |
| Kmr No. 2797. | CCACCCCG |
| Kmr No. 2798. | CCACCCGA |
| Kmr No. 2799. | CCACCCGC |
| Kmr No. 2800. | CCACCCGG |
| Kmr No. 2801. | CCACCCTA |
| Kmr No. 2802. | CCACCCTC |
| Kmr No. 2803. | CCACCGGA |
| Kmr No. 2804. | CCACCTCA |
| Kmr No. 2805. | CCACCTCC |
| Kmr No. 2806. | CCACGACA |
| Kmr No. 2807. | CCACGGGG |
| Kmr No. 2808. | CCACGTAA |
| Kmr No. 2809. | CCACGTAC |
| Kmr No. 2810. | CCACTAGA |
| Kmr No. 2811. | CCACTCAC |
| Kmr No. 2812. | CCACTCTC |
| Kmr No. 2813. | CCACTGAA |
| Kmr No. 2814. | CCACTGAC |
| Kmr No. 2815. | CCACTGGA |
| Kmr No. 2816. | CCACTGTA |
| Kmr No. 2817. | CCAGAATA |
| Kmr No. 2818. | CCAGAATC |
| Kmr No. 2819. | CCAGACCC |
| Kmr No. 2820. | CCAGAGCG |
| Kmr No. 2821. | CCAGATAA |
| Kmr No. 2822. | CCAGATTA |
| Kmr No. 2823. | CCAGATTC |
| Kmr No. 2824. | CCAGCAGC |
| Kmr No. 2825. | CCAGCCAC |
| Kmr No. 2826. | CCAGCCCC |
| Kmr No. 2827. | CCAGCCGC |
| Kmr No. 2828. | CCAGCCTC |
| Kmr No. 2829. | CCAGCGGG |
| Kmr No. 2830. | CCAGGTAC |
| Kmr No. 2831. | CCAGTAAA |
| Kmr No. 2832. | CCAGTAAC |
| Kmr No. 2833. | CCAGTACA |
| Kmr No. 2834. | CCAGTCAA |
| Kmr No. 2835. | CCAGTCAC |
| Kmr No. 2836. | CCAGTCAG |
| Kmr No. 2837. | CCAGTGAA |
| Kmr No. 2838. | CCAGTGAC |
| Kmr No. 2839. | CCAGTGGA |
| Kmr No. 2840. | CCAGTGTA |
| Kmr No. 2841. | CCAGTTAA |
| Kmr No. 2842. | CCAGTTAC |
| Kmr No. 2843. | CCAGTTCA |
| Kmr No. 2844. | CCAGTTTC |
| Kmr No. 2845. | CCATAACA |
| Kmr No. 2846. | CCATAACC |
| Kmr No. 2847. | CCATAACG |
| Kmr No. 2848. | CCATAATC |
| Kmr No. 2849. | CCATACGG |
| Kmr No. 2850. | CCATATTC |
| Kmr No. 2851. | CCATCCAA |
| Kmr No. 2852. | CCATCCCC |
| Kmr No. 2853. | CCATCCTC |
| Kmr No. 2854. | CCATCGGA |
| Kmr No. 2855. | CCATGCAA |
| Kmr No. 2856. | CCATGGAA |
| Kmr No. 2857. | CCATTGGA |
| Kmr No. 2858. | CCCAACAC |
| Kmr No. 2859. | CCCAACCC |
| Kmr No. 2860. | CCCAAGGG |
| Kmr No. 2861. | CCCACAAA |
| Kmr No. 2862. | CCCACAAC |
| Kmr No. 2863. | CCCACAAG |
| Kmr No. 2864. | CCCACACA |
| Kmr No. 2865. | CCCACACC |
| Kmr No. 2866. | CCCACAGC |
| Kmr No. 2867. | CCCACATC |
| Kmr No. 2868. | CCCACCAC |
| Kmr No. 2869. | CCCACCAG |
| Kmr No. 2870. | CCCACCCA |
| Kmr No. 2871. | CCCACCCC |
| Kmr No. 2872. | CCCACCCG |
| Kmr No. 2873. | CCCACCGA |
| Kmr No. 2874. | CCCACCGC |
| Kmr No. 2875. | CCCACCGG |
| Kmr No. 2876. | CCCACCTA |
| Kmr No. 2877. | CCCACCTC |
| Kmr No. 2878. | CCCACGGG |
| Kmr No. 2879. | CCCACGTA |
| Kmr No. 2880. | CCCAGAGC |
| Kmr No. 2881. | CCCAGAGG |
| Kmr No. 2882. | CCCAGCAC |
| Kmr No. 2883. | CCCAGCAG |
| Kmr No. 2884. | CCCAGCCA |
| Kmr No. 2885. | CCCAGCCC |
| Kmr No. 2886. | CCCAGCCG |
| Kmr No. 2887. | CCCAGCGC |
| Kmr No. 2888. | CCCAGCGG |
| Kmr No. 2889. | CCCAGCTA |
| Kmr No. 2890. | CCCAGTCA |
| Kmr No. 2891. | CCCAGTTA |
| Kmr No. 2892. | CCCATAAC |
| Kmr No. 2893. | CCCATCCC |
| Kmr No. 2894. | CCCATCTA |
| Kmr No. 2895. | CCCATCTC |
| Kmr No. 2896. | CCCATTAA |
| Kmr No. 2897. | CCCCAAAA |
| Kmr No. 2898. | CCCCAAAC |
| Kmr No. 2899. | CCCCAACA |
| Kmr No. 2900. | CCCCAACC |
| Kmr No. 2901. | CCCCAAGA |
| Kmr No. 2902. | CCCCAAGC |
| Kmr No. 2903. | CCCCAAGG |
| Kmr No. 2904. | CCCCACAA |
| Kmr No. 2905. | CCCCACAC |
| Kmr No. 2906. | CCCCACAG |
| Kmr No. 2907. | CCCCACCA |
| Kmr No. 2908. | CCCCACCC |
| Kmr No. 2909. | CCCCACCG |
| Kmr No. 2910. | CCCCACGA |
| Kmr No. 2911. | CCCCACGC |
| Kmr No. 2912. | CCCCACGG |
| Kmr No. 2913. | CCCCACTC |
| Kmr No. 2914. | CCCCAGAC |
| Kmr No. 2915. | CCCCAGAG |
| Kmr No. 2916. | CCCCAGCA |
| Kmr No. 2917. | CCCCAGCC |
| Kmr No. 2918. | CCCCAGCG |
| Kmr No. 2919. | CCCCATCA |
| Kmr No. 2920. | CCCCATCC |
| Kmr No. 2921. | CCCCATCG |
| Kmr No. 2922. | CCCCCAAA |
| Kmr No. 2923. | CCCCCAAC |
| Kmr No. 2924. | CCCCCAAG |
| Kmr No. 2925. | CCCCCACA |
| Kmr No. 2926. | CCCCCACC |
| Kmr No. 2927. | CCCCCACG |
| Kmr No. 2928. | CCCCCAGA |
| Kmr No. 2929. | CCCCCAGC |
| Kmr No. 2930. | CCCCCATC |
| Kmr No. 2931. | CCCCCCAA |
| Kmr No. 2932. | CCCCCCAC |
| Kmr No. 2933. | CCCCCCAG |
| Kmr No. 2934. | CCCCCCCA |
| Kmr No. 2935. | CCCCCCCC |
| Kmr No. 2936. | CCCCCCCG |
| Kmr No. 2937. | CCCCCCGA |
| Kmr No. 2938. | CCCCCCGC |
| Kmr No. 2939. | CCCCCCGG |
| Kmr No. 2940. | CCCCCCTA |
| Kmr No. 2941. | CCCCCCTC |
| Kmr No. 2942. | CCCCCGAC |
| Kmr No. 2943. | CCCCCGAG |
| Kmr No. 2944. | CCCCCGCA |
| Kmr No. 2945. | CCCCCGCC |
| Kmr No. 2946. | CCCCCGCG |
| Kmr No. 2947. | CCCCCGGA |
| Kmr No. 2948. | CCCCCGGC |
| Kmr No. 2949. | CCCCCGGG |
| Kmr No. 2950. | CCCCCGTC |
| Kmr No. 2951. | CCCCCTAA |
| Kmr No. 2952. | CCCCCTAC |
| Kmr No. 2953. | CCCCCTAG |
| Kmr No. 2954. | CCCCCTCA |
| Kmr No. 2955. | CCCCCTCC |
| Kmr No. 2956. | CCCCCTCG |
| Kmr No. 2957. | CCCCCTGA |
| Kmr No. 2958. | CCCCCTGC |
| Kmr No. 2959. | CCCCCTTC |
| Kmr No. 2960. | CCCCGAAA |
| Kmr No. 2961. | CCCCGACA |
| Kmr No. 2962. | CCCCGACC |
| Kmr No. 2963. | CCCCGACG |
| Kmr No. 2964. | CCCCGAGA |
| Kmr No. 2965. | CCCCGAGC |
| Kmr No. 2966. | CCCCGAGG |
| Kmr No. 2967. | CCCCGCAA |
| Kmr No. 2968. | CCCCGCAC |
| Kmr No. 2969. | CCCCGCAG |
| Kmr No. 2970. | CCCCGCCA |
| Kmr No. 2971. | CCCCGCCC |
| Kmr No. 2972. | CCCCGCCG |
| Kmr No. 2973. | CCCCGCGA |
| Kmr No. 2974. | CCCCGCGC |
| Kmr No. 2975. | CCCCGCGG |
| Kmr No. 2976. | CCCCGCTA |
| Kmr No. 2977. | CCCCGCTC |
| Kmr No. 2978. | CCCCGGAG |
| Kmr No. 2979. | CCCCGGCA |
| Kmr No. 2980. | CCCCGGCC |
| Kmr No. 2981. | CCCCGGCG |
| Kmr No. 2982. | CCCCGGGA |
| Kmr No. 2983. | CCCCGGGC |
| Kmr No. 2984. | CCCCGGGG |
| Kmr No. 2985. | CCCCGTCC |
| Kmr No. 2986. | CCCCGTCG |
| Kmr No. 2987. | CCCCGTGA |
| Kmr No. 2988. | CCCCGTGC |
| Kmr No. 2989. | CCCCTACA |
| Kmr No. 2990. | CCCCTACC |
| Kmr No. 2991. | CCCCTACG |
| Kmr No. 2992. | CCCCTAGC |
| Kmr No. 2993. | CCCCTATC |
| Kmr No. 2994. | CCCCTCAA |
| Kmr No. 2995. | CCCCTCAC |
| Kmr No. 2996. | CCCCTCAG |
| Kmr No. 2997. | CCCCTCCA |
| Kmr No. 2998. | CCCCTCCC |
| Kmr No. 2999. | CCCCTCCG |
| Kmr No. 3000. | CCCCTCGA |
| Kmr No. 3001. | CCCCTCGC |
| Kmr No. 3002. | CCCCTCGG |
| Kmr No. 3003. | CCCCTCTC |
| Kmr No. 3004. | CCCCTGAC |
| Kmr No. 3005. | CCCCTGAG |
| Kmr No. 3006. | CCCCTGCA |
| Kmr No. 3007. | CCCCTGCC |
| Kmr No. 3008. | CCCCTGCG |
| Kmr No. 3009. | CCCCTTCG |
| Kmr No. 3010. | CCCCTTGA |
| Kmr No. 3011. | CCCCTTGC |
| Kmr No. 3012. | CCCGAAAA |
| Kmr No. 3013. | CCCGACAA |
| Kmr No. 3014. | CCCGACAC |
| Kmr No. 3015. | CCCGACAG |
| Kmr No. 3016. | CCCGACCC |
| Kmr No. 3017. | CCCGAGAG |
| Kmr No. 3018. | CCCGAGGG |
| Kmr No. 3019. | CCCGCAAA |
| Kmr No. 3020. | CCCGCACA |
| Kmr No. 3021. | CCCGCACC |
| Kmr No. 3022. | CCCGCAGA |
| Kmr No. 3023. | CCCGCAGC |
| Kmr No. 3024. | CCCGCAGG |
| Kmr No. 3025. | CCCGCCAA |
| Kmr No. 3026. | CCCGCCAC |
| Kmr No. 3027. | CCCGCCAG |
| Kmr No. 3028. | CCCGCCCA |
| Kmr No. 3029. | CCCGCCCC |
| Kmr No. 3030. | CCCGCCCG |
| Kmr No. 3031. | CCCGCCGA |
| Kmr No. 3032. | CCCGCCGC |
| Kmr No. 3033. | CCCGCCGG |
| Kmr No. 3034. | CCCGCCTA |
| Kmr No. 3035. | CCCGCCTC |
| Kmr No. 3036. | CCCGCGCC |
| Kmr No. 3037. | CCCGCGCG |
| Kmr No. 3038. | CCCGCGGC |
| Kmr No. 3039. | CCCGCGGG |
| Kmr No. 3040. | CCCGCTCA |
| Kmr No. 3041. | CCCGCTCC |
| Kmr No. 3042. | CCCGCTGA |
| Kmr No. 3043. | CCCGCTGC |
| Kmr No. 3044. | CCCGCTTC |
| Kmr No. 3045. | CCCGGAGG |
| Kmr No. 3046. | CCCGGCAG |
| Kmr No. 3047. | CCCGGCCC |
| Kmr No. 3048. | CCCGGCCG |
| Kmr No. 3049. | CCCGGCGA |
| Kmr No. 3050. | CCCGGCGC |
| Kmr No. 3051. | CCCGGCGG |
| Kmr No. 3052. | CCCGGGAG |
| Kmr No. 3053. | CCCGGGCC |
| Kmr No. 3054. | CCCGGGCG |
| Kmr No. 3055. | CCCGGGGC |
| Kmr No. 3056. | CCCGTACG |
| Kmr No. 3057. | CCCGTCCC |
| Kmr No. 3058. | CCCGTCTC |
| Kmr No. 3059. | CCCGTGAG |
| Kmr No. 3060. | CCCGTGCC |
| Kmr No. 3061. | CCCGTGTC |
| Kmr No. 3062. | CCCTAAGC |
| Kmr No. 3063. | CCCTACCC |
| Kmr No. 3064. | CCCTACCG |
| Kmr No. 3065. | CCCTACGA |
| Kmr No. 3066. | CCCTACGC |
| Kmr No. 3067. | CCCTAGCC |
| Kmr No. 3068. | CCCTAGGG |
| Kmr No. 3069. | CCCTCAAA |
| Kmr No. 3070. | CCCTCAAC |
| Kmr No. 3071. | CCCTCAAG |
| Kmr No. 3072. | CCCTCACA |
| Kmr No. 3073. | CCCTCACC |
| Kmr No. 3074. | CCCTCACG |
| Kmr No. 3075. | CCCTCAGA |
| Kmr No. 3076. | CCCTCAGC |
| Kmr No. 3077. | CCCTCATC |
| Kmr No. 3078. | CCCTCCAA |
| Kmr No. 3079. | CCCTCCAC |
| Kmr No. 3080. | CCCTCCAG |
| Kmr No. 3081. | CCCTCCCA |
| Kmr No. 3082. | CCCTCCCC |
| Kmr No. 3083. | CCCTCCCG |
| Kmr No. 3084. | CCCTCCGA |
| Kmr No. 3085. | CCCTCCGC |
| Kmr No. 3086. | CCCTCCGG |
| Kmr No. 3087. | CCCTCCTA |
| Kmr No. 3088. | CCCTCCTC |
| Kmr No. 3089. | CCCTCGCA |
| Kmr No. 3090. | CCCTCGCC |
| Kmr No. 3091. | CCCTCGGA |
| Kmr No. 3092. | CCCTCGTA |
| Kmr No. 3093. | CCCTCTAC |
| Kmr No. 3094. | CCCTCTAG |
| Kmr No. 3095. | CCCTCTCA |
| Kmr No. 3096. | CCCTCTCC |
| Kmr No. 3097. | CCCTCTCG |
| Kmr No. 3098. | CCCTCTGC |
| Kmr No. 3099. | CCCTCTTC |
| Kmr No. 3100. | CCCTGCAG |
| Kmr No. 3101. | CCCTGCCA |
| Kmr No. 3102. | CCCTGCCC |
| Kmr No. 3103. | CCCTGCCG |
| Kmr No. 3104. | CCCTGCGA |
| Kmr No. 3105. | CCCTGCGG |
| Kmr No. 3106. | CCCTGTCG |
| Kmr No. 3107. | CCCTGTGC |
| Kmr No. 3108. | CCCTTAGC |
| Kmr No. 3109. | CCCTTCAC |
| Kmr No. 3110. | CCCTTCGC |
| Kmr No. 3111. | CCCTTCTC |
| Kmr No. 3112. | CCCTTGAA |
| Kmr No. 3113. | CCCTTGCA |
| Kmr No. 3114. | CCCTTGCC |
| Kmr No. 3115. | CCCTTGCG |
| Kmr No. 3116. | CCCTTGTA |
| Kmr No. 3117. | CCCTTGTC |
| Kmr No. 3118. | CCCTTTCG |
| Kmr No. 3119. | CCCTTTGC |
| Kmr No. 3120. | CCCTTTTC |
| Kmr No. 3121. | CCGAAAAG |
| Kmr No. 3122. | CCGAATCA |
| Kmr No. 3123. | CCGAATTC |
| Kmr No. 3124. | CCGACACA |
| Kmr No. 3125. | CCGACAGA |
| Kmr No. 3126. | CCGACATA |
| Kmr No. 3127. | CCGACCCC |
| Kmr No. 3128. | CCGACTAA |
| Kmr No. 3129. | CCGAGAAA |
| Kmr No. 3130. | CCGAGAGA |
| Kmr No. 3131. | CCGAGAGC |
| Kmr No. 3132. | CCGAGAGG |
| Kmr No. 3133. | CCGAGATA |
| Kmr No. 3134. | CCGAGGGA |
| Kmr No. 3135. | CCGAGGTA |
| Kmr No. 3136. | CCGAGTGC |
| Kmr No. 3137. | CCGATCAA |
| Kmr No. 3138. | CCGATCAC |
| Kmr No. 3139. | CCGATGCA |
| Kmr No. 3140. | CCGATTAA |
| Kmr No. 3141. | CCGATTAC |
| Kmr No. 3142. | CCGATTCA |
| Kmr No. 3143. | CCGATTCC |
| Kmr No. 3144. | CCGATTGC |
| Kmr No. 3145. | CCGCAAAA |
| Kmr No. 3146. | CCGCAAAG |
| Kmr No. 3147. | CCGCAAGA |
| Kmr No. 3148. | CCGCAAGG |
| Kmr No. 3149. | CCGCACCC |
| Kmr No. 3150. | CCGCAGAG |
| Kmr No. 3151. | CCGCAGCA |
| Kmr No. 3152. | CCGCAGCG |
| Kmr No. 3153. | CCGCATTA |
| Kmr No. 3154. | CCGCCACA |
| Kmr No. 3155. | CCGCCAGC |
| Kmr No. 3156. | CCGCCCAC |
| Kmr No. 3157. | CCGCCCCA |
| Kmr No. 3158. | CCGCCCCC |
| Kmr No. 3159. | CCGCCCCG |
| Kmr No. 3160. | CCGCCCGC |
| Kmr No. 3161. | CCGCCCGG |
| Kmr No. 3162. | CCGCCCTA |
| Kmr No. 3163. | CCGCCCTC |
| Kmr No. 3164. | CCGCCGCC |
| Kmr No. 3165. | CCGCCTGC |
| Kmr No. 3166. | CCGCGCCC |
| Kmr No. 3167. | CCGCGCGG |
| Kmr No. 3168. | CCGCTCAC |
| Kmr No. 3169. | CCGCTCCA |
| Kmr No. 3170. | CCGCTCCC |
| Kmr No. 3171. | CCGCTCTA |
| Kmr No. 3172. | CCGCTCTC |
| Kmr No. 3173. | CCGCTGAG |
| Kmr No. 3174. | CCGCTGCA |
| Kmr No. 3175. | CCGCTGCC |
| Kmr No. 3176. | CCGCTGCG |
| Kmr No. 3177. | CCGCTTGA |
| Kmr No. 3178. | CCGCTTGC |
| Kmr No. 3179. | CCGGAACC |
| Kmr No. 3180. | CCGGAAGA |
| Kmr No. 3181. | CCGGAATA |
| Kmr No. 3182. | CCGGAATC |
| Kmr No. 3183. | CCGGAGTC |
| Kmr No. 3184. | CCGGATAA |
| Kmr No. 3185. | CCGGATAC |
| Kmr No. 3186. | CCGGATTA |
| Kmr No. 3187. | CCGGATTC |
| Kmr No. 3188. | CCGGCCCC |
| Kmr No. 3189. | CCGGGCCC |
| Kmr No. 3190. | CCGGGGCA |
| Kmr No. 3191. | CCGGGGCC |
| Kmr No. 3192. | CCGGGGCG |
| Kmr No. 3193. | CCGGGGGA |
| Kmr No. 3194. | CCGGGGGC |
| Kmr No. 3195. | CCGGGTGC |
| Kmr No. 3196. | CCGGTAAC |
| Kmr No. 3197. | CCGGTTCC |
| Kmr No. 3198. | CCGGTTTC |
| Kmr No. 3199. | CCGTAATC |
| Kmr No. 3200. | CCGTACGA |
| Kmr No. 3201. | CCGTACGC |
| Kmr No. 3202. | CCGTACTA |
| Kmr No. 3203. | CCGTAGAG |
| Kmr No. 3204. | CCGTATAC |
| Kmr No. 3205. | CCGTATGA |
| Kmr No. 3206. | CCGTATTA |
| Kmr No. 3207. | CCGTATTC |
| Kmr No. 3208. | CCGTCCCC |
| Kmr No. 3209. | CCGTCTAC |
| Kmr No. 3210. | CCGTCTTC |
| Kmr No. 3211. | CCGTGAGG |
| Kmr No. 3212. | CCGTGCTA |
| Kmr No. 3213. | CCGTTCTA |
| Kmr No. 3214. | CCGTTGGA |
| Kmr No. 3215. | CCTAACCC |
| Kmr No. 3216. | CCTAAGCA |
| Kmr No. 3217. | CCTAAGCC |
| Kmr No. 3218. | CCTAATTC |
| Kmr No. 3219. | CCTACAAG |
| Kmr No. 3220. | CCTACACC |
| Kmr No. 3221. | CCTACATC |
| Kmr No. 3222. | CCTACCCC |
| Kmr No. 3223. | CCTACCTC |
| Kmr No. 3224. | CCTACGGC |
| Kmr No. 3225. | CCTAGAAA |
| Kmr No. 3226. | CCTAGCCC |
| Kmr No. 3227. | CCTAGCCG |
| Kmr No. 3228. | CCTAGGCA |
| Kmr No. 3229. | CCTAGGGC |
| Kmr No. 3230. | CCTATCTC |
| Kmr No. 3231. | CCTATGCC |
| Kmr No. 3232. | CCTATGTC |
| Kmr No. 3233. | CCTCAAAG |
| Kmr No. 3234. | CCTCAACA |
| Kmr No. 3235. | CCTCACAC |
| Kmr No. 3236. | CCTCACAG |
| Kmr No. 3237. | CCTCACCA |
| Kmr No. 3238. | CCTCACCC |
| Kmr No. 3239. | CCTCACTC |
| Kmr No. 3240. | CCTCAGAG |
| Kmr No. 3241. | CCTCAGCA |
| Kmr No. 3242. | CCTCAGCC |
| Kmr No. 3243. | CCTCAGCG |
| Kmr No. 3244. | CCTCAGTC |
| Kmr No. 3245. | CCTCATAG |
| Kmr No. 3246. | CCTCCAGC |
| Kmr No. 3247. | CCTCCCAC |
| Kmr No. 3248. | CCTCCCCA |
| Kmr No. 3249. | CCTCCCCC |
| Kmr No. 3250. | CCTCCCCG |
| Kmr No. 3251. | CCTCCCGA |
| Kmr No. 3252. | CCTCCCGC |
| Kmr No. 3253. | CCTCCCTA |
| Kmr No. 3254. | CCTCCCTC |
| Kmr No. 3255. | CCTCCGAG |
| Kmr No. 3256. | CCTCCGCA |
| Kmr No. 3257. | CCTCCGCC |
| Kmr No. 3258. | CCTCCGCG |
| Kmr No. 3259. | CCTCCGTC |
| Kmr No. 3260. | CCTCCTCA |
| Kmr No. 3261. | CCTCCTCC |
| Kmr No. 3262. | CCTCCTGC |
| Kmr No. 3263. | CCTCCTTC |
| Kmr No. 3264. | CCTCGCAA |
| Kmr No. 3265. | CCTCGCCC |
| Kmr No. 3266. | CCTCGCTC |
| Kmr No. 3267. | CCTCGGTA |
| Kmr No. 3268. | CCTCGTAA |
| Kmr No. 3269. | CCTCGTAG |
| Kmr No. 3270. | CCTCTACG |
| Kmr No. 3271. | CCTCTCAC |
| Kmr No. 3272. | CCTCTCAG |
| Kmr No. 3273. | CCTCTCCC |
| Kmr No. 3274. | CCTCTCCG |
| Kmr No. 3275. | CCTCTCGA |
| Kmr No. 3276. | CCTCTCGC |
| Kmr No. 3277. | CCTCTCTC |
| Kmr No. 3278. | CCTCTGCG |
| Kmr No. 3279. | CCTCTTGC |
| Kmr No. 3280. | CCTGAATA |
| Kmr No. 3281. | CCTGACCC |
| Kmr No. 3282. | CCTGATTC |
| Kmr No. 3283. | CCTGCCAC |
| Kmr No. 3284. | CCTGCCCA |
| Kmr No. 3285. | CCTGCCCC |
| Kmr No. 3286. | CCTGCCGC |
| Kmr No. 3287. | CCTGCCTC |
| Kmr No. 3288. | CCTGCGGC |
| Kmr No. 3289. | CCTGGAAC |
| Kmr No. 3290. | CCTGTACA |
| Kmr No. 3291. | CCTGTCGC |
| Kmr No. 3292. | CCTGTGAG |
| Kmr No. 3293. | CCTGTGCC |
| Kmr No. 3294. | CCTGTTTC |
| Kmr No. 3295. | CCTTAGCC |
| Kmr No. 3296. | CCTTAGGC |
| Kmr No. 3297. | CCTTATCG |
| Kmr No. 3298. | CCTTCCAA |
| Kmr No. 3299. | CCTTCCCC |
| Kmr No. 3300. | CCTTCTCC |
| Kmr No. 3301. | CCTTGACA |
| Kmr No. 3302. | CCTTGCAG |
| Kmr No. 3303. | CCTTGCCA |
| Kmr No. 3304. | CCTTGCCC |
| Kmr No. 3305. | CCTTGCTC |
| Kmr No. 3306. | CCTTGGAA |
| Kmr No. 3307. | CCTTGGAC |
| Kmr No. 3308. | CCTTGTAG |
| Kmr No. 3309. | CCTTGTCA |
| Kmr No. 3310. | CCTTGTGA |
| Kmr No. 3311. | CCTTGTGC |
| Kmr No. 3312. | CCTTTCGC |
| Kmr No. 3313. | CCTTTGAG |
| Kmr No. 3314. | CCTTTGCA |
| Kmr No. 3315. | CCTTTGCC |
| Kmr No. 3316. | CCTTTGCG |
| Kmr No. 3317. | CCTTTTAA |
| Kmr No. 3318. | CCTTTTCG |
| Kmr No. 3319. | CGAAAAAA |
| Kmr No. 3320. | CGAAAAGC |
| Kmr No. 3321. | CGAACCCC |
| Kmr No. 3322. | CGAACTAG |
| Kmr No. 3323. | CGAAGGGA |
| Kmr No. 3324. | CGAATCAA |
| Kmr No. 3325. | CGAATCAC |
| Kmr No. 3326. | CGAATCAG |
| Kmr No. 3327. | CGAATCCA |
| Kmr No. 3328. | CGAATTCA |
| Kmr No. 3329. | CGAATTCC |
| Kmr No. 3330. | CGACAAGA |
| Kmr No. 3331. | CGACACAA |
| Kmr No. 3332. | CGACACAC |
| Kmr No. 3333. | CGACACAG |
| Kmr No. 3334. | CGACAGAC |
| Kmr No. 3335. | CGACAGAG |
| Kmr No. 3336. | CGACAGCA |
| Kmr No. 3337. | CGACATAA |
| Kmr No. 3338. | CGACATAC |
| Kmr No. 3339. | CGACATAG |
| Kmr No. 3340. | CGACCCCC |
| Kmr No. 3341. | CGACTAAA |
| Kmr No. 3342. | CGACTATA |
| Kmr No. 3343. | CGACTCTA |
| Kmr No. 3344. | CGACTCTC |
| Kmr No. 3345. | CGACTGAC |
| Kmr No. 3346. | CGAGAAAA |
| Kmr No. 3347. | CGAGAAAG |
| Kmr No. 3348. | CGAGACTC |
| Kmr No. 3349. | CGAGAGAA |
| Kmr No. 3350. | CGAGAGAC |
| Kmr No. 3351. | CGAGAGAG |
| Kmr No. 3352. | CGAGAGCA |
| Kmr No. 3353. | CGAGAGCC |
| Kmr No. 3354. | CGAGAGCG |
| Kmr No. 3355. | CGAGAGGA |
| Kmr No. 3356. | CGAGAGGC |
| Kmr No. 3357. | CGAGAGTA |
| Kmr No. 3358. | CGAGAGTC |
| Kmr No. 3359. | CGAGATAA |
| Kmr No. 3360. | CGAGATAC |
| Kmr No. 3361. | CGAGATAG |
| Kmr No. 3362. | CGAGCCTA |
| Kmr No. 3363. | CGAGGAAA |
| Kmr No. 3364. | CGAGGATA |
| Kmr No. 3365. | CGAGGCAG |
| Kmr No. 3366. | CGAGGCTA |
| Kmr No. 3367. | CGAGGGAA |
| Kmr No. 3368. | CGAGGGAG |
| Kmr No. 3369. | CGAGGGGA |
| Kmr No. 3370. | CGAGGGGC |
| Kmr No. 3371. | CGAGGGTA |
| Kmr No. 3372. | CGAGGTAA |
| Kmr No. 3373. | CGAGGTTA |
| Kmr No. 3374. | CGAGTATA |
| Kmr No. 3375. | CGAGTATC |
| Kmr No. 3376. | CGAGTGCA |
| Kmr No. 3377. | CGAGTGTA |
| Kmr No. 3378. | CGATATAG |
| Kmr No. 3379. | CGATCAAA |
| Kmr No. 3380. | CGATCACA |
| Kmr No. 3381. | CGATCATA |
| Kmr No. 3382. | CGATCCAA |
| Kmr No. 3383. | CGATCCCC |
| Kmr No. 3384. | CGATCGGA |
| Kmr No. 3385. | CGATCGTA |
| Kmr No. 3386. | CGATGCAG |
| Kmr No. 3387. | CGATGTAA |
| Kmr No. 3388. | CGATTAAC |
| Kmr No. 3389. | CGATTACC |
| Kmr No. 3390. | CGATTAGC |
| Kmr No. 3391. | CGATTGAA |
| Kmr No. 3392. | CGATTGGA |
| Kmr No. 3393. | CGATTGTC |
| Kmr No. 3394. | CGCAAAAA |
| Kmr No. 3395. | CGCAAAAG |
| Kmr No. 3396. | CGCAAAGA |
| Kmr No. 3397. | CGCAAAGC |
| Kmr No. 3398. | CGCAACTA |
| Kmr No. 3399. | CGCAAGAG |
| Kmr No. 3400. | CGCAAGGA |
| Kmr No. 3401. | CGCACAAA |
| Kmr No. 3402. | CGCACACA |
| Kmr No. 3403. | CGCACAGA |
| Kmr No. 3404. | CGCACAGC |
| Kmr No. 3405. | CGCACATA |
| Kmr No. 3406. | CGCACCCC |
| Kmr No. 3407. | CGCACCTA |
| Kmr No. 3408. | CGCACTCC |
| Kmr No. 3409. | CGCACTTA |
| Kmr No. 3410. | CGCAGAAA |
| Kmr No. 3411. | CGCAGACA |
| Kmr No. 3412. | CGCAGAGA |
| Kmr No. 3413. | CGCAGAGC |
| Kmr No. 3414. | CGCAGATA |
| Kmr No. 3415. | CGCAGCCC |
| Kmr No. 3416. | CGCAGCTA |
| Kmr No. 3417. | CGCAGGAG |
| Kmr No. 3418. | CGCATAGA |
| Kmr No. 3419. | CGCATCTA |
| Kmr No. 3420. | CGCATTAA |
| Kmr No. 3421. | CGCATTAC |
| Kmr No. 3422. | CGCATTAG |
| Kmr No. 3423. | CGCCAATC |
| Kmr No. 3424. | CGCCACAA |
| Kmr No. 3425. | CGCCCCAA |
| Kmr No. 3426. | CGCCCCAC |
| Kmr No. 3427. | CGCCCCAG |
| Kmr No. 3428. | CGCCCCCA |
| Kmr No. 3429. | CGCCCCCC |
| Kmr No. 3430. | CGCCCCCG |
| Kmr No. 3431. | CGCCCCGC |
| Kmr No. 3432. | CGCCCCTA |
| Kmr No. 3433. | CGCCCCTC |
| Kmr No. 3434. | CGCCCGCC |
| Kmr No. 3435. | CGCCCTAA |
| Kmr No. 3436. | CGCCCTAC |
| Kmr No. 3437. | CGCCCTAG |
| Kmr No. 3438. | CGCCCTCA |
| Kmr No. 3439. | CGCCCTCC |
| Kmr No. 3440. | CGCCCTGA |
| Kmr No. 3441. | CGCCCTGC |
| Kmr No. 3442. | CGCCCTTA |
| Kmr No. 3443. | CGCCGCAG |
| Kmr No. 3444. | CGCCGCCC |
| Kmr No. 3445. | CGCCGGGC |
| Kmr No. 3446. | CGCCTATA |
| Kmr No. 3447. | CGCCTATC |
| Kmr No. 3448. | CGCCTCCC |
| Kmr No. 3449. | CGCCTCTA |
| Kmr No. 3450. | CGCCTCTC |
| Kmr No. 3451. | CGCGAAAA |
| Kmr No. 3452. | CGCGAGAG |
| Kmr No. 3453. | CGCGCCCC |
| Kmr No. 3454. | CGCGGAGA |
| Kmr No. 3455. | CGCGGAGC |
| Kmr No. 3456. | CGCGGGAG |
| Kmr No. 3457. | CGCGGGGA |
| Kmr No. 3458. | CGCGGGGC |
| Kmr No. 3459. | CGCTAATC |
| Kmr No. 3460. | CGCTACAC |
| Kmr No. 3461. | CGCTACCC |
| Kmr No. 3462. | CGCTATGA |
| Kmr No. 3463. | CGCTATGC |
| Kmr No. 3464. | CGCTCAAA |
| Kmr No. 3465. | CGCTCACA |
| Kmr No. 3466. | CGCTCAGA |
| Kmr No. 3467. | CGCTCAGC |
| Kmr No. 3468. | CGCTCATA |
| Kmr No. 3469. | CGCTCCAC |
| Kmr No. 3470. | CGCTCCCC |
| Kmr No. 3471. | CGCTCCCG |
| Kmr No. 3472. | CGCTCCGC |
| Kmr No. 3473. | CGCTCCTC |
| Kmr No. 3474. | CGCTCTAC |
| Kmr No. 3475. | CGCTCTCA |
| Kmr No. 3476. | CGCTCTCC |
| Kmr No. 3477. | CGCTCTGA |
| Kmr No. 3478. | CGCTCTGC |
| Kmr No. 3479. | CGCTCTTC |
| Kmr No. 3480. | CGCTGAGA |
| Kmr No. 3481. | CGCTGCAA |
| Kmr No. 3482. | CGCTGCAC |
| Kmr No. 3483. | CGCTGCCA |
| Kmr No. 3484. | CGCTGCCC |
| Kmr No. 3485. | CGCTGCTA |
| Kmr No. 3486. | CGCTGGAG |
| Kmr No. 3487. | CGCTGTAA |
| Kmr No. 3488. | CGCTGTGA |
| Kmr No. 3489. | CGCTGTGC |
| Kmr No. 3490. | CGCTTATA |
| Kmr No. 3491. | CGCTTATC |
| Kmr No. 3492. | CGCTTCTC |
| Kmr No. 3493. | CGCTTGAA |
| Kmr No. 3494. | CGCTTGCA |
| Kmr No. 3495. | CGCTTGTA |
| Kmr No. 3496. | CGCTTGTC |
| Kmr No. 3497. | CGCTTTGC |
| Kmr No. 3498. | CGCTTTTC |
| Kmr No. 3499. | CGGAAACC |
| Kmr No. 3500. | CGGAAATC |
| Kmr No. 3501. | CGGAACAG |
| Kmr No. 3502. | CGGAACCA |
| Kmr No. 3503. | CGGAACCG |
| Kmr No. 3504. | CGGAACTA |
| Kmr No. 3505. | CGGAAGAA |
| Kmr No. 3506. | CGGAAGAC |
| Kmr No. 3507. | CGGAAGTA |
| Kmr No. 3508. | CGGAAGTC |
| Kmr No. 3509. | CGGAATAC |
| Kmr No. 3510. | CGGAATCA |
| Kmr No. 3511. | CGGAATCC |
| Kmr No. 3512. | CGGAATGA |
| Kmr No. 3513. | CGGAATTC |
| Kmr No. 3514. | CGGACCAA |
| Kmr No. 3515. | CGGAGAGA |
| Kmr No. 3516. | CGGAGAGC |
| Kmr No. 3517. | CGGAGGAC |
| Kmr No. 3518. | CGGAGGAG |
| Kmr No. 3519. | CGGAGGGC |
| Kmr No. 3520. | CGGAGTCA |
| Kmr No. 3521. | CGGATACA |
| Kmr No. 3522. | CGGATACG |
| Kmr No. 3523. | CGGATATC |
| Kmr No. 3524. | CGGATCAA |
| Kmr No. 3525. | CGGATCCA |
| Kmr No. 3526. | CGGATTCA |
| Kmr No. 3527. | CGGATTCC |
| Kmr No. 3528. | CGGATTGC |
| Kmr No. 3529. | CGGCAAAG |
| Kmr No. 3530. | CGGCACAG |
| Kmr No. 3531. | CGGCACTC |
| Kmr No. 3532. | CGGCAGAA |
| Kmr No. 3533. | CGGCAGAG |
| Kmr No. 3534. | CGGCATAG |
| Kmr No. 3535. | CGGCCCCC |
| Kmr No. 3536. | CGGCCCCG |
| Kmr No. 3537. | CGGCGCAG |
| Kmr No. 3538. | CGGCGGAG |
| Kmr No. 3539. | CGGCGGGA |
| Kmr No. 3540. | CGGCGGGC |
| Kmr No. 3541. | CGGCTCTC |
| Kmr No. 3542. | CGGCTGAG |
| Kmr No. 3543. | CGGGAATC |
| Kmr No. 3544. | CGGGACAC |
| Kmr No. 3545. | CGGGACCC |
| Kmr No. 3546. | CGGGAGAG |
| Kmr No. 3547. | CGGGAGCA |
| Kmr No. 3548. | CGGGATAG |
| Kmr No. 3549. | CGGGCAGA |
| Kmr No. 3550. | CGGGCAGC |
| Kmr No. 3551. | CGGGCCCC |
| Kmr No. 3552. | CGGGCCCG |
| Kmr No. 3553. | CGGGCGGA |
| Kmr No. 3554. | CGGGCGGC |
| Kmr No. 3555. | CGGGCTGC |
| Kmr No. 3556. | CGGGGACA |
| Kmr No. 3557. | CGGGGACG |
| Kmr No. 3558. | CGGGGAGA |
| Kmr No. 3559. | CGGGGAGC |
| Kmr No. 3560. | CGGGGATC |
| Kmr No. 3561. | CGGGGCAA |
| Kmr No. 3562. | CGGGGCAC |
| Kmr No. 3563. | CGGGGCAG |
| Kmr No. 3564. | CGGGGCCC |
| Kmr No. 3565. | CGGGGCGA |
| Kmr No. 3566. | CGGGGCGC |
| Kmr No. 3567. | CGGGGCTA |
| Kmr No. 3568. | CGGGGCTC |
| Kmr No. 3569. | CGGGGGAC |
| Kmr No. 3570. | CGGGGGAG |
| Kmr No. 3571. | CGGGGGCA |
| Kmr No. 3572. | CGGGGGCC |
| Kmr No. 3573. | CGGGGGGA |
| Kmr No. 3574. | CGGGGGGC |
| Kmr No. 3575. | CGGGGGTA |
| Kmr No. 3576. | CGGGGGTC |
| Kmr No. 3577. | CGGGGTAG |
| Kmr No. 3578. | CGGGGTCA |
| Kmr No. 3579. | CGGGGTGC |
| Kmr No. 3580. | CGGGTCTC |
| Kmr No. 3581. | CGGGTGCA |
| Kmr No. 3582. | CGGGTGTA |
| Kmr No. 3583. | CGGTAATC |
| Kmr No. 3584. | CGGTATTC |
| Kmr No. 3585. | CGGTCCAA |
| Kmr No. 3586. | CGGTCCGA |
| Kmr No. 3587. | CGGTTAAC |
| Kmr No. 3588. | CGGTTACC |
| Kmr No. 3589. | CGGTTCAG |
| Kmr No. 3590. | CGGTTCCA |
| Kmr No. 3591. | CGGTTGGA |
| Kmr No. 3592. | CGGTTTCC |
| Kmr No. 3593. | CGTAATCC |
| Kmr No. 3594. | CGTAATTC |
| Kmr No. 3595. | CGTACGAA |
| Kmr No. 3596. | CGTACGCA |
| Kmr No. 3597. | CGTACGGA |
| Kmr No. 3598. | CGTACGTA |
| Kmr No. 3599. | CGTACGTC |
| Kmr No. 3600. | CGTACTAA |
| Kmr No. 3601. | CGTACTTC |
| Kmr No. 3602. | CGTAGACA |
| Kmr No. 3603. | CGTAGACC |
| Kmr No. 3604. | CGTAGAGA |
| Kmr No. 3605. | CGTAGAGC |
| Kmr No. 3606. | CGTAGCAG |
| Kmr No. 3607. | CGTAGCCC |
| Kmr No. 3608. | CGTAGGAA |
| Kmr No. 3609. | CGTAGGTC |
| Kmr No. 3610. | CGTAGTCC |
| Kmr No. 3611. | CGTATCCA |
| Kmr No. 3612. | CGTATGAC |
| Kmr No. 3613. | CGTATGGA |
| Kmr No. 3614. | CGTATTAA |
| Kmr No. 3615. | CGTATTAC |
| Kmr No. 3616. | CGTCCCCC |
| Kmr No. 3617. | CGTCCCGC |
| Kmr No. 3618. | CGTCCCTC |
| Kmr No. 3619. | CGTCCGGA |
| Kmr No. 3620. | CGTCCTCC |
| Kmr No. 3621. | CGTCTCTA |
| Kmr No. 3622. | CGTCTCTC |
| Kmr No. 3623. | CGTCTGAA |
| Kmr No. 3624. | CGTCTTCC |
| Kmr No. 3625. | CGTGAAAC |
| Kmr No. 3626. | CGTGAATC |
| Kmr No. 3627. | CGTGAGAG |
| Kmr No. 3628. | CGTGATAG |
| Kmr No. 3629. | CGTGATTA |
| Kmr No. 3630. | CGTGATTC |
| Kmr No. 3631. | CGTGCCCC |
| Kmr No. 3632. | CGTGCCTC |
| Kmr No. 3633. | CGTGCTCC |
| Kmr No. 3634. | CGTGTGAG |
| Kmr No. 3635. | CGTTCCAA |
| Kmr No. 3636. | CGTTCCTA |
| Kmr No. 3637. | CGTTCTAC |
| Kmr No. 3638. | CGTTCTGC |
| Kmr No. 3639. | CGTTGACA |
| Kmr No. 3640. | CGTTGTAG |
| Kmr No. 3641. | CGTTGTGA |
| Kmr No. 3642. | CGTTTGGA |
| Kmr No. 3643. | CTAAACCC |
| Kmr No. 3644. | CTAAGCCC |
| Kmr No. 3645. | CTAAGGGC |
| Kmr No. 3646. | CTAATCCA |
| Kmr No. 3647. | CTAATCGC |
| Kmr No. 3648. | CTAATTCC |
| Kmr No. 3649. | CTACAAGC |
| Kmr No. 3650. | CTACACAC |
| Kmr No. 3651. | CTACACCC |
| Kmr No. 3652. | CTACATAA |
| Kmr No. 3653. | CTACCCCA |
| Kmr No. 3654. | CTACCCCC |
| Kmr No. 3655. | CTACCCTC |
| Kmr No. 3656. | CTACCTCA |
| Kmr No. 3657. | CTACGAGA |
| Kmr No. 3658. | CTACGATC |
| Kmr No. 3659. | CTACGCAC |
| Kmr No. 3660. | CTACGCCC |
| Kmr No. 3661. | CTACGGCA |
| Kmr No. 3662. | CTACTGGA |
| Kmr No. 3663. | CTACTGTA |
| Kmr No. 3664. | CTACTGTC |
| Kmr No. 3665. | CTACTTTA |
| Kmr No. 3666. | CTAGAAAA |
| Kmr No. 3667. | CTAGAAAC |
| Kmr No. 3668. | CTAGCCCC |
| Kmr No. 3669. | CTAGGAAA |
| Kmr No. 3670. | CTAGGGGC |
| Kmr No. 3671. | CTAGTAAA |
| Kmr No. 3672. | CTAGTCAA |
| Kmr No. 3673. | CTAGTGGA |
| Kmr No. 3674. | CTAGTTCA |
| Kmr No. 3675. | CTAGTTCC |
| Kmr No. 3676. | CTAGTTTC |
| Kmr No. 3677. | CTATAAGC |
| Kmr No. 3678. | CTATAGGC |
| Kmr No. 3679. | CTATCTCA |
| Kmr No. 3680. | CTATCTGC |
| Kmr No. 3681. | CTATGAGC |
| Kmr No. 3682. | CTATGCAC |
| Kmr No. 3683. | CTATGCCC |
| Kmr No. 3684. | CTATGCGC |
| Kmr No. 3685. | CTATGCTC |
| Kmr No. 3686. | CTATGGAA |
| Kmr No. 3687. | CTATGGGA |
| Kmr No. 3688. | CTATGTCA |
| Kmr No. 3689. | CTATGTGC |
| Kmr No. 3690. | CTCAAAAA |
| Kmr No. 3691. | CTCAAAGA |
| Kmr No. 3692. | CTCAAAGC |
| Kmr No. 3693. | CTCAACCC |
| Kmr No. 3694. | CTCAACTA |
| Kmr No. 3695. | CTCAAGAG |
| Kmr No. 3696. | CTCAATGC |
| Kmr No. 3697. | CTCACAAA |
| Kmr No. 3698. | CTCACAAC |
| Kmr No. 3699. | CTCACACA |
| Kmr No. 3700. | CTCACACC |
| Kmr No. 3701. | CTCACAGA |
| Kmr No. 3702. | CTCACAGC |
| Kmr No. 3703. | CTCACCCC |
| Kmr No. 3704. | CTCACCTC |
| Kmr No. 3705. | CTCAGACC |
| Kmr No. 3706. | CTCAGAGC |
| Kmr No. 3707. | CTCAGCAC |
| Kmr No. 3708. | CTCAGCCC |
| Kmr No. 3709. | CTCAGCGA |
| Kmr No. 3710. | CTCAGCGC |
| Kmr No. 3711. | CTCAGCTC |
| Kmr No. 3712. | CTCAGGGC |
| Kmr No. 3713. | CTCAGTGC |
| Kmr No. 3714. | CTCATAGA |
| Kmr No. 3715. | CTCATAGC |
| Kmr No. 3716. | CTCCACAC |
| Kmr No. 3717. | CTCCACCC |
| Kmr No. 3718. | CTCCAGCC |
| Kmr No. 3719. | CTCCCACC |
| Kmr No. 3720. | CTCCCATA |
| Kmr No. 3721. | CTCCCCAC |
| Kmr No. 3722. | CTCCCCCA |
| Kmr No. 3723. | CTCCCCCC |
| Kmr No. 3724. | CTCCCCGA |
| Kmr No. 3725. | CTCCCCGC |
| Kmr No. 3726. | CTCCCCTA |
| Kmr No. 3727. | CTCCCCTC |
| Kmr No. 3728. | CTCCCGAG |
| Kmr No. 3729. | CTCCCGCA |
| Kmr No. 3730. | CTCCCGCC |
| Kmr No. 3731. | CTCCCTCC |
| Kmr No. 3732. | CTCCGATC |
| Kmr No. 3733. | CTCCGCAC |
| Kmr No. 3734. | CTCCGCCC |
| Kmr No. 3735. | CTCCGCGA |
| Kmr No. 3736. | CTCCGCGC |
| Kmr No. 3737. | CTCCGCTC |
| Kmr No. 3738. | CTCCGGAA |
| Kmr No. 3739. | CTCCGTGA |
| Kmr No. 3740. | CTCCGTGC |
| Kmr No. 3741. | CTCCTACA |
| Kmr No. 3742. | CTCCTCAC |
| Kmr No. 3743. | CTCCTCAG |
| Kmr No. 3744. | CTCCTCCC |
| Kmr No. 3745. | CTCCTCGC |
| Kmr No. 3746. | CTCCTCTC |
| Kmr No. 3747. | CTCCTGTA |
| Kmr No. 3748. | CTCCTTGA |
| Kmr No. 3749. | CTCCTTGC |
| Kmr No. 3750. | CTCGAAAA |
| Kmr No. 3751. | CTCGACAG |
| Kmr No. 3752. | CTCGCAAA |
| Kmr No. 3753. | CTCGCAGA |
| Kmr No. 3754. | CTCGCCCC |
| Kmr No. 3755. | CTCGCTCC |
| Kmr No. 3756. | CTCGCTGC |
| Kmr No. 3757. | CTCGGGGA |
| Kmr No. 3758. | CTCGGTTA |
| Kmr No. 3759. | CTCGTAGA |
| Kmr No. 3760. | CTCGTCCC |
| Kmr No. 3761. | CTCGTTTC |
| Kmr No. 3762. | CTCTACAC |
| Kmr No. 3763. | CTCTACCC |
| Kmr No. 3764. | CTCTACGA |
| Kmr No. 3765. | CTCTACGC |
| Kmr No. 3766. | CTCTATGA |
| Kmr No. 3767. | CTCTATGC |
| Kmr No. 3768. | CTCTCAAC |
| Kmr No. 3769. | CTCTCACA |
| Kmr No. 3770. | CTCTCACC |
| Kmr No. 3771. | CTCTCAGC |
| Kmr No. 3772. | CTCTCCAC |
| Kmr No. 3773. | CTCTCCCC |
| Kmr No. 3774. | CTCTCCGA |
| Kmr No. 3775. | CTCTCCGC |
| Kmr No. 3776. | CTCTCCTC |
| Kmr No. 3777. | CTCTCGAA |
| Kmr No. 3778. | CTCTCGAC |
| Kmr No. 3779. | CTCTCGCA |
| Kmr No. 3780. | CTCTCGCC |
| Kmr No. 3781. | CTCTCGGA |
| Kmr No. 3782. | CTCTCGGC |
| Kmr No. 3783. | CTCTCGTC |
| Kmr No. 3784. | CTCTCTCA |
| Kmr No. 3785. | CTCTCTCC |
| Kmr No. 3786. | CTCTCTGC |
| Kmr No. 3787. | CTCTGAGC |
| Kmr No. 3788. | CTCTGCAC |
| Kmr No. 3789. | CTCTGCCC |
| Kmr No. 3790. | CTCTGCGA |
| Kmr No. 3791. | CTCTGCGC |
| Kmr No. 3792. | CTCTGCTC |
| Kmr No. 3793. | CTCTGGGC |
| Kmr No. 3794. | CTCTGTGC |
| Kmr No. 3795. | CTCTTCGC |
| Kmr No. 3796. | CTCTTGAC |
| Kmr No. 3797. | CTCTTGCC |
| Kmr No. 3798. | CTCTTGTC |
| Kmr No. 3799. | CTCTTTGC |
| Kmr No. 3800. | CTGAAATC |
| Kmr No. 3801. | CTGAACAC |
| Kmr No. 3802. | CTGAACCC |
| Kmr No. 3803. | CTGAATAA |
| Kmr No. 3804. | CTGAATCA |
| Kmr No. 3805. | CTGAATCC |
| Kmr No. 3806. | CTGAATTA |
| Kmr No. 3807. | CTGAATTC |
| Kmr No. 3808. | CTGACCCA |
| Kmr No. 3809. | CTGACCCC |
| Kmr No. 3810. | CTGACCTA |
| Kmr No. 3811. | CTGACCTC |
| Kmr No. 3812. | CTGACTAC |
| Kmr No. 3813. | CTGACTCC |
| Kmr No. 3814. | CTGACTGA |
| Kmr No. 3815. | CTGACTTA |
| Kmr No. 3816. | CTGACTTC |
| Kmr No. 3817. | CTGAGAGC |
| Kmr No. 3818. | CTGAGCAG |
| Kmr No. 3819. | CTGAGCGA |
| Kmr No. 3820. | CTGAGGGC |
| Kmr No. 3821. | CTGAGGTC |
| Kmr No. 3822. | CTGAGTGC |
| Kmr No. 3823. | CTGATATC |
| Kmr No. 3824. | CTGATCAA |
| Kmr No. 3825. | CTGATCAG |
| Kmr No. 3826. | CTGATTCA |
| Kmr No. 3827. | CTGATTCC |
| Kmr No. 3828. | CTGATTGA |
| Kmr No. 3829. | CTGATTTC |
| Kmr No. 3830. | CTGCAAGC |
| Kmr No. 3831. | CTGCACAA |
| Kmr No. 3832. | CTGCACAC |
| Kmr No. 3833. | CTGCACCC |
| Kmr No. 3834. | CTGCACTA |
| Kmr No. 3835. | CTGCACTC |
| Kmr No. 3836. | CTGCCCAC |
| Kmr No. 3837. | CTGCCCAG |
| Kmr No. 3838. | CTGCCCCA |
| Kmr No. 3839. | CTGCCCCC |
| Kmr No. 3840. | CTGCCCGC |
| Kmr No. 3841. | CTGCCCTA |
| Kmr No. 3842. | CTGCCCTC |
| Kmr No. 3843. | CTGCCGCC |
| Kmr No. 3844. | CTGCCTGC |
| Kmr No. 3845. | CTGCGAAA |
| Kmr No. 3846. | CTGCGACA |
| Kmr No. 3847. | CTGCGAGA |
| Kmr No. 3848. | CTGCGCAG |
| Kmr No. 3849. | CTGCGCCC |
| Kmr No. 3850. | CTGCGCTA |
| Kmr No. 3851. | CTGCGCTC |
| Kmr No. 3852. | CTGCGGCA |
| Kmr No. 3853. | CTGCGGGA |
| Kmr No. 3854. | CTGCGGGC |
| Kmr No. 3855. | CTGCTCCC |
| Kmr No. 3856. | CTGCTCTA |
| Kmr No. 3857. | CTGCTCTC |
| Kmr No. 3858. | CTGCTTGC |
| Kmr No. 3859. | CTGGAAAC |
| Kmr No. 3860. | CTGGAAAG |
| Kmr No. 3861. | CTGGAACC |
| Kmr No. 3862. | CTGGAAGA |
| Kmr No. 3863. | CTGGAATA |
| Kmr No. 3864. | CTGGAATC |
| Kmr No. 3865. | CTGGATAC |
| Kmr No. 3866. | CTGGATTA |
| Kmr No. 3867. | CTGGATTC |
| Kmr No. 3868. | CTGGGAAC |
| Kmr No. 3869. | CTGGGCAA |
| Kmr No. 3870. | CTGGGCTA |
| Kmr No. 3871. | CTGGGGCA |
| Kmr No. 3872. | CTGGGGGA |
| Kmr No. 3873. | CTGGGGGC |
| Kmr No. 3874. | CTGGGTGC |
| Kmr No. 3875. | CTGGTAAA |
| Kmr No. 3876. | CTGGTATC |
| Kmr No. 3877. | CTGGTTAC |
| Kmr No. 3878. | CTGGTTCC |
| Kmr No. 3879. | CTGGTTTC |
| Kmr No. 3880. | CTGTAATA |
| Kmr No. 3881. | CTGTACAA |
| Kmr No. 3882. | CTGTACAG |
| Kmr No. 3883. | CTGTAGAA |
| Kmr No. 3884. | CTGTATCC |
| Kmr No. 3885. | CTGTATTA |
| Kmr No. 3886. | CTGTATTC |
| Kmr No. 3887. | CTGTCAAG |
| Kmr No. 3888. | CTGTCCAA |
| Kmr No. 3889. | CTGTCGCC |
| Kmr No. 3890. | CTGTCGGC |
| Kmr No. 3891. | CTGTGAGC |
| Kmr No. 3892. | CTGTGATC |
| Kmr No. 3893. | CTGTGCCA |
| Kmr No. 3894. | CTGTGCCC |
| Kmr No. 3895. | CTGTGCGC |
| Kmr No. 3896. | CTGTGGAA |
| Kmr No. 3897. | CTGTGGGC |
| Kmr No. 3898. | CTGTGTGC |
| Kmr No. 3899. | CTGTTCAC |
| Kmr No. 3900. | CTGTTCCA |
| Kmr No. 3901. | CTGTTCTA |
| Kmr No. 3902. | CTGTTGGA |
| Kmr No. 3903. | CTTACACC |
| Kmr No. 3904. | CTTACCGC |
| Kmr No. 3905. | CTTACGCC |
| Kmr No. 3906. | CTTACGGC |
| Kmr No. 3907. | CTTAGGCA |
| Kmr No. 3908. | CTTAGGGC |
| Kmr No. 3909. | CTTATAGC |
| Kmr No. 3910. | CTTATCGC |
| Kmr No. 3911. | CTTATGGA |
| Kmr No. 3912. | CTTCAAGC |
| Kmr No. 3913. | CTTCAATC |
| Kmr No. 3914. | CTTCATAA |
| Kmr No. 3915. | CTTCCAAA |
| Kmr No. 3916. | CTTCCAAG |
| Kmr No. 3917. | CTTCCACA |
| Kmr No. 3918. | CTTCCAGA |
| Kmr No. 3919. | CTTCCATA |
| Kmr No. 3920. | CTTCCATC |
| Kmr No. 3921. | CTTCCCCC |
| Kmr No. 3922. | CTTCCGAA |
| Kmr No. 3923. | CTTCCGGA |
| Kmr No. 3924. | CTTCCGTA |
| Kmr No. 3925. | CTTCCTGA |
| Kmr No. 3926. | CTTCGCCC |
| Kmr No. 3927. | CTTCGCTC |
| Kmr No. 3928. | CTTCGGAA |
| Kmr No. 3929. | CTTCTAAA |
| Kmr No. 3930. | CTTCTACA |
| Kmr No. 3931. | CTTCTCGC |
| Kmr No. 3932. | CTTCTGTA |
| Kmr No. 3933. | CTTCTTCC |
| Kmr No. 3934. | CTTCTTTA |
| Kmr No. 3935. | CTTGAAGC |
| Kmr No. 3936. | CTTGACAA |
| Kmr No. 3937. | CTTGACAC |
| Kmr No. 3938. | CTTGACCC |
| Kmr No. 3939. | CTTGATGC |
| Kmr No. 3940. | CTTGCACA |
| Kmr No. 3941. | CTTGCAGA |
| Kmr No. 3942. | CTTGCAGC |
| Kmr No. 3943. | CTTGCCCA |
| Kmr No. 3944. | CTTGCCCC |
| Kmr No. 3945. | CTTGCCTC |
| Kmr No. 3946. | CTTGCGCC |
| Kmr No. 3947. | CTTGCTGC |
| Kmr No. 3948. | CTTGGAAA |
| Kmr No. 3949. | CTTGGAAC |
| Kmr No. 3950. | CTTGGACA |
| Kmr No. 3951. | CTTGGACC |
| Kmr No. 3952. | CTTGGATA |
| Kmr No. 3953. | CTTGGATC |
| Kmr No. 3954. | CTTGGGGC |
| Kmr No. 3955. | CTTGGTCC |
| Kmr No. 3956. | CTTGTAGC |
| Kmr No. 3957. | CTTGTCAA |
| Kmr No. 3958. | CTTGTCCA |
| Kmr No. 3959. | CTTGTCGC |
| Kmr No. 3960. | CTTGTGGA |
| Kmr No. 3961. | CTTGTGGC |
| Kmr No. 3962. | CTTGTGTC |
| Kmr No. 3963. | CTTGTTGC |
| Kmr No. 3964. | CTTTAAAC |
| Kmr No. 3965. | CTTTAAAG |
| Kmr No. 3966. | CTTTAATC |
| Kmr No. 3967. | CTTTAGAA |
| Kmr No. 3968. | CTTTAGTC |
| Kmr No. 3969. | CTTTATTC |
| Kmr No. 3970. | CTTTCCGC |
| Kmr No. 3971. | CTTTCTGC |
| Kmr No. 3972. | CTTTGAGA |
| Kmr No. 3973. | CTTTGAGC |
| Kmr No. 3974. | CTTTGATC |
| Kmr No. 3975. | CTTTGCAA |
| Kmr No. 3976. | CTTTGCAC |
| Kmr No. 3977. | CTTTGCCA |
| Kmr No. 3978. | CTTTGCCC |
| Kmr No. 3979. | CTTTGCGA |
| Kmr No. 3980. | CTTTGCGC |
| Kmr No. 3981. | CTTTGCTC |
| Kmr No. 3982. | CTTTGGAA |
| Kmr No. 3983. | CTTTGGTC |
| Kmr No. 3984. | CTTTGTGC |
| Kmr No. 3985. | CTTTTACA |
| Kmr No. 3986. | CTTTTCGA |
| Kmr No. 3987. | CTTTTCGC |
| Kmr No. 3988. | CTTTTGTA |
| Kmr No. 3989. | CTTTTGTC |
| Kmr No. 3990. | CTTTTTGC |
| Kmr No. 3991. | GAAAAAAA |
| Kmr No. 3992. | GAAAACAC |
| Kmr No. 3993. | GAAAACCC |
| Kmr No. 3994. | GAAAACGA |
| Kmr No. 3995. | GAAAACGC |
| Kmr No. 3996. | GAAAAGCC |
| Kmr No. 3997. | GAAAAGGC |
| Kmr No. 3998. | GAAACCCC |
| Kmr No. 3999. | GAAACGTA |
| Kmr No. 4000. | GAAACGTC |
| Kmr No. 4001. | GAAAGATC |
| Kmr No. 4002. | GAAAGCCC |
| Kmr No. 4003. | GAAATCAC |
| Kmr No. 4004. | GAAATCCA |
| Kmr No. 4005. | GAACAATC |
| Kmr No. 4006. | GAACACAA |
| Kmr No. 4007. | GAACACAC |
| Kmr No. 4008. | GAACACCC |
| Kmr No. 4009. | GAACAGAC |
| Kmr No. 4010. | GAACAGCC |
| Kmr No. 4011. | GAACAGGA |
| Kmr No. 4012. | GAACAGTA |
| Kmr No. 4013. | GAACAGTC |
| Kmr No. 4014. | GAACATGA |
| Kmr No. 4015. | GAACCATA |
| Kmr No. 4016. | GAACCCCA |
| Kmr No. 4017. | GAACCCCC |
| Kmr No. 4018. | GAACCGAC |
| Kmr No. 4019. | GAACCGCC |
| Kmr No. 4020. | GAACCGGA |
| Kmr No. 4021. | GAACCGTA |
| Kmr No. 4022. | GAACCGTC |
| Kmr No. 4023. | GAACGAGA |
| Kmr No. 4024. | GAACGTAC |
| Kmr No. 4025. | GAACGTCC |
| Kmr No. 4026. | GAACGTTA |
| Kmr No. 4027. | GAACGTTC |
| Kmr No. 4028. | GAACTAGC |
| Kmr No. 4029. | GAACTGGA |
| Kmr No. 4030. | GAACTGGC |
| Kmr No. 4031. | GAACTGTA |
| Kmr No. 4032. | GAACTGTC |
| Kmr No. 4033. | GAACTTCC |
| Kmr No. 4034. | GAAGAAGC |
| Kmr No. 4035. | GAAGAATC |
| Kmr No. 4036. | GAAGAGCA |
| Kmr No. 4037. | GAAGCTTC |
| Kmr No. 4038. | GAAGGAAC |
| Kmr No. 4039. | GAAGGGCA |
| Kmr No. 4040. | GAAGGGTC |
| Kmr No. 4041. | GAAGTAAA |
| Kmr No. 4042. | GAAGTAAC |
| Kmr No. 4043. | GAAGTACC |
| Kmr No. 4044. | GAAGTAGA |
| Kmr No. 4045. | GAAGTCAC |
| Kmr No. 4046. | GAAGTGAC |
| Kmr No. 4047. | GAAGTGTA |
| Kmr No. 4048. | GAATAATC |
| Kmr No. 4049. | GAATACCA |
| Kmr No. 4050. | GAATCACA |
| Kmr No. 4051. | GAATCACC |
| Kmr No. 4052. | GAATCAGA |
| Kmr No. 4053. | GAATCAGC |
| Kmr No. 4054. | GAATCATA |
| Kmr No. 4055. | GAATCCAA |
| Kmr No. 4056. | GAATCCCA |
| Kmr No. 4057. | GAATCCGA |
| Kmr No. 4058. | GAATCGGA |
| Kmr No. 4059. | GAATCGTA |
| Kmr No. 4060. | GAATCTGA |
| Kmr No. 4061. | GAATCTTA |
| Kmr No. 4062. | GAATGAAC |
| Kmr No. 4063. | GAATGAGC |
| Kmr No. 4064. | GAATGATC |
| Kmr No. 4065. | GAATGGGA |
| Kmr No. 4066. | GAATTACC |
| Kmr No. 4067. | GAATTCAC |
| Kmr No. 4068. | GAATTCCA |
| Kmr No. 4069. | GAATTCCC |
| Kmr No. 4070. | GAATTGAC |
| Kmr No. 4071. | GAATTGGA |
| Kmr No. 4072. | GAATTGGC |
| Kmr No. 4073. | GACAACCC |
| Kmr No. 4074. | GACAAGCA |
| Kmr No. 4075. | GACAAGGA |
| Kmr No. 4076. | GACACAAA |
| Kmr No. 4077. | GACACACA |
| Kmr No. 4078. | GACACACC |
| Kmr No. 4079. | GACACAGC |
| Kmr No. 4080. | GACACCCC |
| Kmr No. 4081. | GACACCTC |
| Kmr No. 4082. | GACACGTA |
| Kmr No. 4083. | GACACTCA |
| Kmr No. 4084. | GACAGAGC |
| Kmr No. 4085. | GACAGCCA |
| Kmr No. 4086. | GACAGCGA |
| Kmr No. 4087. | GACAGGAA |
| Kmr No. 4088. | GACAGGTA |
| Kmr No. 4089. | GACAGTAA |
| Kmr No. 4090. | GACAGTAC |
| Kmr No. 4091. | GACAGTCA |
| Kmr No. 4092. | GACAGTTA |
| Kmr No. 4093. | GACATAGA |
| Kmr No. 4094. | GACATAGC |
| Kmr No. 4095. | GACATTCC |
| Kmr No. 4096. | GACCAAAC |
| Kmr No. 4097. | GACCAATC |
| Kmr No. 4098. | GACCACCC |
| Kmr No. 4099. | GACCAGTC |
| Kmr No. 4100. | GACCATAC |
| Kmr No. 4101. | GACCATTA |
| Kmr No. 4102. | GACCCACA |
| Kmr No. 4103. | GACCCACC |
| Kmr No. 4104. | GACCCAGA |
| Kmr No. 4105. | GACCCAGC |
| Kmr No. 4106. | GACCCCAA |
| Kmr No. 4107. | GACCCCAC |
| Kmr No. 4108. | GACCCCCA |
| Kmr No. 4109. | GACCCCCC |
| Kmr No. 4110. | GACCCCGC |
| Kmr No. 4111. | GACCCCTA |
| Kmr No. 4112. | GACCCGCC |
| Kmr No. 4113. | GACCCTCA |
| Kmr No. 4114. | GACCCTCC |
| Kmr No. 4115. | GACCCTGA |
| Kmr No. 4116. | GACCCTGC |
| Kmr No. 4117. | GACCGAGA |
| Kmr No. 4118. | GACCGGAA |
| Kmr No. 4119. | GACCGTAC |
| Kmr No. 4120. | GACCTACA |
| Kmr No. 4121. | GACCTATC |
| Kmr No. 4122. | GACCTCCC |
| Kmr No. 4123. | GACCTCTA |
| Kmr No. 4124. | GACCTCTC |
| Kmr No. 4125. | GACGAAAA |
| Kmr No. 4126. | GACGAATC |
| Kmr No. 4127. | GACGCAAA |
| Kmr No. 4128. | GACGCAGA |
| Kmr No. 4129. | GACGGAAC |
| Kmr No. 4130. | GACGGGGC |
| Kmr No. 4131. | GACGTATC |
| Kmr No. 4132. | GACGTTCC |
| Kmr No. 4133. | GACTAATC |
| Kmr No. 4134. | GACTAGTA |
| Kmr No. 4135. | GACTAGTC |
| Kmr No. 4136. | GACTCACA |
| Kmr No. 4137. | GACTCCCA |
| Kmr No. 4138. | GACTCTGC |
| Kmr No. 4139. | GACTGAAC |
| Kmr No. 4140. | GACTGACA |
| Kmr No. 4141. | GACTGATC |
| Kmr No. 4142. | GACTGCAA |
| Kmr No. 4143. | GACTGGAA |
| Kmr No. 4144. | GACTGGAC |
| Kmr No. 4145. | GACTGGCA |
| Kmr No. 4146. | GACTGGGA |
| Kmr No. 4147. | GACTGGTA |
| Kmr No. 4148. | GACTGTAC |
| Kmr No. 4149. | GACTGTTA |
| Kmr No. 4150. | GACTTCCA |
| Kmr No. 4151. | GACTTGGA |
| Kmr No. 4152. | GACTTGGC |
| Kmr No. 4153. | GAGAACCC |
| Kmr No. 4154. | GAGAATCC |
| Kmr No. 4155. | GAGAATGC |
| Kmr No. 4156. | GAGACACA |
| Kmr No. 4157. | GAGACCCA |
| Kmr No. 4158. | GAGACCCC |
| Kmr No. 4159. | GAGACCTA |
| Kmr No. 4160. | GAGACGCA |
| Kmr No. 4161. | GAGACTCA |
| Kmr No. 4162. | GAGAGACA |
| Kmr No. 4163. | GAGAGACC |
| Kmr No. 4164. | GAGAGAGA |
| Kmr No. 4165. | GAGAGAGC |
| Kmr No. 4166. | GAGAGATA |
| Kmr No. 4167. | GAGAGCAA |
| Kmr No. 4168. | GAGAGCAC |
| Kmr No. 4169. | GAGAGCCA |
| Kmr No. 4170. | GAGAGCCC |
| Kmr No. 4171. | GAGAGCGA |
| Kmr No. 4172. | GAGAGCGC |
| Kmr No. 4173. | GAGAGCTA |
| Kmr No. 4174. | GAGAGGAA |
| Kmr No. 4175. | GAGAGGCA |
| Kmr No. 4176. | GAGAGGGC |
| Kmr No. 4177. | GAGAGTCC |
| Kmr No. 4178. | GAGAGTGA |
| Kmr No. 4179. | GAGAGTGC |
| Kmr No. 4180. | GAGATAGC |
| Kmr No. 4181. | GAGATGCA |
| Kmr No. 4182. | GAGATTCC |
| Kmr No. 4183. | GAGCAAAA |
| Kmr No. 4184. | GAGCAAAC |
| Kmr No. 4185. | GAGCAAGA |
| Kmr No. 4186. | GAGCAAGC |
| Kmr No. 4187. | GAGCACAA |
| Kmr No. 4188. | GAGCACCC |
| Kmr No. 4189. | GAGCACTA |
| Kmr No. 4190. | GAGCAGCC |
| Kmr No. 4191. | GAGCATAC |
| Kmr No. 4192. | GAGCATCA |
| Kmr No. 4193. | GAGCCACA |
| Kmr No. 4194. | GAGCCCCC |
| Kmr No. 4195. | GAGCCCTA |
| Kmr No. 4196. | GAGCCGCA |
| Kmr No. 4197. | GAGCCTAA |
| Kmr No. 4198. | GAGCCTTA |
| Kmr No. 4199. | GAGCGAAA |
| Kmr No. 4200. | GAGCGACA |
| Kmr No. 4201. | GAGCGAGA |
| Kmr No. 4202. | GAGCGATA |
| Kmr No. 4203. | GAGCGCAA |
| Kmr No. 4204. | GAGCGGAA |
| Kmr No. 4205. | GAGCGGGA |
| Kmr No. 4206. | GAGCGGTA |
| Kmr No. 4207. | GAGCGTAA |
| Kmr No. 4208. | GAGCTATA |
| Kmr No. 4209. | GAGCTCTA |
| Kmr No. 4210. | GAGCTGAA |
| Kmr No. 4211. | GAGCTGCA |
| Kmr No. 4212. | GAGGAATC |
| Kmr No. 4213. | GAGGACAA |
| Kmr No. 4214. | GAGGACAC |
| Kmr No. 4215. | GAGGACCC |
| Kmr No. 4216. | GAGGACTA |
| Kmr No. 4217. | GAGGAGAA |
| Kmr No. 4218. | GAGGAGCA |
| Kmr No. 4219. | GAGGAGGA |
| Kmr No. 4220. | GAGGAGTA |
| Kmr No. 4221. | GAGGCAAA |
| Kmr No. 4222. | GAGGCACA |
| Kmr No. 4223. | GAGGCCCC |
| Kmr No. 4224. | GAGGCTGA |
| Kmr No. 4225. | GAGGCTGC |
| Kmr No. 4226. | GAGGCTTA |
| Kmr No. 4227. | GAGGGACA |
| Kmr No. 4228. | GAGGGAGA |
| Kmr No. 4229. | GAGGGAGC |
| Kmr No. 4230. | GAGGGCAA |
| Kmr No. 4231. | GAGGGCAC |
| Kmr No. 4232. | GAGGGCCC |
| Kmr No. 4233. | GAGGGCGA |
| Kmr No. 4234. | GAGGGCTA |
| Kmr No. 4235. | GAGGGGAC |
| Kmr No. 4236. | GAGGGGCA |
| Kmr No. 4237. | GAGGGGCC |
| Kmr No. 4238. | GAGGGGGA |
| Kmr No. 4239. | GAGGGGGC |
| Kmr No. 4240. | GAGGGGTA |
| Kmr No. 4241. | GAGGGTAA |
| Kmr No. 4242. | GAGGGTAC |
| Kmr No. 4243. | GAGGGTCA |
| Kmr No. 4244. | GAGGGTCC |
| Kmr No. 4245. | GAGGGTGA |
| Kmr No. 4246. | GAGGGTGC |
| Kmr No. 4247. | GAGGGTTA |
| Kmr No. 4248. | GAGGTAGC |
| Kmr No. 4249. | GAGGTCAA |
| Kmr No. 4250. | GAGGTCTA |
| Kmr No. 4251. | GAGGTGAA |
| Kmr No. 4252. | GAGGTGCA |
| Kmr No. 4253. | GAGGTGGA |
| Kmr No. 4254. | GAGGTGTA |
| Kmr No. 4255. | GAGGTTGA |
| Kmr No. 4256. | GAGTACAA |
| Kmr No. 4257. | GAGTACAC |
| Kmr No. 4258. | GAGTCCAA |
| Kmr No. 4259. | GAGTCCCC |
| Kmr No. 4260. | GAGTCGGA |
| Kmr No. 4261. | GAGTGAGA |
| Kmr No. 4262. | GAGTGAGC |
| Kmr No. 4263. | GAGTGATC |
| Kmr No. 4264. | GAGTGCAA |
| Kmr No. 4265. | GAGTGCCA |
| Kmr No. 4266. | GAGTGCCC |
| Kmr No. 4267. | GAGTGCGC |
| Kmr No. 4268. | GAGTGTAA |
| Kmr No. 4269. | GAGTGTGA |
| Kmr No. 4270. | GAGTGTGC |
| Kmr No. 4271. | GAGTTCAA |
| Kmr No. 4272. | GAGTTTGC |
| Kmr No. 4273. | GATAATCC |
| Kmr No. 4274. | GATACGTA |
| Kmr No. 4275. | GATAGAGC |
| Kmr No. 4276. | GATAGCCC |
| Kmr No. 4277. | GATAGTGC |
| Kmr No. 4278. | GATATCAC |
| Kmr No. 4279. | GATATCCA |
| Kmr No. 4280. | GATATGGA |
| Kmr No. 4281. | GATATGGC |
| Kmr No. 4282. | GATCAAAC |
| Kmr No. 4283. | GATCAATA |
| Kmr No. 4284. | GATCAATC |
| Kmr No. 4285. | GATCACTA |
| Kmr No. 4286. | GATCAGAA |
| Kmr No. 4287. | GATCAGAC |
| Kmr No. 4288. | GATCAGGA |
| Kmr No. 4289. | GATCAGTA |
| Kmr No. 4290. | GATCATAA |
| Kmr No. 4291. | GATCATAC |
| Kmr No. 4292. | GATCATTA |
| Kmr No. 4293. | GATCCAAC |
| Kmr No. 4294. | GATCCATA |
| Kmr No. 4295. | GATCCCAA |
| Kmr No. 4296. | GATCCCCC |
| Kmr No. 4297. | GATCCGAA |
| Kmr No. 4298. | GATCCGGA |
| Kmr No. 4299. | GATCCGTA |
| Kmr No. 4300. | GATCCTTA |
| Kmr No. 4301. | GATCGATA |
| Kmr No. 4302. | GATCGATC |
| Kmr No. 4303. | GATCGCTA |
| Kmr No. 4304. | GATCGGAA |
| Kmr No. 4305. | GATCGGAC |
| Kmr No. 4306. | GATCGGTA |
| Kmr No. 4307. | GATCGTAA |
| Kmr No. 4308. | GATCGTAC |
| Kmr No. 4309. | GATCGTTA |
| Kmr No. 4310. | GATCTGAA |
| Kmr No. 4311. | GATCTGTA |
| Kmr No. 4312. | GATCTTTA |
| Kmr No. 4313. | GATGACCC |
| Kmr No. 4314. | GATGCACA |
| Kmr No. 4315. | GATGCACC |
| Kmr No. 4316. | GATGCAGC |
| Kmr No. 4317. | GATGCCCC |
| Kmr No. 4318 | GATGCTGC |
| Kmr No. 4319. | GATGGAAC |
| Kmr No. 4320. | GATGGGCA |
| Kmr No. 4321. | GATGGGGC |
| Kmr No. 4322. | GATGTAGC |
| Kmr No. 4323. | GATGTGAC |
| Kmr No. 4324. | GATGTGCA |
| Kmr No. 4325. | GATGTTCC |
| Kmr No. 4326. | GATTAATC |
| Kmr No. 4327. | GATTATGA |
| Kmr No. 4328. | GATTCACA |
| Kmr No. 4329. | GATTCACC |
| Kmr No. 4330. | GATTCAGC |
| Kmr No. 4331. | GATTCATA |
| Kmr No. 4332. | GATTCCAA |
| Kmr No. 4333. | GATTCCCA |
| Kmr No. 4334. | GATTCCGA |
| Kmr No. 4335. | GATTCCTA |
| Kmr No. 4336. | GATTCGAA |
| Kmr No. 4337. | GATTCGGA |
| Kmr No. 4338. | GATTCGTA |
| Kmr No. 4339. | GATTGAAC |
| Kmr No. 4340. | GATTGGAA |
| Kmr No. 4341. | GATTGGAC |
| Kmr No. 4342. | GATTGGCC |
| Kmr No. 4343. | GATTGGGA |
| Kmr No. 4344. | GATTTACC |
| Kmr No. 4345. | GATTTCAC |
| Kmr No. 4346. | GATTTCCA |
| Kmr No. 4347. | GATTTGAC |
| Kmr No. 4348. | GATTTGGA |
| Kmr No. 4349. | GATTTGGC |
| Kmr No. 4350. | GCAAAAAA |
| Kmr No. 4351. | GCAAACAC |
| Kmr No. 4352. | GCAAACCC |
| Kmr No. 4353. | GCAAAGAC |
| Kmr No. 4354. | GCAAAGCA |
| Kmr No. 4355. | GCAAAGCC |
| Kmr No. 4356. | GCAAAGGC |
| Kmr No. 4357. | GCAACCCC |
| Kmr No. 4358. | GCAAGAGC |
| Kmr No. 4359. | GCAAGCCC |
| Kmr No. 4360. | GCAAGGAC |
| Kmr No. 4361. | GCAAGGCA |
| Kmr No. 4362. | GCAAGGGA |
| Kmr No. 4363. | GCAAGGGC |
| Kmr No. 4364. | GCAATCAC |
| Kmr No. 4365. | GCAATTCC |
| Kmr No. 4366. | GCACAAAA |
| Kmr No. 4367. | GCACAAAC |
| Kmr No. 4368. | GCACAACA |
| Kmr No. 4369. | GCACAAGC |
| Kmr No. 4370. | GCACACAA |
| Kmr No. 4371. | GCACACAC |
| Kmr No. 4372. | GCACACCC |
| Kmr No. 4373. | GCACAGAA |
| Kmr No. 4374. | GCACAGCA |
| Kmr No. 4375. | GCACAGCC |
| Kmr No. 4376. | GCACATAC |
| Kmr No. 4377. | GCACATCA |
| Kmr No. 4378. | GCACCCAC |
| Kmr No. 4379. | GCACCCCA |
| Kmr No. 4380. | GCACCCCC |
| Kmr No. 4381. | GCACCCGC |
| Kmr No. 4382. | GCACCCTA |
| Kmr No. 4383. | GCACCTCA |
| Kmr No. 4384. | GCACCTCC |
| Kmr No. 4385. | GCACCTTA |
| Kmr No. 4386. | GCACGTAA |
| Kmr No. 4387. | GCACGTAC |
| Kmr No. 4388. | GCACTACA |
| Kmr No. 4389. | GCACTCAA |
| Kmr No. 4390. | GCACTCAC |
| Kmr No. 4391. | GCACTCCA |
| Kmr No. 4392. | GCACTCTA |
| Kmr No. 4393. | GCACTTCC |
| Kmr No. 4394. | GCAGACAA |
| Kmr No. 4395. | GCAGACAC |
| Kmr No. 4396. | GCAGACCC |
| Kmr No. 4397. | GCAGAGCA |
| Kmr No. 4398. | GCAGAGCC |
| Kmr No. 4399. | GCAGAGGC |
| Kmr No. 4400. | GCAGCACA |
| Kmr No. 4401. | GCAGCACC |
| Kmr No. 4402. | GCAGCATA |
| Kmr No. 4403. | GCAGCCCC |
| Kmr No. 4404. | GCAGCCTA |
| Kmr No. 4405. | GCAGCGCA |
| Kmr No. 4406. | GCAGCGCC |
| Kmr No. 4407. | GCAGCGGC |
| Kmr No. 4408. | GCAGCGTA |
| Kmr No. 4409. | GCAGCTCC |
| Kmr No. 4410. | GCAGGACA |
| Kmr No. 4411. | GCAGGAGC |
| Kmr No. 4412. | GCAGGCAC |
| Kmr No. 4413. | GCAGGGCA |
| Kmr No. 4414. | GCAGGGGA |
| Kmr No. 4415. | GCAGGGGC |
| Kmr No. 4416. | GCAGGGTA |
| Kmr No. 4417. | GCAGTGAA |
| Kmr No. 4418. | GCAGTGAC |
| Kmr No. 4419. | GCAGTGCC |
| Kmr No. 4420. | GCATAGCA |
| Kmr No. 4421. | GCATCAGC |
| Kmr No. 4422. | GCATCCAA |
| Kmr No. 4423. | GCATCCCC |
| Kmr No. 4424. | GCATTAGA |
| Kmr No. 4425. | GCATTGGA |
| Kmr No. 4426. | GCCAAAAA |
| Kmr No. 4427. | GCCAATCA |
| Kmr No. 4428. | GCCAATCC |
| Kmr No. 4429. | GCCACAAA |
| Kmr No. 4430. | GCCACACA |
| Kmr No. 4431. | GCCACCCC |
| Kmr No. 4432. | GCCACGTA |
| Kmr No. 4433. | GCCACTGA |
| Kmr No. 4434. | GCCAGTAA |
| Kmr No. 4435. | GCCAGTAC |
| Kmr No. 4436. | GCCAGTCA |
| Kmr No. 4437. | GCCAGTGA |
| Kmr No. 4438. | GCCAGTTA |
| Kmr No. 4439. | GCCATAAC |
| Kmr No. 4440. | GCCATCCC |
| Kmr No. 4441. | GCCATTAA |
| Kmr No. 4442. | GCCCAAAA |
| Kmr No. 4443. | GCCCACAA |
| Kmr No. 4444. | GCCCACAC |
| Kmr No. 4445. | GCCCACCC |
| Kmr No. 4446. | GCCCAGCC |
| Kmr No. 4447. | GCCCCAAA |
| Kmr No. 4448. | GCCCCAAC |
| Kmr No. 4449. | GCCCCACA |
| Kmr No. 4450. | GCCCCACC |
| Kmr No. 4451. | GCCCCAGA |
| Kmr No. 4452. | GCCCCAGC |
| Kmr No. 4453. | GCCCCCAA |
| Kmr No. 4454. | GCCCCCAC |
| Kmr No. 4455. | GCCCCCCA |
| Kmr No. 4456. | GCCCCCCC |
| Kmr No. 4457. | GCCCCCGA |
| Kmr No. 4458. | GCCCCCGC |
| Kmr No. 4459. | GCCCCCTA |
| Kmr No. 4460. | GCCCCGAC |
| Kmr No. 4461. | GCCCCGCA |
| Kmr No. 4462. | GCCCCGCC |
| Kmr No. 4463. | GCCCCGGC |
| Kmr No. 4464. | GCCCCTCA |
| Kmr No. 4465. | GCCCCTCC |
| Kmr No. 4466. | GCCCCTGA |
| Kmr No. 4467. | GCCCGCCA |
| Kmr No. 4468. | GCCCGCCC |
| Kmr No. 4469. | GCCCGGGC |
| Kmr No. 4470. | GCCCTAAC |
| Kmr No. 4471. | GCCCTACA |
| Kmr No. 4472. | GCCCTACC |
| Kmr No. 4473. | GCCCTCAA |
| Kmr No. 4474. | GCCCTCAC |
| Kmr No. 4475. | GCCCTCCA |
| Kmr No. 4476. | GCCCTCCC |
| Kmr No. 4477. | GCCCTCTA |
| Kmr No. 4478. | GCCCTGCA |
| Kmr No. 4479. | GCCCTGCC |
| Kmr No. 4480. | GCCCTTCA |
| Kmr No. 4481. | GCCGAAAA |
| Kmr No. 4482. | GCCGCAAA |
| Kmr No. 4483. | GCCGCAGA |
| Kmr No. 4484. | GCCGCAGC |
| Kmr No. 4485. | GCCGCCCC |
| Kmr No. 4486. | GCCGCTCC |
| Kmr No. 4487. | GCCGGGGA |
| Kmr No. 4488. | GCCGTAGA |
| Kmr No. 4489. | GCCTAAGC |
| Kmr No. 4490. | GCCTCAAA |
| Kmr No. 4491. | GCCTCACA |
| Kmr No. 4492. | GCCTCAGC |
| Kmr No. 4493. | GCCTCCCC |
| Kmr No. 4494. | GCCTCTAC |
| Kmr No. 4495. | GCCTCTCA |
| Kmr No. 4496. | GCCTGCAA |
| Kmr No. 4497. | GCCTGCCC |
| Kmr No. 4498. | GCCTGCTA |
| Kmr No. 4499. | GCCTGGTA |
| Kmr No. 4500. | GCCTTGCA |
| Kmr No. 4501. | GCCTTGTA |
| Kmr No. 4502. | GCGAAAAA |
| Kmr No. 4503. | GCGACAAA |
| Kmr No. 4504. | GCGACACA |
| Kmr No. 4505. | GCGACAGA |
| Kmr No. 4506. | GCGACATA |
| Kmr No. 4507. | GCGACCCC |
| Kmr No. 4508. | GCGACTAA |
| Kmr No. 4509. | GCGAGAAA |
| Kmr No. 4510. | GCGAGAGA |
| Kmr No. 4511. | GCGAGAGC |
| Kmr No. 4512. | GCGAGATA |
| Kmr No. 4513. | GCGAGGGA |
| Kmr No. 4514. | GCGAGGTA |
| Kmr No. 4515. | GCGATAGA |
| Kmr No. 4516. | GCGATATA |
| Kmr No. 4517. | GCGATGTA |
| Kmr No. 4518. | GCGATTAA |
| Kmr No. 4519. | GCGCAAAA |
| Kmr No. 4520. | GCGCACAA |
| Kmr No. 4521. | GCGCACCC |
| Kmr No. 4522. | GCGCAGAA |
| Kmr No. 4523. | GCGCAGTA |
| Kmr No. 4524. | GCGCCCCA |
| Kmr No. 4525. | GCGCCCCC |
| Kmr No. 4526. | GCGCCCTA |
| Kmr No. 4527. | GCGCGGGA |
| Kmr No. 4528. | GCGCTACA |
| Kmr No. 4529. | GCGCTCTA |
| Kmr No. 4530. | GCGGAGCA |
| Kmr No. 4531. | GCGGAGGA |
| Kmr No. 4532. | GCGGCAAA |
| Kmr No. 4533. | GCGGCACA |
| Kmr No. 4534. | GCGGCAGA |
| Kmr No. 4535. | GCGGCATA |
| Kmr No. 4536. | GCGGCCCC |
| Kmr No. 4537. | GCGGCTCA |
| Kmr No. 4538. | GCGGCTGA |
| Kmr No. 4539. | GCGGGAAA |
| Kmr No. 4540. | GCGGGACA |
| Kmr No. 4541. | GCGGGAGA |
| Kmr No. 4542. | GCGGGAGC |
| Kmr No. 4543. | GCGGGATA |
| Kmr No. 4544. | GCGGGCAA |
| Kmr No. 4545. | GCGGGCCC |
| Kmr No. 4546. | GCGGGCTA |
| Kmr No. 4547. | GCGGGGAA |
| Kmr No. 4548. | GCGGGGAC |
| Kmr No. 4549. | GCGGGGCA |
| Kmr No. 4550. | GCGGGGCC |
| Kmr No. 4551. | GCGGGGGA |
| Kmr No. 4552. | GCGGGGTA |
| Kmr No. 4553. | GCGTAGTA |
| Kmr No. 4554. | GCGTCCCC |
| Kmr No. 4555. | GCGTGGTA |
| Kmr No. 4556. | GCTAATCC |
| Kmr No. 4557. | GCTACACA |
| Kmr No. 4558. | GCTACACC |
| Kmr No. 4559. | GCTACCCC |
| Kmr No. 4560. | GCTAGTCA |
| Kmr No. 4561. | GCTATCCC |
| Kmr No. 4562. | GCTATGCC |
| Kmr No. 4563. | GCTCAAAA |
| Kmr No. 4564. | GCTCAAAC |
| Kmr No. 4565. | GCTCACAA |
| Kmr No. 4566. | GCTCACAC |
| Kmr No. 4567. | GCTCACCC |
| Kmr No. 4568. | GCTCAGCA |
| Kmr No. 4569. | GCTCAGCC |
| Kmr No. 4570. | GCTCCACA |
| Kmr No. 4571. | GCTCCAGC |
| Kmr No. 4572. | GCTCCCCC |
| Kmr No. 4573. | GCTCCCTA |
| Kmr No. 4574. | GCTCCGCA |
| Kmr No. 4575. | GCTCCGCC |
| Kmr No. 4576. | GCTCCTCA |
| Kmr No. 4577. | GCTCCTCC |
| Kmr No. 4578. | GCTCGAAA |
| Kmr No. 4579. | GCTCTACA |
| Kmr No. 4580. | GCTCTACC |
| Kmr No. 4581. | GCTCTCAA |
| Kmr No. 4582. | GCTCTCAC |
| Kmr No. 4583. | GCTCTCCA |
| Kmr No. 4584. | GCTCTCCC |
| Kmr No. 4585. | GCTCTCGA |
| Kmr No. 4586. | GCTCTCTA |
| Kmr No. 4587. | GCTCTGAC |
| Kmr No. 4588. | GCTCTGCA |
| Kmr No. 4589. | GCTCTGCC |
| Kmr No. 4590. | GCTCTTCA |
| Kmr No. 4591. | GCTCTTGA |
| Kmr No. 4592. | GCTGACCC |
| Kmr No. 4593. | GCTGAGCA |
| Kmr No. 4594. | GCTGAGGA |
| Kmr No. 4595. | GCTGCAAA |
| Kmr No. 4596. | GCTGCACA |
| Kmr No. 4597. | GCTGCACC |
| Kmr No. 4598. | GCTGCCCA |
| Kmr No. 4599. | GCTGCCCC |
| Kmr No. 4600. | GCTGCGCC |
| Kmr No. 4601. | GCTGCTCC |
| Kmr No. 4602. | GCTGCTGA |
| Kmr No. 4603. | GCTGGACA |
| Kmr No. 4604. | GCTGGCAC |
| Kmr No. 4605. | GCTGGGCA |
| Kmr No. 4606. | GCTGGGGA |
| Kmr No. 4607. | GCTGTGCA |
| Kmr No. 4608. | GCTGTGCC |
| Kmr No. 4609. | GCTTACCC |
| Kmr No. 4610. | GCTTCCAA |
| Kmr No. 4611. | GCTTCCGA |
| Kmr No. 4612. | GCTTCGAA |
| Kmr No. 4613. | GCTTCTAC |
| Kmr No. 4614. | GCTTGACA |
| Kmr No. 4615. | GCTTGCAA |
| Kmr No. 4616. | GCTTGCCC |
| Kmr No. 4617. | GCTTGTCA |
| Kmr No. 4618. | GCTTTGAC |
| Kmr No. 4619. | GCTTTGCA |
| Kmr No. 4620. | GCTTTGCC |
| Kmr No. 4621. | GGAAACCA |
| Kmr No. 4622. | GGAAATCA |
| Kmr No. 4623. | GGAAATGA |
| Kmr No. 4624. | GGAACAAC |
| Kmr No. 4625. | GGAACCAA |
| Kmr No. 4626. | GGAACCAC |
| Kmr No. 4627. | GGAACCGA |
| Kmr No. 4628. | GGAACCTA |
| Kmr No. 4629. | GGAACGTA |
| Kmr No. 4630. | GGAAGAAC |
| Kmr No. 4631. | GGAAGACA |
| Kmr No. 4632. | GGAAGTAA |
| Kmr No. 4633. | GGAAGTAC |
| Kmr No. 4634. | GGAAGTCA |
| Kmr No. 4635. | GGAAGTGA |
| Kmr No. 4636. | GGAATCAC |
| Kmr No. 4637. | GGAATCCA |
| Kmr No. 4638. | GGAATGAC |
| Kmr No. 4639. | GGAATGCC |
| Kmr No. 4640. | GGAATTAC |
| Kmr No. 4641. | GGAATTCA |
| Kmr No. 4642. | GGAATTCC |
| Kmr No. 4643. | GGACAACA |
| Kmr No. 4644. | GGACACAA |
| Kmr No. 4645. | GGACACAC |
| Kmr No. 4646. | GGACACCC |
| Kmr No. 4647. | GGACAGTA |
| Kmr No. 4648. | GGACCAAA |
| Kmr No. 4649. | GGACCATA |
| Kmr No. 4650. | GGACCCAC |
| Kmr No. 4651. | GGACCCCC |
| Kmr No. 4652. | GGACCGAA |
| Kmr No. 4653. | GGACCGGA |
| Kmr No. 4654. | GGACCGTA |
| Kmr No. 4655. | GGACGAGA |
| Kmr No. 4656. | GGACGTCC |
| Kmr No. 4657. | GGACGTTA |
| Kmr No. 4658. | GGACTACA |
| Kmr No. 4659. | GGAGACCC |
| Kmr No. 4660. | GGAGAGCA |
| Kmr No. 4661. | GGAGAGCC |
| Kmr No. 4662. | GGAGCACA |
| Kmr No. 4663. | GGAGCACC |
| Kmr No. 4664. | GGAGCCCC |
| Kmr No. 4665. | GGAGCGCA |
| Kmr No. 4666. | GGAGGACA |
| Kmr No. 4667. | GGAGGAGA |
| Kmr No. 4668. | GGAGGCAC |
| Kmr No. 4669. | GGAGGGAC |
| Kmr No. 4670. | GGAGGGCA |
| Kmr No. 4671. | GGAGGGCC |
| Kmr No. 4672. | GGAGGGGA |
| Kmr No. 4673. | GGAGGGTA |
| Kmr No. 4674. | GGAGGTCA |
| Kmr No. 4675. | GGAGGTGA |
| Kmr No. 4676. | GGAGTCAC |
| Kmr No. 4677. | GGAGTGCA |
| Kmr No. 4678. | GGAGTTCA |
| Kmr No. 4679. | GGATACAC |
| Kmr No. 4680. | GGATACGA |
| Kmr No. 4681. | GGATATCA |
| Kmr No. 4682. | GGATATGA |
| Kmr No. 4683. | GGATCAAA |
| Kmr No. 4684. | GGATCATA |
| Kmr No. 4685. | GGATCCAA |
| Kmr No. 4686. | GGATCGGA |
| Kmr No. 4687. | GGATCGTA |
| Kmr No. 4688. | GGATCTAC |
| Kmr No. 4689. | GGATTAAA |
| Kmr No. 4690. | GGATTAGA |
| Kmr No. 4691. | GGATTCAC |
| Kmr No. 4692. | GGATTCCA |
| Kmr No. 4693. | GGATTGAC |
| Kmr No. 4694. | GGATTGCC |
| Kmr No. 4695. | GGATTTAC |
| Kmr No. 4696. | GGATTTGA |
| Kmr No. 4697. | GGCAAAAA |
| Kmr No. 4698. | GGCAAAGA |
| Kmr No. 4699. | GGCAACAA |
| Kmr No. 4700. | GGCAAGGA |
| Kmr No. 4701. | GGCACAAA |
| Kmr No. 4702. | GGCACACA |
| Kmr No. 4703. | GGCACACC |
| Kmr No. 4704. | GGCACAGA |
| Kmr No. 4705. | GGCACATA |
| Kmr No. 4706. | GGCACCCC |
| Kmr No. 4707. | GGCACGTA |
| Kmr No. 4708. | GGCACTCA |
| Kmr No. 4709. | GGCACTTA |
| Kmr No. 4710. | GGCAGAAA |
| Kmr No. 4711. | GGCAGAAC |
| Kmr No. 4712. | GGCAGACA |
| Kmr No. 4713. | GGCAGAGA |
| Kmr No. 4714. | GGCAGCAA |
| Kmr No. 4715. | GGCAGCAC |
| Kmr No. 4716. | GGCAGCCC |
| Kmr No. 4717. | GGCAGCGA |
| Kmr No. 4718. | GGCAGGCA |
| Kmr No. 4719. | GGCAGTGA |
| Kmr No. 4720. | GGCATAGA |
| Kmr No. 4721. | GGCCCACC |
| Kmr No. 4722. | GGCCCCAC |
| Kmr No. 4723. | GGCCCCCA |
| Kmr No. 4724. | GGCCCCCC |
| Kmr No. 4725. | GGCCCGCC |
| Kmr No. 4726. | GGCGAAAA |
| Kmr No. 4727. | GGCGCAAA |
| Kmr No. 4728. | GGCGCACA |
| Kmr No. 4729. | GGCGCAGA |
| Kmr No. 4730. | GGCGCCCC |
| Kmr No. 4731. | GGCGGAGA |
| Kmr No. 4732. | GGCGGGAC |
| Kmr No. 4733. | GGCGGGCA |
| Kmr No. 4734. | GGCGGGGA |
| Kmr No. 4735. | GGCGGGTA |
| Kmr No. 4736. | GGCGGTTA |
| Kmr No. 4737. | GGCGTAAA |
| Kmr No. 4738. | GGCGTAGA |
| Kmr No. 4739. | GGCGTATA |
| Kmr No. 4740. | GGCTACAC |
| Kmr No. 4741. | GGCTCAAA |
| Kmr No. 4742. | GGCTCACA |
| Kmr No. 4743. | GGCTCCCC |
| Kmr No. 4744. | GGCTCTCA |
| Kmr No. 4745. | GGCTCTGA |
| Kmr No. 4746. | GGCTGCAA |
| Kmr No. 4747. | GGCTGCAC |
| Kmr No. 4748. | GGCTGCCC |
| Kmr No. 4749. | GGCTTGCA |
| Kmr No. 4750. | GGCTTTGA |
| Kmr No. 4751. | GGGAACCA |
| Kmr No. 4752. | GGGAAGTA |
| Kmr No. 4753. | GGGAATCA |
| Kmr No. 4754. | GGGAATTA |
| Kmr No. 4755. | GGGACACA |
| Kmr No. 4756. | GGGACACC |
| Kmr No. 4757. | GGGACCCA |
| Kmr No. 4758. | GGGACCCC |
| Kmr No. 4759. | GGGACGCA |
| Kmr No. 4760. | GGGAGAGA |
| Kmr No. 4761. | GGGAGCAC |
| Kmr No. 4762. | GGGAGCGA |
| Kmr No. 4763. | GGGAGGAC |
| Kmr No. 4764. | GGGAGGGA |
| Kmr No. 4765. | GGGCAAAA |
| Kmr No. 4766. | GGGCAACA |
| Kmr No. 4767. | GGGCAAGA |
| Kmr No. 4768. | GGGCACAA |
| Kmr No. 4769. | GGGCACAC |
| Kmr No. 4770. | GGGCACCC |
| Kmr No. 4771. | GGGCAGAA |
| Kmr No. 4772. | GGGCAGAC |
| Kmr No. 4773. | GGGCAGCA |
| Kmr No. 4774. | GGGCAGGA |
| Kmr No. 4775. | GGGCATCA |
| Kmr No. 4776. | GGGCCCAC |
| Kmr No. 4777. | GGGCCCCA |
| Kmr No. 4778. | GGGCCCCC |
| Kmr No. 4779. | GGGCCCTA |
| Kmr No. 4780. | GGGCGAAA |
| Kmr No. 4781. | GGGCGACA |
| Kmr No. 4782. | GGGCGAGA |
| Kmr No. 4783. | GGGCGATA |
| Kmr No. 4784. | GGGCGCAA |
| Kmr No. 4785. | GGGCGCAC |
| Kmr No. 4786. | GGGCGCCC |
| Kmr No. 4787. | GGGCGGAC |
| Kmr No. 4788. | GGGCGGCA |
| Kmr No. 4789. | GGGCGGGA |
| Kmr No. 4790. | GGGCTACA |
| Kmr No. 4791. | GGGCTATA |
| Kmr No. 4792. | GGGCTCTA |
| Kmr No. 4793. | GGGCTGCA |
| Kmr No. 4794. | GGGCTTGA |
| Kmr No. 4795. | GGGGACAC |
| Kmr No. 4796. | GGGGACCC |
| Kmr No. 4797. | GGGGACGA |
| Kmr No. 4798. | GGGGAGAA |
| Kmr No. 4799. | GGGGAGAC |
| Kmr No. 4800. | GGGGAGCA |
| Kmr No. 4801. | GGGGAGGA |
| Kmr No. 4802. | GGGGCAAA |
| Kmr No. 4803. | GGGGCAAC |
| Kmr No. 4804. | GGGGCACA |
| Kmr No. 4805. | GGGGCACC |
| Kmr No. 4806. | GGGGCAGA |
| Kmr No. 4807. | GGGGCATA |
| Kmr No. 4808. | GGGGCCAC |
| Kmr No. 4809. | GGGGCCCA |
| Kmr No. 4810. | GGGGCCCC |
| Kmr No. 4811. | GGGGCGAA |
| Kmr No. 4812. | GGGGCGAC |
| Kmr No. 4813. | GGGGCGCA |
| Kmr No. 4814. | GGGGCGGA |
| Kmr No. 4815. | GGGGCGTA |
| Kmr No. 4816. | GGGGCTAC |
| Kmr No. 4817. | GGGGCTCA |
| Kmr No. 4818. | GGGGCTGA |
| Kmr No. 4819. | GGGGGAAA |
| Kmr No. 4820. | GGGGGAAC |
| Kmr No. 4821. | GGGGGACA |
| Kmr No. 4822. | GGGGGACC |
| Kmr No. 4823. | GGGGGAGA |
| Kmr No. 4824. | GGGGGCAA |
| Kmr No. 4825. | GGGGGCAC |
| Kmr No. 4826. | GGGGGCCA |
| Kmr No. 4827. | GGGGGCGA |
| Kmr No. 4828. | GGGGGCTA |
| Kmr No. 4829. | GGGGGGAA |
| Kmr No. 4830. | GGGGGGAC |
| Kmr No. 4831. | GGGGGGCA |
| Kmr No. 4832. | GGGGGGGA |
| Kmr No. 4833. | GGGGGGTA |
| Kmr No. 4834. | GGGGGTAA |
| Kmr No. 4835. | GGGGGTAC |
| Kmr No. 4836. | GGGGGTCA |
| Kmr No. 4837. | GGGGGTGA |
| Kmr No. 4838. | GGGGGTTA |
| Kmr No. 4839. | GGGGTAGA |
| Kmr No. 4840. | GGGGTCAA |
| Kmr No. 4841. | GGGGTCAC |
| Kmr No. 4842. | GGGGTCTA |
| Kmr No. 4843. | GGGGTGAC |
| Kmr No. 4844. | GGGGTGCA |
| Kmr No. 4845. | GGGGTGGA |
| Kmr No. 4846. | GGGGTGTA |
| Kmr No. 4847. | GGGGTTCA |
| Kmr No. 4848. | GGGGTTGA |
| Kmr No. 4849. | GGGGTTTA |
| Kmr No. 4850. | GGGTACCC |
| Kmr No. 4851. | GGGTCAAA |
| Kmr No. 4852. | GGGTCAAC |
| Kmr No. 4853. | GGGTCACA |
| Kmr No. 4854. | GGGTCAGA |
| Kmr No. 4855. | GGGTCATA |
| Kmr No. 4856. | GGGTCCTA |
| Kmr No. 4857. | GGGTCTCA |
| Kmr No. 4858. | GGGTCTGA |
| Kmr No. 4859. | GGGTGACA |
| Kmr No. 4860. | GGGTGAGA |
| Kmr No. 4861. | GGGTGCAA |
| Kmr No. 4862. | GGGTGCAC |
| Kmr No. 4863. | GGGTGGAC |
| Kmr No. 4864. | GGGTGGCA |
| Kmr No. 4865. | GGGTGGGA |
| Kmr No. 4866. | GGGTGTAA |
| Kmr No. 4867. | GGGTGTAC |
| Kmr No. 4868. | GGGTGTCA |
| Kmr No. 4869. | GGGTGTGA |
| Kmr No. 4870. | GGGTTCAA |
| Kmr No. 4871. | GGTAAATA |
| Kmr No. 4872. | GGTATTAA |
| Kmr No. 4873. | GGTATTTA |
| Kmr No. 4874. | GGTCCAAC |
| Kmr No. 4875. | GGTCCATA |
| Kmr No. 4876. | GGTCCGAA |
| Kmr No. 4877. | GGTCCGGA |
| Kmr No. 4878. | GGTCCGTA |
| Kmr No. 4879. | GGTCTCAA |
| Kmr No. 4880. | GGTGAGGA |
| Kmr No. 4881. | GGTGCAAA |
| Kmr No. 4882. | GGTGCAGA |
| Kmr No. 4883. | GGTGCTGA |
| Kmr No. 4884. | GGTGGAAC |
| Kmr No. 4885. | GGTGGGAC |
| Kmr No. 4886. | GGTGGGCA |
| Kmr No. 4887. | GGTGGGGA |
| Kmr No. 4888. | GGTTATGA |
| Kmr No. 4889. | GGTTCCAA |
| Kmr No. 4890. | GGTTCCAC |
| Kmr No. 4891. | GGTTCCGA |
| Kmr No. 4892. | GGTTGACA |
| Kmr No. 4893. | GGTTGGAC |
| Kmr No. 4894. | GGTTTCCA |
| Kmr No. 4895. | GGTTTTGA |
| Kmr No. 4896. | GTAAAAGA |
| Kmr No. 4897. | GTAAAGAA |
| Kmr No. 4898. | GTAAAGTA |
| Kmr No. 4899. | GTAAATAA |
| Kmr No. 4900. | GTAAATAC |
| Kmr No. 4901. | GTAACGTA |
| Kmr No. 4902. | GTAAGCGA |
| Kmr No. 4903. | GTAATCAC |
| Kmr No. 4904. | GTAATGCA |
| Kmr No. 4905. | GTACACAC |
| Kmr No. 4906. | GTACAGAA |
| Kmr No. 4907. | GTACAGGA |
| Kmr No. 4908. | GTACATGA |
| Kmr No. 4909. | GTACCAAA |
| Kmr No. 4910. | GTACCGGA |
| Kmr No. 4911. | GTACCGTA |
| Kmr No. 4912. | GTACGGAA |
| Kmr No. 4913. | GTACGGTA |
| Kmr No. 4914. | GTACGTAA |
| Kmr No. 4915. | GTACGTAC |
| Kmr No. 4916. | GTACGTGA |
| Kmr No. 4917. | GTACGTTA |
| Kmr No. 4918. | GTACTGAC |
| Kmr No. 4919. | GTACTGGA |
| Kmr No. 4920. | GTACTGTA |
| Kmr No. 4921. | GTACTTTA |
| Kmr No. 4922. | GTAGAAGA |
| Kmr No. 4923. | GTAGAGCA |
| Kmr No. 4924. | GTAGCCCA |
| Kmr No. 4925. | GTAGGAAC |
| Kmr No. 4926. | GTAGGAGA |
| Kmr No. 4927. | GTAGGGCA |
| Kmr No. 4928. | GTAGGGGA |
| Kmr No. 4929. | GTAGGTCA |
| Kmr No. 4930. | GTAGTGCA |
| Kmr No. 4931. | GTATACGA |
| Kmr No. 4932. | GTATAGCA |
| Kmr No. 4933. | GTATCCAA |
| Kmr No. 4934. | GTATCCCA |
| Kmr No. 4935. | GTATCCGA |
| Kmr No. 4936. | GTATCGGA |
| Kmr No. 4937. | GTATGGAA |
| Kmr No. 4938. | GTATGGGA |
| Kmr No. 4939. | GTATTAAA |
| Kmr No. 4940. | GTATTAGA |
| Kmr No. 4941. | GTATTCCA |
| Kmr No. 4942. | GTATTGGA |
| Kmr No. 4943. | GTCAAAAC |
| Kmr No. 4944. | GTCAATCA |
| Kmr No. 4945. | GTCACGTA |
| Kmr No. 4946. | GTCACTAA |
| Kmr No. 4947. | GTCACTGA |
| Kmr No. 4948. | GTCAGCAC |
| Kmr No. 4949. | GTCAGGAA |
| Kmr No. 4950. | GTCAGGTA |
| Kmr No. 4951. | GTCAGTAA |
| Kmr No. 4952. | GTCAGTCA |
| Kmr No. 4953. | GTCAGTGA |
| Kmr No. 4954. | GTCATGAA |
| Kmr No. 4955. | GTCATGTA |
| Kmr No. 4956. | GTCCAAAC |
| Kmr No. 4957. | GTCCAAGA |
| Kmr No. 4958. | GTCCAATA |
| Kmr No. 4959. | GTCCAGTA |
| Kmr No. 4960. | GTCCATAA |
| Kmr No. 4961. | GTCCATTA |
| Kmr No. 4962. | GTCCCATA |
| Kmr No. 4963. | GTCCCCAC |
| Kmr No. 4964. | GTCCCCCA |
| Kmr No. 4965. | GTCCCCGA |
| Kmr No. 4966. | GTCCGATA |
| Kmr No. 4967. | GTCCGGAA |
| Kmr No. 4968. | GTCCGGAC |
| Kmr No. 4969. | GTCCTCAC |
| Kmr No. 4970. | GTCCTGTA |
| Kmr No. 4971. | GTCGCAGA |
| Kmr No. 4972. | GTCGCTTA |
| Kmr No. 4973. | GTCGGAAC |
| Kmr No. 4974. | GTCGGGGA |
| Kmr No. 4975. | GTCTCCTA |
| Kmr No. 4976. | GTCTGGAA |
| Kmr No. 4977. | GTCTGGTA |
| Kmr No. 4978. | GTCTTCCA |
| Kmr No. 4979. | GTCTTGGA |
| Kmr No. 4980. | GTGAAACA |
| Kmr No. 4981. | GTGAATCA |
| Kmr No. 4982. | GTGACATA |
| Kmr No. 4983. | GTGACCAA |
| Kmr No. 4984. | GTGACCTA |
| Kmr No. 4985. | GTGACTAA |
| Kmr No. 4986. | GTGAGAGA |
| Kmr No. 4987. | GTGAGATA |
| Kmr No. 4988. | GTGAGGCA |
| Kmr No. 4989. | GTGATCAA |
| Kmr No. 4990. | GTGATTCA |
| Kmr No. 4991. | GTGATTGA |
| Kmr No. 4992. | GTGCAAAC |
| Kmr No. 4993. | GTGCATCA |
| Kmr No. 4994. | GTGCATTA |
| Kmr No. 4995. | GTGCCCCA |
| Kmr No. 4996. | GTGCCCTA |
| Kmr No. 4997. | GTGCCTCA |
| Kmr No. 4998. | GTGCCTTA |
| Kmr No. 4999. | GTGCGGGA |
| Kmr No. 5000. | GTGCTATA |
| Kmr No. 5001. | GTGCTCCA |
| Kmr No. 5002. | GTGCTCTA |
| Kmr No. 5003. | GTGGAACA |
| Kmr No. 5004. | GTGGAAGA |
| Kmr No. 5005. | GTGGAGCA |
| Kmr No. 5006. | GTGGATTA |
| Kmr No. 5007. | GTGGCACA |
| Kmr No. 5008. | GTGGGCAA |
| Kmr No. 5009. | GTGGGCTA |
| Kmr No. 5010. | GTGGGGCA |
| Kmr No. 5011. | GTGGGGGA |
| Kmr No. 5012. | GTGGGGTA |
| Kmr No. 5013. | GTGGGTGA |
| Kmr No. 5014. | GTGTGAGA |
| Kmr No. 5015. | GTGTGCAA |
| Kmr No. 5016. | GTGTGCCA |
| Kmr No. 5017. | GTGTGCTA |
| Kmr No. 5018. | GTGTGGAA |
| Kmr No. 5019. | GTGTGTGA |
| Kmr No. 5020. | GTGTTGGA |
| Kmr No. 5021. | GTTACGGA |
| Kmr No. 5022. | GTTATGGA |
| Kmr No. 5023. | GTTCAGGA |
| Kmr No. 5024. | GTTCCAAA |
| Kmr No. 5025. | GTTCCAAC |
| Kmr No. 5026. | GTTCCACA |
| Kmr No. 5027. | GTTCCATA |
| Kmr No. 5028. | GTTCCGAA |
| Kmr No. 5029. | GTTCCGGA |
| Kmr No. 5030. | GTTCCGTA |
| Kmr No. 5031. | GTTCCTGA |
| Kmr No. 5032. | GTTCTCAA |
| Kmr No. 5033. | GTTCTTTA |
| Kmr No. 5034. | GTTGCCCA |
| Kmr No. 5035. | GTTTCTTA |
| Kmr No. 5036. | GTTTGAGA |
| Kmr No. 5037. | TAAAAGAA |
| Kmr No. 5038. | TAAAGTAA |
| Kmr No. 5039. | TAAAGTCA |
| Kmr No. 5040. | TAAATCCA |
| Kmr No. 5041. | TAAATGGA |
| Kmr No. 5042. | TAACCGGA |
| Kmr No. 5043. | TAACTGGA |
| Kmr No. 5044. | TAAGCACA |
| Kmr No. 5045. | TAAGCTCA |
| Kmr No. 5046. | TAAGGGCA |
| Kmr No. 5047. | TAATACGA |
| Kmr No. 5048. | TAATCCAA |
| Kmr No. 5049. | TAATCGCA |
| Kmr No. 5050. | TAATCGGA |
| Kmr No. 5051. | TAATCTGA |
| Kmr No. 5052. | TAATGCGA |
| Kmr No. 5053. | TAATGGGA |
| Kmr No. 5054. | TAATGTGA |
| Kmr No. 5055. | TACACACA |
| Kmr No. 5056. | TACACCCA |
| Kmr No. 5057. | TACACGTA |
| Kmr No. 5058. | TACACTGA |
| Kmr No. 5059. | TACAGCCA |
| Kmr No. 5060. | TACAGCGA |
| Kmr No. 5061. | TACAGGAA |
| Kmr No. 5062. | TACAGTAA |
| Kmr No. 5063. | TACAGTCA |
| Kmr No. 5064. | TACAGTGA |
| Kmr No. 5065. | TACATCCA |
| Kmr No. 5066. | TACATGGA |
| Kmr No. 5067. | TACCAGTA |
| Kmr No. 5068. | TACCCTCA |
| Kmr No. 5069. | TACCGAGA |
| Kmr No. 5070. | TACCGCAA |
| Kmr No. 5071. | TACCGCCA |
| Kmr No. 5072. | TACCGGAA |
| Kmr No. 5073. | TACCTCAA |
| Kmr No. 5074. | TACCTGAA |
| Kmr No. 5075. | TACCTGCA |
| Kmr No. 5076. | TACCTGGA |
| Kmr No. 5077. | TACGACAA |
| Kmr No. 5078. | TACGATCA |
| Kmr No. 5079. | TACGCCTA |
| Kmr No. 5080. | TACGCGTA |
| Kmr No. 5081. | TACGGTCA |
| Kmr No. 5082. | TACGTCCA |
| Kmr No. 5083. | TACGTGCA |
| Kmr No. 5084. | TACGTGGA |
| Kmr No. 5085. | TACTGGAA |
| Kmr No. 5086. | TACTGGCA |
| Kmr No. 5087. | TACTGGGA |
| Kmr No. 5088. | TACTGTAA |
| Kmr No. 5089. | TACTGTCA |
| Kmr No. 5090. | TACTGTGA |
| Kmr No. 5091. | TACTTCCA |
| Kmr No. 5092. | TACTTGGA |
| Kmr No. 5093. | TACTTTAA |
| Kmr No. 5094. | TAGAAGAA |
| Kmr No. 5095. | TAGACGCA |
| Kmr No. 5096. | TAGAGCGA |
| Kmr No. 5097. | TAGGAAAA |
| Kmr No. 5098. | TAGGCACA |
| Kmr No. 5099. | TAGGCAGA |
| Kmr No. 5100. | TAGGCGCA |
| Kmr No. 5101. | TAGGGCGA |
| Kmr No. 5102. | TAGGGGGA |
| Kmr No. 5103. | TAGTACGA |
| Kmr No. 5104. | TATACAGA |
| Kmr No. 5105. | TATCCGGA |
| Kmr No. 5106. | TATCGGAA |
| Kmr No. 5107. | TATCTCAA |
| Kmr No. 5108. | TATCTCGA |
| Kmr No. 5109. | TATCTGGA |
| Kmr No. 5110. | TATGATCA |
| Kmr No. 5111. | TATGCGCA |
| Kmr No. 5112. | TATGCTCA |
| Kmr No. 5113. | TATGGAAA |
| Kmr No. 5114. | TATGGGAA |
| Kmr No. 5115. | TATGTCGA |
| Kmr No. 5116. | TATTCAGA |
| Kmr No. 5117. | TATTCGAA |
| Kmr No. 5118. | TATTGGAA |
| Kmr No. 5119. | TATTTCCA |
| Kmr No. 5120. | TCAATCCA |
| Kmr No. 5121. | TCAATGGA |
| Kmr No. 5122. | TCACAGGA |
| Kmr No. 5123. | TCACATGA |
| Kmr No. 5124. | TCACCGGA |
| Kmr No. 5125. | TCACGACA |
| Kmr No. 5126. | TCACGTAA |
| Kmr No. 5127. | TCACGTGA |
| Kmr No. 5128. | TCACTAGA |
| Kmr No. 5129. | TCACTGAA |
| Kmr No. 5130. | TCACTGCA |
| Kmr No. 5131. | TCACTGGA |
| Kmr No. 5132. | TCACTTCA |
| Kmr No. 5133. | TCAGACGA |
| Kmr No. 5134. | TCAGATAA |
| Kmr No. 5135. | TCAGCACA |
| Kmr No. 5136. | TCAGGAAA |
| Kmr No. 5137. | TCAGGCAA |
| Kmr No. 5138. | TCAGGTAA |
| Kmr No. 5139. | TCAGTACA |
| Kmr No. 5140. | TCAGTCCA |
| Kmr No. 5141. | TCAGTGAA |
| Kmr No. 5142. | TCAGTGGA |
| Kmr No. 5143. | TCATCCCA |
| Kmr No. 5144. | TCATCGGA |
| Kmr No. 5145. | TCATGAAA |
| Kmr No. 5146. | TCATGGAA |
| Kmr No. 5147. | TCATGGGA |
| Kmr No. 5148. | TCATTCCA |
| Kmr No. 5149. | TCATTGGA |
| Kmr No. 5150. | TCCAAAGA |
| Kmr No. 5151. | TCCAACCA |
| Kmr No. 5152. | TCCAAGAA |
| Kmr No. 5153. | TCCAAGGA |
| Kmr No. 5154. | TCCAATCA |
| Kmr No. 5155. | TCCACCAA |
| Kmr No. 5156. | TCCAGCAA |
| Kmr No. 5157. | TCCAGGAA |
| Kmr No. 5158. | TCCAGTAA |
| Kmr No. 5159. | TCCAGTCA |
| Kmr No. 5160. | TCCATAAA |
| Kmr No. 5161. | TCCATAGA |
| Kmr No. 5162. | TCCATCAA |
| Kmr No. 5163. | TCCATCCA |
| Kmr No. 5164. | TCCATGAA |
| Kmr No. 5165. | TCCATGCA |
| Kmr No. 5166. | TCCATGGA |
| Kmr No. 5167. | TCCATTAA |
| Kmr No. 5168. | TCCCAGGA |
| Kmr No. 5169. | TCCCATAA |
| Kmr No. 5170. | TCCCATCA |
| Kmr No. 5171. | TCCCCCCA |
| Kmr No. 5172. | TCCCCCGA |
| Kmr No. 5173. | TCCCCGCA |
| Kmr No. 5174. | TCCCTGAA |
| Kmr No. 5175. | TCCGAACA |
| Kmr No. 5176. | TCCGAAGA |
| Kmr No. 5177. | TCCGATAA |
| Kmr No. 5178. | TCCGATCA |
| Kmr No. 5179. | TCCGCCAA |
| Kmr No. 5180. | TCCGCTCA |
| Kmr No. 5181. | TCCGGAAA |
| Kmr No. 5182. | TCCGGACA |
| Kmr No. 5183. | TCCGGAGA |
| Kmr No. 5184. | TCCGGCAA |
| Kmr No. 5185. | TCCGGTAA |
| Kmr No. 5186. | TCCGGTCA |
| Kmr No. 5187. | TCCGTACA |
| Kmr No. 5188. | TCCGTCAA |
| Kmr No. 5189. | TCCGTGAA |
| Kmr No. 5190. | TCCTACAA |
| Kmr No. 5191. | TCCTACGA |
| Kmr No. 5192. | TCCTAGAA |
| Kmr No. 5193. | TCCTAGGA |
| Kmr No. 5194. | TCCTGGAA |
| Kmr No. 5195. | TCCTGTAA |
| Kmr No. 5196. | TCCTGTCA |
| Kmr No. 5197. | TCCTTCCA |
| Kmr No. 5198. | TCGACACA |
| Kmr No. 5199. | TCGACAGA |
| Kmr No. 5200. | TCGAGAAA |
| Kmr No. 5201. | TCGAGAGA |
| Kmr No. 5202. | TCGATCAA |
| Kmr No. 5203. | TCGGAACA |
| Kmr No. 5204. | TCGGACCA |
| Kmr No. 5205. | TCGGATCA |
| Kmr No. 5206. | TCGGGGCA |
| Kmr No. 5207. | TCGGTTCA |
| Kmr No. 5208. | TCTCGAAA |
| Kmr No. 5209. | TCTCGTCA |
| Kmr No. 5210. | TCTGATCA |
| Kmr No. 5211. | TCTGCCCA |
| Kmr No. 5212. | TCTGCGCA |
| Kmr No. 5213. | TCTGGAAA |
| Kmr No. 5214. | TCTGTAAA |
| Kmr No. 5215. | TCTGTACA |
| Kmr No. 5216. | TCTTCCAA |
| Kmr No. 5217. | TCTTGGAA |
| Kmr No. 5218. | TCTTTAAA |
| Kmr No. 5219. | TCTTTCCA |
| Kmr No. 5220. | TCTTTTAA |
| Kmr No. 5221. | TGAACACA |
| Kmr No. 5222. | TGACCAAA |
| Kmr No. 5223. | TGACCCCA |
| Kmr No. 5224. | TGACCTCA |
| Kmr No. 5225. | TGACTGAA |
| Kmr No. 5226. | TGAGAGCA |
| Kmr No. 5227. | TGAGCACA |
| Kmr No. 5228. | TGAGCGCA |
| Kmr No. 5229. | TGAGGACA |
| Kmr No. 5230. | TGAGGGCA |
| Kmr No. 5231. | TGAGTGCA |
| Kmr No. 5232. | TGATCAAA |
| Kmr No. 5233. | TGATCACA |
| Kmr No. 5234. | TGATCCAA |
| Kmr No. 5235. | TGATCCCA |
| Kmr No. 5236. | TGATCGAA |
| Kmr No. 5237. | TGATCGCA |
| Kmr No. 5238. | TGATGGAA |
| Kmr No. 5239. | TGATTGAA |
| Kmr No. 5240. | TGCAAGCA |
| Kmr No. 5241. | TGCACACA |
| Kmr No. 5242. | TGCACCCA |
| Kmr No. 5243. | TGCAGGAA |
| Kmr No. 5244. | TGCCCACA |
| Kmr No. 5245. | TGCCCCAA |
| Kmr No. 5246. | TGCCCCCA |
| Kmr No. 5247. | TGCCCGCA |
| Kmr No. 5248. | TGCCGGAA |
| Kmr No. 5249. | TGCCGTAA |
| Kmr No. 5250. | TGCGAAAA |
| Kmr No. 5251. | TGCGCAAA |
| Kmr No. 5252. | TGCGCACA |
| Kmr No. 5253. | TGCTCAAA |
| Kmr No. 5254. | TGCTCACA |
| Kmr No. 5255. | TGCTTCCA |
| Kmr No. 5256. | TGGAACAA |
| Kmr No. 5257. | TGGAACCA |
| Kmr No. 5258. | TGGAAGAA |
| Kmr No. 5259. | TGGACCAA |
| Kmr No. 5260. | TGGATACA |
| Kmr No. 5261. | TGGATCAA |
| Kmr No. 5262. | TGGATGAA |
| Kmr No. 5263. | TGGGAACA |
| Kmr No. 5264. | TGGGACCA |
| Kmr No. 5265. | TGGGCAAA |
| Kmr No. 5266. | TGGGCACA |
| Kmr No. 5267. | TGGGGCAA |
| Kmr No. 5268. | TGGTCCAA |
| Kmr No. 5269. | TGGTGGAA |
| Kmr No. 5270. | TGTCTGAA |
| Kmr No. 5271. | TGTGCAAA |
| Kmr No. 5272. | TGTGCACA |
| Kmr No. 5273. | TGTTCCAA |
| Kmr No. 5274. | TGTTCGAA |
| Kmr No. 5275. | TGTTGGAA |
| Kmr No. 5276. | TTAAAGAA |
| Kmr No. 5277. | TTAATGAA |
| Kmr No. 5278. | TTACAGAA |
| Kmr No. 5279. | TTACGGAA |
| Kmr No. 5280. | TTATGGAA |
| Kmr No. 5281. | TTCACGAA |
| Kmr No. 5282. | TTCAGGAA |
| Kmr No. 5283. | TTCATGAA |
| Kmr No. 5284. | TTCCAGAA |
| Kmr No. 5285. | TTCCGGAA |
| Kmr No. 5286. | TTGATCAA |
| Kmr No. 5287. | TTGGCCAA |

TABLE 4

| | A | B | C | D | E | F | G |
|---|---|---|---|---|---|---|---|
| 1 | kmers | Sens. | Spec. | PPV | NBV | Acc. | FPV |
| 2 | 610; 1555; 2244; 2926; 3741 | 100 | 100 | 100 | 100 | 100 | 7.77E−11 |
| 3 | 53; 511; 689; 1875; 3256; 3360; 3391; 3838; 5021 | 100 | 100 | 100 | 100 | 100 | 7.77E−11 |
| 4 | 379; 675; 965; 1062; 1365; 1523; 2706; 4729 | 100 | 100 | 100 | 100 | 100 | 7.77E−11 |
| 5 | 127; 164; 280; 421; 523; 586; 711; 734; 742; 1068; 1202; 1293; 1310; 1739; 1955; 2389; 2401; 2448; | 100 | 100 | 100 | 100 | 100 | 7.77E−11 |
| | 2626; 2837; 3022; 3103; 3222; 3248; 3401; 3443; 3489; 3516; 3654; 3677; 3866; 4207; 4344; 4403; 4439; 4445; 4689; 4885; 4886; 4983 | | | | | | |
| 6 | 2327; 2577; 2583; 2711; 3178; 3497; 3582; 3728 | 100 | 100 | 100 | 100 | 100 | 7.77E−11 |
| 7 | 243; 417; 444; 519; 990; 1074; 1222; 1364; 1655; 1718; 1890; 2048; 2227; 2670; 3834; 3875; 4437; 4793; 4967 | 100 | 100 | 100 | 100 | 100 | 7.77E−11 |
| 8 | 558; 1058; 1161; 1281; 1766; 2066; 2520; 3339; 4063; 4641; 4911 | 100 | 100 | 100 | 100 | 100 | 7.77E−11 |
| 9 | 591; 778; 1111; 1442; 1707; 1742; 1818; 2004; 2090; 2485; 2707; 2795; 2799; 3506; 3570; 3835; 4281; 4697; 4976; 5031; 5240 | 100 | 100 | 100 | 100 | 100 | 7.77E−11 |
| 10 | 49; 153; 350; 544; 570; 678; 685; 805; 832; 1089; 1599; 1808; 2079; 2220; 2471; 2486; 2514; 2964; | 100 | 100 | 100 | 100 | 100 | 7.77E−11 |
| | 3106; 3179; 3195; 3204; 3291; 3414; 3419; 3465; 3554; 3614; 3725; 4227; 4439; 4496; 4748; 4792; 4818 | | | | | | |
| 11 | 1565; 1735; 2297; 2485; 2647; 2935; 3096; 3487; 3526; 5086; 5274 | 100 | 100 | 100 | 100 | 100 | 7.77E−11 |
| 12 | 356; 800; 961; 1716; 1845; 1862; 4435 | 100 | 100 | 100 | 100 | 100 | 7.77E−11 |
| 13 | 136; 187; 986; 1067; 1858; 1993; 2046; 2435; 2904; 3060; 3182; 3718; 3953; 4087; 4472; 4491; 4656; 4842; 5094; 5111 | 100 | 100 | 100 | 100 | 100 | 7.77E−11 |
| 14 | 934; 979; 1079; 1536; 1656; 4008; 4301 | 100 | 100 | 100 | 100 | 100 | 7.77E−11 |
| 15 | 103; 199; 823; 1497; 2374; 2384; 2455; 2652; 3031; 3783; 3974; 5062 | 100 | 100 | 100 | 100 | 100 | 7.77E−11 |
| 16 | 1; 152; 599; 606; 675; 707; 1075; 1258; 1324; 1415; 1416; 1603; 1623; 1904; 2010; 2128; 2219; 2307; | 100 | 100 | 100 | 100 | 100 | 7.77E−11 |
| | 2392; 2431; 2668; 2697; 2736; 2740; 2779; 2897; 3194; 3196; 3619; 3673; 3746; 3837; 3865; 3887; | | | | | | |
| | 3901; 3912; 3976; 3977; 4266; 4311; 4407; 4618; 4624; 5087; 5132 | | | | | | |
| 17 | 66; 280; 283; 571; 608; 726; 796; 948; 963; 1029; 1038; 1061; 1067; 1151; 1376; 1528; 1633; 1638; | 100 | 100 | 100 | 100 | 100 | 7.77E−11 |
| | 1682; 2031; 2274; 2477; 2671; 2676; 2689; 2741; 2882; 2931; 3220; 3228; 3236; 3308; 3456; 3576; | | | | | | |
| | 3690; 3730; 3800; 3857; 4461; 4548; 4625; 4764; 4800; 4845; 4939; 4948; 4956; 5051; 5147; 5265 | | | | | | |
| 18 | 15; 337; 367; 533; 815; 856; 857; 1231; 1290; 1500; 1639; 1770; 1907; 2054; 2559; 2675; 4017; 4489; 4507; 4823; 4861; 4877; 4893; 4910; 4945; | 100 | 100 | 100 | 100 | 100 | 7.77E−11 |
| | 4988; 5121 | | | | | | |
| 19 | 207; 1625; 2116; 2201; 4176 | 100 | 100 | 100 | 100 | 100 | 7.77E−11 |
| 20 | 473; 497; 551; 842; 957; 1094; 1720; 1741; 2279; 2494; 2673; 3335; 3586; 3827 | 100 | 100 | 100 | 100 | 100 | 7.77E−11 |
| 21 | 65; 518; 549; 651; 772; 1176; 1187; 1207; 1209; 1339; 1362; 1801; 2575; 2592; 2615; 2837; 3039; | 100 | 100 | 100 | 100 | 100 | 7.77E−11 |
| | 3115; 3264; 3354; 3895; 3959; 3998; 4408; 4630; 4725; 4822; 4845; 4981 | | | | | | |
| 22 | 87; 237; 301; 444; 466; 517; 756; 792; 870; 1188; 1471; 1808; 1874; 1884; 1893; 2052; 2163; 2562; | 100 | 100 | 100 | 100 | 100 | 7.77E−11 |
| | 2653; 2760; 2856; 3139; 3145; 3171; 3184; 3494; 3524; 3689; 3869; 3909; 3946; 3982; 4013; 4674; 4750; 4786; 4843; 5226 | | | | | | |
| 23 | 2; 369; 433; 575; 719; 784; 794; 838; 920; 1371; 1547; 1581; 1719; 1993; 2217; 2274; 2364; 2395; 2478; 2488; 2537; | 100 | 100 | 100 | 100 | 100 | 7.77E−11 |
| | 2684; 2706; 2758; 2866; 2997; 3185; 3226; 3430; 3583; 3590; 4014; 4112; 4288; 4637; 4806; 4890; 4989; 5061; 5251 | | | | | | |
| 24 | 287; 2079; 3083; 3141; 3351; 3358; 4095; 4751; 4910; 4951 | 100 | 100 | 100 | 100 | 100 | 7.77E−11 |
| 25 | 2472; 3716; 4029; 4089; 4363 | 100 | 100 | 100 | 100 | 100 | 7.77E−11 |
| 26 | 2305; 2547; 2744; 2853; 4859 | 100 | 100 | 100 | 100 | 100 | 7.77E−11 |
| 27 | 99; 143; 218; 538; 573; 629; 632; 636; 826; 1010; 1130; 1252; 1393; 1662; 1704; 1706; 1721; 1949; | 100 | 100 | 100 | 100 | 100 | 7.77E−11 |
| | 2031; 2207; 2564; 2707; 2911; 3130; 3516; 3521; 4374; 4936; 5195; 5257 | | | | | | |
| 28 | 15; 238; 241; 336; 939; 1065; 1068; 1157; 1178; 1567; 1686; 2463; 2497; 2707; 3697; 3895; 4235; 4323; 4326; 4392; 4433; 4672; 4978; 5091 | 100 | 100 | 100 | 100 | 100 | 7.77E−11 |
| 29 | 20; 83; 103; 353; 382; 605; 952; 956; 1591; 1630; 1714; 1751; 2056; 2369; 2468; 2648; 2703; 2928; 2953; 3010; 3047; | 100 | 100 | 100 | 100 | 100 | 7.77E−11 |
| | 3227; 3487; 3799; 3812; 3886; 3942; 3992; 4100; 4144; 4169; 4647; 4771; 4787; 4863; 4864; 4871; 4993; 5285 | | | | | | |
| 30 | 102; 255; 394; 397; 530; 537; 907; 1047; 1066; 1212; 1239; 1421; 1572; 1600; 1729; 1829; 1843; 2082; 2277; 2394; | 100 | 100 | 100 | 100 | 100 | 7.77E−11 |
| | 2479; 2507; 2565; 2567; 2675; 2712; 2739; 2804; 2816; 2831; 2941; 3328; 3456; 3582; 3596; 3658; 3916; 4089; 4126; 4386; 4424; 4628; 5002; 5042; 5218 | | | | | | |
| 31 | 145; 301; 569; 1747; 2190; 3215; 4376; 4397; 5007; 5258 | 100 | 100 | 100 | 100 | 100 | 7.77E−11 |
| 32 | 2161; 2485; 2694; 3206; 3933; 4476 | 100 | 100 | 100 | 100 | 100 | 7.77E−11 |
| 33 | 70; 486; 675; 1148; 1377; 1382; 1492; 1949; 2482; 2575; 2651; 2832; 3022; 3115; 3134; 3148; 3381; | 100 | 100 | 100 | 100 | 100 | 7.77E−11 |
| | 3462; 3826; 3919; 3967; 4018; 4244; 4252; 4370; 4623; 4637; 4776; 5161; 5235 | | | | | | |
| 34 | 119; 444; 1613; 1644; 2074; 2328; 2937; 3974; 5019; 5233 | 100 | 100 | 100 | 100 | 100 | 7.77E−11 |
| 35 | 35; 163; 164; 468; 520; 549; 616; 767; 940; 1098; 1162; 1296; 1551; 1822; 1832; 1951; 2101; 2169; 2364; 2389; 2463; 2520; | 100 | 100 | 100 | 100 | 100 | 7.77E−11 |
| | 2556; 2755; 2779; 2993; 3014; 3027; 3544; 3645; 3688; 3723; 3875; 3901; 4206; 4211; 4254; 4313; 4466; 4526; 4544; 4572; 4804; 4942; 5017; 5021; 5085 | | | | | | |
| 36 | 2453; 2486; 2730; 3749; 3862; 4933; 5268 | 100 | 100 | 100 | 100 | 100 | 7.77E−11 |
| 37 | 131; 526; 1280; 1500; 1759; 3068; 3388; 5037 | 100 | 100 | 100 | 100 | 100 | 7.77E−11 |
| 38 | 155; 461; 567; 1066; 1477; 1822; 2257; 2281; 2625; 2993; 3246; 3323; 3498; 4600; 4835; 5105 | 100 | 100 | 100 | 100 | 100 | 7.77E−11 |
| 39 | 51; 207; 216; 219; 389; 408; 561; 629; 720; 1196; 1220; 1348; 1451; 1463; 1500; 1565; 1586; 1625; | 100 | 100 | 100 | 100 | 100 | 7.77E−11 |
| | 1687; 1860; 2103; 2247; 2313; 2330; 2499; 2748; 2907; 3038; 3063; 3191; 3586; 3721; 3758; 4006; | | | | | | |
| | 4173; 4330; 4343; 4510; 4535; 4558; 4645; 4650; 4706; 4779; 4986; 5083; 5107 | | | | | | |
| 40 | 116; 564; 591; 1263; 2794; 2955; 3236; 3586; 3817; 4654 | 100 | 100 | 100 | 100 | 100 | 7.77E−11 |
| 41 | 623; 1114; 2751; 3984 | 100 | 100 | 100 | 100 | 100 | 7.77E−11 |
| 42 | 286; 719; 1542; 2148; 2695; 3033; 4122 | 100 | 100 | 100 | 100 | 100 | 7.77E−11 |
| 43 | 417; 916; 1057; 2345; 2412; 2797; 4161 | 100 | 100 | 100 | 100 | 100 | 7.77E−11 |
| 44 | 1145; 3158; 3257; 3959; 4877 | 100 | 100 | 100 | 100 | 100 | 7.77E−11 |
| 45 | 1246; 1793; 2318; 2903; 2959; 3341; 3721; 4022; 4613 | 100 | 100 | 100 | 100 | 100 | 7.77E−11 |
| 46 | 303; 383; 424; 637; 999; 1180; 1267; 1308; 1646; 2217; 2453; 3009; 3054; 3185; 3538; 3575; 3677; 3763; 4008; 4158; 4184; 4831; | 100 | 100 | 100 | 100 | 100 | 7.77E−11 |
| | 4873; 5176 | | | | | | |
| 47 | 1985; 2113; 2807; 2981 | 100 | 100 | 100 | 100 | 100 | 7.77E−11 |
| 48 | 173; 177; 291; 521; 526; 529; 568; 784; 815; 819; 824; 857; 967; 1080; 1099; 1208; 1403; 1686; | 100 | 100 | 100 | 100 | 100 | 7.77E−11 |
| | 1711; 1900; 2106; 2137; 2219; 2267; 2451; 2460; 2493; 2576; 2684; 2840; 2872; 3025; 3251; 3265; | | | | | | |
| | 3603; 3607; 3640; 3932; 4026; 4153; 4516; 4613; 4642; 4880; 5001; 5225; 5261 | | | | | | |
| 49 | 8; 30; 108; 152; 257; 688; 717; 1041; 1107; 1324; 1397; 1764; 1837; 1968; 1987; 2035; 2325; 2352; | 100 | 100 | 100 | 100 | 100 | 7.77E−11 |
| | 2359; 2376; 2386; 2409; 2498; 2515; 3082; 3288; 3492; 3505; 3553; 3624; 3776; 3837; 3850; 3922; | | | | | | |
| | 3966; 3977; 4097; 4462; 4646; 4758; 4769; 4856; 4942; 4995; 5205 | | | | | | |
| 50 | 933; 1007; 3125; 3914; 4203; 4698; 4865; 5181 | 100 | 100 | 100 | 100 | 100 | 7.77E−11 |
| 51 | 400; 1734; 2433; 2523; 2732; 3832; 4838 | 100 | 100 | 100 | 100 | 100 | 7.77E−11 |
| 52 | 1433; 1481; 2239; 2321; 3308; 3696; 4153; 4960; 5074 | 100 | 100 | 100 | 100 | 100 | 7.77E−11 |
| 53 | 1; 197; 215; 267; 754; 757; 788; 911; 1428; 1607; 1626; 1798; 1800; 1837; 2006; 2063; 2169; 2254; | 100 | 100 | 100 | 100 | 100 | 7.77E−11 |
| | 2529; 2907; 3305; 3317; 3762; 3836; 3874; 4010; 4037; 4252; 4413; 4513; 4736; 4747; 4758; 4948; 5097; 5220; 5234 | | | | | | |
| 54 | 0; 128; 350; 463; 563; 630; 767; 847; 986; 1125; 1151; 1338; 1389; 1406; 1584; 2255; 2389; 2425; | 100 | 100 | 100 | 100 | 100 | 7.77E−11 |
| | 2619; 2811; 2840; 2912; 2951; 2976; 3025; 3183; 3235; 3276; 3402; 3505; 3764; 3778; 4096; 4114; 4307; 4804; 4952 | | | | | | |
| 55 | 185; 424; 841; 963; 1136; 1559; 1830; 2665; 4767; 4838 | 100 | 100 | 100 | 100 | 100 | 7.77E−11 |
| 56 | 582; 713; 1171; 1590; 1995; 2046; 2281; 2727; 2936; 3370; 5098; 5163 | 100 | 100 | 100 | 100 | 100 | 7.77E−11 |
| 57 | 859; 2815 | 100 | 100 | 100 | 100 | 100 | 7.77E−11 |
| 58 | 661; 845; 1086; 2211; 2697; 3586; 5033 | 100 | 100 | 100 | 100 | 100 | 7.77E−11 |
| 59 | 192; 1370; 1701; 2455; 2658; 2983; 3098; 3166; 3219; 3636; 3691; 4595 | 100 | 100 | 100 | 100 | 100 | 7.77E−11 |
| 60 | 444; 2326; 2512; 2971; 4630 | 100 | 100 | 100 | 100 | 100 | 7.77E−11 |
| 61 | 331; 346; 388; 529; 779; 815; 830; 1019; 1281; 1285; 1368; 1389; 1665; 2035; 2482; 2604; 2798; | 100 | 100 | 100 | 100 | 100 | 7.77E−11 |
| | 2842; 3018; 3170; 3326; 3364; 3399; 3408; 3460; 4018; 4099; 4346 | | | | | | |
| 62 | 2676; 2932; 3079; 3240 | 100 | 100 | 100 | 100 | 100 | 7.77E−11 |
| 63 | 227; 280; 366; 411; 510; 626; 638; 651; 791; 949; 988; 1029; 1151; 1328; 1469; 1498; 1519; 1604; | 100 | 100 | 100 | 100 | 100 | 7.77E−11 |
| | 1617; 1764; 1920; 1934; 2228; 2528; 2757; 2872; 2873; 2880; 2912; 2940; 3082; 3114; 3488; 3649; | | | | | | |
| | 3697; 3787; 3802; 4007; 4323; 4491; 4561; 4660; 4700; 4723; 4837; 4868; 4982; 5068; 5277 | | | | | | |
| 64 | 68; 405; 521; 796; 897; 970; 1132; 1149; 1242; 1435; 1521; 1680; 1704; 1729; 1800; 1917; 2121; | 100 | 100 | 100 | 100 | 100 | 7.77E−11 |
| | 2152; 2244; 2327; 2370; 2387; 2394; 2909; 2995; 3004; 3069; 3167; 3569; 3586; 3620; 3652; 3814; | | | | | | |
| | 3860; 4035; 4325; 4412; 4612; 4652; 4733; 4758; 4861; 5002; 5094; 5278; 5283 | | | | | | |
| 65 | 36; 223; 350; 1105; 1532; 1644; 1702; 1706; 1989; 2416; 2443; 3036; 3130; 3143; 3210; 3211; 3221; 3679; 3950; 4402; 4575; 4580; 4735; 4858 | 100 | 100 | 100 | 100 | 100 | 7.77E−11 |
| 66 | 1233; 1316; 1883; 2040; 2358; 2615; 3414; 3454; 3519; 3587; 3747; 3774; 4367; 4410; 4794; 5011; 5105 | 100 | 100 | 100 | 100 | 100 | 7.77E−11 |
| 67 | 134; 299; 324; 555; 925; 1352; 1471; 1477; 1661; 1928; 1941; 2011; 2064; 2179; 2563; 2751; 2902; | 100 | 100 | 100 | 100 | 100 | 7.77E−11 |
| | 3274; 3452; 3477; 3583; 3623; 3679; 3822; 4220; 4284; 4389; 4478; 4537; 4875 | | | | | | |
| 68 | 135; 432; 587; 724; 786; 1081; 1197; 1343; 1405; 1591; 1653; 1688; 1797; 1820; 2002; 2005; 2127; | 100 | 100 | 100 | 100 | 100 | 7.77E−11 |
| | 2220; 2312; 2463; 2466; 2558; 2641; 2642; 2699; 2815; 3005; 3412; 3433; 3437; 3456; 3858; 3946; | | | | | | |
| | 3982; 4121; 4128; 4704; 4888; 5006; 5067; 5218; 5254; 5256 | | | | | | |
| 69 | 22; 86; 527; 540; 860; 1046; 1179; 1365; 1368; 1558; 2032; 2052; 2136; 2188; 2227; 2332; 2458; | 100 | 100 | 100 | 100 | 100 | 7.77E−11 |
| | 2641; 2701; 2873; 3199; 3340; 3479; 3653; 3830; 3923; 4102; 4191; 4520; 4921; 4981; 5069; 5214; 5265 | | | | | | |
| 70 | 380; 1846; 1945; 2046; 2386; 2584; 4124; 4250; 4633 | 100 | 100 | 100 | 100 | 100 | 7.77E−11 |
| 71 | 1596; 2467 | 100 | 100 | 100 | 100 | 100 | 7.77E−11 |
| 72 | 458; 1592; 1767; 2782; 2846; 3653; 3751; 3930; 4440; 5098 | 100 | 100 | 100 | 100 | 100 | 7.77E−11 |
| 73 | 42; 1232; 2751; 3368; 4700; 5007 | 100 | 100 | 100 | 100 | 100 | 7.77E−11 |
| 74 | 304; 326; 344; 547; 586; 865; 961; 1007; 1019; 1079; 1363; 1686; 1710; 1764; 1769; 1784; 1785; | 100 | 100 | 100 | 100 | 100 | 7.77E−11 |
| | 1860; 2024; 2655; 3397; 3540; 3582; 3674; 3676; 3816; 3860; 4005; 4121 | | | | | | |
| 75 | 1290; 2126; 3628; 3958; 4388; 4488; 4800; 5076 | 100 | 100 | 100 | 100 | 100 | 7.77E−11 |
| 76 | 261; 679; 738; 758; 919; 1007; 1232; 1553; 1573; 1687; 1734; 1810; 2048; 2194; 2345; 2584; | 100 | 100 | 100 | 100 | 100 | 7.77E−11 |
| | 2611; 2616; 2817; 2845; 3124; 3209; 3518; 3637; 3764; 4073; 4247; 4489; 4724; 4810; 5012; 5116; 5230 | | | | | | |
| 77 | 1000; 1029; 1178; 1592; 2625; 3194; 3886; 5077; 5178 | 100 | 100 | 100 | 100 | 100 | 7.77E−11 |
| 78 | 27; 240; 348; 493; 568; 701; 995; 1172; 1222; 1238; 1421; 1524; 1779; 1790; 1804; 1823; 2031; | 100 | 100 | 100 | 100 | 100 | 7.77E−11 |
| | 2271; 2563; 2756; 2938; 3453; 3542; 3588; 3634; 3651; 3738; 3772; 3821; 4026; 4028; 4312; 4318; 4323; 4468; 4473; 5273 | | | | | | |
| 79 | 397; 1401; 1766; 2134; 2485; 2612; 2820; 3178; 3667; 4010; 4542; 4754; 5214 | 100 | 100 | 100 | 100 | 100 | 7.77E−11 |
| 80 | 196; 1880; 1902; 2318; 2622; 4662; 4711; 4772; 4863; 5284 | 100 | 100 | 100 | 100 | 100 | 7.77E−11 |
| 81 | 1115; 2196; 2418; 2909; 3468; 3582; 3593; 3997; 4126; 4621; 4832 | 100 | 100 | 100 | 100 | 100 | 7.77E−11 |
| 82 | 874; 941; 1304; 2671; 3587; 4124 | 100 | 100 | 100 | 100 | 100 | 7.77E−11 |
| 83 | 301; 3948; 4128 | 100 | 100 | 100 | 100 | 100 | 7.77E−11 |
| 84 | 299; 1081; 1567; 2125; 2162; 2239; 4494; 4640 | 100 | 100 | 100 | 100 | 100 | 7.77E−11 |
| 85 | 548; 1419; 1568; 2452; 3018; 3920; 4399; 4907 | 100 | 100 | 100 | 100 | 100 | 7.77E−11 |
| 86 | 7; 41; 83; 193; 199; 215; 247; 510; 525; 685; 758; 818; 835; 1100; 1207; 1353; 1737; 1799; 1819; | 100 | 100 | 100 | 100 | 100 | 7.77E−11 |
| | 1920; 2036; 2105; 2107; 2240; 2445; 2474; 2688; 3054; 3406; 3429; 3650; 3827; 3986; 4256; 4599; 4727; 5131; 5205 | | | | | | |
| 87 | 33; 86; 378; 402; 489; 575; 630; 720; 726; 755; 857; 864; 957; 993; 1117; 1199; 1282; 1286; 1687; 2023; 2031; 2344; 2372; | 100 | 100 | 100 | 100 | 100 | 7.77E−11 |
| | 2537; 2549; 2614; 2651; 2760; 3281; 3365; 3598; 3746; 4031; 4033; 4118; 4490; 4519; 4672; 4767; 4780; 4836; 4837; 4974; 5042; 5139; 5173 | | | | | | |
| 88 | 112; 730; 749; 1556; 2922; 3283; 3705; 4151; 4437; 4652; 4966; 5075 | 100 | 100 | 100 | 100 | 100 | 7.77E−11 |
| 89 | 440; 701; 869; 1029; 1264; 1357; 1652; 2004; 2024; 2110; 2372; 2407; 2813; 3012; 3043; 3542; 3698; | 100 | 100 | 100 | 100 | 100 | 7.77E−11 |
| | 3772; 4280; 4585; 4629; 4826; 4899; 5162 | | | | | | |
| 90 | 38; 41; 229; 379; 401; 419; 646; 675; 1170; 2051; 2499; 2740; 3003; 3178; 3422; 3490; 3625; 3877; | 100 | 100 | 100 | 100 | 100 | 7.77E−11 |
| | 3905; 4097; 4486; 4553; 4719; 4946; 4976; 5114 | | | | | | |
| 91 | 55; 227; 422; 439; 1081; 1120; 1595; 1730; 2268; 2319; 2567; 2598; 3855; 3949; 4367; 4444; 4801; 5098; 5267 | 100 | 100 | 100 | 100 | 100 | 7.77E−11 |
| 92 | 1865; 2766; 2878; 2934; 3152; 3291; 3619; 3639; 5057 | 100 | 100 | 100 | 100 | 100 | 7.77E−11 |
| 93 | 2333; 2362; 3004; 4304; 4676; 5080 | 100 | 100 | 100 | 100 | 100 | 7.77E−11 |
| 94 | 44; 220; 626; 689; 1034; 1088; 1180; 1362; 1424; 1441; 1638; 1846; 1978; 2100; 2196; 2465; 2636; | 100 | 100 | 100 | 100 | 100 | 7.77E−11 |
| | 2752; 3356; 3579; 3974; 4017; 4166; 4192; 4968; 5090 | | | | | | |
| 95 | 146; 152; 181; 199; 522; 545; 622; 741; 981; 1001; 1055; 1246; 1400; 1493; 1587; 1618; 1655; 1859; | 100 | 100 | 100 | 100 | 100 | 7.77E−11 |
| | 1984; 2040; 2050; 2245; 2387; 2424; 2469; 2826; 3292; 3319; 3326; 3582; 3832; 3894; 3982; 4013; | | | | | | |
| | 4089; 4099; 4246; 4319; 4403; 4431; 4462; 4572; 4730; 4907; 5087; 5222 | | | | | | |
| 96 | 776; 839; 1325; 1582; 2674; 2723; 3479; 4225; 4893; 5023 | 100 | 100 | 100 | 100 | 100 | 7.77E−11 |
| 97 | 21; 164; 2065; 2520; 3972; 4417; 4873; 4902 | 100 | 100 | 100 | 100 | 100 | 7.77E−11 |
| 98 | 578; 760; 1117; 1860; 2013; 2092; 2378; 2550; 2551; 2919; 3086; 3196; 3406; 3728; 4009; 4416; 4436; 4618; 4852; 4894; 4949; 4978 | 100 | 100 | 100 | 100 | 100 | 7.77E−11 |
| 99 | 65; 432; 817; 833; 1071; 1766; 1821; 2938; 3236; 3388; 3740; 3955; 4004; 4298; 4528; 4717; 4778; 5063 | 100 | 100 | 100 | 100 | 100 | 7.77E−11 |
| 100 | 816; 1176; 1725; 2340; 3286; 3649; 4167; 4909; 5173 | 100 | 100 | 100 | 100 | 100 | 7.77E−11 |
| 101 | 673; 1244; 1773; 1816; 1910; 2087; 2340; 2569; 2665; 2831; 2878; 2991; 3138; 3174; 3262; 3324; | 100 | 100 | 100 | 100 | 100 | 7.77E−11 |
| | 3595; 3606; 3745; 3975; 4108; 4379; 4446; 4732; 4774; 4974; 5105; 5107; 5141 | | | | | | |
| 102 | 219; 680; 1795; 1951; 2024; 2515; 2523; 4561; 5009 | 100 | 100 | 100 | 100 | 100 | 7.77E−11 |
| 103 | 39; 48; 404; 432; 519; 687; 742; 1150; 1340; 1497; 1766; 2042; 2050; 2230; 2301; 2434; 2505; 2668; | 100 | 100 | 100 | 100 | 100 | 7.77E−11 |
| | 2924; 3877; 4102; 4260; 4494; 4625; 5081 | | | | | | |
| 104 | 207; 479; 706; 2052; 2566; 2857; 4634 | 100 | 100 | 100 | 100 | 100 | 7.77E−11 |
| 105 | 147; 160; 631; 696; 1049; 1404; 1883; 2073; 2522; 2733; 3307; 3623; 4515; 4756; 4765 | 100 | 100 | 100 | 100 | 100 | 7.77E−11 |
| 106 | 208; 762; 803; 1089; 1149; 1171; 1476; 1492; 1556; 1719; 1958; 2033; 2153; 2887; 3076; 3912; 3955; 4023; 4029; 4084; 4809; 5198 | 100 | 100 | 100 | 100 | 100 | 7.77E−11 |
| 107 | 301; 385; 402; 600; 684; 714; 745; 858; 890; 954; 975; 1275; 1558; 1567; 1582; 1878; 1922; 1987; | 100 | 100 | 100 | 100 | 100 | 7.77E−11 |
| | 2137; 2435; 2522; 2815; 2846; 2911; 3098; 3388; 3846; 3883; 4058; 4187; 4436; 4547; 4637; 4991; 5151; 5185; 5260 | | | | | | |
| 108 | 42; 127; 136; 558; 604; 791; 883; 929; 1018; 1057; 1369; 1400; 1653; 1775; 1831; 1930; 2201; 2258; | 100 | 100 | 100 | 100 | 100 | 7.77E−11 |
| | 2297; 2488; 2674; 2960; 2981; 3070; 3980; 4134; 4197; 4334; 4633; 4661; 4688; 4872 | | | | | | |
| 109 | 240; 722; 1055; 1115; 1118; 1201; 1298; 1657; 2816; 3213; 3807; 3911; 4045; 4084; 4097; 4391; 4630; 4950; 4963; 5144 | 100 | 100 | 100 | 100 | 100 | 7.77E−11 |
| 110 | 58; 76; 353; 507; 599; 1658; 1810; 1946; 1982; 2028; 2145; 2323; 2353; 2424; 2521; 2575; 2701; 2721; | 100 | 100 | 100 | 100 | 100 | 7.77E−11 |
| | 2966; 3173; 3537; 4252; 4486; 4565; 4637; 4880; 4957; 4969; 4986; 5156 | | | | | | |
| 111 | 163; 386; 530; 564; 634; 1168; 1606; 1621; 2498; 4863 | 100 | 100 | 100 | 100 | 100 | 7.77E−11 |
| 112 | 73; 87; 109; 187; 217; 227; 330; 437; 634; 643; 727; 797; 959; 1022; 1095; 1377; 1554; 1667; 1761; | 100 | 100 | 100 | 100 | 100 | 7.77E−11 |
| | 1823; 1900; 1938; 2615; 2625; 2636; 2638; 2748; 2923; 2960; 3472; 3564; 3704; 3739; 3757; 3820; | | | | | | |
| | 3849; 3918; 4056; 4076; 4141; 4197; 4376; 4547; 4565; 4572; 4944; 4995; 5165; 5232 | | | | | | |
| 113 | 1024; 1080; 1936; 2116; 2239; 2263; 2658; 3588; 3961; 3986 | 100 | 100 | 100 | 100 | 100 | 7.77E−11 |
| 114 | 3; 283; 488; 678; 1059; 1946; 2162; 2495; 2722; 3005; 3357; 3495; 3523; 4068; 5058; 5202 | 100 | 100 | 100 | 100 | 100 | 7.77E−11 |
| 115 | 209; 511; 647; 1476; 1524; 2025; 2136; 2382; 2697; 2775; 2922; 3236; 3374; 3375; 3634; 3645; 3765; | 100 | 100 | 100 | 100 | 100 | 7.77E−11 |
| | 3961; 4110; 4310; 4387; 4467; 4590; 4859; 5015 | | | | | | |
| 116 | 391; 782; 1801; 2890; 4084; 4316; 4703 | 100 | 100 | 100 | 100 | 100 | 7.77E−11 |
| 117 | 569; 676; 687; 809; 1027; 1219; 1434; 1446; 1467; 1716; 1759; 1857; 2330; 2502; 2617; 2903; 3376; | 100 | 100 | 100 | 100 | 100 | 7.77E−11 |
| | 3399; 3655; 3760; 4173; 4266; 4283; 4584; 4655; 4678; 4698; 4953; 5146 | | | | | | |
| 118 | 67; 1356; 3177; 3898; 3979; 4291; 4678; 4741 | 100 | 100 | 100 | 100 | 100 | 7.77E−11 |
| 119 | 114; 236; 304; 669; 764; 831; 836; 886; 919; 968; 1110; 1613; 1789; 1824; 2232; 2346; 2438; 2616; | 100 | 100 | 100 | 100 | 100 | 7.77E−11 |
| | 2760; 2823; 3195; 3284; 3295; 3310; 3326; 3387; 3495; 3518; 3581; 3605; 3671; 3876; 3906; 4094; | | | | | | |
| | 4146; 4387; 4665; 4842; 4907; 4928; 4960; 5002; 5038; 5178; 5285 | | | | | | |
| 120 | 1172; 1272; 1903; 2425; 2511; 2738; 3037; 3375; 3663; 4114; 4311; 4353; 5015 | 100 | 100 | 100 | 100 | 100 | 7.77E−11 |
| 121 | 1285; 1546; 3103; 3547; 3623; 3966; 4917; 5236 | 100 | 100 | 100 | 100 | 100 | 7.77E−11 |
| 122 | 784; 1062; 2165; 2262; 2334; 2598; 3379; 3938; 4089; 4910 | 100 | 100 | 100 | 100 | 100 | 7.77E−11 |
| 123 | 87; 164; 342; 599; 629; 734; 843; 853; 1069; 1087; 1094; 1174; 1179; 1259; 1450; 1667; 1691; 1821; | 100 | 100 | 100 | 100 | 100 | 7.77E−11 |
| | 1916; 2179; 2219; 2374; 2457; 2839; 2963; 3057; 3102; 3167; 3476; 3479; 3552; 3658; 3805; 3918; | | | | | | |
| | 3939; 3967; 3989; 4001; 4135; 4300; 4356; 4423; 4460; 4554; 4630; 4667; 4682; 4819; 5071; 5100 | | | | | | |
| 124 | 88; 221; 303; 378; 407; 692; 906; 950; 1019; 1123; 1256; 1555; 1619; 1670; 1771; 1814; 1935; 1984; | 100 | 100 | 100 | 100 | 100 | 7.77E−11 |
| | 2207; 2408; 2438; 2686; 2787; 3341; 3483; 3718; 3891; 3894; 4029; 4138; 4258; 4336; 4363; 4380; 4413; 5119 | | | | | | |
| 125 | 58; 60; 380; 795; 1133; 1361; 1435; 1481; 1601; 1869; 1893; 2390; 2415; 2607; 3124; 3157; 3983; 4004; 4027; 4313; 4323; 4477; 4627; 5185 | 100 | 100 | 100 | 100 | 100 | 7.77E−11 |
| 126 | 155; 410; 435; 442; 588; 931; 1118; 1155; 1398; 1591; 1605; 1799; 2581; 2683; 2836; 2837; 3017; 3057; | 100 | 100 | 100 | 100 | 100 | 7.77E−11 |
| | 3209; 3237; 3309; 3551; 3561; 3586; 3808; 3921; 4546 | | | | | | |
| 127 | 3388; 4183; 4265; 4888 | 100 | 100 | 100 | 100 | 100 | 7.77E−11 |
| 128 | 211; 1445; 2358; 2677; 4305; 4723 | 100 | 100 | 100 | 100 | 100 | 7.77E−11 |
| 129 | 449; 876; 1830; 2704; 3139; 3205; 3829; 4149; 4571 | 100 | 100 | 100 | 100 | 100 | 7.77E−11 |
| 130 | 42; 51; 101; 376; 614; 732; 864; 1227; 1386; 1562; 2045; 2051; 2181; 2383; 2389; 2483; 2847; 2864; | 100 | 100 | 100 | 100 | 100 | 7.77E−11 |
| | 2878; 2970; 2982; 3104; 3565; 3631; 3701; 3743; 3790; 3879; 3940; 4226; 4276; 4343; 4400; 4484; | | | | | | |
| | 4495; 4529; 4539; 4556; 4618; 4622; 4675; 4759; 4785; 4853; 5206 | | | | | | |
| 131 | 219; 428; 1376; 2180; 2337; 2340; 3060; 3168; 3713; 4290; 4292; 4349; 4552; 4718; 4987; 5108; 5176 | 100 | 100 | 100 | 100 | 100 | 7.77E−11 |
| 132 | 164; 183; 226; 365; 459; 636; 707; 894; 984; 1003; 1024; 1081; 1218; 1324; 1827; 2281; 2524; 2651; | 100 | 100 | 100 | 100 | 100 | 7.77E−11 |
| | 3101; 3269; 3375; 3498; 3549; 3707; 4170; 4573; 4618; 4655; 4711; 4780; 4858; 5173; 5226 | | | | | | |
| 133 | 780; 944; 1740; 1760; 2077; 3361; 3701; 3916; 4443; 4908 | 100 | 100 | 100 | 100 | 100 | 7.77E−11 |
| 134 | 2642; 3667; 4633; 4942 | 100 | 100 | 100 | 100 | 100 | 7.77E−11 |
| 135 | 434; 700; 739; 1376; 1501; 1896; 2262; 2458; 2731; 2839; 2851; 3335; 3462; 4032; 4610; 4619; 4735; 4788; 4890 | 100 | 100 | 100 | 100 | 100 | 7.77E−11 |
| 136 | 230; 299; 353; 680; 720; 944; 1224; 1251; 1320; 1505; 1548; 1555; 2136; 2197; 2223; 2390; 2479; 2568; 2573; 2837; 2843; 2884; | 100 | 100 | 100 | 100 | 100 | 7.77E−11 |
| | 3002; 3109; 3135; 3163; 3492; 3557; 3569; 3634; 3703; 4227; 4260; 4411; 4507; 4707; 4933; 4985; 5021; 5054; 5061 | | | | | | |
| 137 | 206; 1120; 1354; 2154; 2592; 3637; 4803; 4954; 5201; 5207 | 100 | 100 | 100 | 100 | 100 | 7.77E−11 |
| 138 | 951; 1070; 1479; 2522; 2824; 3176; 3301; 3315; 3731; 3834; 4187; 4474; 5093; 5200 | 100 | 100 | 100 | 100 | 100 | 7.77E−11 |
| 139 | 549; 897; 1180; 1739; 4062; 4319; 4633; 4897; 5037 | 100 | 100 | 100 | 100 | 100 | 7.77E−11 |
| 140 | 314; 2302; 2352; 3032; 5014; 5165; 5212 | 100 | 100 | 100 | 100 | 100 | 7.77E−11 |
| 141 | 137; 1317; 1870; 2239; 2526; 2587; 4847 | 100 | 100 | 100 | 100 | 100 | 7.77E−11 |
| 142 | 104; 658; 667; 758; 875; 907; 1163; 1265; 1748; 1875; 2071; 2951; 2971; 2978; 3115; 3177; 3281; | 100 | 100 | 100 | 100 | 100 | 7.77E−11 |
| | 3360; 3368; 3394; 3436; 3674; 3999; 4047; 4176; 4253; 4747; 4783; 4828 | | | | | | |
| 143 | 439; 623; 1096; 1453; 1730; 2545; 2602; 2899; 4140; 4668; 4873 | 100 | 100 | 100 | 100 | 100 | 7.77E−11 |
| 144 | 82; 117; 262; 482; 594; 606; 698; 703; 711; 1021; 1039; 1091; 1179; 1193; 1203; 1309; 1367; | 100 | 100 | 100 | 100 | 100 | 7.77E−11 |
| | 1475; 1684; 2054; 2170; 2355; 2356; 2649; 2727; 2937; 3185; 3438; 3443; 3583; 3614; 3656; 3752; | | | | | | |
| | 4003; 4074; 4080; 4091; 4144; 4645; 4719; 4822; 4888; 5005; 5205 | | | | | | |
| 145 | 538; 842; 1334; 1472; 2190; 2665; 2835; 3062; 3826; 5235 | 100 | 100 | 100 | 100 | 100 | 7.77E−11 |
| 146 | 1238; 1639; 2019; 4075; 5252; 5260 | 100 | 100 | 100 | 100 | 100 | 7.77E−11 |
| 147 | 62; 828; 1330; 1940; 1961; 2444; 2995; 3209; 4068 | 100 | 100 | 100 | 100 | 100 | 7.77E−11 |
| 148 | 491; 553; 932; 1125; 1352; 1789; 2870; 3299; 3496; 3916; 4144; 4341; 4436 | 100 | 100 | 100 | 100 | 100 | 7.77E−11 |
| 149 | 361; 967; 1380; 4009; 4832 | 100 | 100 | 100 | 100 | 100 | 7.77E−11 |
| 150 | 44; 74; 77; 109; 206; 374; 546; 606; 675; 736; 1054; 1258; 1572; 1608; 1990; 1991; 2017; 2230; 2477; 2654; | 100 | 100 | 100 | 100 | 100 | 7.77E−11 |
| | 2658; 2912; 3187; 3209; 3309; 3666; 3796; 3797; 3812; 3923; 3953; 4039; 4117; 4156; 4194; 4405; 4715; 4732; 4806; 4844; 4861; 4919 | | | | | | |
| 151 | 417; 818; 976; 2019; 2722; 3423; 3842; 4828; 5120 | 100 | 100 | 100 | 100 | 100 | 7.77E−11 |
| 152 | 274; 675; 1016; 1061; 1171; 1258; 1831; 1860; 2096; 2465; 2580; 2783; 2920; 3301; 3368; 4290; 4564; 4693; 4816; 4986 | 100 | 100 | 100 | 100 | 100 | 7.77E−11 |
| 153 | 35; 117; 428; 472; 647; 693; 719; 720; 963; 1103; 1433; 1443; 1479; 1513; 1933; 1983; 2143; 2252; | 100 | 100 | 100 | 100 | 100 | 7.77E−11 |
| | 2270; 2339; 2488; 2519; 2520; 2554; 2652; 2658; 2864; 2913; 3042; 3113; 3131; 3136; 3440; 3623; | | | | | | |
| | 3932; 3954; 4110; 4144; 4418; 4513; 4622; 4847; 4904; 4963; 5161 | | | | | | |
| 154 | 55; 432; 788; 919; 1003; 1106; 1129; 1224; 1321; 1452; 1582; 1722; 1746; 1800; 1827; 1891; 1925; | 100 | 100 | 100 | 100 | 100 | 7.77E−11 |
| | 1981; 2400; 2458; 2575; 2593; 2730; 2919; 2960; 2962; 3158; 3167; 3218; 3333; 3395; 3420; 3764; | | | | | | |
| | 4125; 4260; 4719; 4808; 4865; 5112; 5201; 5260 | | | | | | |
| 155 | 148; 246; 284; 293; 409; 621; 635; 823; 927; 939; 946; 1343; 1376; 1446; 1686; 1763; 1947; 2013; 2036; 2081; | 100 | 100 | 100 | 100 | 100 | 7.77E−11 |
| | 2342; 2498; 2617; 2639; 2684; 3068; 3344; 3612; 3700; 3813; 3861; 3903; 3929; 3966; 4055; 4080; 4430; 4437; 4603 | | | | | | |
| 156 | 171; 639; 1245; 1759; 1774; 1864; 2285; 2329 | 100 | 100 | 100 | 100 | 100 | 7.77E−11 |
| 157 | 187; 220; 382; 392; 595; 689; 1166; 1260; 1276; 1341; 2001; 2269; 2376; 2427; 2443; 2545; 2826; 2889; 2891; 2969; | 100 | 100 | 100 | 100 | 100 | 7.77E−11 |
| | 3097; 3118; 3202; 3344; 3400; 3544; 3649; 4041; 4468; 4585; 4685; 4765; 4785; 4823; 4898; 4996; 5054; 5166; 5254 | | | | | | |
| 158 | 1324; 1722; 2411; 3041; 3119; 4237 | 100 | 100 | 100 | 100 | 100 | 7.77E−11 |
| 159 | 209; 378; 618; 967; 2335; 2795; 2931; 4464; 4591 | 100 | 100 | 100 | 100 | 100 | 7.77E−11 |
| 160 | 1397; 5021; 5265 | 100 | 100 | 100 | 100 | 100 | 7.77E−11 |
| 161 | 110; 182; 317; 324; 392; 529; 1077; 1171; 1204; 1279; 1552; 1645; 2067; 2317; 2677; 3216; 3235; | 100 | 100 | 100 | 100 | 100 | 7.77E−11 |
| | 3296; 3648; 3668; 3750; 3988; 3989; 4842; 5173 | | | | | | |
| 162 | 56; 309; 380; 616; 1639; 2333; 2691; 3805 | 100 | 100 | 100 | 100 | 100 | 7.77E−11 |
| 163 | 37; 84; 449; 459; 924; 1147; 1205; 1311; 2026; 2034; 2274; 3445; 4079; 4218; 4661; 5281 | 100 | 100 | 100 | 100 | 100 | 7.77E−11 |
| 164 | 102; 220; 418; 814; 939; 1062; 1171; 1300; 1513; 1726; 1943; 2286; 2458; 2642; 2966; 3057; 3078; | 100 | 100 | 100 | 100 | 100 | 7.77E−11 |
| | 3236; 3457; 3529; 3570; 3628; 3759; 3922; 4074; 4107; 4262; 4476; 4498; 4830; 4903; 4942; 5011; 5190 | | | | | | |
| 165 | 2; 196; 227; 273; 300; 408; 622; 777; 1004; 1014; 1067; 1170; 1244; 1778; 1881; 2039; 2041; 2103; | 100 | 100 | 100 | 100 | 100 | 7.77E−11 |
| | 2683; 2721; 2730; 3090; 3115; 3193; 3360; 3493; 3654; 3722; 3815; 4051; 4221; 4437; 4474; 5076; 5244 | | | | | | |
| 166 | 152; 488; 1155; 1612; 1693; 3659 | 100 | 100 | 100 | 100 | 100 | 7.77E−11 |
| 167 | 371; 608; 758; 2184; 2735; 2842; 3127; 3358; 3645; 3860 | 100 | 100 | 100 | 100 | 100 | 7.77E−11 |
| 168 | 633; 3623; 3731; 4007; 4568; 4825; 4975; 5156 | 100 | 100 | 100 | 100 | 100 | 7.77E−11 |
| 169 | 1; 46; 124; 185; 199; 292; 393; 675; 752; 818; 871; 962; 997; 1307; 1339; 1423; 1526; 1617; 1645; | 100 | 100 | 100 | 100 | 100 | 7.77E−11 |
| | 1667; 1820; 1876; 1943; 2170; 2202; 2209; 2432; 2542; 3179; 3199; 3204; 3277; 3564; 3573; 3771; | | | | | | |
| | 3820; 3845; 3854; 4172; 4400; 4429; 4638; 4830; 5011; 5152 | | | | | | |
| 170 | 696; 962; 2009; 2491; 2842; 3240; 3815; 3871; 4162; 5102 | 100 | 100 | 100 | 100 | 100 | 7.77E−11 |
| 171 | 86; 757; 1257; 1261; 1272; 1413; 1662; 2235; 2306; 2445; 2457; 3390; 3737; 4282; 4303; 4527; 4539; 5197; 5200 | 100 | 100 | 100 | 100 | 100 | 7.77E−11 |
| 172 | 5240 | 100 | 100 | 100 | 100 | 100 | 7.77E−11 |
| 173 | 424; 4265; 4672 | 100 | 100 | 100 | 100 | 100 | 7.77E−11 |
| 174 | 8; 1538; 2247; 2848; 3421; 4392; 4806; 5033 | 100 | 100 | 100 | 100 | 100 | 7.77E−11 |
| 175 | 1051; 1318; 1551; 1573; 2062; 2324; 2887; 3227; 3583; 4126; 4350; 4474; 4656 | 100 | 100 | 100 | 100 | 100 | 7.77E−11 |
| 176 | 507; 1046; 1490; 1909; 2348; 4113; 4172 | 100 | 100 | 100 | 100 | 100 | 7.77E−11 |
| 177 | 114; 147; 372; 475; 573; 679; 752; 928; 986; 1102; 1323; 1493; 1793; 2035; 2041; 2227; 2242; 2836; 3519; 3902; 4144; 4474; 4748; 4755; 4783; 5220 | 100 | 100 | 100 | 100 | 100 | 7.77E−11 |
| 178 | 215; 315; 758; 1164; 1390; 1471; 1568; 2036; 2325; 2657; 2781; 3067; 3244; 3429; 3460; 3573; 3632; 3873; 4265; 4528; 4885; 4921; 5156 | 100 | 100 | 100 | 100 | 100 | 7.77E−11 |
| 179 | 160; 385; 1200; 1335; 3580; 4154; 4289; 5271 | 100 | 100 | 100 | 100 | 100 | 7.77E−11 |
| 180 | 8; 34; 235; 286; 476; 498; 773; 1016; 1233; 1388; 1441; 1891; 1937; 2814; 2834; 3455; 3557; 3678; 4080; 4155; 4500; 4659; 4669 | 100 | 100 | 100 | 100 | 100 | 7.77E−11 |
| 181 | 595; 1032; 1099; 2677; 3074; 3863; 4175; 4683 | 100 | 100 | 100 | 100 | 100 | 7.77E−11 |
| 182 | 0; 519; 963; 1330; 1402; 1480; 1520; 1681; 1744; 1749; 1831; 1883; 2019; 2320; 2483; 2814; 3208; | 100 | 100 | 100 | 100 | 100 | 7.77E−11 |
| | 3530; 3847; 3851; 3943; 3983; 4067; 4130; 4720; 5168 | | | | | | |
| 183 | 418; 1104; 1149; 1423; 1477; 1789; 2657; 2922; 4097; 4140; 4333; 4630; 4792; 4988; 5075; 5203; 5250 | 100 | 100 | 100 | 100 | 100 | 7.77E−11 |
| 184 | 223; 290; 404; 449; 452; 636; 762; 1072; 1106; 1342; 1563; 1930; 1937; 1998; 2058; 2343; 2485; | 100 | 100 | 100 | 100 | 100 | 7.77E−11 |
| | 2761; 3014; 3029; 3085; 3088; 3196; 3209; 3932; 4040; 4184; 4282; 4494; 4535; 5092; 5233 | | | | | | |
| 185 | 282; 290; 1152; 1379; 1933; 2890; 3128; 4621 | 100 | 100 | 100 | 100 | 100 | 7.77E−11 |
| 186 | 1351; 1503; 1876; 2607; 2670; 2748; 2781; 3079; 3160 | 100 | 100 | 100 | 100 | 100 | 7.77E−11 |
| 187 | 16; 194; 748; 927; 1201; 1202; 1977; 3120; 3639; 4018 | 100 | 100 | 100 | 100 | 100 | 7.77E−11 |
| 188 | 818; 838; 1020; 2642; 3283; 3382; 4272 | 100 | 100 | 100 | 100 | 100 | 7.77E−11 |
| 189 | 162; 380; 639; 909; 958; 1136; 2088; 2476; 3497; 4026; 4735 | 100 | 100 | 100 | 100 | 100 | 7.77E−11 |
| 190 | 201; 242; 258; 260; 496; 933; 1223; 1258; 1538; 1610; 2624; 2820; 2834; 2904; 3088; 3209; 3681; 3949; 4340; 4934 | 100 | 100 | 100 | 100 | 100 | 7.77E−11 |
| 191 | 139; 432; 810; 1101; 1249; 1453; 1657; 1658; 1865; 2349; 2430; 2731; 2905; 3236; 3284; 3288; 3293; | 100 | 100 | 100 | 100 | 100 | 7.77E−11 |
| | 3302; 3428; 3516; 3623; 3741; 3758; 3937; 4185; 4200; 4211; 4381; 4511; 4664; 4695; 5118 | | | | | | |
| 192 | 128; 850; 1107; 1261; 1589; 1610; 1881; 2297; 2345; 2457; 2477; 2626; 2658; 2845; 3027; 3067; 3514; | 100 | 100 | 100 | 100 | 100 | 7.77E−11 |
| | 3543; 3767; 4013; 4257; 4471; 4557; 4590; 4621; 5008 | | | | | | |
| 193 | 644; 699; 759; 887; 1452; 1778; 1887; 2138; 2162; 2597; 2830; 3586; 4017; 4198; 4569; 4632; 4639; 4867; 4990; 5198 | 100 | 100 | 100 | 100 | 100 | 7.77E−11 |
| 194 | 933; 1144; 1145; 1371; 1937; 2047; 3385; 4263; 5134 | 100 | 100 | 100 | 100 | 100 | 7.77E−11 |
| 195 | 1636; 1700; 2128; 2820 | 100 | 100 | 100 | 100 | 100 | 7.77E−11 |
| 196 | 319; 551; 777; 800; 1044; 1127; 1292; 1375; 1433; 1456; 1485; 1613; 1660; 2246; 2419; 2633; 2689; | 100 | 100 | 100 | 100 | 100 | 7.77E−11 |
| | 2780; 2965; 3333; 3454; 3558; 3910; 4003; 4019; 4097; 4114; 4145; 4690; 4771; 4774; 5201 | | | | | | |
| 197 | 10; 164; 179; 402; 561; 1252; 1739; 2626; 2831; 2957; 3427; 3579; 3958; 4223; 4308; 4740; 5014; 5063; 5108 | 100 | 100 | 100 | 100 | 100 | 7.77E−11 |
| 198 | 655; 850; 2817; 4069; 4529; 4880 | 100 | 100 | 100 | 100 | 100 | 7.77E−11 |
| 199 | 16; 305; 330; 1470; 2855; 4388 | 100 | 100 | 100 | 100 | 100 | 7.77E−11 |
| 200 | 160; 163; 263; 303; 346; 393; 637; 816; 950; 1138; 1319; 1369; 1687; 1889; 1945; 1948; 1971; 2162; | 100 | 100 | 100 | 100 | 100 | 7.77E−11 |
| | 2169; 2305; 2356; 2432; 2463; 2505; 2537; 2824; 2940; 3056; 3139; 3194; 3340; 3342; 3494; 3507; | | | | | | |
| | 3635; 3697; 3820; 3891; 4086; 4299; 4485; 4714; 4922; 4983; 5068; 5152; 5160; 5175; 5184; 5268 | | | | | | |
| 201 | 546; 3115; 3140; 3449; 3745; 3862; 3954; 3957; 5096; 5253 | 100 | 100 | 100 | 100 | 100 | 7.77E−11 |
| 202 | 552; 1320; 1421; 1436; 1884; 2677; 2891; 3288; 3418; 3971; 4057; 4154; 4713; 5129 | 100 | 100 | 100 | 100 | 100 | 7.77E−11 |
| 203 | 530; 630; 728; 780; 917; 1176; 1229; 1375; 1635; 1793; 2392; 2569; 2595; 2673; 2783; 3266; 3358; | 100 | 100 | 100 | 100 | 100 | 7.77E−11 |
| | 3479; 3616; 3621; 3881; 4022; 4105; 4322; 4356; 4557; 4706; 4745; 4836; 4876 | | | | | | |
| 204 | 399; 975; 1682; 2955; 3042; 3225; 3309; 3501; 4490 | 100 | 100 | 100 | 100 | 100 | 7.77E−11 |
| 205 | 309; 319; 445; 1178; 1293; 1305; 1385; 1511; 2363; 2445; 2933; 3086; 3093; 3143; 3320; 3425; 4211; 4683; 4985; 5006 | 100 | 100 | 100 | 100 | 100 | 7.77E−11 |
| 206 | 563; 1245; 2093; 3432; 3741; 3863; 4147; 4982 | 100 | 100 | 100 | 100 | 100 | 7.77E−11 |
| 207 | 1964; 4090; 4115; 4239; 4430 | 100 | 100 | 100 | 100 | 100 | 7.77E−11 |
| 208 | 79; 481; 583; 595; 653; 861; 868; 967; 1145; 1166; 1206; 1704; 2049; 2173; 2255; 2265; 2363; 2520; | 100 | 100 | 100 | 100 | 100 | 7.77E−11 |
| | 2557; 2622; 2655; 2666; 2752; 2911; 3042; 3388; 3431; 3438; 3515; 3580; 3839; 3935; 4219; 4308; | | | | | | |
| | 4353; 4487; 4517; 4626; 4691; 4925; 4972; 5143; 5167; 5207; 5221 | | | | | | |
| 209 | 21; 159; 219; 407; 424; 498; 544; 709; 789; 1092; 1122; 1502; 1690; 1726; 1859; 1868; 1894; 2315; | 100 | 100 | 100 | 100 | 100 | 7.77E−11 |
| | 2535; 2755; 2929; 3020; 3190; 3205; 3431; 3581; 3920; 4001; 4244; 4441; 4730; 5220 | | | | | | |
| 210 | 163; 1414; 1637; 2190; 3388; 4022 | 100 | 100 | 100 | 100 | 100 | 7.77E−11 |
| 211 | 161; 332; 410; 438; 907; 911; 1021; 1035; 1138; 1228; 1265; 1327; 1332; 1342; 1501; 1657; 1744; | 100 | 100 | 100 | 100 | 100 | 7.77E−11 |
| | 1745; 1901; 1994; 2021; 2236; 2296; 2481; 2555; 2668; 2839; 2841; 2928; 3209; 3279; 3329; 3377; | | | | | | |
| | 3385; 3508; 3911; 3986; 4026; 4068; 4133; 4276; 4708; 4714; 4733; 4856; 4869; 5176; 5192; 5215 | | | | | | |
| 212 | 188; 1165; 1876; 2272; 2677; 2886; 3172; 3341; 3588; 4179; 4300; 5104; 5156 | 100 | 100 | 100 | 100 | 100 | 7.77E−11 |
| 213 | 1808; 3452; 4655; 5255 | 100 | 100 | 100 | 100 | 100 | 7.77E−11 |
| 214 | 212; 959; 1086; 1457; 1799; 1996; 2122; 2263; 2896; 4242; 4318; 4434; 4492; 4872; 5058; 5228 | 100 | 100 | 100 | 100 | 100 | 7.77E−11 |
| 215 | 285; 304; 503; 622; 862; 966; 1170; 1366; 1507; 1614; 1683; 1942; 2208; 2237; 2276; 2335; 2542; | 100 | 100 | 100 | 100 | 100 | 7.77E−11 |
| | 2566; 2675; 2796; 2798; 2824; 2862; 2924; 3064; 3203; 3336; 3469; 3502; 3721; 3744; 3900; 3902; | | | | | | |
| | 3909; 3924; 3982; 4247; 4332; 4544; 4564; 4678; 4989; 5025; 5072; 5084; 5147; 5223 | | | | | | |
| 216 | 1286; 1864; 2126; 2266; 2368; 3773; 4693; 5054 | 100 | 100 | 100 | 100 | 100 | 7.77E−11 |
| 217 | 887; 1999; 4086; 4394; 4478; 5003 | 100 | 100 | 100 | 100 | 100 | 7.77E−11 |
| 218 | 769; 824; 941; 1472; 1817; 4139; 5068 | 100 | 100 | 100 | 100 | 100 | 7.77E−11 |
| 219 | 84; 152; 652; 1374; 2245; 2760; 3548; 3907; 4008; 4194 | 100 | 100 | 100 | 100 | 100 | 7.77E−11 |
| 220 | 207; 648; 774; 922; 952; 1089; 1140; 1212; 1344; 1424; 1612; 1841; 2099; 2132; 2535; 2555; 3010; | 100 | 100 | 100 | 100 | 100 | 7.77E−11 |
| | 3492; 3639; 3690; 3748; 3819; 3885; 3962; 3976; 4193; 4221; 4249; 4732; 4764; 4829; 5235; 5237; 5284 | | | | | | |
| 221 | 762; 850; 889; 947; 1232; 2127; 2520; 3219; 3524; 5168 | 100 | 100 | 100 | 100 | 100 | 7.77E−11 |
| 222 | 335; 2288; 4382; 4387 | 100 | 100 | 100 | 100 | 100 | 7.77E−11 |
| 223 | 1293; 1864; 2279; 2902; 3011; 3922; 4705; 4917 | 100 | 100 | 100 | 100 | 100 | 7.77E−11 |
| 224 | 87; 498; 532; 674; 779; 932; 1066; 1098; 1180; 1301; 1334; 1472; 1486; 1820; 2125; 2224; 2472; | 100 | 100 | 100 | 100 | 100 | 7.77E−11 |
| | 2492; 2581; 2863; 2868; 2884; 3055; 3237; 3332; 3376; 3626; 3632; 3709; 3852; 3969; 4237; 4323; | | | | | | |
| | 4358; 4375; 4422; 4520; 4653; 4798; 4860; 4962; 5144; 5156; 5260; 5277 | | | | | | |
| 225 | 135; 316; 3207; 3279; 3654; 4642; 4735; 4823 | 100 | 100 | 100 | 100 | 100 | 7.77E−11 |
| 226 | 344; 850; 1846; 2047; 3649; 4679 | 100 | 100 | 100 | 100 | 100 | 7.77E−11 |
| 227 | 130; 152; 300; 311; 362; 505; 558; 883; 986; 989; 1068; 1346; 1548; 1549; 1621; 2035; 2078; 2111; | 100 | 100 | 100 | 100 | 100 | 7.77E−11 |
| | 2264; 2280; 2323; 2517; 2649; 3006; 3018; 3109; 3314; 3362; 3377; 3464; 3523; 3578; 3616; 3687; | | | | | | |
| | 3859; 3976; 4069; 4373; 4536; 4548; 4661; 4756; 4764; 4885; 5084; 5141; 5253 | | | | | | |
| 228 | 39; 89; 128; 304; 353; 648; 781; 784; 1155; 1170; 1190; 1226; 1544; 1683; 1745; 2058; 2145; 2197; 2325; 2381; | 100 | 100 | 100 | 100 | 100 | 7.77E−11 |
| | 2396; 2457; 2482; 2506; 2523; 2662; 2859; 2895; 2966; 3057; 3076; 3090; 3762; 3886; 3946; 4056; 4099; 4349; 4364; 4557; 4564; 4679 | | | | | | |
| 229 | 863; 1498; 1524; 2266; 2326; 2580; 2642; 3286; 4177; 5014 | 100 | 100 | 100 | 100 | 100 | 7.77E−11 |
| 230 | 174; 270; 389; 538; 585; 620; 1046; 1429; 2371; 2549; 3125; 3245; 3419; 3701; 3784; 4266; 4283; | 100 | 100 | 100 | 100 | 100 | 7.77E−11 |
| | 4296; 4310; 4395; 4487; 4535; 4701; 5006; 5050; 5253 | | | | | | |
| 231 | 31; 69; 219; 519; 775; 782; 872; 1212; 1436; 1577; 1579; 1887; 1896; 1902; 2299; 2496; 2562; 2620; 2636; 2665; | 100 | 100 | 100 | 100 | 100 | 7.77E−11 |
| | 2947; 2960; 3004; 3232; 3261; 3298; 3432; 3629; 3724; 3788; 4134; 4186; 4428; 4677; 4756; 4817; 4890; 5110; 5231; 5254 | | | | | | |
| 232 | 808; 1311; 2222; 2567; 2939; 3070; 3208; 3991; 4820; 4917; 5190 | 100 | 100 | 100 | 100 | 100 | 7.77E−11 |
| 233 | 390; 410; 750; 1227; 1446; 2049; 2275; 2511; 3062; 3081; 4045; 4654; 4902; 5014; 5023; 5274 | 100 | 100 | 100 | 100 | 100 | 7.77E−11 |
| 234 | 106; 293; 384; 783; 838; 1123; 1216; 1568; 1758; 1856; 2519; 2657; 2707; 2772; 2775; 2849; 3139; | 100 | 100 | 100 | 100 | 100 | 7.77E−11 |
| | 3540; 3919; 4022; 4294; 4340; 4403; 4587; 4744; 4983; 5075 | | | | | | |
| 235 | 442; 1448; 1590; 1870; 4026; 4207 | 100 | 100 | 100 | 100 | 100 | 7.77E−11 |
| 236 | 96; 158; 203; 671; 852; 986; 1220; 1293; 1467; 1505; 1897; 1993; 2036; 2046; 2092; 2209; 2266; | 100 | 100 | 100 | 100 | 100 | 7.77E−11 |
| | 2289; 2292; 2429; 2451; 2520; 2794; 3064; 3282; 3464; 3869; 3955; 4208; 4210; 4245; 4266; 4288; | | | | | | |
| | 4324; 4326; 4331; 4450; 4533; 4630; 4705; 4750; 4843; 4861; 4976; 5031; 5138 | | | | | | |
| 237 | 3; 530; 1088; 1702; 3652; 5070 | 100 | 100 | 100 | 100 | 100 | 7.77E−11 |
| 238 | 2799; 3350; 4752 | 100 | 100 | 100 | 100 | 100 | 7.77E−11 |
| 239 | 1497; 1522; 2937; 3111; 3181; 5180 | 100 | 100 | 100 | 100 | 100 | 7.77E−11 |
| 240 | 67; 106; 168; 303; 529; 568; 869; 1413; 1794; 1796; 1842; 1875; 2051; 2121; 2206; 2722; 2767; | 100 | 100 | 100 | 100 | 100 | 7.77E−11 |
| | 2790; 2853; 2901; 2970; 3192; 3215; 3819; 3867; 3948; 4354; 4373; 4489; 4665; 4747; 4752; 4949; 4970; 4982 | | | | | | |
| 241 | 163; 207; 857; 3979 | 100 | 100 | 100 | 100 | 100 | 7.77E−11 |
| 242 | 242; 776; 3124; 3347; 3899; 3990; 4068; 4160; 4437; 4762; 5085; 5199 | 100 | 100 | 100 | 100 | 100 | 7.77E−11 |
| 243 | 1182; 3359; 3923; 4978 | 100 | 100 | 100 | 100 | 100 | 7.77E−11 |
| 244 | 176; 586; 667; 851; 885; 1109; 1188; 1545; 1551; 1929; 2063; 2834; 2963; 3205; 3370; 3495; 3831; | 100 | 100 | 100 | 100 | 100 | 7.77E−11 |
| | 3982; 4017; 4055; 4056; 4288; 4391; 4750; 5006; 5147 | | | | | | |
| 245 | 184; 265; 869; 2281; 3867; 4576 | 100 | 100 | 100 | 100 | 100 | 7.77E−11 |
| 246 | 165; 221; 229; 370; 417; 518; 569; 595; 1085; 1229; 1230; 1257; 1344; 1503; 1535; 1612; 1631; 1801; | 100 | 100 | 100 | 100 | 100 | 7.77E−11 |
| | 1950; 1976; 2105; 2300; 2564; 2732; 2871; 2950; 3054; 3340; 3575; 3658; 3709; 4190; 4383; 5012; 5030; 5197; 5213; 5251 | | | | | | |
| 247 | 114; 140; 175; 207; 272; 445; 738; 901; 935; 952; 1037; 1321; 1345; 1427; 1495; 1691; 1695; 1748; | 100 | 100 | 100 | 100 | 100 | 7.77E−11 |
| | 1822; 2636; 2742; 3046; 3188; 3455; 3757; 3942; 3957; 4016; 4030; 4118; 4252; 4608; 4780; 4835; 4887; 4999; 5067; 5071; 5248; 5251; 5259 | | | | | | |
| 248 | 48; 127; 237; 506; 1120; 1160; 1283; 1519; 1582; 1869; 1909; 2453; 3356; 3450; 3692; 3960; 4223; 4287; 4288; 5205 | 100 | 100 | 100 | 100 | 100 | 7.77E−11 |
| 249 | 47; 49; 236; 363; 623; 660; 675; 842; 1061; 1126; 1140; 1252; 1501; 1690; 1696; 1851; 2143; 2572; 2707; 2956; 2959; | 100 | 100 | 100 | 100 | 100 | 7.77E−11 |
| | 3113; 3363; 3659; 3663; 3825; 3881; 3969; 3995; 4106; 4240; 4267; 4291; 4380; 4433; 4767; 4969; 4983; 5240; 5266 | | | | | | |
| 250 | 118; 482; 603; 651; 815; 847; 850; 1056; 1070; 1134; 1185; 1196; 1270; 1474; 1511; 1766; 1841; 2074; | 100 | 100 | 100 | 100 | 100 | 7.77E−11 |
| | 2093; 2118; 2172; 2204; 2241; 2346; 2371; 2516; 2874; 2876; 2960; 3071; 3503; 3535; 3589; 3665; | | | | | | |
| | 3796; 3814; 3860; 3890; 4181; 4482; 4569; 4631; 4693; 4909; 5192 | | | | | | |
| 251 | 16; 33; 87; 133; 147; 221; 620; 741; 746; 782; 972; 1782; 1994; 2273; 2469; 2567; 2701; 2749; 2779; 2839; 2931; | 100 | 100 | 100 | 100 | 100 | 7.77E−11 |
| | 2938; 3270; 3384; 3388; 3446; 3490; 3640; 3643; 3832; 4010; 4175; 4246; 4334; 4742; 4772; 4786; 4903; 4909; 4923; 5140; 5170 | | | | | | |
| 252 | 178; 3256; 4024; 4613; 5001; 5167 | 100 | 100 | 100 | 100 | 100 | 7.77E−11 |
| 253 | 114; 327; 729; 1420; 2122; 2123; 2742; 3376; 3564; 3757; 3837; 4510; 4658; 4750; 5113; 5216 | 100 | 100 | 100 | 100 | 100 | 7.77E−11 |
| 254 | 865; 1469; 1579; 1852; 2297; 2494; 3388; 3446; 4054; 4400 | 100 | 100 | 100 | 100 | 100 | 7.77E−11 |
| 255 | 161; 837; 1277; 1461; 1619; 1938; 2506; 3770; 4365; 5136 | 100 | 100 | 100 | 100 | 100 | 7.77E−11 |
| 256 | 425; 464; 614; 957; 1552; 1943; 4139; 4341; 4840 | 100 | 100 | 100 | 100 | 100 | 7.77E−11 |
| 257 | 398; 1455; 1963; 2335; 3782; 4012; 4047; 4304; 4863 | 100 | 100 | 100 | 100 | 100 | 7.77E−11 |
| 258 | 13; 148; 176; 218; 358; 395; 489; 539; 856; 862; 1127; 1190; 1244; 1797; 2006; 2100; 2143; 2253; 2350; | 100 | 100 | 100 | 100 | 100 | 7.77E−11 |
| | 2357; 2432; 2516; 2530; 2620; 2652; 2730; 2771; 2880; 2919; 2937; 3247; 3553; 3579; 3854; 4059; | | | | | | |
| | 4230; 4232; 4236; 4617; 4645; 4664; 4679; 4717; 4894; 4947; 4959; 4974; 5100; 5167; 5270 | | | | | | |
| 259 | 450; 1800; 1914; 2996; 4590 | 100 | 100 | 100 | 100 | 100 | 7.77E−11 |
| 260 | 10; 41; 481; 515; 972; 1299; 1445; 2024; 2642; 3005; 3042; 3565; 3796; 3966; 4089; 4149; 4300; 4534; 5238; 5251 | 100 | 100 | 100 | 100 | 100 | 7.77E−11 |
| 261 | 146; 840; 1140; 1748; 2698; 2931; 3956; 4861; 4912 | 100 | 100 | 100 | 100 | 100 | 7.77E−11 |
| 262 | 502; 2787; 2827; 4269; 5250 | 100 | 100 | 100 | 100 | 100 | 7.77E−11 |
| 263 | 134; 235; 378; 712; 775; 1003; 1023; 1163; 1228; 1500; 1568; 1653; 1719; 1925; 1937; 2047; 2272; 2359; 2554; 2620; | 100 | 100 | 100 | 100 | 100 | 7.77E−11 |
| | 2667; 2815; 2918; 2996; 3159; 3196; 3494; 3610; 3796; 3929; 3931; 4142; 4373; 4395; 4473; 4786; 4794; 4986; 5141 | | | | | | |
| 264 | 144; 1518; 1710; 1961; 2184; 2304; 2442; 3697; 3729; 3902 | 100 | 100 | 100 | 100 | 100 | 7.77E−11 |
| 265 | 423; 463; 657; 847; 915; 924; 1109; 1122; 1261; 1341; 1563; 1610; 2042; 2246; 2429; 2600; 3159; 3440; 4247; 4589; 4648; 4750; 5100 | 100 | 100 | 100 | 100 | 100 | 7.77E−11 |
| 266 | 294; 485; 1218; 1707; 2340; 2640; 3043; 3341; 4234; 4438 | 100 | 100 | 100 | 100 | 100 | 7.77E−11 |
| 267 | 103; 194; 286; 320; 501; 692; 716; 1376; 1443; 1563; 1746; 1853; 2032; 2121; 2333; 2835; 3166; 3305; | 100 | 100 | 100 | 100 | 100 | 7.77E−11 |
| | 3415; 3463; 3496; 3497; 3533; 3801; 3900; 3990; 4001; 4094; 4108; 4143; 4194; 4219; 4798; 5151 | | | | | | |
| 268 | 101; 308; 387; 2046; 2365; 4028; 4095; 4508; 5010; 5210 | 100 | 100 | 100 | 100 | 100 | 7.77E−11 |
| 269 | 27; 99; 151; 178; 238; 369; 384; 771; 1065; 1271; 1277; 1322; 1454; 1480; 1507; 1574; 1618; 1659; 1662; | 100 | 100 | 100 | 100 | 100 | 7.77E−11 |
| | 1716; 1822; 1836; 1840; 1934; 1980; 1984; 2163; 2580; 2601; 2621; 2678; 3250; 3253; 3431; 3438; 3633; | | | | | | |
| | 3688; 3863; 3904; 4085; 4313; 4448; 4630; 4741; 4839; 4958; 5083 | | | | | | |
| 270 | 219; 889; 2595; 2943; 2990; 3413; 3708; 4318; 4519 | 100 | 100 | 100 | 100 | 100 | 7.77E−11 |
| 271 | 1104; 1386; 1766; 1868; 2588; 3209; 3390; 4228 | 100 | 100 | 100 | 100 | 100 | 7.77E−11 |
| 272 | 5; 525; 950; 1111; 1308; 2610; 3865; 5156 | 100 | 100 | 100 | 100 | 100 | 7.77E−11 |
| 273 | 599; 1263; 2763; 3240; 3502; 4228; 4830; 5284 | 100 | 100 | 100 | 100 | 100 | 7.77E−11 |
| 274 | 31; 595; 2628; 3051; 3273; 3577; 3917; 3936; 5025; 5265 | 100 | 100 | 100 | 100 | 100 | 7.77E−11 |
| 275 | 85; 236; 257; 1218; 1271; 1706; 1846; 2242; 2276; 2477; 2897; 2924; 3501; 3721; 4019; 4430; 4755; 4966 | 100 | 100 | 100 | 100 | 100 | 7.77E−11 |
| 276 | 592; 1082; 1595; 1756; 2114; 3673; 3710; 4095; 4166; 5224 | 100 | 100 | 100 | 100 | 100 | 7.77E−11 |
| 277 | 0; 207; 324; 343; 472; 850; 3588; 3636; 4261; 4566; 4618; 4733; 4848; 5098 | 100 | 100 | 100 | 100 | 100 | 7.77E−11 |
| 278 | 212; 749; 1581; 4057; 4292; 5014; 5099 | 100 | 100 | 100 | 100 | 100 | 7.77E−11 |
| 279 | 563; 3174; 4417; 4626; 4998 | 100 | 100 | 100 | 100 | 100 | 7.77E−11 |
| 280 | 75; 532; 1046; 1657; 2276; 2748; 2889; 2930; 4278; 4420 | 100 | 100 | 100 | 100 | 100 | 7.77E−11 |
| 281 | 207; 3113; 3509; 3778; 4376 | 100 | 100 | 100 | 100 | 100 | 7.77E−11 |
| 282 | 69; 152; 207; 242; 266; 326; 571; 598; 667; 721; 785; 910; 929; 1029; 1044; 1050; 1212; 1330; 1376; 1377; | 100 | 100 | 100 | 100 | 100 | 7.77E−11 |
| | 1718; 1795; 1848; 1993; 2049; 2141; 2351; 2499; 2680; 2815; 3017; 3082; 3176; 3219; 3282; 3454; 3480; | | | | | | |
| | 3535; 3588; 3687; 3791; 3923; 4066; 4104; 4206; 4835; 4854; 4942; 5014; 5251 | | | | | | |
| 283 | 105; 194; 266; 752; 976; 1096; 1299; 1633; 1732; 1787; 1872; 2103; 2257; 2457; 2819; 2931; 2954; 3419; | 100 | 100 | 100 | 100 | 100 | 7.77E−11 |
| | 3644; 3762; 3887; 4059; 4134; 4265; 4365; 4513; 4638; 4977; 5094; 5217; 5234 | | | | | | |
| 284 | 240; 268; 407; 491; 614; 777; 785; 814; 1050; 1114; 1215; 1268; 1309; 1311; 1595; 1604; 1684; 1971; | 100 | 100 | 100 | 100 | 100 | 7.77E−11 |
| | 2404; 3082; 3195; 3478; 3496; 4018; 4144; 4194; 4295; 4394; 4419; 4628; 4757; 4844; 5033; 5157; 5193 | | | | | | |
| 285 | 421; 832; 854; 957; 1890; 3146; 3876; 4360; 4651 | 100 | 100 | 100 | 100 | 100 | 7.77E−11 |
| 286 | 381; 1160; 1292; 1375; 1731; 1804; 1886; 1911; 1961; 2171; 2200; 2346; 2408; 2491; 2513; 2528; 2650; | 100 | 100 | 100 | 100 | 100 | 7.77E−11 |
| | 2820; 2846; 3030; 3309; 3596; 3685; 3784; 3788; 3865; 3900; 3913; 3959; 3975; 4218; 4331; 4596; 4912; 5275 | | | | | | |
| 287 | 135; 368; 369; 650; 1063; 1081; 1202; 1517; 1575; 2817; 3971; 4981 | 100 | 100 | 100 | 100 | 100 | 7.77E−11 |
| 288 | 94; 1708; 2087; 2236; 2297; 2492; 2742; 2846; 2867; 2900; 2907; 2986; 3128; 3769; 3835; 3847; 4026; 4633; 4925; 5267; 5280 | 100 | 100 | 100 | 100 | 100 | 7.77E−11 |
| 289 | 2715; 3259; 4224; 4498 | 100 | 100 | 100 | 100 | 100 | 7.77E−11 |
| 290 | 266; 1632; 3131; 3953; 5200 | 100 | 100 | 100 | 100 | 100 | 7.77E−11 |
| 291 | 213; 504; 617; 2852; 3086 | 100 | 100 | 100 | 100 | 100 | 7.77E−11 |
| 292 | 152; 345; 713; 1057; 2108; 2628; 3762; 4593; 5070; 5229 | 100 | 100 | 100 | 100 | 100 | 7.77E−11 |
| 293 | 889; 1186; 1446; 1456; 2206; 2303; 2534; 3059; 3131; 3542; 3697; 4396; 4419; 4717 | 100 | 100 | 100 | 100 | 100 | 7.77E−11 |
| 294 | 57; 318; 1125; 2120; 2363; 2932; 3340; 3528; 3583; 4146 | 100 | 100 | 100 | 100 | 100 | 7.77E−11 |
| 295 | 206; 296; 688; 1163; 1260; 1269; 1504; 1951; 2207; 2255; 2287; 2471; 2498; 2528; 2570; 2721; 2919; | 100 | 100 | 100 | 100 | 100 | 7.77E−11 |
| | 2970; 3202; 3661; 3738; 3745; 3894; 3916; 4076; 4114; 4161; 4171; 4332; 4497 | | | | | | |
| 296 | 266; 423; 506; 680; 1151; 1286; 1320; 1398; 1427; 1593; 1777; 1810; 2037; 2295; 2306; 3246; 3378; | 100 | 100 | 100 | 100 | 100 | 7.77E−11 |
| | 3550; 3759; 3792; 4078; 4350; 4787; 4901; 5028; 5257 | | | | | | |
| 297 | 199; 1206; 1593; 3948; 4284; 4377; 4676 | 100 | 100 | 100 | 100 | 100 | 7.77E−11 |
| 298 | 423; 1676; 1766; 2058; 2523; 2979; 3361; 4627; 5260 | 100 | 100 | 100 | 100 | 100 | 7.77E−11 |
| 299 | 8; 46; 303; 526; 941; 971; 1388; 1420; 1803; 1915; 2074; 2125; 2318; 2336; 2542; 2803; 2911; 3251; | 100 | 100 | 100 | 100 | 100 | 7.77E−11 |
| | 3267; 3297; 3365; 3567; 3886; 4143; 4187; 4423; 4861; 5201 | | | | | | |
| 300 | 120; 123; 405; 438; 682; 721; 1206; 1396; 1887; 2082; 2201; 2231; 2633; 2761; 2955; 3283; 3403; 4035; | 100 | 100 | 100 | 100 | 100 | 7.77E−11 |
| | 4115; 4298; 4324; 4367; 4515; 4523; 4604; 4633; 4639; 4678; 4739; 4862; 4953; 5090 | | | | | | |
| 301 | 1331; 2539; 2552; 2604; 2708; 2858; 4057; 4556; 4977; 5096 | 100 | 100 | 100 | 100 | 100 | 7.77E−11 |
| 302 | 267; 522; 691; 859; 945; 1121; 1232; 1726; 2406; 3221; 3332; 3549; 3824; 4272; 4357; 4976; 5030; 5178 | 100 | 100 | 100 | 100 | 100 | 7.77E−11 |
| 303 | 407; 920; 1081; 1292; 1662; 2183; 2464; 2515; 3019; 3289; 3454; 4032; 4876 | 100 | 100 | 100 | 100 | 100 | 7.77E−11 |
| 304 | 146; 357; 415; 547; 647; 666; 1435; 1472; 1474; 1799; 2311; 2318; 2375; 2412; 2416; 2444; 2498; 2910; 2977; 3139; | 100 | 100 | 100 | 100 | 100 | 7.77E−11 |
| | 3147; 3179; 3269; 3370; 3455; 3488; 3522; 4125; 4201; 4252; 4379; 4389; 4597; 4622; 4709; 4896; 4937; 5156; 5272 | | | | | | |
| 305 | 564; 1686; 1935; 2897; 3350; 4267; 4431; 4837; 5267 | 100 | 100 | 100 | 100 | 100 | 7.77E−11 |
| 306 | 50; 286; 293; 338; 421; 624; 749; 798; 1254; 1398; 1578; 1587; 1936; 2124; 2168; 2392; 2545; 2713; | 100 | 100 | 100 | 100 | 100 | 7.77E−11 |
| | 3078; 3141; 3421; 3550; 3734; 3759; 3857; 3905; 4002; 4564; 4899; 5048 | | | | | | |
| 307 | 508; 984; 1422; 2490; 3067; 3611; 3658; 4108; 4226; 4593 | 100 | 100 | 100 | 100 | 100 | 7.77E−11 |
| 308 | 354; 501; 610; 1247; 1659; 1841; 2173; 2371; 4473; 4984 | 100 | 100 | 100 | 100 | 100 | 7.77E−11 |
| 309 | 12; 15; 679; 715; 843; 910; 2236; 2514; 2826; 3295; 3381; 3862; 4498; 5018 | 100 | 100 | 100 | 100 | 100 | 7.77E−11 |
| 310 | 331; 424; 743; 1366; 1386; 1388; 1683; 1898; 1909; 1943; 2344; 2475; 2503; 2611; 2759; 3554; 3601; | 100 | 100 | 100 | 100 | 100 | 7.77E−11 |
| | 3720; 3982; 4009; 4178; 4544; 4923; 4984 | | | | | | |
| 311 | 2097; 3496; 4401; 4585; 4735 | 100 | 100 | 100 | 100 | 100 | 7.77E−11 |
| 312 | 890; 1273; 1903; 1926; 2031; 3620; 4284; 5196 | 100 | 100 | 100 | 100 | 100 | 7.77E−11 |
| 313 | 207; 465; 496; 654; 1000; 1004; 1008; 1206; 1283; 1374; 1966; 2138; 2211; 2753; 2780; 2861; 2951; 3482; 3988; 4768; 4917; 4931; 4933 | 100 | 100 | 100 | 100 | 100 | 7.77E−11 |
| 314 | 44; 49; 90; 203; 213; 346; 461; 804; 1199; 1264; 1378; 1473; 1557; 1566; 1589; 1686; 1913; 1975; 2037; | 100 | 100 | 100 | 100 | 100 | 7.77E−11 |
| | 2170; 2308; 2499; 2507; 2517; 2595; 2626; 2633; 2686; 2730; 2926; 2973; 3006; 3023; 3078; 3293; | | | | | | |
| | 3305; 3483; 3626; 3707; 3776; 3915; 3951; 3993; 4046; 4323; 4439; 4773; 4887; 5084; 5163 | | | | | | |
| 315 | 527; 696; 847; 889; 1049; 1251; 1276; 1377; 1702; 2109; 2395; 2691; 2733; 2734; 2860; 2863; 3096; | 100 | 100 | 100 | 100 | 100 | 7.77E−11 |
| | 3144; 3166; 3303; 3336; 3364; 3422; 3693; 3732; 3880; 3882; 3990; 4004; 4036; 4146; 4169; 4237; | | | | | | |
| | 4402; 4408; 4436; 4468; 4783; 5002; 5068; 5142; 5213; 5233; 5253; 5259 | | | | | | |
| 316 | 219; 2240; 2616; 4081 | 100 | 100 | 100 | 100 | 100 | 7.77E−11 |
| 317 | 74; 77; 410; 588; 603; 804; 811; 832; 868; 974; 976; 986; 1395; 1401; 1406; 1474; 1570; 1681; 1706; | 100 | 100 | 100 | 100 | 100 | 7.77E−11 |
| | 1891; 2108; 2115; 2360; 2450; 2505; 2851; 2878; 3210; 3455; 3461; 3628; 3676; 3707; 3789; 3828; | | | | | | |
| | 3860; 4050; 4134; 4159; 4207; 4217; 4510; 4571; 4637; 4672; 4677; 4885; 4960; 5106; 5205 | | | | | | |
| 318 | 141; 415; 454; 640; 648; 1130; 1384; 1477; 1560; 1879; 1890; 1900; 2101; 2237; 2698; 2714; 2824; | 100 | 100 | 100 | 100 | 100 | 7.77E−11 |
| | 2845; 2978; 3166; 3221; 3298; 3302; 3435; 3690; 3720; 3906; 4057; 4176; 4355; 4669; 4754; 4942; 5122 | | | | | | |
| 319 | 382; 461; 1191; 1903; 2078; 2720; 2760 | 100 | 100 | 100 | 100 | 100 | 7.77E−11 |
| 320 | 51; 174; 717; 1106; 3264; 4488 | 100 | 100 | 100 | 100 | 100 | 7.77E−11 |
| 321 | 83; 320; 367; 527; 678; 1323; 1695; 1828; 1880; 1991; 2083; 2092; 2390; 2596; 2723; 2836; 2942; 3441; | 100 | 100 | 100 | 100 | 100 | 7.77E−11 |
| | 3969; 4190; 4346; 4394; 4637; 4687; 5135 | | | | | | |
| 322 | 64; 177; 574; 987; 1203; 1564; 1758; 1991; 2011; 2030; 2163; 2620; 2792; 3494; 3710; 4143; 4252; 4447; 4708; 5070; 5186 | 100 | 100 | 100 | 100 | 100 | 7.77E−11 |
| 323 | 83; 519; 1009; 1178; 3527; 3640; 3925; 4259; 4580; 4672 | 100 | 100 | 100 | 100 | 100 | 7.77E−11 |
| 324 | 50; 929; 1735; 2755; 4023; 4225; 4746; 4769; 5214 | 100 | 100 | 100 | 100 | 100 | 7.77E−11 |
| 325 | 26; 75; 112; 140; 254; 322; 325; 332; 437; 482; 775; 887; 1304; 1508; 1509; 1617; 1810; 2120; 2190; 2534; 2578; | 100 | 100 | 100 | 100 | 100 | 7.77E−11 |
| | 2612; 2871; 2939; 2954; 3185; 3190; 3297; 3369; 3420; 3572; 3817; 3828; 3937; 4040; 4785; 5002; 5021; 5177 | | | | | | |
| 326 | 670; 1339; 1815; 3152; 3584; 3891; 4202; 4237; 4777; 5074 | 100 | 100 | 100 | 100 | 100 | 7.77E−11 |
| 327 | 564; 1439; 1513; 1893; 2624; 2863; 3515; 4004; 4945; 5006 | 100 | 100 | 100 | 100 | 100 | 7.77E−11 |
| 328 | 640; 742; 881; 1370; 2383; 2397; 3083; 3966; 4138 | 100 | 100 | 100 | 100 | 100 | 7.77E−11 |
| 329 | 152; 430; 532; 1538; 2830; 3194; 3507; 3699 | 100 | 100 | 100 | 100 | 100 | 7.77E−11 |
| 330 | 526; 1143; 1373; 3202; 3321; 3610; 4159; 5070; 5088; 5165 | 100 | 100 | 100 | 100 | 100 | 7.77E−11 |
| 331 | 941; 962; 1868; 2757; 3913; 4228 | 100 | 100 | 100 | 100 | 100 | 7.77E−11 |
| 332 | 106; 266; 334; 540; 626; 672; 777; 823; 833; 1106; 1323; 1621; 2063; 2125; 2458; 2577; 2584; 2686; | 100 | 100 | 100 | 100 | 100 | 7.77E−11 |
| | 3037; 3042; 3355; 3408; 3578; 3706; 3788; 3813; 3833; 3838; 3878; 3968; 4061; 4112; 4358; 4602 4633; 4648 | | | | | | |
| 333 | 1175; 1335; 3940; 4077; 5260 | 100 | 100 | 100 | 100 | 100 | 7.77E−11 |
| 334 | 227; 235; 915; 1095; 1474; 2155; 2211; 2246; 2386; 2424; 2613; 2752; 3328; 3380; 3505; 3732; 3932; 4088; 4126; 4631; 5077 | 100 | 100 | 100 | 100 | 100 | 7.77E−11 |
| 335 | 176; 179; 352; 496; 529; 577; 594; 640; 704; 1106; 1214; 1251; 1267; 1335; 1354; 1454; 1457; 1563; | 100 | 100 | 100 | 100 | 100 | 7.77E−11 |
| | 1678; 1815; 1906; 1923; 2029; 2257; 2564; 2818; 2822; 3106; 3315; 3627; 3828; 3871; 3939; 4101; | | | | | | |
| | 4162; 4180; 4366; 4464; 4547; 4618; 4742; 4760; 4844; 5009; 5214; 5220 | | | | | | |
| 336 | 534; 571; 581; 701; 718; 1220; 1241; 1720; 1770; 2250; 2265; 2572; 2714; 2732; 2972; 3198; 3270; | 100 | 100 | 100 | 100 | 100 | 7.77E−11 |
| | 3341; 3464; 3510; 3555; 3599; 3722; 3739; 3939; 3941; 3994; 4159; 4173; 4188; 4262; 4426; 4551; | | | | | | |
| | 4607; 4633; 4760; 4815; 4832; 4877; 4933; 4981 | | | | | | |
| 337 | 84; 120; 232; 330; 397; 417; 520; 544; 601; 611; 690; 719; 816; 975; 1124; 1276; 1289; 1361; 1406; | 100 | 100 | 100 | 100 | 100 | 7.77E−11 |
| | 1501; 1526; 1669; 1683; 1746; 1844; 1875; 1888; 2058; 2202; 2618; 2653; 3110; 3148; 3209; 3485; | | | | | | |
| | 3530; 3551; 3577; 3595; 3977; 4082; 4122; 4318; 4495; 4634; 4753; 4849; 5013; 5223 | | | | | | |
| 338 | 4; 194; 774; 1147; 1292; 1391; 1599; 1809; 1874; 2153; 2162; 2211; 2440; 2542; 2651; 2761; 2807; | 100 | 100 | 100 | 100 | 100 | 7.77E−11 |
| | 2810; 2845; 3066; 3193; 3310; 3445; 3471; 3536; 3932; 4314; 4613; 4651; 4705; 4838; 4883; 5046; 5109; 5142; 5189 | | | | | | |
| 339 | 250; 500; 505; 643; 744; 1208; 1307; 1323; 1412; 1413; 1598; 1745; 1799; 1841; 1858; 1930; 2125; | 100 | 100 | 100 | 100 | 100 | 7.77E−11 |
| | 2218; 2227; 2294; 2326; 2475; 2611; 2712; 2837; 2930; 2955; 2971; 3008; 3205; 3537; 3885; 3913; | | | | | | |
| | 4130; 4497; 4693; 4730; 4861; 4980; 5015 | | | | | | |
| 340 | 358; 2291; 2534; 2742; 3012; 3998; 4530; 4839; 4868 | 100 | 100 | 100 | 100 | 100 | 7.77E−11 |
| 341 | 488; 703; 751; 869; 910; 920; 1358; 1368; 1413; 1875; 1883; 1991; 2134; 2193; 2501; 2527; 2579; | 100 | 100 | 100 | 100 | 100 | 7.77E−11 |
| | 2642; 2672; 2686; 2759; 2852; 2976; 3114; 3420; 3702; 3748; 3829; 3986; 4186; 4325; 4434; 4443; 4610; 4841; 4997; 5227; 5257 | | | | | | |
| 342 | 287; 478; 919; 1751; 2118; 2266; 2796; 3216; 4064; 4767; 4886; 5008 | 100 | 100 | 100 | 100 | 100 | 7.77E−11 |
| 343 | 36; 144; 319; 586; 688; 896; 1189; 1411; 1420; 1499; 1794; 1961; 2051; 2564; 3108; 3238; 3298; 3381; | 100 | 100 | 100 | 100 | 100 | 7.77E−11 |
| | 3502; 3781; 3801; 3809; 3931; 4318; 4473; 4523; 4642; 4798; 4834; 5028 | | | | | | |
| 344 | 94; 181; 219; 233; 442; 481; 546; 682; 1523; 1557; 1562; 2065; 2071; 2171; 2210; 2214; 2239; 2301; | 100 | 100 | 100 | 100 | 100 | 7.77E−11 |
| | 2317; 2846; 2923; 2982; 3240; 3248; 3691; 3987; 4364; 4417; 4505; 4603; 4635; 4873; 5123 | | | | | | |
| 345 | 110; 191; 225; 315; 378; 612; 1416; 1491; 1541; 1649; 1724; 1784; 2045; 2056; 2116; 2133; 2194; 2221; | 100 | 100 | 100 | 100 | 100 | 7.77E−11 |
| | 2241; 2344; 2514; 2566; 2910; 3290; 3454; 3641; 3867; 3912; 4049; 4245; 4291; 4299; 4390; 4434; | | | | | | |
| | 4473; 4499; 4578; 4604; 4717; 4735; 4756; 4794; 4884; 4894; 5156 | | | | | | |
| 346 | 62; 1800; 2373; 2783; 2880; 2957; 3087; 3619; 4205; 4735 | 100 | 100 | 100 | 100 | 100 | 7.77E−11 |
| 347 | 100; 352; 384; 603; 939; 1421; 1914; 2602; 2732; 4530 | 100 | 100 | 100 | 100 | 100 | 7.77E−11 |
| 348 | 141; 298; 343; 552; 553; 731; 899; 920; 1268; 1384; 1876; 1889; 2031; 2128; 2170; 2199; 2227; 2327; | 100 | 100 | 100 | 100 | 100 | 7.77E−11 |
| | 2560; 2656; 2989; 3014; 3086; 3397; 3432; 3608; 3760; 3778; 3885; 3927; 4031; 4096; 4187; 4195; | | | | | | |
| | 4257; 4318; 4328; 4390; 4602; 4685; 4742; 4826; 4864; 5005; 5063; 5099; 5157; 5221 | | | | | | |
| 349 | 1004; 1069; 1652; 1840; 1980; 2279; 2762; 3095; 3292; 4044; 4172; 4175; 4206; 4261; 4498; 4645; 4669; 4887; 5004 | 100 | 100 | 100 | 100 | 100 | 7.77E−11 |
| 350 | 3; 67; 507; 824; 911; 1506; 1856; 1963; 2809; 3759; 3769; 4018; 4075; 4077; 4351; 4387; 4865; 5065; 5178; 5192 | 100 | 100 | 100 | 100 | 100 | 7.77E−11 |
| 351 | 173; 219; 242; 249; 411; 437; 593; 649; 794; 845; 850; 881; 908; 992; 1089; 1175; 1342; 1386; 1683; | 100 | 100 | 100 | 100 | 100 | 7.77E−11 |
| | 1712; 1917; 1940; 2204; 2272; 2362; 2459; 2629; 2633; 2667; 2863; 2938; 3128; 3209; 3559; 3677; | | | | | | |
| | 3730; 3812; 4028; 4138; 4207; 4412; 4513; 4706 | | | | | | |
| 352 | 152; 1167; 4476 | 100 | 100 | 100 | 100 | 100 | 7.77E−11 |
| 353 | 83; 655; 1472; 2660; 4869 | 100 | 100 | 100 | 100 | 100 | 7.77E−11 |
| 354 | 289; 332; 401; 452; 573; 603; 605; 612; 619; 937; 1023; 1195; 1345; 1496; 1498; 1548; 1622; 1650; 1678; 1875; 2064; | 100 | 100 | 100 | 100 | 100 | 7.77E−11 |
| | 2168; 2352; 2549; 2677; 2715; 2746; 3051; 3053; 3287; 3391; 3586; 3595; 3815; 3958; 4435; 4792; 4795; 5033; 5156 | | | | | | |
| 355 | 377; 784; 1126; 1228; 1388; 1606; 4203; 4465 | 100 | 100 | 100 | 100 | 100 | 7.77E−11 |
| 356 | 1894; 2019; 2770; 3064; 4361; 4942; 4963 | 100 | 100 | 100 | 100 | 100 | 7.77E−11 |
| 357 | 160; 506; 1401; 2810; 2960; 4435; 4514; 4646; 4982 | 100 | 100 | 100 | 100 | 100 | 7.77E−11 |
| 358 | 43; 75; 463; 467; 961; 1300; 1356; 1722; 1848; 1890; 1892; 2158; 2237; 2323; 2329; 2458; 3517; 3549; | 100 | 100 | 100 | 100 | 100 | 7.77E−11 |
| | 3901; 4378; 4406; 4457; 4604; 4644; 4841; 4854; 4991; 5009; 5140 | | | | | | |
| 359 | 46; 64; 113; 230; 240; 437; 564; 566; 578; 604; 663; 929; 933; 1180; 1240; 1957; 2079; 2307; 2665; 2757; | 100 | 100 | 100 | 100 | 100 | 7.77E−11 |
| | 2809; 2837; 2868; 3050; 3088; 3181; 3214; 3247; 3257; 3528; 3587; 3617; 3656; 3760; 4041; 4298; | | | | | | |
| | 4470; 4732; 4754; 4913; 4940; 4953; 5021; 5084; 5107; 5109; 5140 | | | | | | |
| 360 | 152; 233; 314; 366; 435; 1476; 1712; 2144; 2234; 2374; 2429; 2568; 2695; 2937; 3803; 4115; 4438; 4475; 4707; 5111; 5277 | 100 | 100 | 100 | 100 | 100 | 7.77E−11 |
| 361 | 77; 158; 489; 601; 967; 1338; 1451; 1507; 1745; 2170; 2539; 2564; 2623; 2682; 2760; 2940; 3016; 3116; | 100 | 100 | 100 | 100 | 100 | 7.77E−11 |
| | 3223; 3455; 3474; 3493; 3736; 3870; 4144; 4310; 4328; 4659 | | | | | | |
| 362 | 95; 342; 1029; 1248; 1251; 1346; 2267; 3075; 3309; 3557; 3867; 4076; 4864 | 100 | 100 | 100 | 100 | 100 | 7.77E−11 |
| 363 | 218; 611; 1255; 1429; 2036; 2070; 2706; 3446; 4918 | 100 | 100 | 100 | 100 | 100 | 7.77E−11 |
| 364 | 622; 923; 1565; 1716; 2773; 2821; 4363; 5022 | 100 | 100 | 100 | 100 | 100 | 7.77E−11 |
| 365 | 28; 34; 139; 181; 310; 417; 586; 685; 717; 844; 1171; 1288; 1461; 1477; 1503; 1798; 2021; 2377; 2948; | 100 | 100 | 100 | 100 | 100 | 7.77E−11 |
| | 2965; 3116; 3209; 3355; 3417; 3569; 3619; 3767; 3820; 3937; 4100; 4144; 4216; 4559; 4625; 4927; 5054; 5158; 5269 | | | | | | |
| 366 | 146; 896; 1336; 1408; 1409; 1880; 2096; 2136; 3219; 3975; 5036; 5241 | 100 | 100 | 100 | 100 | 100 | 7.77E−11 |
| 367 | 220; 238; 369; 1428; 2437; 3559; 4238; 4297; 4475; 4602; 4630; 4770; 4863; 5101 | 100 | 100 | 100 | 100 | 100 | 7.77E−11 |
| 368 | 331; 694; 786; 1080; 1371; 1754; 2003; 2056; 2925; 3685; 4180; 4474; 4723; 4859; 4903; 4979 | 100 | 100 | 100 | 100 | 100 | 7.77E−11 |
| 369 | 74; 87; 287; 438; 470; 570; 577; 1265; 1433; 1759; 2041; 2413; 2462; 2583; 2740; 3197; 3209; 3312; | 100 | 100 | 100 | 100 | 100 | 7.77E−11 |
| | 3343; 3595; 4060; 4279; 4296; 4394; 4444; 4567 | | | | | | |
| 370 | 45; 47; 122; 189; 348; 356; 392; 958; 1194; 1211; 1372; 1391; 1672; 1729; 1906; 1935; 2321; 2444; | 100 | 100 | 100 | 100 | 100 | 7.77E−11 |
| | 2804; 3009; 3395; 3515; 4011; 4313; 4316; 4566; 4630; 4652; 4998; 5248 | | | | | | |
| 371 | 277; 363; 648; 892; 1229; 1253; 1283; 1335; 1598; 1631; 1680; 1730; 1780; 1911; 1935; 2115; 2172; | 100 | 100 | 100 | 100 | 100 | 7.77E−11 |
| | 2198; 2278; 2476; 2483; 2599; 2642; 2843; 2954; 2978; 3061; 3165; 3195; 3369; 3421; 3512; 3674; | | | | | | |
| | 3744; 3869; 3899; 3929; 4058; 4255; 4274; 4364; 4437; 4472; 4545; 4846; 4930; 4963; 4995 | | | | | | |
| 372 | 146; 1152; 3167; 4026; 4942; 5068 | 100 | 100 | 100 | 100 | 100 | 7.77E−11 |
| 373 | 269; 634; 1632; 3962; 4073; 4888 | 100 | 100 | 100 | 100 | 100 | 7.77E−11 |
| 374 | 1758; 2257; 2784; 3202 | 100 | 100 | 100 | 100 | 100 | 7.77E−11 |
| 375 | 1152; 4587 | 100 | 100 | 100 | 100 | 100 | 7.77E−11 |
| 376 | 32; 346; 379; 388; 491; 515; 585; 917; 965; 1202; 1244; 1269; 1298; 1356; 1413; 1488; 1882; 1927; | 100 | 100 | 100 | 100 | 100 | 7.77E−11 |
| | 1935; 2300; 2431; 2641; 2735; 3182; 3209; 3234; 4054; 4086; 4140; 4164; 4217; 4263; 4271; 4283; | | | | | | |
| | 4472; 4572; 4649; 4826; 4846; 4894; 4992; 5094 | | | | | | |
| 377 | 961; 1203; 1591; 1782; 2217; 3012; 3075; 4517 | 100 | 100 | 100 | 100 | 100 | 7.77E−11 |
| 378 | 1903; 2125; 3814; 4141; 5004 | 100 | 100 | 100 | 100 | 100 | 7.77E−11 |
| 379 | 77; 746; 2211; 2304; 2928; 3587; 3677 | 100 | 100 | 100 | 100 | 100 | 7.77E−11 |
| 380 | 345; 784; 1169; 1606; 2717; 2858; 3082; 3324; 3725; 3754; 3784; 3920; 4213; 4563; 4774; 4862; 5057 | 100 | 100 | 100 | 100 | 100 | 7.77E−11 |
| 381 | 219; 287; 1125; 2779; 3237; 3317; 3620; 3689; 3967 | 100 | 100 | 100 | 100 | 100 | 7.77E−11 |
| 382 | 37; 78; 107; 208; 282; 369; 410; 417; 424; 432; 450; 469; 645; 735; 1020; 1279; 1302; 1387; 1398; 1460; | 100 | 100 | 100 | 100 | 100 | 7.77E−11 |
| | 1715; 2014; 2031; 2047; 2391; 2684; 2811; 2916; 3111; 3127; 3379; 3459; 3632; 3643; 3750; 3973; | | | | | | |
| | 4060; 4314; 4376; 4593; 4641; 4648; 4827; 4898; 5115 | | | | | | |
| 383 | 153; 278; 392; 538; 554; 716; 1168; 1441; 1550; 1676; 1729; 1732; 1764; 1838; 1921; 1990; 2016; 2046; | 100 | 100 | 100 | 100 | 100 | 7.77E−11 |
| | 2255; 2341; 2406; 2619; 2833; 2910; 3073; 3093; 3183; 3306; 3388; 3401; 3674; 3854; 3903; 3943; | | | | | | |
| | 3949; 4084; 4113; 4272; 4290; 4452; 4476; 4633; 4667; 4766; 4790; 4809; 4956; 5208 | | | | | | |
| 384 | 166; 249; 256; 379; 498; 506; 584; 680; 785; 1262; 1375; 1591; 1681; 1813; 1943; 2073; 2164; 2255; 2287; 2428; | 100 | 100 | 100 | 100 | 100 | 7.77E−11 |
| | 3110; 3198; 3265; 3311; 3906; 3916; 3964; 3976; 3977; 4152; 4217; 4319; 4423; 4504; 4520; 4644; 4755; 4870; 5258; 5272; 5285 | | | | | | |
| 385 | 177; 659; 1089; 3079; 3285; 3288; 4006; 4222; 4657; 4721 | 100 | 100 | 100 | 100 | 100 | 7.77E−11 |
| 386 | 451; 1255; 1766; 2686; 2765 | 100 | 100 | 100 | 100 | 100 | 7.77E−11 |
| 387 | 147; 228; 237; 333; 377; 380; 455; 574; 782; 1117; 1129; 1548; 1603; 1777; 1853; 2021; 2042; 2419; | 100 | 100 | 100 | 100 | 100 | 7.77E−11 |
| | 2752; 2874; 3108; 3339; 3420; 3462; 3736; 3763; 4388; 4556; 4679; 4835; 4903; 5239; 5258 | | | | | | |
| 388 | 922; 1005; 1108; 1678; 2210; 2244; 2516; 2630; 2665; 3371; 3623; 5193 | 100 | 100 | 100 | 100 | 100 | 7.77E−11 |
| 389 | 1908; 2920; 3209; 4497; 5011; 5078; 5137 | 100 | 100 | 100 | 100 | 100 | 7.77E−11 |
| 390 | 131; 221; 444; 823; 1010; 1138; 1259; 1328; 1874; 1955; 2851; 2959; 3032; 3033; 3076; 3086; 3154; | 100 | 100 | 100 | 100 | 100 | 7.77E−11 |
| | 3316; 3420; 3602; 3640; 3646; 3949; 4149; 4207; 4208; 4543; 4841 | | | | | | |
| 391 | 326; 526; 937; 1009; 1083; 1297; 1398; 1405; 1484; 1695; 1704; 1780; 1950; 2033; 2052; 2137; 2289; | 100 | 100 | 100 | 100 | 100 | 7.77E−11 |
| | 2341; 2643; 2671; 2830; 2944; 3043; 3093; 3166; 3238; 3586; 3852; 3860; 3921; 3976; 4157; 4191; | | | | | | |
| | 4384; 4504; 4529; 4659; 4805; 4944; 4953; 4982; 5153 | | | | | | |
| 392 | 578; 2333; 2559; 2995; 4488; 4980 | 100 | 100 | 100 | 100 | 100 | 7.77E−11 |
| 393 | 772; 801; 1085; 2051; 2154; 3310; 3532; 3740 | 100 | 100 | 100 | 100 | 100 | 7.77E−11 |
| 394 | 3; 21; 41; 118; 422; 528; 974; 1002; 1181; 1365; 1453; 1697; 1722; 1742; 1946; 1965; 2029; 2344; 2375; | 100 | 100 | 100 | 100 | 100 | 7.77E−11 |
| | 2607; 2834; 2859; 2949; 2954; 3118; 3244; 3325; 3343; 3386; 3564; 3695; 3894; 3959; 4095; 4144; | | | | | | |
| | 4158; 4314; 4332; 4532; 4540; 4760; 4837; 4921; 4966; 5032 | | | | | | |
| 395 | 456; 633; 750; 1261; 1705; 1904; 3138; 3209; 3462 | 100 | 100 | 100 | 100 | 100 | 7.77E−11 |
| 396 | 660; 1181; 1390; 1445; 2335; 2564; 2979; 3112; 3143; 3298; 3359; 3529; 3563; 3795; 4080; 4227; 4388; 4744 | 100 | 100 | 100 | 100 | 100 | 7.77E−11 |
| 397 | 647; 1151; 1347; 1420; 2671; 2692; 2827; 3701; 4286 | 100 | 100 | 100 | 100 | 100 | 7.77E−11 |
| 398 | 1186; 2014; 2451; 3666; 3782; 4770; 5201 | 100 | 100 | 100 | 100 | 100 | 7.77E−11 |
| 399 | 75; 111; 250; 412; 433; 506; 800; 1056; 1350; 1401; 1454; 1588; 1812; 1829; 1952; 2081; 2183; 2403; 2433; 2435; | 100 | 100 | 100 | 100 | 100 | 7.77E−11 |
| | 2696; 2821; 3198; 3216; 3304; 3511; 3568; 3745; 3772; 4037; 4214; 4371; 4388; 4503; 4670; 4779; 4807; 4935; 5075; 5193; 5221 | | | | | | |
| 400 | 27; 203; 325; 422; 431; 493; 523; 1129; 1162; 1192; 1280; 1449; 1469; 1607; 1764; 1868; 1878; 1880; | 100 | 100 | 100 | 100 | 100 | 7.77E−11 |
| | 1946; 2044; 2080; 2091; 2237; 2244; 2290; 2314; 2427; 2624; 2723; 2781; 2900; 3027; 3336; 3488; | | | | | | |
| | 3606; 3755; 3859; 4069; 4314; 4376; 4533; 4632; 4657; 4836; 4884; 4969; 5190; 5216; 5251 | | | | | | |
| 401 | 231; 753; 859; 1623; 1854; 2227; 3129; 3586; 3710 | 100 | 100 | 100 | 100 | 100 | 7.77E−11 |
| 402 | 219; 1178; 1347; 1818; 2904; 5070 | 100 | 100 | 100 | 100 | 100 | 7.77E−11 |
| 403 | 397; 913; 1690; 1935; 2153; 2415; 2447; 2460; 3226; 4371 | 100 | 100 | 100 | 100 | 100 | 7.77E−11 |
| 404 | 46; 89; 397; 569; 601; 621; 646; 711; 719; 1226; 1504; 2067; 2260; 2339; 2404; 2607; 3075; 3165; 3209; | 100 | 100 | 100 | 100 | 100 | 7.77E−11 |
| | 3296; 3363; 3443; 3509; 4034; 4089; 4166; 4389; 4435; 4548; 4698; 4836; 4986; 5111; 5284 | | | | | | |
| 405 | 178; 415; 579; 591; 924; 965; 1039; 1098; 1534; 1548; 1634; 1874; 2150; 2208; 2246; 2665; 2886; 2938; | 100 | 100 | 100 | 100 | 100 | 7.77E−11 |
| | 3096; 3391; 3397; 3773; 3971; 4006; 4378; 4394; 4545; 4559; 4666; 4766; 5043; 5063 | | | | | | |
| 406 | 127; 265; 646; 1561; 2403; 2870; 2971; 3591; 4865; 5201 | 100 | 100 | 100 | 100 | 100 | 7.77E−11 |
| 407 | 629; 937; 1521; 1731; 1928; 2075; 2240; 3589; 4057; 4541; 5239; 5286 | 100 | 100 | 100 | 100 | 100 | 7.77E−11 |
| 408 | 135; 906; 1665; 3347; 5231 | 100 | 100 | 100 | 100 | 100 | 7.77E−11 |
| 409 | 581; 1167; 1375; 2667; 3422; 4418 | 100 | 100 | 100 | 100 | 100 | 7.77E−11 |
| 410 | 205; 282; 478; 553; 774; 985; 1103; 1351; 1662; 1782; 2375; 2451; 2628; 2806; 3196; 3357; 3364; | 100 | 100 | 100 | 100 | 100 | 7.77E−11 |
| | 3518; 3662; 3699; 3715; 3776; 3893; 4012; 4442; 4630; 4769; 5231 | | | | | | |
| 411 | 8; 234; 475; 510; 529; 611; 618; 713; 1809; 2158; 2506; 2520; 2538; 2676; 2809; 2820; 2889; 3064; | 100 | 100 | 100 | 100 | 100 | 7.77E−11 |
| | 3227; 3259; 3271; 3285; 3313; 3383; 3500; 3508; 3575; 3677; 3860; 3868; 3916; 4071; 4078; 4138; | | | | | | |
| | 4348; 4580; 4597; 4637; 4923; 5048; 5208 | | | | | | |
| 412 | 126; 188; 207; 217; 560; 580; 727; 949; 1135; 1604; 1701; 1800; 2340; 2494; 2510; 2650; 2680; 2714; | 100 | 100 | 100 | 100 | 100 | 7.77E−11 |
| | 2795; 2822; 3045; 3209; 3295; 3315; 3416; 3926; 3954; 3972; 4139; 4169; 4389; 4723; 4859; 5148; 5204; 5259 | | | | | | |
| 413 | 392; 794; 1231; 2208; 2863; 3498; 3724; 4186; 4580 | 100 | 100 | 100 | 100 | 100 | 7.77E−11 |
| 414 | 44; 135; 320; 393; 429; 560; 675; 692; 698; 934; 984; 1323; 1399; 1577; 1580; 1585; 1609; 2128; 2207; | 100 | 100 | 100 | 100 | 100 | 7.77E−11 |
| | 2234; 2508; 2587; 2617; 2650; 2687; 2691; 2910; 2917; 3209; 3233; 3371; 3564; 3659; 3864; 3875; | | | | | | |
| | 4025; 4068; 4176; 4197; 4309; 4340; 4462; 4474; 4574; 4613; 4690; 4860; 5000; 5053 | | | | | | |
| 415 | 180; 325; 437; 544; 634; 902; 1775; 1957; 2007; 2027; 2082; 2476; 2941; 3384; 3893; 4096; 4546; 4654; 4857; 4967 | 100 | 100 | 100 | 100 | 100 | 7.77E−11 |
| 416 | 129; 539; 549; 614; 877; 1282; 1721; 1928; 2474; 2482; 2849; 3592; 4058; 4067; 4564; 4603; 4668; 4903; 5028 | 100 | 100 | 100 | 100 | 100 | 7.77E−11 |
| 417 | 98; 241; 287; 498; 561; 834; 861; 1044; 1124; 1171; 1518; 1947; 1994; 2180; 2194; 2623; 2941; 3402; | 100 | 100 | 100 | 100 | 100 | 7.77E−11 |
| | 3450; 3840; 4225; 4440; 4457; 4613; 4709; 4855; 4945; 4950; 5010 | | | | | | |
| 418 | 672; 927; 1243; 1927; 2399; 2599; 3102; 3974; 4010; 4048 | 100 | 100 | 100 | 100 | 100 | 7.77E−11 |
| 419 | 118; 630; 3976; 4360; 4434; 4652 | 100 | 100 | 100 | 100 | 100 | 7.77E−11 |
| 420 | 138; 1307; 1712; 2297; 2623; 3552; 3782; 4294; 4324; 4490 | 100 | 100 | 100 | 100 | 100 | 7.77E−11 |
| 421 | 1396; 2523; 4250; 5043 | 100 | 100 | 100 | 100 | 100 | 7.77E−11 |
| 422 | 46; 529; 1143; 1401; 1573; 1868; 3214; 3259; 3381; 3760; 4130; 4156; 4611; 4714; 5054; 5205; 5206 | 100 | 100 | 100 | 100 | 100 | 7.77E−11 |
| 423 | 25; 62; 71; 152; 203; 218; 371; 851; 1047; 1549; 1610; 1736; 1739; 1868; 1906; 2234; 2307; 2378; 2450; | 100 | 100 | 100 | 100 | 100 | 7.77E−11 |
| | 2468; 2792; 2869; 3152; 3153; 3217; 3277; 3444; 3539; 3618; 3630; 3745; 4012; 4230; 4404; 4523; 4562; 4592; 4669; 4695; 4735; 4757 | | | | | | |
| 424 | 1796; 2850; 3747; 4191; 4434; 4831 | 100 | 100 | 100 | 100 | 100 | 7.77E−11 |
| 425 | 607; 982; 1701; 2807; 4028 | 100 | 100 | 100 | 100 | 100 | 7.77E−11 |
| 426 | 4; 747; 2125; 2613; 2831; 3009; 4084; 4927 | 100 | 100 | 100 | 100 | 100 | 7.77E−11 |
| 427 | 1461; 1665; 2004; 2084; 2427; 3656; 4763 | 100 | 100 | 100 | 100 | 100 | 7.77E−11 |
| 428 | 407; 531; 1259; 1785; 2344; 2490; 4972; 5106 | 100 | 100 | 100 | 100 | 100 | 7.77E−11 |
| 429 | 965; 1143; 1529; 2489; 3033; 3293; 3508; 3582; 3706 | 100 | 100 | 100 | 100 | 100 | 7.77E−11 |
| 430 | 27; 178; 1024; 1046; 1086; 1133; 1395; 1428; 1763; 1929; 1963; 2155; 2220; 2424; 2643; 2787; 2828; | 100 | 100 | 100 | 100 | 100 | 7.77E−11 |
| | 2857; 2922; 2928; 2943; 3388; 3726; 3960; 4109; 4132; 4272; 4316; 4383; 4769; 4978; 5020; 5128; 5177 | | | | | | |
| 431 | 289; 711; 749; 819; 865; 1032; 1376; 1439; 1818; 1934; 2158; 2234; 2257; 2440; 2477; 2639; 2708; | 100 | 100 | 100 | 100 | 100 | 7.77E−11 |
| | 3243; 3643; 3748; 3773; 3815; 3914; 4118; 4254; 4298; 4460; 4855 | | | | | | |
| 432 | 687; 1208; 1375; 2944; 4214; 4312; 5054 | 100 | 100 | 100 | 100 | 100 | 7.77E−11 |
| 433 | 268; 330; 656; 1239; 1269; 1271; 1276; 2290; 2513; 2607 | 100 | 100 | 100 | 100 | 100 | 7.77E−11 |
| 434 | 1242; 2024; 2128; 2640; 2702; 3948; 3975; 4669; 4846 | 100 | 100 | 100 | 100 | 100 | 7.77E−11 |
| 435 | 161; 519; 723; 737; 768; 1097; 1219; 1549; 1894; 2072; 2463; 2615; 3059; 3067; 3800; 3923; 3986; 5036 | 100 | 100 | 100 | 100 | 100 | 7.77E−11 |
| 436 | 336; 1171; 1593; 2621; 2898; 2907; 3612; 3651; 3822; 4112; 4117; 4206; 4281; 4975; 5181; 5283 | 100 | 100 | 100 | 100 | 100 | 7.77E−11 |
| 437 | 345; 348; 353; 442; 530; 606; 1104; 1182; 1241; 1448; 1487; 1624; 2028; 2191; 2610; 2624; 2806; | 100 | 100 | 100 | 100 | 100 | 7.77E−11 |
| | 3093; 3128; 3232; 3308; 3343; 3381; 3505; 3542; 3581; 3771; 3779; 4354; 4441; 4586; 4658; 4714; 4724; 4814 | | | | | | |
| 438 | 784; 2566; 2765; 3945; 4943 | 100 | 100 | 100 | 100 | 100 | 7.77E−11 |
| 439 | 37; 617; 710; 925; 1240; 1387; 2080; 2669; 2802; 2855; 3008; 3463; 3524; 4368; 4597; 5286 | 100 | 100 | 100 | 100 | 100 | 7.77E−11 |
| 440 | 285; 341; 356; 468; 607; 915; 918; 968; 1004; 1066; 1180; 1428; 1669; 1892; 2436; 2456; 2468; 2833; | 100 | 100 | 100 | 100 | 100 | 7.77E−11 |
| | 2959; 3035; 3289; 3446; 3450; 3496; 3533; 3563; 3616; 3665; 3777; 3898; 4202; 4226; 4316; 4377; | | | | | | |
| | 4382; 4444; 4534; 4546; 4614; 4735; 5062; 5155 | | | | | | |
| 441 | 220; 235; 331; 355; 368; 581; 602; 699; 819; 1639; 1683; 1717; 1846; 2306; 2307; 2456; 2551; 2582; | 100 | 100 | 100 | 100 | 100 | 7.77E−11 |
| | 2772; 3100; 3156; 3396; 3524; 3597; 4013; 4095; 4225; 4284; 4567; 4653; 4678; 4916; 4993; 5001 | | | | | | |
| 442 | 953; 2447; 3194; 3876; 3950; 4124; 4441; 4626; 4879; 5263 | 100 | 100 | 100 | 100 | 100 | 7.77E−11 |
| 443 | 1347; 1542; 2194; 2200; 2208; 3178; 3352; 3357; 4665; 4931 | 100 | 100 | 100 | 100 | 100 | 7.77E−11 |
| 444 | 647; 713; 1551; 2338; 2993; 3340; 3589; 4149 | 100 | 100 | 100 | 100 | 100 | 7.77E−11 |
| 445 | 788; 1417; 1629; 3399; 4353; 4835; 5157; 5197 | 100 | 100 | 100 | 100 | 100 | 7.77E−11 |
| 446 | 333; 623; 629; 899; 2113; 2333; 2516; 2672; 2686; 3223; 4207; 4341; 4386; 4451; 4648; 4884; 5025 | 100 | 100 | 100 | 100 | 100 | 7.77E−11 |
| 447 | 176; 421; 629; 726; 1100; 1476; 1673; 1741; 2151; 2595; 2926; 3218; 3281; 3840; 4526; 4594; 4768; 4954; 4975 | 100 | 100 | 100 | 100 | 100 | 7.77E−11 |
| 448 | 2314; 2760; 2831; 2930; 2983; 4469; 4704; 5233 | 100 | 100 | 100 | 100 | 100 | 7.77E−11 |
| 449 | 2640; 3252; 3416; 3509; 3829; 4805; 4950 | 100 | 100 | 100 | 100 | 100 | 7.77E−11 |
| 450 | 672; 1285; 2420; 2492; 2525; 3374; 3758; 4126; 4433; 5010 | 100 | 100 | 100 | 100 | 100 | 7.77E−11 |
| 451 | 68; 171; 352; 928; 972; 1312; 1482; 1527; 1537; 1589; 1640; 1826; 2077; 2283; 2380; 2415; 2507; 2950; | 100 | 100 | 100 | 100 | 100 | 7.77E−11 |
| | 3202; 3232; 3342; 3426; 3558; 3664; 3701; 3803; 3909; 3939; 3984; 4035; 4046; 4134; 4519; 4710; 4757; 4882; 5087; 5094; 5195 | | | | | | |
| 452 | 4; 1606; 5000; 5063; 5254 | 100 | 100 | 100 | 100 | 100 | 7.77E−11 |
| 453 | 380; 1050; 3136; 3415; 3972; 4994; 5037 | 100 | 100 | 100 | 100 | 100 | 7.77E−11 |
| 454 | 1238; 1550; 1905; 2313; 2929; 3939; 5018 | 100 | 100 | 100 | 100 | 100 | 7.77E−11 |
| 455 | 175; 270; 276; 297; 495; 545; 705; 859; 1046; 1192; 1358; 1388; 1475; 1616; 1769; 1994; 2017; 2429; | 100 | 100 | 100 | 100 | 100 | 7.77E−11 |
| | 2453; 2800; 2994; 3387; 3659; 3779; 4218; 4697; 4738; 5180; 5199 | | | | | | |
| 456 | 103; 164; 448; 519; 592; 606; 640; 646; 741; 934; 1090; 1178; 1714; 2262; 2299; 2350; 2359; 2434; | 100 | 100 | 100 | 100 | 100 | 7.77E−11 |
| | 2674; 2878; 2933; 3023; 3060; 3138; 3352; 3560; 3594; 3838; 3926; 4033; 4098; 4109; 4110; 4343; | | | | | | |
| | 4381; 4395; 4542; 4563; 4609; 4767; 4841; 4872; 5008; 5050; 5130; 5172 | | | | | | |
| 457 | 173; 248; 349; 559; 636; 667; 850; 876; 1012; 1452; 1481; 1499; 1520; 1608; 1743; 2070; 2105; 2141; | 100 | 100 | 100 | 100 | 100 | 7.77E−11 |
| | 2181; 2186; 2275; 2400; 2819; 2830; 2896; 2998; 3570; 3582; 3701; 4572; 4574; 5267 | | | | | | |
| 458 | 325; 424; 647; 991; 1193; 1686; 2989; 4038; 4093; 4438; 4693; 4725; 4763; 5220 | 100 | 100 | 100 | 100 | 100 | 7.77E−11 |
| 459 | 427; 1761; 2058; 2962; 3318; 4434; 4600 | 100 | 100 | 100 | 100 | 100 | 7.77E−11 |
| 460 | 627; 1036; 1206; 1474; 1555; 2100; 2271; 2331; 3289; 3743; 4669; 4891 | 100 | 100 | 100 | 100 | 100 | 7.77E−11 |
| 461 | 806; 1153; 1892; 2009; 2096; 3195; 3282; 3542; 4018; 4095 | 100 | 100 | 100 | 100 | 100 | 7.77E−11 |
| 462 | 514; 679; 727; 966; 1064; 1147; 1192; 1348; 1664; 1749; 1800; 1807; 1841; 1912; 1929; 1949; 2073; | 100 | 100 | 100 | 100 | 100 | 7.77E−11 |
| | 2144; 2153; 2234; 2286; 2581; 2757; 2862; 3076; 3081; 3109; 3244; 3378; 3460; 4106; 4144; 4178; | | | | | | |
| | 4457; 4653; 4772; 5004; 5041; 5160; 5210; 5241 | | | | | | |
| 463 | 8; 306; 437; 831; 1062; 1106; 1215; 1402; 1567; 1634; 1795; 1829; 1873; 2045; 2383; 2820; 3111; 3408; | 100 | 100 | 100 | 100 | 100 | 7.77E−11 |
| | 3567; 4589; 4608; 4692; 4735; 4817; 4918; 5107 | | | | | | |
| 464 | 56; 515; 822; 1612; 1661; 1838; 1904; 2528; 2628; 2797; 3209; 3256; 3318; 3539; 3974; 4135; 4374; | 100 | 100 | 100 | 100 | 100 | 7.77E−11 |
| | 4444; 4499; 4587; 5005; 5050 | | | | | | |
| 465 | 536; 738; 1335; 3561; 3927; 3949; 4094; 4498; 5001 | 100 | 100 | 100 | 100 | 100 | 7.77E−11 |
| 466 | 382; 398; 548; 619; 743; 774; 1064; 1196; 1200; 1293; 1378; 1399; 1506; 1590; 1724; 2257; 2313; 2363; | 100 | 100 | 100 | 100 | 100 | 7.77E−11 |
| | 2710; 3028; 3258; 3259; 3339; 3363; 3436; 3453; 3615; 3696; 3796; 4069; 4134; 4238; 4286; 4618; 4655; 4666; 4905; 4932 | | | | | | |
| 467 | 138; 541; 604; 678; 737; 738; 887; 1108; 1162; 1366; 1499; 1789; 2103; 2516; 2625; 3643; 4000; 4035; | 100 | 100 | 100 | 100 | 100 | 7.77E−11 |
| | 4196; 4360; 4474; 4681; 4705; 4875; 5000; 5002; 5058; 5105 | | | | | | |
| 468 | 140; 205; 456; 559; 905; 1086; 1355; 1563; 1745; 1767; 2484; 2667; 2845; 3276; 3346; 3785; 4183; | 100 | 100 | 100 | 100 | 100 | 7.77E−11 |
| | 4334; 4366; 4485; 4596; 4935; 5075; 5212; 5224; 5241 | | | | | | |
| 469 | 189; 213; 259; 537; 544; 725; 1541; 1946; 2677; 2935; 3028; 3580; 3661; 3756; 4208; 4281; 4506; 4533; 5112; 5142 | 100 | 100 | 100 | 100 | 100 | 7.77E−11 |
| 470 | 1456; 1562; 4263 | 100 | 100 | 100 | 100 | 100 | 7.77E−11 |
| 471 | 62; 218; 551; 683; 775; 777; 964; 1573; 1807; 1935; 2924; 3421; 3715; 3812; 4416; 4517; 4777; 5149; 5157 | 100 | 100 | 100 | 100 | 100 | 7.77E−11 |
| 472 | 3; 819; 835; 2210; 3142; 3346; 3639; 4915 | 100 | 100 | 100 | 100 | 100 | 7.77E−11 |
| 473 | 689; 943; 1457; 4384; 4735 | 100 | 100 | 100 | 100 | 100 | 7.77E−11 |
| 474 | 284; 383; 1340; 1366; 2488; 3128; 3491; 4239; 4240; 4254; 4863; 5156 | 100 | 100 | 100 | 100 | 100 | 7.77E−11 |
| 475 | 133; 185; 539; 615; 822; 1035; 1156; 1169; 1212; 1352; 1557; 2062; 2332; 2667; 2704; 3239; 3936; | 100 | 100 | 100 | 100 | 100 | 7.77E−11 |
| | 4186; 4214; 4380; 4399; 4414; 4903; 4961; 5009; 5133; 5177 | | | | | | |
| 476 | 827; 1271; 1282; 1343; 2138; 2971; 3542; 3644; 4100; 4820 | 100 | 100 | 100 | 100 | 100 | 7.77E−11 |
| 477 | 132; 222; 466; 1273; 1475; 1611; 2442; 2512; 2720; 3052; 3121; 3123; 3317; 3506; 3982; 4152; 4745; 5286 | 100 | 100 | 100 | 100 | 100 | 7.77E−11 |
| 478 | 497; 956; 4029 | 100 | 100 | 100 | 100 | 100 | 7.77E−11 |
| 479 | 216; 305; 559; 671; 1024; 1373; 1517; 2320; 2346; 2856; 2867; 2921; 2939; 2980; 3146; 3423; 3461; | 100 | 100 | 100 | 100 | 100 | 7.77E−11 |
| | 3808; 3861; 3883; 3891; 4186; 4263; 4561; 4772; 4894; 5249 | | | | | | |
| 480 | 825; 1317; 1938; 2320; 4300; 5268 | 100 | 100 | 100 | 100 | 100 | 7.77E−11 |
| 481 | 98; 835; 879; 1048; 1429; 1915; 1980; 3172; 4096; 4542; 4683; 4775; 4868 | 100 | 100 | 100 | 100 | 100 | 7.77E−11 |
| 482 | 146; 207; 246; 476; 572; 659; 749; 868; 892; 1162; 1600; 1655; 1799; 1956; 2274; 2351; 2431; 2615; | 100 | 100 | 100 | 100 | 100 | 7.77E−11 |
| | 2820; 2855; 2991; 3035; 3144; 3280; 3469; 3521; 3557; 3748; 3769; 3902; 4077; 4086; 4123; 4153; | | | | | | |
| | 4301; 4420; 4486; 4520; 4594; 4604; 4723; 4744; 4924; 5141; 5184; 5254; 5267 | | | | | | |
| 483 | 24; 152; 168; 214; 267; 484; 586; 743; 932; 1201; 1457; 1459; 1551; 1683; 1807; 2002; 2037; 2181; | 100 | 100 | 100 | 100 | 100 | 7.77E−11 |
| | 2182; 2264; 2887; 3014; 3200; 3327; 3396; 3535; 3558; 3743; 3962; 4017; 4043; 4147; 4266; 4467; | | | | | | |
| | 4470; 4488; 4508; 4510; 4835; 4838; 4983; 4995; 5021; 5078; 5170; 5214; 5215; 5262 | | | | | | |
| 484 | 50; 78; 110; 164; 320; 386; 495; 612; 788; 1144; 1283; 1365; 1401; 1496; 1677; 1709; 1740; 1937; 1980; | 100 | 100 | 100 | 100 | 100 | 7.77E−11 |
| | 2167; 2334; 2447; 2464; 2761; 2837; 2954; 2963; 3143; 3307; 3505; 3577; 3749; 3837; 3916; 4046; | | | | | | |
| | 4089; 4513; 4833; 4851; 4861; 5148; 5153; 5206 | | | | | | |
| 485 | 1663; 2656; 2889; 3173; 3252; 3538; 4324; 4797; 4918; 4994; 5208 | 100 | 100 | 100 | 100 | 100 | 7.77E−11 |
| 486 | 49; 218; 640; 734; 1646; 1755; 1802; 2307; 2835; 3180; 3578; 3960; 3991; 4086; 4202; 4240; 4584; 4948 | 100 | 100 | 100 | 100 | 100 | 7.77E−11 |
| 487 | 411; 454; 812; 1771; 2032; 2698; 3897; 4031; 5085 | 100 | 100 | 100 | 100 | 100 | 7.77E−11 |
| 488 | 8; 2669; 3172; 3414; 3505; 4194; 4632 | 100 | 100 | 100 | 100 | 100 | 7.77E−11 |
| 489 | 1297; 1901; 2049; 4117; 4909; 5101 | 100 | 100 | 100 | 100 | 100 | 7.77E−11 |
| 490 | 460; 630; 1022; 1447; 1803; 2089; 2948; 3082; 4468; 4630 | 100 | 100 | 100 | 100 | 100 | 7.77E−11 |
| 491 | 208; 958; 1057; 1468; 1550; 1703; 2073; 2388; 2561; 2959; 2986; 3087; 4670; 4686; 4950; 4985; 5086; 5206 | 100 | 100 | 100 | 100 | 100 | 7.77E−11 |
| 492 | 735; 4618; 4632 | 100 | 100 | 100 | 100 | 100 | 7.77E−11 |
| 493 | 2014; 2367; 2508; 3132; 3972 | 100 | 100 | 100 | 100 | 100 | 7.77E−11 |
| 494 | 44; 133; 587; 854; 999; 1175; 1226; 1473; 1771; 1859; 1917; 2183; 2212; 2335; 2397; 2432; 2665; | 100 | 100 | 100 | 100 | 100 | 7.77E−11 |
| | 2756; 3003; 3040; 3153; 3181; 3518; 3556; 3628; 3974; 4080; 4103; 4538; 4821; 4842; 5277 | | | | | | |
| 495 | 304; 430; 435; 590; 617; 681; 788; 930; 936; 940; 1103; 1169; 1327; 1343; 1375; 1509; 1518; 1792; | 100 | 100 | 100 | 100 | 100 | 7.77E−11 |
| | 2015; 2062; 2246; 2273; 2342; 2361; 2515; 2653; 2671; 2731; 2808; 2855; 3147; 3234; 3242; 3433; | | | | | | |
| | 3526; 3776; 4356; 4502; 4722; 4810; 4837; 4944; 5035; 5186; 5195; 5238; 5239; 5259 | | | | | | |
| 496 | 4952 | 100 | 100 | 100 | 100 | 100 | 7.77E−11 |
| 497 | 190; 498; 981; 1317; 1371; 2029; 2512; 2794; 2909; 3100; 3419; 3449; 3776; 3918; 4501; 4784; 4978 | 100 | 100 | 100 | 100 | 100 | 7.77E−11 |
| 498 | 115; 1155; 2277; 2346; 2471; 2542; 2649; 2659; 3991; 4178; 4179 | 100 | 100 | 100 | 100 | 100 | 7.77E−11 |
| 499 | 129; 327; 1764 | 100 | 100 | 100 | 100 | 100 | 7.77E−11 |
| 500 | 1376; 1448; 1719; 1748; 1985; 2558; 4693 | 100 | 100 | 100 | 100 | 100 | 7.77E−11 |
| 501 | 1380; 1658; 1702; 2397; 2980; 3270; 3329; 3798; 4600; 4712 | 100 | 100 | 100 | 100 | 100 | 7.77E−11 |
| 502 | 42; 478; 731; 765; 857; 983; 1977; 2487; 2870; 4144; 4800; 5270 | 100 | 100 | 100 | 100 | 100 | 7.77E−11 |
| 503 | 848; 1001; 1686; 2348; 3326; 4854; 5198 | 100 | 100 | 100 | 100 | 100 | 7.77E−11 |
| 504 | 80; 219; 2043; 2395; 3102; 3216; 3946; 4241 | 100 | 100 | 100 | 100 | 100 | 7.77E−11 |
| 505 | 1439; 2194; 2993; 3641; 4083; 4353 | 100 | 100 | 100 | 100 | 100 | 7.77E−11 |
| 506 | 246; 972; 1245; 2052; 2141; 2888; 2936; 3861; 4523; 4590; 5076 | 100 | 100 | 100 | 100 | 100 | 7.77E−11 |
| 507 | 646; 931; 1988; 3657; 3829; 3972 | 100 | 100 | 100 | 100 | 100 | 7.77E−11 |
| 508 | 362; 786; 980; 1378; 2023; 2026; 2373; 2648; 3457; 3535; 4679; 4898; 4899; 4954; 5039; 5048; 5082 | 100 | 100 | 100 | 100 | 100 | 7.77E−11 |
| 509 | 14; 334; 397; 957; 1226; 2125; 2920; 3209; 4545; 4561 | 100 | 100 | 100 | 100 | 100 | 7.77E−11 |
| 510 | 1870; 3401; 3823; 4781 | 100 | 100 | 100 | 100 | 100 | 7.77E−11 |
| 511 | 106; 427; 1309; 1401; 1935; 2338; 2370; 2657; 2741; 3071; 3232; 4360; 4712; 4994 | 100 | 100 | 100 | 100 | 100 | 7.77E−11 |
| 512 | 36; 215; 237; 494; 525; 635; 716; 753; 1062; 1152; 1290; 1459; 1717; 1745; 1802; 2209; 2215; 2511; | 100 | 100 | 100 | 100 | 100 | 7.77E−11 |
| | 2539; 2909; 2912; 3348; 3536; 3630; 3773; 3815; 4112; 4159; 4301; 4493; 4542; 4571; 4609; 4880; 4927; 5109 | | | | | | |
| 513 | 612; 899; 1095; 4282; 4755 | 100 | 100 | 100 | 100 | 100 | 7.77E−11 |
| 514 | 735; 1235; 1809; 1812; 2785; 3109; 3257; 3373; 3507; 3626; 3674; 4530; 4768 | 100 | 100 | 100 | 100 | 100 | 7.77E−11 |
| 515 | 34; 288; 416; 432; 726; 825; 905; 1194; 1215; 1566; 1777; 2096; 2403; 2454; 2831; 2878; 2918; 3108; | 100 | 100 | 100 | 100 | 100 | 7.77E−11 |
| | 3198; 3474; 3522; 3676; 4017; 4430; 4750; 4772; 4907 | | | | | | |
| 516 | 19; 219; 1181; 2250; 2455; 2682; 2691; 3985; 4873; 4927 | 100 | 100 | 100 | 100 | 100 | 7.77E−11 |
| 517 | 85; 336; 495; 581; 644; 663; 1056; 1345; 1350; 1382; 1460; 1576; 1633; 1748; 1894; 1980; 2024; 2267; | 100 | 100 | 100 | 100 | 100 | 7.77E−11 |
| | 2466; 2477; 2493; 2592; 2612; 2698; 2789; 3035; 3253; 3320; 3446; 3534; 3586; 3625; 3970; 4087; | | | | | | |
| | 4354; 4447; 4590; 4618; 4634; 4763; 4843; 4845; 5158; 5222 | | | | | | |
| 518 | 1202; 2795; 2922; 3835; 3983; 4489 | 100 | 100 | 100 | 100 | 100 | 7.77E−11 |
| 519 | 212; 556; 833; 871; 1013; 1024; 1224; 1465; 1869; 2070; 2146; 2451; 3034; 3325; 3490; 3570; 3621; | 100 | 100 | 100 | 100 | 100 | 7.77E−11 |
| | 3704; 3788; 3888; 4242; 4948; 5101; 5205 | | | | | | |
| 520 | 1276; 1314; 2146; 3034; 4900 | 100 | 100 | 100 | 100 | 100 | 7.77E−11 |
| 521 | 209; 392; 475; 551; 938; 1176; 1469; 1624; 1953; 2220; 2762; 2900; 3139; 3238; 3477; 4157; 4256; 4421; 4468; 4517; 4954 | 100 | 100 | 100 | 100 | 100 | 7.77E−11 |
| 522 | 546; 865; 2290; 2702; 2878; 3334; 3554; 4467 | 100 | 100 | 100 | 100 | 100 | 7.77E−11 |
| 523 | 249; 1517; 1655; 2326; 2846; 3064; 3247; 3655; 5082; 5253 | 100 | 100 | 100 | 100 | 100 | 7.77E−11 |
| 524 | 387; 2026; 2210; 2257; 2404; 4684 | 100 | 100 | 100 | 100 | 100 | 7.77E−11 |
| 525 | 195; 1061; 1111; 1217; 1850; 2400; 3200; 3209; 3216; 3690; 4012; 4233; 4276; 4646; 4735; 4890; 5153; 5230 | 100 | 100 | 100 | 100 | 100 | 7.77E−11 |
| 526 | 529; 1292; 1577; 2305; 2487; 3044; 4844; 5253 | 100 | 100 | 100 | 100 | 100 | 7.77E−11 |
| 527 | 465; 619; 2083; 2969; 3319; 3895; 3949; 4388; 4496 | 100 | 100 | 100 | 100 | 100 | 7.77E−11 |
| 528 | 944; 3748 | 100 | 100 | 100 | 100 | 100 | 7.77E−11 |
| 529 | 152; 409; 803; 1173; 1669; 1731; 1875; 1947; 1952; 2448; 3131; 3260; 3546; 3709; 4332; 4841; 4951 | 100 | 100 | 100 | 100 | 100 | 7.77E−11 |
| 530 | 311; 519; 546; 1446; 2873; 3034; 3184; 3299; 3346; 3547; 3679; 4134; 4143; 4305; 5097 | 100 | 100 | 100 | 100 | 100 | 7.77E−11 |
| 531 | 235; 526; 1375; 1435; 1676; 3633; 3822; 4149; 5060 | 100 | 100 | 100 | 100 | 100 | 7.77E−11 |
| 532 | 912; 2338; 3310; 4691 | 100 | 100 | 100 | 100 | 100 | 7.77E−11 |
| 533 | 748; 1001; 1224; 2024; 2234; 4018; 4868; 4992; 5202 | 100 | 100 | 100 | 100 | 100 | 7.77E−11 |
| 534 | 784; 1033; 3807; 4589 | 100 | 100 | 100 | 100 | 100 | 7.77E−11 |
| 535 | 385; 985; 1250; 1830; 1904; 2664; 4048; 4230; 4606; 4629; 4643; 4901; 4908; 4927; 4945; 5153; 5222 | 100 | 100 | 100 | 100 | 100 | 7.77E−11 |
| 536 | 219; 495; 685; 892; 1183; 2128; 2274; 2846; 2987; 3193; 3333; 3454; 4180; 5018 | 100 | 100 | 100 | 100 | 100 | 7.77E−11 |
| 537 | 20; 300; 507; 556; 712; 794; 1092; 1214; 1384; 1401; 1403; 1606; 1615; 1730; 1751; 1786; 2004; 2224; | 100 | 100 | 100 | 100 | 100 | 7.77E−11 |
| | 2234; 2357; 3463; 3545; 3757; 3819; 3983; 4695; 4703; 4820; 4955; 4960; 5057; 5202 | | | | | | |
| 538 | 391; 704; 1405; 1727; 3024; 3403; 4098; 4160; 5265 | 100 | 100 | 100 | 100 | 100 | 7.77E−11 |
| 539 | 417; 546; 1080; 1505; 2716; 3992; 5125; 5137 | 100 | 100 | 100 | 100 | 100 | 7.77E−11 |
| 540 | 160; 961; 1043; 1134; 1352; 1979; 2166; 2208; 3747; 3976; 4166; 4478; 4683; 5098; 5141 | 100 | 100 | 100 | 100 | 100 | 7.77E−11 |
| 541 | 137; 173; 240; 353; 470; 641; 893; 924; 961; 1049; 1058; 1072; 1100; 1169; 1170; 1220; 1297; 1419; | 100 | 100 | 100 | 100 | 100 | 7.77E−11 |
| | 1552; 1561; 1642; 2005; 2132; 2140; 2172; 2237; 2248; 2299; 2506; 2597; 2749; 2982; 3035; 3147; | | | | | | |
| | 3200; 3260; 3570; 3722; 3785; 3796; 3832; 4109; 4157; 4331; 4416; 4472; 4530; 4647; 4844; 5099 | | | | | | |
| 542 | 149; 1004; 1031; 1087; 1680; 2088; 3007; 4511; 4976 | 100 | 100 | 100 | 100 | 100 | 7.77E−11 |
| 543 | 260; 267; 495; 569; 657; 830; 1639; 2012; 2084; 2135; 3064; 3077; 3871; 3940; 4058; 4318; 4470; 4474; 4873; 5025 | 100 | 100 | 100 | 100 | 100 | 7.77E−11 |
| 544 | 2515; 2875; 4350; 5081; 5215 | 100 | 100 | 100 | 100 | 100 | 7.77E−11 |
| 545 | 913; 2675; 2821; 5132 | 100 | 100 | 100 | 100 | 100 | 7.77E−11 |
| 546 | 53; 106; 198; 300; 301; 804; 1248; 1552; 1821; 1872; 1985; 2447; 2656; 2676; 2838; 3136; 3137; 3471; | 100 | 100 | 100 | 100 | 100 | 7.77E−11 |
| | 3505; 3558; 3897; 4008; 4520; 4640; 4674; 4988; 5007; 5089 | | | | | | |
| 547 | 318; 515; 871; 906; 911; 989; 1099; 1206; 1416; 1487; 1668; 1752; 2145; 2163; 2178; 2241; 2462; 2639; | 100 | 100 | 100 | 100 | 100 | 7.77E−11 |
| | 2682; 2686; 2720; 3093; 3098; 3224; 3343; 3379; 3626; 3751; 4011; 4175; 4246; 4309; 4348; 4565; 4839; 5170; 5230; 5266 | | | | | | |
| 548 | 1910; 2048; 2671; 2922; 2936; 3620; 4021 | 100 | 100 | 100 | 100 | 100 | 7.77E−11 |
| 549 | 1570; 2931; 3160; 3684; 4196; 4563; 4772; 4977 | 100 | 100 | 100 | 100 | 100 | 7.77E−11 |
| 550 | 50; 91; 116; 363; 414; 489; 613; 615; 630; 712; 750; 806; 991; 1117; 1203; 1378; 1404; 1630; 1686; | 100 | 100 | 100 | 100 | 100 | 7.77E−11 |
| | 1727; 1897; 1909; 2024; 2167; 2203; 2647; 2648; 2682; 2837; 3200; 3300; 3360; 3433; 3453; 3459; | | | | | | |
| | 3463; 3525; 3579; 3582; 3767; 3975; 4108; 4532; 4539; 4698; 4933; 5001; 5058; 5220 | | | | | | |
| 551 | 1027; 1080; 1114; 1143; 1169; 1391; 1400; 1477; 1581; 1925; 3434; 3694; 4049; 4277; 4334; 4471; 4528; 5277 | 100 | 100 | 100 | 100 | 100 | 7.77E−11 |
| 552 | 130; 553; 1004; 1488; 1947; 2204; 2300; 2527; 3586; 3589; 3705; 4000; 4055; 4618; 4717; 4732; 4745; 5087; 5093; 5146 | 100 | 100 | 100 | 100 | 100 | 7.77E−11 |
| 553 | 43; 310; 347; 634; 834; 1513; 1589; 1648; 2113; 2184; 2185; 2197; 2201; 2246; 2319; 2760; 2817; | 100 | 100 | 100 | 100 | 100 | 7.77E−11 |
| | 3195; 3317; 3431; 3549; 4098; 4133; 4205; 4233; 4241; 4260; 4313; 4587; 4831; 4905; 4909; 4941; 5176; 5257 | | | | | | |
| 554 | 130; 849; 1280; 1576; 1793; 2037; 2291; 2313; 2432; 2459; 2670; 2798; 3373; 3975; 4072; 4086; 4148; 4307; 4441; 5213 | 100 | 100 | 100 | 100 | 100 | 7.77E−11 |
| 555 | 183; 219; 528; 672; 1336; 1463; 2249; 2826; 2849; 3022; 3589; 3740 | 100 | 100 | 100 | 100 | 100 | 7.77E−11 |
| 556 | 378; 1014; 1938; 2843; 4256; 4717; 4952 | 100 | 100 | 100 | 100 | 100 | 7.77E−11 |
| 557 | 249; 379; 768; 933; 1029; 1207; 1250; 1325; 1570; 1615; 1626; 1739; 1740; 1830; 1860; 1926; 2348; 2533; 2666; | 100 | 100 | 100 | 100 | 100 | 7.77E−11 |
| | 2685; 3026; 3171; 3224; 3279; 3308; 3313; 3405; 3496; 3536; 3721; 3879; 4146; 4257; 4280; 4379; 4762; 4830; 5088; 5126; 5243 | | | | | | |
| 558 | 379; 563; 884; 1303; 1944; 2795; 3822; 3922; 4017; 4163 | 100 | 100 | 100 | 100 | 100 | 7.77E−11 |
| 559 | 309; 1500; 1669; 1724; 2363; 2738; 3836; 4381; 4634; 5190 | 100 | 100 | 100 | 100 | 100 | 7.77E−11 |
| 560 | 207; 2564; 3763 | 100 | 100 | 100 | 100 | 100 | 7.77E−11 |
| 561 | 37; 634; 703; 707; 728; 772; 928; 1110; 1126; 1217; 1449; 1864; 2206; 3828; 3876 | 100 | 100 | 100 | 100 | 100 | 7.77E−11 |
| 562 | 177; 709; 981; 1387; 1606; 2263; 2334; 2382; 2447; 2822; 2973; 3077; 3326; 3692; 4197; 4220; 4661; 4899 | 100 | 100 | 100 | 100 | 100 | 7.77E−11 |
| 563 | 349; 376; 413; 582; 925; 1366; 1372; 1795; 2886; 3083; 3138; 3357; 3813; 4006; 4675 | 100 | 100 | 100 | 100 | 100 | 7.77E−11 |
| 564 | 252; 438; 3286; 4278; 4353; 4473 | 100 | 100 | 100 | 100 | 100 | 7.77E−11 |
| 565 | 96; 99; 323; 378; 458; 729; 775; 862; 882; 1096; 1232; 1263; 1606; 1632; 1901; 1906; 2170; 2261; 2300; | 100 | 100 | 100 | 100 | 100 | 7.77E−11 |
| | 3260; 3276; 3429; 3460; 3517; 3546; 3752; 3878; 3881; 3906; 4081; 4220; 5004; 5028; 5096 | | | | | | |
| 566 | 435; 719; 769; 782; 823; 1753; 2845; 2975; 3482; 4123; 4160; 4332; 4574; 5011 | 100 | 100 | 100 | 100 | 100 | 7.77E−11 |
| 567 | 163; 187; 227; 346; 579; 781; 1132; 1721; 2001; 2009; 2025; 2191; 2266; 2270; 2443; 2665; 2697; | 100 | 100 | 100 | 100 | 100 | 7.77E−11 |
| | 2699; 2730; 2768; 2916; 2951; 2989; 3011; 3097; 3209; 3216; 3439; 3662; 3729; 3743; 3784; 3836; | | | | | | |
| | 3956; 4090; 4097; 4100; 4104; 4128; 4467; 4713; 4766; 4789; 4841; 4939; 4956 | | | | | | |
| 568 | 41; 185; 1251; 2842; 3312; 3669; 3678; 4377; 4684; 4959 | 100 | 100 | 100 | 100 | 100 | 7.77E−11 |
| 569 | 636; 933; 1638; 2220; 2242; 2730; 2906; 4069; 4440 | 100 | 100 | 100 | 100 | 100 | 7.77E−11 |
| 570 | 103; 121; 160; 177; 218; 229; 258; 265; 281; 696; 728; 733; 855; 939; 1098; 1408; 1938; 2275; 2360; | 100 | 100 | 100 | 100 | 100 | 7.77E−11 |
| | 2490; 2524; 2596; 2718; 2764; 2973; 2988; 3092; 3209; 3212; 3216; 3217; 3342; 3364; 3431; 3453; | | | | | | |
| | 3665; 3683; 3701; 3957; 4097; 4244; 4297; 4322; 4334; 4699; 4795; 4833; 4869; 4888; 5232 | | | | | | |
| 571 | 69; 191; 365; 578; 591; 660; 698; 780; 808; 819; 835; 1247; 1251; 1255; 1553; 1589; 1661; 2037; 2050; | 100 | 100 | 100 | 100 | 100 | 7.77E−11 |
| | 2289; 2532; 2587; 2593; 2612; 2668; 2760; 3388; 3436; 3582; 4127; 4507; 4544; 4759; 4963; 5097; 5251 | | | | | | |
| 572 | 207; 478; 1321; 1405; 1461; 2201; 2455; 2471; 3196; 4476; 4755 | 100 | 100 | 100 | 100 | 100 | 7.77E−11 |
| 573 | 27; 102; 218; 408; 419; 636; 879; 1112; 1766; 1777; 1998; 3286; 3335; 3403; 3405; 3433; 3586; 3701; 3931; 4271; 4435; 5018; 5154 | 100 | 100 | 100 | 100 | 100 | 7.77E−11 |
| 574 | 168; 660; 1155; 1186; 1232; 1273; 1647; 1735; 1961; 2257; 3129; 3212; 3260; 3473; 3982; 4306; 4620; 4804; 4928; 5286 | 100 | 100 | 100 | 100 | 100 | 7.77E−11 |
| 575 | 109; 210; 274; 940; 2246; 2846; 3126; 4009; 4497; 4598; 5074 | 100 | 100 | 100 | 100 | 100 | 7.77E−11 |
| 576 | 86; 94; 114; 208; 242; 244; 252; 407; 598; 657; 1023; 1088; 1182; 1530; 1551; 1606; 1818; 1925; 2030; | 100 | 100 | 100 | 100 | 100 | 7.77E−11 |
| | 2032; 2075; 2128; 2221; 2336; 2423; 2485; 2529; 2602; 2958; 3175; 3204; 3378; 3387; 3798; 3864; | | | | | | |
| | 3990; 4235; 4257; 4324; 4589; 4633; 4645; 4816; 4942; 5045; 5161; 5279 | | | | | | |
| 577 | 505; 642; 818; 858; 924; 957; 1046; 1739; 2065; 2149; 2222; 2525; 2543; 2731; 3312; 3561; 3574; 4071; | 100 | 100 | 100 | 100 | 100 | 7.77E−11 |
| | 4095; 4105; 4115; 4366; 4405; 4743; 5094 | | | | | | |
| 578 | 233; 276; 348; 403; 444; 673; 740; 1146; 1437; 1474; 1513; 1780; 1799; 1874; 2124; 2135; 2172; 2179; | 100 | 100 | 100 | 100 | 100 | 7.77E−11 |
| | 2811; 3519; 3623; 3630; 3663; 3772; 3919; 3952; 4412; 4417; 4553; 4720; 4734; 4753; 4953; 5010; 5019 | | | | | | |
| 579 | 7; 14; 318; 347; 379; 830; 1056; 1176; 1250; 1332; 1810; 1888; 2106; 2160; 2443; 2460; 2560; 2842; | 100 | 100 | 100 | 100 | 100 | 7.77E−11 |
| | 2902; 2925; 2936; 3011; 3121; 3302; 3451; 3545; 3567; 3676; 3732; 3784; 3881; 3940; 4319; 4374; | | | | | | |
| | 4477; 4481; 4498; 4524; 4592; 4745; 4796; 4934; 4950; 5130; 5233; 5265 | | | | | | |
| 580 | 254; 755; 1521; 2890; 2916; 2937; 3070; 3205; 4545 | 100 | 100 | 100 | 100 | 100 | 7.77E−11 |
| 581 | 207; 1519; 1590; 1974; 2150; 2154; 2874; 4289; 4724; 4871 | 100 | 100 | 100 | 100 | 100 | 7.77E−11 |
| 582 | 330; 362; 442; 615; 689; 820; 1005; 1152; 1416; 1619; 1987; 2507; 2996; 3009; 3150; 3420; 3653; | 100 | 100 | 100 | 100 | 100 | 7.77E−11 |
| | 3688; 3810; 4100; 4662; 4833; 4956; 5036; 5102; 5227 | | | | | | |
| 583 | 147; 856; 962; 2885; 3375; 3419; 3516; 3677; 4659 | 100 | 100 | 100 | 100 | 100 | 7.77E−11 |
| 584 | 80; 242; 1302; 2381; 2642; 3202; 3403; 3649; 4492 | 100 | 100 | 100 | 100 | 100 | 7.77E−11 |
| 585 | 82; 219; 254; 977; 1192; 1331; 1558; 1914; 1918; 2851; 3343; 3649; 3822; 4786; 4859 | 100 | 100 | 100 | 100 | 100 | 7.77E−11 |
| 586 | 122; 203; 648; 1729; 2636; 3510; 3687; 3944; 4202; 4619; 4980 | 100 | 100 | 100 | 100 | 100 | 7.77E−11 |
| 587 | 19; 29; 181; 224; 394; 440; 477; 774; 779; 1078; 1142; 1152; 1438; 1519; 1559; 1717; 1858; 2022; | 100 | 100 | 100 | 100 | 100 | 7.77E−11 |
| | 2120; 2136; 2436; 2609; 2625; 2696; 2746; 2802; 2843; 2845; 2872; 2929; 2964; 3042; 3363; 3507; | | | | | | |
| | 3921; 3967; 4149; 4151; 4189; 4312; 4462; 4596; 4787; 4925; 5070; 5090; 5190; 5199; 5202 | | | | | | |
| 588 | 3195; 3524; 3566 | 100 | 100 | 100 | 100 | 100 | 7.77E−11 |
| 589 | 118; 140; 628; 731; 793; 1026; 1097; 1198; 1267; 1305; 1358; 1506; 1617; 1719; 1735; 2047; 2694; 2735; 2822; 2868; | 100 | 100 | 100 | 100 | 100 | 7.77E−11 |
| | 2963; 3137; 3156; 3343; 3402; 3501; 3670; 3698; 4035; 4042; 4114; 4299; 4325; 4345; 4693; 4711; 4746; 5214; 5238; 5258 | | | | | | |
| 590 | 265; 671; 1301; 2997; 4956 | 100 | 100 | 100 | 100 | 100 | 7.77E−11 |
| 591 | 151; 304; 312; 333; 791; 1057; 1209; 1316; 2008; 2677; 3149; 3198; 3239; 3311; 3497; 3571; 4412; 4657; 5003; 5145; 5270 | 100 | 100 | 100 | 100 | 100 | 7.77E−11 |
| 592 | 496; 757; 765; 799; 1365; 1862; 1928; 1948; 2084; 2239; 2722; 2764; 2788; 3222; 3448; 3805; 4050; | 100 | 100 | 100 | 100 | 100 | 7.77E−11 |
| | 4065; 4702; 4789; 4894; 5070; 5243 | | | | | | |
| 593 | 529; 812; 1572; 1989; 3011; 4635; 4787; 4943; 5004; 5141 | 100 | 100 | 100 | 100 | 100 | 7.77E−11 |
| 594 | 12; 23; 44; 220; 395; 530; 784; 914; 926; 933; 981; 1121; 1306; 1337; 1343; 1673; 1686; 1744; 1784; | 100 | 100 | 100 | 100 | 100 | 7.77E−11 |
| | 1793; 1807; 2190; 2218; 2262; 2335; 2375; 2658; 2739; 2860; 2950; 3236; 3432; 3596; 3637; 3655; | | | | | | |
| | 3716; 3936; 4314; 4510; 4512; 4515; 4621; 5004; 5220 | | | | | | |
| 595 | 391; 459; 795; 971; 1111; 1443; 2175; 4698; 4719 | 100 | 100 | 100 | 100 | 100 | 7.77E−11 |
| 596 | 424; 727; 2427; 3304; 3990; 4028; 5010; 5284 | 100 | 100 | 100 | 100 | 100 | 7.77E−11 |
| 597 | 1366; 1631; 1657; 1661; 2005; 2043; 2783; 4410; 4691; 4714 | 100 | 100 | 100 | 100 | 100 | 7.77E−11 |
| 598 | 8; 775; 937; 1450; 2019; 2086; 2327 | 100 | 100 | 100 | 100 | 100 | 7.77E−11 |
| 599 | 404; 1149; 2670; 2909; 3552; 3561; 4300; 4633 | 100 | 100 | 100 | 100 | 100 | 7.77E−11 |
| 600 | 5; 48; 81; 301; 330; 378; 775; 1070; 1800; 1935; 2278; 2403; 2944; 4283; 4478; 4759; 4878 | 100 | 100 | 100 | 100 | 100 | 7.77E−11 |
| 601 | 323; 958; 991; 1058; 2478; 2642 | 100 | 100 | 100 | 100 | 100 | 7.77E−11 |
| 602 | 100; 207; 352; 464; 522; 715; 736; 1024; 1100; 1148; 1178; 1256; 1616; 1683; 1826; 1914; 2130; 2201; | 100 | 100 | 100 | 100 | 100 | 7.77E−11 |
| | 2221; 2465; 2471; 2657; 2908; 3518; 3542; 3562; 3957; 4189; 4325; 4358; 4546; 5004; 5038; 5111; 5209; 5273 | | | | | | |
| 603 | 203; 210; 213; 311; 657; 816; 882; 1477; 1510; 2335; 2390; 2646; 3265; 3575; 3682; 4140; 4163; | 100 | 100 | 100 | 100 | 100 | 7.77E−11 |
| | 4223; 4349; 4745; 4984; 5039; 5051; 5072 | | | | | | |
| 604 | 1556; 1863; 2081; 2559; 2617; 2690; 3134; 3580; 3718; 4713 | 100 | 100 | 100 | 100 | 100 | 7.77E−11 |
| 605 | 146; 397; 1060; 1110; 2333; 2530; 2715; 3171; 3454; 3585; 3818; 3861; 4803; 4908; 5278 | 100 | 100 | 100 | 100 | 100 | 7.77E−11 |
| 606 | 163; 298; 495; 1038; 1060; 1519; 1552; 1562; 1671; 1849; 2003; 2732; 2961; 3571; 3584; 3995; 4229; 4269; 4383; 4502; 4735 | 100 | 100 | 100 | 100 | 100 | 7.77E−11 |
| 607 | 39; 299; 683; 1168; 1888; 2124; 2218; 2485; 2674; 2675; 2937; 3020; 3181; 3715; 3835; 3894; 4489; 4498; 5055 | 100 | 100 | 100 | 100 | 100 | 7.77E−11 |
| 608 | 369; 1955; 2357; 3339; 4557; 4745; 4890; 4916 | 100 | 100 | 100 | 100 | 100 | 7.77E−11 |
| 609 | 403; 452; 783; 1643; 1645; 1707; 1916; 2084; 2209; 2248; 2310; 2842; 2868; 2927; 3155; 3311; 3347; | 100 | 100 | 100 | 100 | 100 | 7.77E−11 |
| | 3571; 3672; 3746; 3776; 3800; 3885; 3887; 4129; 4243; 4318; 5039; 5092; 5200; 5275 | | | | | | |
| 610 | 314; 813; 872; 940; 1278; 1409; 1585; 1643; 1916; 1952; 2180; 2227; 2397; 2657; 2688; 2712; 2737; | 100 | 100 | 100 | 100 | 100 | 7.77E−11 |
| | 2813; 2857; 2878; 2896; 2936; 2944; 3015; 3162; 3214; 3242; 3277; 3478; 3586; 3911; 3932; 3998; | | | | | | |
| | 4132; 4172; 4229; 4339; 4387; 4474; 4524; 4693; 4711; 4996; 5000; 5046; 5190; 5193 | | | | | | |
| 611 | 44; 72; 132; 169; 230; 366; 424; 562; 1473; 1476; 1600; 2200; 2331; 2481; 2484; 2648; 2658; 2817; 2865; 2963; 3069; | 100 | 100 | 100 | 100 | 100 | 7.77E−11 |
| | 3209; 3279; 3403; 3429; 3454; 3742; 4010; 4082; 4194; 4378; 4440; 4453; 4624; 4626; 4838; 4923; 4944; 5088; 5097; 5106; 5222 | | | | | | |
| 612 | 405; 2289; 3589 | 100 | 100 | 100 | 100 | 100 | 7.77E−11 |
| 613 | 501; 785; 986; 1171; 1710; 2307; 3388; 4392 | 100 | 100 | 100 | 100 | 100 | 7.77E−11 |
| 614 | 530; 922; 975; 1419; 1640; 3359; 4221; 4888 | 100 | 100 | 100 | 100 | 100 | 7.77E−11 |
| 615 | 78; 1678; 3529; 3841; 4874 | 100 | 100 | 100 | 100 | 100 | 7.77E−11 |
| 616 | 323; 1448; 2689; 2721; 2837; 3778; 4232 | 100 | 100 | 100 | 100 | 100 | 7.77E−11 |
| 617 | 125; 171; 194; 317; 1441; 1720; 1887; 2301; 2306; 2383; 2431; 2509; 3185; 3240; 3265; 3412; 3690; 3888; 4247; 4325; 4933; 4951; 5078 | 100 | 100 | 100 | 100 | 100 | 7.77E−11 |
| 618 | 207; 232; 373; 532; 835; 909; 1392; 2084; 2338; 2785; 2988; 3339; 3361; 3612; 3840; 4892; 4975; 5107; 5239 | 100 | 100 | 100 | 100 | 100 | 7.77E−11 |
| 619 | 769; 903; 1102; 1910; 2750; 4267; 4334; 4550; 5148 | 100 | 100 | 100 | 100 | 100 | 7.77E−11 |
| 620 | 193; 743; 789; 1173; 1475; 1666; 1817; 2348; 2533; 2534; 2553; 2677; 3345; 3623; 3653; 3952; 4017; 4193; 4516; 4685; 4788; 4826; | 100 | 100 | 100 | 100 | 100 | 7.77E−11 |
| | 4845; 4861 | | | | | | |
| 621 | 185; 345; 520; 810; 1180; 1442; 1689; 1855; 1865; 1904; 1984; 2303; 2690; 3249; 3313; 3388; 3536; 3832; 3862; 3890; 4255; 4922; 5033 | 100 | 100 | 100 | 100 | 100 | 7.77E−11 |
| 622 | 879; 2083; 2565; 3347; 4288 | 100 | 100 | 100 | 100 | 100 | 7.77E−11 |
| 623 | 707; 1172; 1571; 2007; 2329; 2437; 2837; 3368; 4641; 4889 | 100 | 100 | 100 | 100 | 100 | 7.77E−11 |
| 624 | 1558; 1697; 1882; 3139; 4024; 4181 | 100 | 100 | 100 | 100 | 100 | 7.77E−11 |
| 625 | 183; 280; 348; 432; 651; 774; 791; 1075; 1093; 1165; 1323; 1350; 1376; 1508; 1752; 1795; 2240; 2322; | 100 | 100 | 100 | 100 | 100 | 7.77E−11 |
| | 2493; 2500; 2620; 2665; 2701; 2721; 2830; 3023; 3313; 3344; 3780; 3814; 3850; 3881; 3939; 4126; | | | | | | |
| | 4281; 4418; 4444; 4492; 4542; 4751; 4908; 4922; 5001; 5004; 5077; 5081; 5242; 5268 | | | | | | |
| 626 | 159; 320; 543; 656; 742; 883; 1207; 1510; 1726; 1799; 1939; 1994; 2136; 2598; 2727; 2752; 2978; | 100 | 100 | 100 | 100 | 100 | 7.77E−11 |
| | 3483; 3827; 4163; 4166; 4796; 5067; 5115; 5266 | | | | | | |
| 627 | 992; 1295; 1957; 2217; 2907; 4091 | 100 | 100 | 100 | 100 | 100 | 7.77E−11 |
| 628 | 44; 198; 584; 982; 1096; 1312; 1684; 1883; 1967; 1982; 2078; 2175; 2284; 2291; 2616; 2930; 3026; 3050; 3134; | 100 | 100 | 100 | 100 | 100 | 7.77E−11 |
| | 3147; 3159; 3206; 3463; 3615; 3621; 3654; 3688; 3753; 3794; 3891; 4160; 4493; 4574; 4630; 4795; 4948; 5069; 5107; 5208 | | | | | | |
| 629 | 171; 319; 1046; 2036; 3253; 3362; 4102; 5208 | 100 | 100 | 100 | 100 | 100 | 7.77E−11 |
| 630 | 493; 574; 788; 1337; 1458; 1594; 2033; 3079; 3299; 3969; 4149; 4268; 4505; 4658; 4702; 5080; 5205 | 100 | 100 | 100 | 100 | 100 | 7.77E−11 |
| 631 | 106; 2469; 2658; 4572; 4700; 5210 | 100 | 100 | 100 | 100 | 100 | 7.77E−11 |
| 632 | 146; 221; 293; 420; 581; 656; 803; 844; 880; 991; 1368; 1762; 1973; 2104; 2220; 2298; 2301; 2322; 2324; 2492; 2493; 2580; | 100 | 100 | 100 | 100 | 100 | 7.77E−11 |
| | 2602; 2666; 2690; 2853; 3065; 3075; 3183; 3211; 3360; 3386; 3430; 3582; 3656; 3815; 3849; 4082; 4084; 4852; 4902; 5101 | | | | | | |
| 633 | 13; 153; 355; 480; 615; 650; 946; 1176; 1264; 1341; 1477; 1559; 1668; 1803; 1902; 1933; 2098; 2148; | 100 | 100 | 100 | 100 | 100 | 7.77E−11 |
| | 2278; 2446; 2716; 2723; 2744; 2891; 2909; 2942; 2980; 3003; 3019; 3122; 3570; 3609; 3688; 3695; | | | | | | |
| | 3737; 3843; 3885; 3903; 3939; 3986; 4236; 4276; 4540; 4951; 4953; 5001; 5160; 5210 | | | | | | |
| 634 | 331; 550; 1321; 2064; 2831; 3897; 4731; 5106 | 100 | 100 | 100 | 100 | 100 | 7.77E−11 |
| 635 | 780; 935; 1662; 1742; 1938; 2061; 4095; 4128; 4700 | 100 | 100 | 100 | 100 | 100 | 7.77E−11 |
| 636 | 947; 2194; 2465; 4151; 4735; 5131; 5270 | 100 | 100 | 100 | 100 | 100 | 7.77E−11 |
| 637 | 208; 266; 480; 502; 515; 545; 742; 754; 756; 850; 1138; 1178; 1289; 1375; 1381; 1384; 1714; 1788; 2062; | 100 | 100 | 100 | 100 | 100 | 7.77E−11 |
| | 2098; 2099; 2202; 2623; 2648; 2694; 2984; 3355; 3396; 3913; 4072; 4097; 4147; 4210; 4243; 4477; 4549; 4846; 5076; 5093 | | | | | | |
| 638 | 132; 216; 318; 356; 548; 577; 670; 821; 854; 1004; 1061; 1393; 1539; 1629; 1712; 1791; 1971; 2144; 2237; 2253; | 100 | 100 | 100 | 100 | 100 | 7.77E−11 |
| | 2526; 2705; 2758; 2774; 2969; 3257; 3775; 4048; 4393; 4478; 4488; 4497; 4507; 4637; 4652; 4684; 4891; 5172; 5203; 5267 | | | | | | |
| 639 | 105; 267; 379; 1192; 1497; 1655; 1670; 2305; 2347; 2411; 2480; 2482; 2689; 2730; 3034; 3093; 3202; | 100 | 100 | 100 | 100 | 100 | 7.77E−11 |
| | 3312; 3443; 3673; 3945; 4006; 4043; 4106; 4765; 4775; 4849; 4951; 5100; 5156; 5157 | | | | | | |
| 640 | 360; 396; 403; 457; 465; 1121; 2140; 2761; 3171; 3195; 4107; 4529; 4722 | 100 | 100 | 100 | 100 | 100 | 7.77E−11 |
| 641 | 87; 201; 234; 424; 511; 597; 661; 936; 1027; 1421; 1558; 1820; 1861; 2057; 2607; 2837; 3135; 3156; 3277; 3865; 4547; 4637; 4657; 5186 | 100 | 100 | 100 | 100 | 100 | 7.77E−11 |
| 642 | 213; 379; 444; 583; 625; 838; 1024; 1113; 1161; 1339; 1430; 1769; 2150; 2312; 2357; 2767; 3173; 3490; 3868; 5028; 5115 | 100 | 100 | 100 | 100 | 100 | 7.77E−11 |
| 643 | 280; 526; 616; 697; 731; 824; 986; 1001; 1029; 1071; 1162; 1242; 1371; 1375; 1490; 1588; 1616; 1618; | 100 | 100 | 100 | 100 | 100 | 7.77E−11 |
| | 1638; 1704; 1827; 1905; 2031; 2152; 2212; 2726; 2893; 3167; 3210; 3234; 3365; 3388; 3394; 3895; | | | | | | |
| | 3963; 3978; 3985; 4096; 4230; 4319; 4429; 4457; 4610; 4735; 4846; 5044 | | | | | | |
| 644 | 330; 383; 889; 920; 948; 1785; 1794; 1911; 2329; 2592; 3163; 3420; 3530; 3566; 4489; 4970 | 100 | 100 | 100 | 100 | 100 | 7.77E−11 |
| 645 | 291; 716; 1583; 2011; 2532; 3566; 4250; 4656; 5135 | 100 | 100 | 100 | 100 | 100 | 7.77E−11 |
| 646 | 1329; 2237; 2646; 3864; 4096; 4263 | 100 | 100 | 100 | 100 | 100 | 7.77E−11 |
| 647 | 803; 1129; 1595; 2716; 2783; 3104; 4138; 4214; 4253; 4840 | 100 | 100 | 100 | 100 | 100 | 7.77E−11 |
| 648 | 1342; 3045; 4098; 4336 | 100 | 100 | 100 | 100 | 100 | 7.77E−11 |
| 649 | 526; 601; 609; 734; 791; 1016; 1150; 1155; 1212; 1232; 1422; 1490; 1815; 1982; 2164; 2336; 2361; 2376; 2499; | 100 | 100 | 100 | 100 | 100 | 7.77E−11 |
| | 2571; 2677; 2753; 2882; 2931; 3275; 3276; 3346; 3564; 3607; 4020; 4162; 4250; 4303; 4391; 4718; 4907; 5225 | | | | | | |
| 650 | 133; 1585; 2005; 2213; 2281; 2596 | 100 | 100 | 100 | 100 | 100 | 7.77E−11 |
| 651 | 430; 782; 934; 1388; 1766; 1860; 2458; 4087; 4695 | 100 | 100 | 100 | 100 | 100 | 7.77E−11 |
| 652 | 143; 149; 159; 376; 455; 503; 641; 889; 913; 991; 1207; 1338; 1766; 1991; 2175; 2186; 2321; 2359; | 100 | 100 | 100 | 100 | 100 | 7.77E−11 |
| | 2366; 2435; 2576; 2577; 2646; 2734; 2860; 2996; 3435; 3471; 3756; 3796; 4077; 4297; 4431; 4822; 4983; 4987; 5179 | | | | | | |
| 653 | 51; 240; 263; 342; 379; 532; 607; 825; 833; 1306; 1491; 1676; 1824; 1910; 1998; 2709; 2714; 2883; | 100 | 100 | 100 | 100 | 100 | 7.77E−11 |
| | 3330; 3674; 3821; 3943; 4108; 4332; 4564; 4944; 4981; 5014 | | | | | | |
| 654 | 342; 859; 1169; 1268; 1624; 2424; 2454; 2618; 3131; 3593; 3608; 3723; 3815; 3820; 3860; 3907; 3913; | 100 | 100 | 100 | 100 | 100 | 7.77E−11 |
| | 3979; 4148; 4153; 4242; 4365; 4753; 4944; 5026 | | | | | | |
| 655 | 44; 294; 385; 1121; 1236; 1519; 1929; 1978; 2080; 2247; 2270; 2343; 2720; 3874; 3892; 3923; 4013; 4084; 4148; 4448; 4907 | 100 | 100 | 100 | 100 | 100 | 7.77E−11 |
| 656 | 75; 478; 496; 529; 1811; 1859; 3378; 3432; 3734; 3748; 3963; 4125; 4319; 4576; 4707; 4729; 4843; 4880; 4928; 5068; 5107 | 100 | 100 | 100 | 100 | 100 | 7.77E−11 |
| 657 | 638; 789; 809; 1016; 1293; 1517; 2079; 2817; 2862; 3034; 3378; 3701; 3764; 4030; 4281; 4323; 4578; 4975; 5054; 5264 | 100 | 100 | 100 | 100 | 100 | 7.77E−11 |
| 658 | 58; 171; 684; 931; 1400; 1792; 2220; 2261; 2380; 2665; 2813; 2873; 2883; 2892; 3059; 3102; 3403; | 100 | 100 | 100 | 100 | 100 | 7.77E−11 |
| | 4018; 4314; 4406; 4486; 4511; 4543; 4655; 4916; 5041; 5092 | | | | | | |
| 659 | 219; 1126; 1393; 2129; 2436; 2607; 2724; 2822; 4612; 4908 | 100 | 100 | 100 | 100 | 100 | 7.77E−11 |
| 660 | 283; 838; 1129; 1260; 1605; 1765; 2122; 2137; 2670; 2781; 2928; 3033; 3745; 3795; 3867; 3999; 4181; 4294; 4645 | 100 | 100 | 100 | 100 | 100 | 7.77E−11 |
| 661 | 30; 68; 164; 1509; 1517; 1533; 1736; 1935; 2140; 2381; 2890; 3266; 3333; 3390; 3676; 3716; 4102; 4880; 5124 | 100 | 100 | 100 | 100 | 100 | 7.77E−11 |
| 662 | 742; 999; 1765; 3308; 3361; 3530; 4144; 5062 | 100 | 100 | 100 | 100 | 100 | 7.77E−11 |
| 663 | 37; 255; 757; 954; 1107; 1124; 1152; 1688; 2052; 2137; 3229; 3586; 4395; 4914 | 100 | 100 | 100 | 100 | 100 | 7.77E−11 |
| 664 | 536; 704; 939; 1057; 1649; 3430; 3911; 4630; 4804; 5069 | 100 | 100 | 100 | 100 | 100 | 7.77E−11 |
| 665 | 966; 1658; 1691; 1783; 2049; 2336; 2865; 3190; 3975; 4177; 4591; 5118 | 100 | 100 | 100 | 100 | 100 | 7.77E−11 |
| 666 | 498; 938; 1261; 1629; 2763; 3209; 4091; 4378; 4843 | 100 | 100 | 100 | 100 | 100 | 7.77E−11 |
| 667 | 988; 1195; 1568; 1861; 2230; 2433; 3377; 3473; 3747; 3890; 4238; 4283; 4367; 4792; 4843 | 100 | 100 | 100 | 100 | 100 | 7.77E−11 |
| 668 | 407; 948; 1663; 1812; 3725; 4825 | 100 | 100 | 100 | 100 | 100 | 7.77E−11 |
| 669 | 169; 556; 780; 1236; 1295; 1331; 1361; 1630; 4682; 4962 | 100 | 100 | 100 | 100 | 100 | 7.77E−11 |
| 670 | 583; 943; 1841; 2049; 2252; 2280; 2358; 2475; 2488; 3606; 4633; 4861; 4973 | 100 | 100 | 100 | 100 | 100 | 7.77E−11 |
| 671 | 377; 407; 549; 833; 887; 909; 917; 949; 1280; 1378; 1411; 1419; 1590; 1837; 1862; 2210; 2607; 2658; | 100 | 100 | 100 | 100 | 100 | 7.77E−11 |
| | 2759; 2868; 3286; 3338; 3556; 3568; 3588; 3995; 4106; 4132; 4198; 4305; 4409; 4493; 4583; 4637; 4908; 4988; 5062; 5220 | | | | | | |
| 672 | 626; 651; 832; 904; 1095; 1140; 1206; 2399; 2477; 2567; 2605; 2674; 2759; 3477; 3488; 3955; 4006; 4149; 4321; 4520; 5073 | 100 | 100 | 100 | 100 | 100 | 7.77E−11 |
| 673 | 346; 967; 1344; 2082; 2837; 3122; 3558 | 100 | 100 | 100 | 100 | 100 | 7.77E−11 |
| 674 | 218; 528; 1136; 3317; 4861 | 100 | 100 | 100 | 100 | 100 | 7.77E−11 |
| 675 | 8; 192; 219; 818; 848; 1034; 1402; 1484; 1587; 1730; 2573; 2776; 3692; 3703; 3763; 4147; 4215; 4898; 4956 | 100 | 100 | 100 | 100 | 100 | 7.77E−11 |
| 676 | 418; 537; 1814; 1932; 2174; 2267; 3514; 3586; 3876; 4068; 4099; 4166; 4225; 4579; 4688 | 100 | 100 | 100 | 100 | 100 | 7.77E−11 |
| 677 | 519; 883; 998; 2197; 3912 | 100 | 100 | 100 | 100 | 100 | 7.77E−11 |
| 678 | 44; 554; 855; 1328; 1453; 2255; 2564; 2580; 2673; 2784; 2829; 2864 | 100 | 100 | 100 | 100 | 100 | 7.77E−11 |
| 679 | 202; 432; 434; 530; 838; 878; 1301; 1321; 1375; 1394; 1470; 1506; 1660; 1800; 1909; 1982; 2080; | 100 | 100 | 100 | 100 | 100 | 7.77E−11 |
| | 2154; 2431; 2442; 2450; 2459; 2577; 2632; 2814; 3396; 3440; 3520; 3740; 3872; 3996; 4211; 4605; 4835; 5132; 5139; 5256 | | | | | | |
| 680 | 8; 153; 377; 525; 655; 680; 705; 726; 786; 946; 1029; 1159; 1173; 1195; 1380; 1520; 1572; 1640; 1666; | 100 | 100 | 100 | 100 | 100 | 7.77E−11 |
| | 1833; 1873; 2312; 2327; 2524; 2607; 2620; 2677; 2702; 2706; 2914; 3120; 3279; 3477; 3511; 3542; | | | | | | |
| | 3577; 3908; 4099; 4121; 4249; 4265; 4269; 4453; 4603; 4720 | | | | | | |
| 681 | 329; 604; 755; 811; 1064; 1764; 1767; 1967; 2043; 2045; 2458; 2831; 2842; 3136; 3586; 3973; 4356; 4460; 4498; 5041; 5221; 5273 | 100 | 100 | 100 | 100 | 100 | 7.77E−11 |
| 682 | 916; 999; 1419; 1929; 2876; 4405 | 100 | 100 | 100 | 100 | 100 | 7.77E−11 |
| 683 | 787; 1391; 2276; 2403; 2505; 2831; 3313; 3988; 4018 | 100 | 100 | 100 | 100 | 100 | 7.77E−11 |
| 684 | 115; 527; 2136; 2989; 5252 | 100 | 100 | 100 | 100 | 100 | 7.77E−11 |
| 685 | 311; 515; 622; 1091; 1405; 2186; 2281; 2389; 2458; 2537; 2685; 2829; 2954; 2992; 3426; 3432; 3467; | 100 | 100 | 100 | 100 | 100 | 7.77E−11 |
| | 3815; 4156; 4246; 4735; 4853; 5089; 5101 | | | | | | |
| 686 | 178; 292; 314; 324; 396; 481; 531; 558; 715; 719; 741; 995; 1088; 1092; 1145; 1152; 1284; 1293; 1405; 1432; 1701; 1725; 1908; 2013; | 100 | 100 | 100 | 100 | 100 | 7.77E−11 |
| | 2014; 2042; 2231; 2893; 2894; 3027; 3257; 3300; 3420; 3475; 3512; 3638; 3670; 3906; 4132; 4175; 4278; 4457; 4537; 4656; 4775; 4919; 5122 | | | | | | |
| 687 | 451; 759; 1711; 1901; 1976; 2728; 2890; 4233; 4735; 5082 | 100 | 100 | 100 | 100 | 100 | 7.77E−11 |
| 688 | 824; 993; 1373; 1573; 1802; 2423; 2649; 2729; 2911; 3081; 3784; 3876; 4064; 4376; 5215 | 100 | 100 | 100 | 100 | 100 | 7.77E−11 |
| 689 | 365; 520; 3011; 3245; 3590; 3592; 4581 | 100 | 100 | 100 | 100 | 100 | 7.77E−11 |
| 690 | 127; 195; 224; 327; 365; 504; 681; 789; 793; 798; 992; 1027; 1059; 1335; 1614; 1626; 1646; 1655; 1754; | 100 | 100 | 100 | 100 | 100 | 7.77E−11 |
| | 1795; 1810; 1896; 1940; 2333; 2382; 2448; 2525; 2581; 2600; 2611; 2620; 2801; 2969; 3032; 3176; | | | | | | |
| | 3382; 3832; 3898; 4006; 4231; 4526; 4563; 4654; 4814; 5075 | | | | | | |
| 691 | 95; 336; 726; 1381; 1435; 1609; 1765; 1939; 2727; 2929; 2977; 3166; 3439; 3481; 3569; 3974; 4437; 4633 | 100 | 100 | 100 | 100 | 100 | 7.77E−11 |
| 692 | 385; 404; 778; 1408; 1456; 1944; 2396; 2657; 3489; 3599; 3776; 3988; 4154; 4362; 4365; 4397; 4406; 4915 | 100 | 100 | 100 | 100 | 100 | 7.77E−11 |
| 693 | 229; 243; 494; 1029; 1176; 1354; 1462; 1519; 1532; 1537; 1659; 1694; 2126; 2200; 2213; 2311; 2413; | 100 | 100 | 100 | 100 | 100 | 7.77E−11 |
| | 2531; 2640; 3217; 3257; 3284; 3327; 3414; 3644; 3747; 3977; 4176; 4218; 4226; 4345; 4370; 4441; | | | | | | |
| | 4447; 4469; 4590; 4750; 4977; 5012; 5014; 5060; 5065; 5088 | | | | | | |
| 694 | 222; 716; 772; 940; 1146; 1270; 1476; 2432; 2861; 3381; 3586; 3733; 3736; 3874; 4021; 4080; 4738; 4770; 4822; 4928 | 100 | 100 | 100 | 100 | 100 | 7.77E−11 |
| 695 | 1561; 2645; 3813; 4018; 4172 | 100 | 100 | 100 | 100 | 100 | 7.77E−11 |
| 696 | 47; 137; 1099; 2493; 2690; 2814; 2970; 4165; 4304; 5221 | 100 | 100 | 100 | 100 | 100 | 7.77E−11 |
| 697 | 451; 548; 1702; 2255; 2534; 4289 | 100 | 100 | 100 | 100 | 100 | 7.77E−11 |
| 698 | 800; 1049; 1539; 2046; 2842; 4766 | 100 | 100 | 100 | 100 | 100 | 7.77E−11 |
| 699 | 42; 168; 218; 796; 853; 1291; 1332; 1904; 2318; 2557; 2902; 2916; 3421; 3452; 3715; 4055; 4153; 4734; 4917; 5213 | 100 | 100 | 100 | 100 | 100 | 7.77E−11 |
| 700 | 219; 259; 1066; 1117; 2477; 2688 | 100 | 100 | 100 | 100 | 100 | 7.77E−11 |
| 701 | 57; 879; 2348; 3380; 3989; 4639 | 100 | 100 | 100 | 100 | 100 | 7.77E−11 |
| 702 | 215; 316; 596; 985; 1275; 1651; 2164; 2544; 2642; 2994; 3191; 3266; 3701; 4389; 4438; 4663; 4845; 5070; 5203 | 100 | 100 | 100 | 100 | 100 | 7.77E−11 |
| 703 | 213; 785; 1070; 1630; 2587; 2956; 3040; 4074; 4230; 4292; 4376 | 100 | 100 | 100 | 100 | 100 | 7.77E−11 |
| 704 | 199; 295; 300; 322; 374; 848; 1118; 1771; 3839; 4022; 5095 | 100 | 100 | 100 | 100 | 100 | 7.77E−11 |
| 705 | 25; 2701; 2805; 3864; 4377; 5239 | 100 | 100 | 100 | 100 | 100 | 7.77E−11 |
| 706 | 219; 2195; 3051; 4162; 4205; 5268 | 100 | 100 | 100 | 100 | 100 | 7.77E−11 |
| 707 | 220; 304; 692; 1222; 1481; 2308; 2479; 2525; 2635; 2727; 2957; 3148; 3336; 3383; 3517; 3893; 5243 | 100 | 100 | 100 | 100 | 100 | 7.77E−11 |
| 708 | 133; 240; 611; 1097; 1425; 2244; 2296; 2406; 2427; 2530; 2557; 2662; 3720; 3751; 4143; 4346; 4434; 4436; 4497; 4579; 4592; 4828 | 100 | 100 | 100 | 100 | 100 | 7.77E−11 |
| 709 | 57; 147; 182; 219; 385; 649; 709; 965; 1037; 1742; 1804; 1901; 2210; 2249; 2465; 2686; 3386; 3534; | 100 | 100 | 100 | 100 | 100 | 7.77E−11 |
| | 3572; 3608; 3824; 3919; 4136; 4231; 4361; 4411; 4442; 4559; 4591; 4849; 4866; 5069; 5125; 5200; 5274 | | | | | | |
| 710 | 379; 1496; 1676; 1740; 1800; 1969; 2008; 2281; 2385; 2482; 2648; 2939; 3310; 3337; 3911; 4008; 4233; 4353; 4538; 4579; 4676; 5236 | 100 | 100 | 100 | 100 | 100 | 7.77E−11 |
| 711 | 477; 657; 843; 1149; 1323; 1416; 1967; 2119; 2474; 2768; 2871; 3192; 3209; 3438; 3504; 3580; 4013; 4867 | 100 | 100 | 100 | 100 | 100 | 7.77E−11 |
| 712 | 752; 1350; 2340; 2670; 3757; 4604; 4913 | 100 | 100 | 100 | 100 | 100 | 7.77E−11 |
| 713 | 66; 230; 549; 1095; 2152; 2257; 3076; 3101; 3508; 3586; 4233; 5070; 5227 | 100 | 100 | 100 | 100 | 100 | 7.77E−11 |
| 714 | 111; 186; 193; 421; 575; 636; 706; 832; 1412; 1902; 2861; 2943; 3139; 4300; 4386; 4639; 5062; 5088 | 100 | 100 | 100 | 100 | 100 | 7.77E−11 |
| 715 | 122; 272; 727; 1580; 1766; 2151; 2442; 2475; 2645; 2760; 3231; 3507; 3698; 4683; 4733 | 100 | 100 | 100 | 100 | 100 | 7.77E−11 |
| 716 | 207; 621; 961; 2137; 3367; 3576; 4702 | 100 | 100 | 100 | 100 | 100 | 7.77E−11 |
| 717 | 252; 401; 1121; 1164; 1226; 1411; 1424; 1445; 1651; 2442; 2760; 2934; 3046; 3232; 3316; 3404; 3536; | 100 | 100 | 100 | 100 | 100 | 7.77E−11 |
| | 3581; 3731; 3750; 3838; 4107; 4315; 4625; 4663; 4750; 4820; 5239 | | | | | | |
| 718 | 201; 461; 589; 871; 873; 1090; 1266; 1323; 1401; 1758; 2035; 2691; 2806; 2973; 3288; 3483; 3562; | 100 | 100 | 100 | 100 | 100 | 7.77E−11 |
| | 3602; 3888; 4022; 4154; 4249; 4362; 4522; 4549; 4633; 4650; 4694; 4900; 4971; 5117; 5161; 5205; 5271 | | | | | | |
| 719 | 625; 679; 783; 1126; 2269; 2813; 3993; 4493; 4669; 4698 | 100 | 100 | 100 | 100 | 100 | 7.77E−11 |
| 720 | 36; 202; 217; 286; 869; 1180; 2039; 2110; 2417; 2472; 2488; 2519; 3631; 3747; 4954; 5268 | 100 | 100 | 100 | 100 | 100 | 7.77E−11 |
| 721 | 353; 2899; 3521; 4201; 4281; 4356; 4907; 4912; 4944 | 100 | 100 | 100 | 100 | 100 | 7.77E−11 |
| 722 | 92; 106; 165; 355; 541; 763; 796; 883; 954; 964; 1059; 1283; 1486; 1512; 1555; 1561; 1588; 1811; 1859; | 100 | 100 | 100 | 100 | 100 | 7.77E−11 |
| | 2017; 2257; 2266; 2552; 2583; 2696; 2829; 3004; 3185; 3244; 3558; 3586; 3755; 3866; 4022; 4394; | | | | | | |
| | 4497; 4711; 4724; 4769; 4856; 4874; 4939; 5156; 5205; 5263 | | | | | | |
| 723 | 1733; 2845 | 100 | 100 | 100 | 100 | 100 | 7.77E−11 |
| 724 | 66; 85; 128; 558; 657; 686; 690; 692; 727; 823; 999; 1049; 1277; 1379; 1386; 1568; 1667; 1744; 1876; | 100 | 100 | 100 | 100 | 100 | 7.77E−11 |
| | 1936; 1943; 1983; 2154; 2356; 2378; 2643; 2655; 2681; 2858; 2901; 3068; 3332; 3420; 3481; 3612; | | | | | | |
| | 3763; 4072; 4198; 4208; 4303; 4371; 4509; 4543; 4786; 4846; 4888; 4895; 4930; 4949; 5112 | | | | | | |
| 725 | 173; 470; 577; 761; 915; 1152; 1160; 1584; 1677; 1795; 1824; 2225; 2555; 2660; 2806; 2836; 2847; 2856; 2881; | 100 | 100 | 100 | 100 | 100 | 7.77E−11 |
| | 2976; 3057; 3240; 3244; 3586; 3613; 3662; 3904; 4417; 4492; 4552; 4665; 4741; 4743; 4914; 4929; 5031; 5039; 5060; 5076 | | | | | | |
| 726 | 90; 110; 378; 413; 415; 464; 524; 538; 615; 636; 669; 790; 1010; 1232; 1307; 1549; 1611; 1865; 1869; | 100 | 100 | 100 | 100 | 100 | 7.77E−11 |
| | 2163; 2265; 2374; 2584; 2656; 3025; 3421; 3588; 3672; 3729; 3927; 3940; 4031; 4050; 4360; 4403; 4868; 5055; 5260 | | | | | | |
| 727 | 42; 119; 206; 272; 274; 596; 616; 702; 812; 889; 922; 933; 1110; 1808; 1838; 1874; 1907; 1912; 1919; | 100 | 100 | 100 | 100 | 100 | 7.77E−11 |
| | 2027; 2051; 2096; 2138; 2464; 2877; 2988; 3014; 3161; 3231; 3244; 3331; 3473; 3544; 3586; 3712; | | | | | | |
| | 3719; 3761; 3776; 4158; 4427; 4492; 4521; 4630; 4693; 4866; 4932; 4949; 4950; 5075 | | | | | | |
| 728 | 296; 1276; 1606; 1782; 2455; 3087; 3986; 4217; 4473 | 100 | 100 | 100 | 100 | 100 | 7.77E−11 |
| 729 | 146; 850; 966; 1908; 2642; 3138; 3663; 3733; 3800; 3971; 4530; 4972; 5240 | 100 | 100 | 100 | 100 | 100 | 7.77E−11 |
| 730 | 220; 1294; 2819; 3698 | 100 | 100 | 100 | 100 | 100 | 7.77E−11 |
| 731 | 62; 207; 1100; 1571; 2134; 2154; 3255 | 100 | 100 | 100 | 100 | 100 | 7.77E−11 |
| 732 | 53; 152; 898; 1654; 2699; 3000; 3576; 3628; 3795; 5208 | 100 | 100 | 100 | 100 | 100 | 7.77E−11 |
| 733 | 168; 261; 757; 788; 918; 1272; 1300; 1565; 1824; 1970; 2229; 2351; 2388; 2391; 2458; 2829; 3222; | 100 | 100 | 100 | 100 | 100 | 7.77E−11 |
| | 3428; 3452; 4399; 4631; 4852; 4873; 5242; 5257 | | | | | | |
| 734 | 31; 967; 1573; 1816; 2360; 3592; 5133 | 100 | 100 | 100 | 100 | 100 | 7.77E−11 |
| 735 | 64; 299; 326; 391; 545; 612; 719; 1110; 1115; 1229; 1270; 1323; 1478; 1505; 1638; 1964; 2075; 2130; | 100 | 100 | 100 | 100 | 100 | 7.77E−11 |
| | 2496; 2622; 2687; 2701; 2702; 2931; 3038; 3304; 3754; 3768; 3878; 4031; 4186; 4241; 4362; 4555; 4771; 4787; 5007 | | | | | | |
| 736 | 127; 206; 1784; 4593; 4888 | 100 | 100 | 100 | 100 | 100 | 7.77E−11 |
| 737 | 771; 2039; 2241; 2900; 3152; 3214; 4952 | 100 | 100 | 100 | 100 | 100 | 7.77E−11 |
| 738 | 485; 1276; 2689; 2761; 3636; 5007; 5141 | 100 | 100 | 100 | 100 | 100 | 7.77E−11 |
| 739 | 1226; 2572; 2757; 3182; 4186; 4359 | 100 | 100 | 100 | 100 | 100 | 7.77E−11 |
| 740 | 314; 366; 542; 1012; 1439; 1800; 1888; 2171; 2436; 2516; 2599; 2673; 2722; 2920; 3182; 3876; 3955; 4511; 4821 | 100 | 100 | 100 | 100 | 100 | 7.77E−11 |
| 741 | 407; 459; 529; 684; 1472; 1575; 3035; 3165; 4309; 4570; 5000; 5151 | 100 | 100 | 100 | 100 | 100 | 7.77E−11 |
| 742 | 43; 218; 372; 739; 935; 1030; 1035; 1273; 1317; 1607; 1731; 2096; 2476; 2529; 2702; 2780; 2867; 2937; | 100 | 100 | 100 | 100 | 100 | 7.77E−11 |
| | 2945; 3096; 3209; 3536; 3548; 3567; 3580; 3597; 3795; 4145; 4332; 4377; 4618; 5002; 5013; 5276 | | | | | | |
| 743 | 426; 432; 475; 493; 532; 547; 607; 864; 984; 1171; 1229; 1619; 1726; 1776; 1825; 1837; 1877; 1948; 2043; 2410; 2584; | 100 | 100 | 100 | 100 | 100 | 7.77E−11 |
| | 2651; 2685; 2707; 2941; 3017; 3572; 3784; 3802; 3869; 4003; 4026; 4084; 4120; 4207; 4367; 4384; 4390; 4498; 4811; 4814; 4872 | | | | | | |
| 744 | 1551; 1826; 2331; 2528; 3009; 4766 | 100 | 100 | 100 | 100 | 100 | 7.77E−11 |
| 745 | 378; 757; 830; 921; 973; 1447; 1803; 2078; 2199; 2304; 2675; 2686; 2897; 3586; 3711; 3998; 4102; 4685; 4941; 5018; 5020; 5268 | 100 | 100 | 100 | 100 | 100 | 7.77E−11 |
| 746 | 58; 222; 424; 458; 595; 788; 1019; 1060; 1696; 1723; 1788; 1848; 1901; 2039; 2055; 2081; 2435; 2739; | 100 | 100 | 100 | 100 | 100 | 7.77E−11 |
| | 3115; 3910; 3995; 4060; 4535; 4543; 4630; 4831; 4922; 4940; 4960; 5262; 5280 | | | | | | |
| 747 | 152; 175; 208; 243; 426; 1343; 1855; 2088; 2122; 2231; 2465; 2657; 2706; 2821; 2842; 2989; 3208; 3380; 3512; 3621; 4115; 4177; 4208 | 100 | 100 | 100 | 100 | 100 | 7.77E−11 |
| 748 | 154; 545; 1401; 1609; 1842; 1843; 1875; 1883; 1935; 2009; 2333; 2412; 2835; 2904; 3107; 3515; | 100 | 100 | 100 | 100 | 100 | 7.77E−11 |
| | 3519; 3563; 3949; 4252; 4381; 4441; 4523; 4557; 4583; 4785; 4903; 4943; 4984; 5182; 5221 | | | | | | |
| 749 | 46; 1171; 3252; 3261; 3866; 4578; 4970 | 100 | 100 | 100 | 100 | 100 | 7.77E−11 |
| 750 | 86; 248; 1190; 1786; 2157; 2534; 2846; 3170; 3324; 3903; 3993; 4065; 4169; 4492; 4520; 4603; 4772; 4779; 4967; 5075; 5153; 5205 | 100 | 100 | 100 | 100 | 100 | 7.77E−11 |
| 751 | 295; 617; 1046; 1047; 1138; 1257; 1440; 1954; 2267; 2335; 2608; 2753; 3443; 3602; 4605; 4868 | 100 | 100 | 100 | 100 | 100 | 7.77E−11 |
| 752 | 37; 181; 287; 298; 391; 400; 401; 411; 885; 913; 965; 1203; 1398; 1497; 1720; 1766; 1884; 1933; 2375; | 100 | 100 | 100 | 100 | 100 | 7.77E−11 |
| | 2410; 2510; 2648; 2689; 2703; 2871; 2872; 2891; 3014; 3154; 3266; 3344; 3384; 3417; 3603; 3625; | | | | | | |
| | 3663; 3742; 4013; 4043; 4151; 4464; 4542; 4792; 4804; 4935; 4955; 5147; 5202 | | | | | | |
| 753 | 79; 784; 1699; 3132; 3523; 3808; 4815 | 100 | 100 | 100 | 100 | 100 | 7.77E−11 |
| 754 | 348; 423; 916; 1090; 1268; 1564; 1681; 1777; 1808; 1837; 2042; 2651; 2807; 3001; 3412; 3603; 3769; | 100 | 100 | 100 | 100 | 100 | 7.77E−11 |
| | 3826; 4059; 4085; 4111; 4182; 4368; 4402; 4493; 5217 | | | | | | |
| 755 | 783; 984; 1640; 2241; 2649; 2784 | 100 | 100 | 100 | 100 | 100 | 7.77E−11 |
| 756 | 81; 576; 718; 772; 1005; 1401; 2494; 2807; 2958; 3404; 3526; 4075; 4101; 4964; 5027; 5102 | 100 | 100 | 100 | 100 | 100 | 7.77E−11 |
| 757 | 255; 442; 521; 525; 771; 876; 1016; 1814; 2431; 2465; 2821; 2867; 3178; 3209; 3338; 3408; 3528; | 100 | 100 | 100 | 100 | 100 | 7.77E−11 |
| | 3615; 3674; 3758; 4008; 4046; 4341; 4618; 4720; 4861; 4922; 5154; 5198 | | | | | | |
| 758 | 1739; 2080; 2369; 2376; 3305; 3314; 3579; 4276; 5069 | 100 | 100 | 100 | 100 | 100 | 7.77E−11 |
| 759 | 133; 935; 1493; 2358; 2767; 3495; 4158; 4735; 5224 | 100 | 100 | 100 | 100 | 100 | 7.77E−11 |
| 760 | 2027; 2838; 3747; 4121; 4643; 5030 | 100 | 100 | 100 | 100 | 100 | 7.77E−11 |
| 761 | 88; 120; 179; 461; 578; 680; 874; 1127; 1192; 1290; 1380; 1466; 1617; 1631; 1678; 1696; 1705; 1744; | 100 | 100 | 100 | 100 | 100 | 7.77E−11 |
| | 1800; 2116; 2363; 2666; 2684; 2732; 2828; 2982; 3083; 3286; 3493; 3602; 3721; 3796; 3861; 3923; | | | | | | |
| | 3998; 4348; 4389; 4408; 4585; 4711; 4792; 4855; 5156 | | | | | | |
| 762 | 1113; 2402; 2732; 3264 | 100 | 100 | 100 | 100 | 100 | 7.77E−11 |
| 763 | 520; 706; 750; 841; 933; 1330; 1700; 1897; 1982; 2042; 2450; 2492; 2521; 2676; 2703; 3178; 3326; 3578; 3623; 3877; 3919; 4178; 4788 | 100 | 100 | 100 | 100 | 100 | 7.77E−11 |
| 764 | 180; 358; 559; 835; 1678; 1765; 1864; 2053; 2738; 3937; 3971; 5020 | 100 | 100 | 100 | 100 | 100 | 7.77E−11 |
| 765 | 157; 517; 646; 1381; 1592; 1800; 1980; 2167; 2202; 2231; 2290; 2361; 2447; 2760; 2808; 2995; 3061; 4317; 4453; 4694; 4705; 5118; 5121 | 100 | 100 | 100 | 100 | 100 | 7.77E−11 |
| 766 | 230; 372; 721; 725; 763; 1305; 1482; 1504; 1755; 1929; 1949; 2238; 2331; 2427; 2545; 2727; 2790; | 100 | 100 | 100 | 100 | 100 | 7.77E−11 |
| | 2889; 3090; 3101; 3248; 3528; 3586; 3722; 3756; 4118; 4656; 4767; 4793; 4897; 4997; 5018 | | | | | | |
| 767 | 393; 1800; 1825; 2581; 2731; 2792; 3733; 4420; 4429; 4640; 4793 | 100 | 100 | 100 | 100 | 100 | 7.77E−11 |
| 768 | 244; 613; 784; 926; 1051; 1648; 2484; 2885; 3170; 4679 | 100 | 100 | 100 | 100 | 100 | 7.77E−11 |
| 769 | 104; 147; 229; 354; 628; 973; 1293; 1551; 1658; 1899; 1942; 2266; 2301; 2426; 2515; 2582; 2728; 2761; | 100 | 100 | 100 | 100 | 100 | 7.77E−11 |
| | 2959; 2976; 3137; 3586; 3993; 4095; 4263; 4332; 4346; 4369; 4549; 4638; 4794; 5009; 5088; 5138; 5149 | | | | | | |
| 770 | 2675; 3008; 3075; 3602; 3760; 4515; 5043; 5193 | 100 | 100 | 100 | 100 | 100 | 7.77E−11 |
| 771 | 97; 166; 497; 606; 612; 735; 750; 778; 1125; 1680; 1702; 1962; 2045; 2114; 2247; 2839; 2946; 3041; | 100 | 100 | 100 | 100 | 100 | 7.77E−11 |
| | 3829; 3865; 3905; 4211; 4430; 4434; 4637; 4952 | | | | | | |
| 772 | 377; 2450; 2815; 3024; 3476; 4126; 4191; 4508; 4654; 4771; 4820; 4951; 4982 | 100 | 100 | 100 | 100 | 100 | 7.77E−11 |
| 773 | 275; 432; 1279; 1377; 1583; 3317; 4298; 4980; 5265 | 100 | 100 | 100 | 100 | 100 | 7.77E−11 |
| 774 | 209; 227; 416; 445; 619; 1799; 2073; 2290; 2701; 2835; 3391; 3862; 3929; 4195; 5000; 5025 | 100 | 100 | 100 | 100 | 100 | 7.77E−11 |
| 775 | 529; 598; 609; 1891; 2052; 2573; 2762; 3166; 3285; 3312; 3558; 3665; 4133; 4630; 4731; 4811; 4848; 5022; 5031; 5261 | 100 | 100 | 100 | 100 | 100 | 7.77E−11 |
| 776 | 4; 201; 602; 805; 889; 960; 1201; 1490; 1762; 2458; 2619; 2791; 2869; 2898; 3425; 3546; 3760; 3845; | 100 | 100 | 100 | 100 | 100 | 7.77E−11 |
| | 3958; 3976; 4339; 4918; 4948; 4995; 5260 | | | | | | |
| 777 | 219; 699; 778; 900; 987; 990; 1073; 1367; 1405; 1501; 1518; 1624; 1712; 1984; 2247; 2474; 2553; | 100 | 100 | 100 | 100 | 100 | 7.77E−11 |
| | 2617; 2785; 3365; 3372; 3431; 3977; 4126; 4149; 4151; 4303; 4356; 4375; 4394; 4633; 4961 | | | | | | |
| 778 | 0; 64; 691; 1138; 1198; 1916; 2146; 2260; 2715; 3411; 3462; 3956; 4191; 4299; 4346; 4476; 4665 | 100 | 100 | 100 | 100 | 100 | 7.77E−11 |
| 779 | 133; 384; 1417; 4012; 4099; 4835; 4909 | 100 | 100 | 100 | 100 | 100 | 7.77E−11 |
| 780 | 1669; 3209; 5076; 5241 | 100 | 100 | 100 | 100 | 100 | 7.77E−11 |
| 781 | 548; 597; 696; 770; 904; 2033; 2129; 3384; 3444; 4121; 4641; 5142; 5199 | 100 | 100 | 100 | 100 | 100 | 7.77E−11 |
| 782 | 1140; 1288 | 100 | 100 | 100 | 100 | 100 | 7.77E−11 |
| 783 | 91; 957; 1031; 1978 | 100 | 100 | 100 | 100 | 100 | 7.77E−11 |
| 784 | 152; 257; 611; 831; 860; 889; 899; 942; 1346; 1365; 1555; 1944; 2034; 2051; 2313; 2340; 2388; 2600; | 100 | 100 | 100 | 100 | 100 | 7.77E−11 |
| | 2676; 2704; 2944; 3153; 3268; 3388; 3405; 3573; 3825; 3846; 4238; 4474; 4484; 4720; 4750; 4863; 5089; 5092; 5116; 5210 | | | | | | |
| 785 | 4759; 5181 | 100 | 100 | 100 | 100 | 100 | 7.77E−11 |
| 786 | 104; 252; 420; 441; 613; 951; 1252; 1297; 1321; 1528; 1547; 1563; 1580; 1787; 1864; 1927; 2062; 2233; | 100 | 100 | 100 | 100 | 100 | 7.77E−11 |
| | 2299; 2420; 2583; 2677; 2678; 2727; 2810; 2845; 2864; 2960; 3226; 3229; 3318; 3330; 3386; | | | | | | |
| | 3477; 3544; 3548; 3775; 3873; 3895; 3994; 4164; 4202; 4672; 5077; 5169 | | | | | | |
| 787 | 17; 485; 676; 830; 899; 987; 1191; 1595; 1754; 1787; 1805; 1830; 1937; 2184; 2324; 2329; 2351; 2416; | 100 | 100 | 100 | 100 | 100 | 7.77E−11 |
| | 2480; 2482; 2697; 2996; 3105; 3511; 3557; 3704; 3788; 3794; 4143; 4152; 4218; 4589; 4620; 4651; 4676; 4734; 4774 | | | | | | |
| 788 | 348; 421; 567; 774; 820; 965; 1382; 1393; 1394; 1712; 1919; 2053; 2375; 2401; 2576; 2707; 3261; | 100 | 100 | 100 | 100 | 100 | 7.77E−11 |
| | 3438; 3471; 3589; 3647; 3668; 3830; 3908; 3966; 4237; 4517; 4530; 4765; 4895; 5115; 5198 | | | | | | |
| 789 | 401; 890; 2137; 2570; 3505; 4149; 4292 | 100 | 100 | 100 | 100 | 100 | 7.77E−11 |
| 790 | 253; 266; 449; 517; 548; 821; 1166; 1255; 1403; 1859; 1978; 2029; 2046; 2407; 3052; 3080; 3250; | 100 | 100 | 100 | 100 | 100 | 7.77E−11 |
| | 3337; 3856; 3865; 3940; 4209; 4459; 4574; 4903; 5026; 5060; 5225; 5235 | | | | | | |
| 791 | 215; 749; 905; 967; 1152; 1182; 1443; 1556; 1896; 1931; 2054; 2140; 2289; 2354; 2394; 2876; 3033; | 100 | 100 | 100 | 100 | 100 | 7.77E−11 |
| | 3258; 3338; 3358; 3424; 3582; 3611; 3722; 4615; 4645; 4918; 5217; 5221 | | | | | | |
| 792 | 215; 1422; 2660; 3247; 3331; 3362; 3371; 3734; 3897; 4009 | 100 | 100 | 100 | 100 | 100 | 7.77E−11 |
| 793 | 219; 883; 1637; 2085; 2128; 2691; 3801; 4028; 4234; 4974 | 100 | 100 | 100 | 100 | 100 | 7.77E−11 |
| 794 | 883; 1147; 1755; 2257; 2409; 2413; 2516; 2627; 2962; 3145; 3211; 3628; 4049; 4172; 4257; 4833; 4914; 5205 | 100 | 100 | 100 | 100 | 100 | 7.77E−11 |
| 795 | 229; 367; 514; 550; 601; 630; 759; 821; 893; 925; 1124; 1206; 1278; 1365; 1505; 1576; 2738; 2845; | 100 | 100 | 100 | 100 | 100 | 7.77E−11 |
| | 2932; 3083; 3089; 3136; 3172; 3183; 3243; 3367; 3873; 4082; 4249; 4275; 4354; 4362; 4433; 4455; 4548 | | | | | | |
| 796 | 192; 257; 611; 822; 1092; 1123; 1408; 1609; 1731; 1845; 2048; 2067; 2083; 2219; 2348; 2591; 2612; | 100 | 100 | 100 | 100 | 100 | 7.77E−11 |
| | 3193; 3209; 3321; 3531; 3624; 3715; 3815; 3866; 3941; 3958; 3962; 4118; 4150; 4179; 4189; 4390; | | | | | | |
| | 4453; 4496; 4635; 4773; 4872; 4967; 5016; 5057; 5070; 5239 | | | | | | |
| 797 | 21; 75; 287; 610; 789; 809; 869; 933; 944; 1093; 1264; 1583; 1805; 1836; 2216; 2405; 2460; 2467; 2477; 2686; 2938; 3052; | 100 | 100 | 100 | 100 | 100 | 7.77E−11 |
| | 3315; 3325; 3334; 3488; 3526; 3551; 3720; 3927; 3955; 4125; 4141; 4150; 4382; 4402; 4457; 4638; 4782; 4798; 5108 | | | | | | |
| 798 | 49; 146; 292; 334; 348; 373; 392; 777; 835; 960; 1332; 1620; 1676; 1679; 1761; 1762; 1769; 1940; | 100 | 100 | 100 | 100 | 100 | 7.77E−11 |
| | 1984; 2314; 2357; 2372; 2382; 2498; 2636; 2691; 2820; 2896; 3145; 3468; 3611; 3616; 3856; 3865; | | | | | | |
| | 3920; 4159; 4228; 4289; 4330; 4331; 4379; 4436; 4604; 4643; 4678; 4692; 4727; 4760; 4992; 5073 | | | | | | |
| 799 | 379; 522; 920; 1543; 3068; 4196; 4833; 5001; 5018 | 100 | 100 | 100 | 100 | 100 | 7.77E−11 |
| 800 | 197; 348; 500; 914; 1074; 1115; 1192; 1387; 1573; 1897; 2308; 2405; 2488; 2623; 2724; 3190; 3622; 4089; 5014 | 100 | 100 | 100 | 100 | 100 | 7.77E−11 |
| 801 | 139; 803; 1104; 1317; 1786; 2342; 2755; 2885; 2916; 3539; 4321; 5027 | 100 | 100 | 100 | 100 | 100 | 7.77E−11 |
| 802 | 226; 1716; 1820; 2963; 3691; 3877; 4216; 4237; 4837; 5071; 5196 | 100 | 100 | 100 | 100 | 100 | 7.77E−11 |
| 803 | 58; 81; 88; 207; 425; 456; 809; 867; 1067; 1232; 1277; 1287; 1328; 2238; 2348; 2481; 2509; 2624; | 100 | 100 | 100 | 100 | 100 | 7.77E−11 |
| | 2641; 2733; 2764; 3006; 3066; 3251; 3347; 3487; 3619; 3861; 4063; 4109; 4300; 4319; 4425; 4498; 4946; 4962; 5125 | | | | | | |
| 804 | 858; 1424; 1509; 1553; 1709; 2426; 2481; 2626; 2677; 3648; 3701; 3910; 3993; 4269; 4562; 4672; 4976 | 100 | 100 | 100 | 100 | 100 | 7.77E−11 |
| 805 | 387; 546; 655; 812; 857; 881; 1232; 1472; 1831; 2017; 2227; 2474; 2690; 3702; 3948; 4088; 4317; 4672; 4817; 4906 | 100 | 100 | 100 | 100 | 100 | 7.77E−11 |
| 806 | 19; 1329; 2138; 2236; 3315; 3930; 4151; 4179; 5054 | 100 | 100 | 100 | 100 | 100 | 7.77E−11 |
| 807 | 284; 593; 620; 1146; 1316; 2116; 2855; 3090; 3495; 3584; 3766; 4408; 4774; 5201 | 100 | 100 | 100 | 100 | 100 | 7.77E−11 |
| 808 | 264; 388; 662; 991; 1096; 1114; 1157; 1213; 1481; 1530; 1617; 1951; 2206; 2525; 2840; 3206; 3356; | 100 | 100 | 100 | 100 | 100 | 7.77E−11 |
| | 3834; 3842; 3902; 4006; 4144; 4655; 4735; 5119 | | | | | | |
| 809 | 1444; 3148; 3195; 3873; 4643; 4990; 5189; 5242 | 100 | 100 | 100 | 100 | 100 | 7.77E−11 |
| 810 | 19; 62; 1315; 1372; 2287; 3227; 3866; 3954; 4363; 4630 | 100 | 100 | 100 | 100 | 100 | 7.77E−11 |
| 811 | 27; 945; 1329; 4909 | 100 | 100 | 100 | 100 | 100 | 7.77E−11 |
| 812 | 49; 742; 933; 1300; 1476; 2780; 4030 | 100 | 100 | 100 | 100 | 100 | 7.77E−11 |
| 813 | 137; 190; 252; 387; 424; 523; 566; 602; 629; 685; 822; 887; 942; 944; 1000; 1008; 1047; 1092; 1166; | 100 | 100 | 100 | 100 | 100 | 7.77E−11 |
| | 1175; 1177; 1216; 1459; 1507; 1534; 1685; 1721; 1842; 2073; 2147; 2757; 2781; 2787; 2816; 2836; | | | | | | |
| | 3039; 3246; 3341; 3369; 3720; 3776; 3877; 4199; 4434; 4908; 5202 | | | | | | |
| 814 | 1294; 1357; 1721; 4942; 5184 | 100 | 100 | 100 | 100 | 100 | 7.77E−11 |
| 815 | 58; 127; 208; 573; 601; 807; 1276; 1310; 1379; 1801; 2162; 2189; 2228; 2524; 3052; 3120; 3913; 4169; 4267; 4552; 4911; 5157 | 100 | 100 | 100 | 100 | 100 | 7.77E−11 |
| 816 | 953; 1302; 1437; 1946; 2291; 3033; 4318; 4607 | 100 | 100 | 100 | 100 | 100 | 7.77E−11 |
| 817 | 309; 318; 1633; 2272; 2456; 4583; 4871 | 100 | 100 | 100 | 100 | 100 | 7.77E−11 |
| 818 | 53; 467; 636; 759; 768; 804; 902; 1046; 1255; 1651; 1724; 1746; 1749; 1793; 1874; 1921; 1930; 2047; | 100 | 100 | 100 | 100 | 100 | 7.77E−11 |
| | 2097; 2446; 2454; 2456; 2631; 2653; 3032; 3428; 3465; 3468; 3730; 3778; 3881; 3895; 4101; 4126; | | | | | | |
| | 4318; 4322; 4563; 4633; 4680; 4682; 4744; 4822; 5110 | | | | | | |
| 819 | 493; 3203; 3388; 3768; 4216; 4946 | 100 | 100 | 100 | 100 | 100 | 7.77E−11 |
| 820 | 208; 541; 630; 966; 1224; 1658; 2007; 2555; 2816; 3256; 3288; 3610; 3655; 3791; 3882; 4450; 4506; | 100 | 100 | 100 | 100 | 100 | 7.77E−11 |
| | 4521; 4637; 4819; 4837; 4985; 5014; 5130 | | | | | | |
| 821 | 156; 398; 901; 920; 1110; 1547; 1926; 2180; 2846; 2871; 3055; 3225; 3294; 3443; 3609; 3865; 3872; 4185; 4447; 4982; 5000; 5002 | 100 | 100 | 100 | 100 | 100 | 7.77E−11 |
| 822 | 782; 1023; 1155; 1233; 1331; 1548; 2208; 2730; 2979; 3348; 3627; 3990; 4395; 4884; 5286 | 100 | 100 | 100 | 100 | 100 | 7.77E−11 |
| 823 | 3390; 3403; 3749; 4095; 4437; 4477; 4564; 4953; 5023 | 100 | 100 | 100 | 100 | 100 | 7.77E−11 |
| 824 | 344; 937; 1035; 1345; 2614; 2624; 3988; 5070 | 100 | 100 | 100 | 100 | 100 | 7.77E−11 |
| 825 | 14; 67; 153; 289; 461; 1420; 1755; 2814; 3918; 5137 | 100 | 100 | 100 | 100 | 100 | 7.77E−11 |
| 826 | 391; 661; 857; 1800; 2706; 2893; 4962; 4976 | 100 | 100 | 100 | 100 | 100 | 7.77E−11 |
| 827 | 1283; 1375; 1447; 2013; 2667; 2814; 2838; 3207; 3496; 3553; 3671; 4373; 4840; 4875 | 100 | 100 | 100 | 100 | 100 | 7.77E−11 |
| 828 | 1557; 2181; 4050; 4196 | 100 | 100 | 100 | 100 | 100 | 7.77E−11 |
| 829 | 219; 737; 827; 1244; 1306; 1529; 2699; 3036; 3303; 3509; 4503; 4975; 5252 | 100 | 100 | 100 | 100 | 100 | 7.77E−11 |
| 830 | 161; 313; 411; 660; 903; 1163; 1356; 1564; 2329; 2405; 2473; 2539; 3219; 3580; 3623; 4685; 4720; 5068 | 100 | 100 | 100 | 100 | 100 | 7.77E−11 |
| 831 | 318; 1279; 1565; 1785; 2410; 2853; 2934; 3360; 4700 | 100 | 100 | 100 | 100 | 100 | 7.77E−11 |
| 832 | 424; 459; 981; 1227; 1316; 1467; 1615; 1632; 1640; 1744; 1799; 1978; 2160; 2349; 2537; 2592; 2620; | 100 | 100 | 100 | 100 | 100 | 7.77E−11 |
| | 2648; 2914; 3110; 3216; 3284; 3392; 3492; 3560; 3688; 4089; 4165; 4649; 4873; 4941 | | | | | | |
| 833 | 229; 1210; 1420; 1960; 2154; 2530; 3941; 4834; 5034 | 100 | 100 | 100 | 100 | 100 | 7.77E−11 |
| 834 | 665; 1199; 1570; 1766; 4826 | 100 | 100 | 100 | 100 | 100 | 7.77E−11 |
| 835 | 121; 1790; 2328; 3593; 4092; 4210; 4955; 5080; 5243 | 100 | 100 | 100 | 100 | 100 | 7.77E−11 |
| 836 | 186; 949; 1382; 2583; 2685; 3056; 3330; 4074; 4952; 5260 | 100 | 100 | 100 | 100 | 100 | 7.77E−11 |
| 837 | 461; 857; 1308; 1445; 1808; 2335; 2757; 3312; 4011; 4294 | 100 | 100 | 100 | 100 | 100 | 7.77E−11 |
| 838 | 215; 644; 848; 1212; 1879; 2123; 2411; 2457; 3056; 3546; 4363; 4379; 4829 | 100 | 100 | 100 | 100 | 100 | 7.77E−11 |
| 839 | 109; 597; 815; 1134; 1538; 1875; 2180; 2378; 3145; 3651; 3864; 3915; 4217 | 100 | 100 | 100 | 100 | 100 | 7.77E−11 |
| 840 | 614; 1568; 3158; 3875; 3974 | 100 | 100 | 100 | 100 | 100 | 7.77E−11 |
| 841 | 2082; 3611; 4956; 5207 | 100 | 100 | 100 | 100 | 100 | 7.77E−11 |
| 842 | 1245; 3694; 4360; 4819; 5001 | 100 | 100 | 100 | 100 | 100 | 7.77E−11 |
| 843 | 25; 462; 507; 1815; 2153; 2211; 3447; 3698; 4332; 4488 | 100 | 100 | 100 | 100 | 100 | 7.77E−11 |
| 844 | 969; 3064; 4125; 4809; 4966 | 100 | 100 | 100 | 100 | 100 | 7.77E−11 |
| 845 | 2670; 2687; 3378; 3452; 3605; 3611; 4247; 4641; 5253 | 100 | 100 | 100 | 100 | 100 | 7.77E−11 |
| 846 | 52; 85; 141; 592; 604; 834; 879; 1093; 1226; 1254; 1313; 1331; 1687; 1761; 1808; 2160; 2198; 2375; 2485; 2577; 2753; | 100 | 100 | 100 | 100 | 100 | 7.77E−11 |
| | 2963; 3053; 3087; 3111; 3151; 3209; 3285; 3507; 3576; 3932; 3993; 4018; 4051; 4200; 4209; 4232; 4246; 4417; 4510; 4869; 5181; 5280 | | | | | | |
| 847 | 128; 165; 166; 174; 422; 561; 739; 746; 1098; 1403; 1432; 1494; 1535; 1573; 1843; 1927; 1932; 2026; | 100 | 100 | 100 | 100 | 100 | 7.77E−11 |
| | 2092; 2301; 2385; 2573; 2771; 2788; 2796; 2854; 2855; 2880; 2957; 3122; 3208; 3267; 3564; 3668; | | | | | | |
| | 3780; 3830; 3849; 3901; 4095; 4214; 4251; 4351; 4371; 4647; 4776; 4841; 5070; 5133; 5286 | | | | | | |
| 848 | 1186; 1291; 2252; 2396; 3704 | 100 | 100 | 100 | 100 | 100 | 7.77E−11 |
| 849 | 1348; 2933; 3807; 3907; 4294; 4400; 4478; 4517; 4713; 5021 | 100 | 100 | 100 | 100 | 100 | 7.77E−11 |
| 850 | 365; 662; 809; 990; 1091; 1118; 1457; 1480; 1554; 1599; 1701; 1780; 2143; 2159; 2346; 2406; 2426; | 100 | 100 | 100 | 100 | 100 | 7.77E−11 |
| | 2441; 2567; 2620; 2701; 2770; 2955; 3019; 3078; 3115; 3131; 3169; 3241; 3268; 3319; 3369; 3477; | | | | | | |
| | 3505; 3550; 3586; 3644; 4087; 4163; 4190; 4201; 4293; 4413; 4531; 4536; 4945; 5194 | | | | | | |
| 851 | 239; 1766; 3793; 4577; 4607; 4665; 4918 | 100 | 100 | 100 | 100 | 100 | 7.77E−11 |
| 852 | 61; 344; 381; 404; 428; 698; 1088; 1123; 1293; 1438; 1446; 1474; 1640; 2203; 2618; 2653; 2708; 2883; | 100 | 100 | 100 | 100 | 100 | 7.77E−11 |
| | 3063; 3402; 3492; 3692; 3851; 3982; 4618; 4723; 5014 | | | | | | |
| 853 | 58; 377; 893; 1272; 1432; 1776; 1821; 2081; 2207; 2998; 3300; 3338; 3517; 3568; 3726; 3737; 3860; | 100 | 100 | 100 | 100 | 100 | 7.77E−11 |
| | 4003; 4143; 4302; 4382; 4601; 4666; 5016; 5081; 5169 | | | | | | |
| 854 | 178; 2580; 3598; 4088 | 100 | 100 | 100 | 100 | 100 | 7.77E−11 |
| 855 | 7; 26; 110; 178; 333; 353; 731; 1192; 1249; 1535; 1596; 1868; 2061; 2638; 4012; 4013; 4314; 4500; 5164 | 100 | 100 | 100 | 100 | 100 | 7.77E−11 |
| 856 | 33; 85; 172; 389; 692; 724; 937; 1197; 1367; 1393; 1582; 1692; 1737; 1811; 1922; 1929; 2065; 2113; 2116; 2164; 2420; | 100 | 100 | 100 | 100 | 100 | 7.77E−11 |
| | 2677; 2712; 3025; 3038; 3063; 4133; 4163; 4184; 4249; 4272; 4325; 4359; 4488; 4519; 4521; 4680; 4842; 5014 | | | | | | |
| 857 | 1064; 1165; 1192; 1506; 1594; 1836; 1866; 1981; 2143; 3398; 3582; 3586; 4074; 4337; 4558; 5003 | 100 | 100 | 100 | 100 | 100 | 7.77E−11 |
| 858 | 1194; 1326; 1618; 2506; 2715; 2988; 3737; 4435 | 100 | 100 | 100 | 100 | 100 | 7.77E−11 |
| 859 | 207; 350; 1020; 1864; 2440; 3586 | 100 | 100 | 100 | 100 | 100 | 7.77E−11 |
| 860 | 124; 136; 202; 386; 645; 845; 846; 907; 1083; 1106; 1168; 1169; 1212; 1338; 1343; 1359; 1527; 1795; | 100 | 100 | 100 | 100 | 100 | 7.77E−11 |
| | 1894; 1910; 2245; 2266; 2899; 3168; 3177; 3374; 3772; 4308; 4525; 4688; 4735; 4929; 5119 | | | | | | |
| 861 | 243; 1637; 2217; 4046; 4690; 4735; 4844 | 100 | 100 | 100 | 100 | 100 | 7.77E−11 |
| 862 | 286; 815; 1525; 2324; 3034; 3137; 3924; 3935; 5190 | 100 | 100 | 100 | 100 | 100 | 7.77E−11 |
| 863 | 234; 348; 355; 459; 803; 1007; 1400; 1938; 1942; 2241; 2446; 2447; 2836; 2877; 2925; 3084; 3180; | 100 | 100 | 100 | 100 | 100 | 7.77E−11 |
| | 3517; 3609; 3690; 3738; 3877; 3890; 3905; 4359 | | | | | | |
| 864 | 628; 1319; 1928; 3420; 3521 | 100 | 100 | 100 | 100 | 100 | 7.77E−11 |
| 865 | 274; 332; 520; 606; 643; 680; 712; 725; 774; 933; 1094; 1243; 1721; 1738; 1739; 1973; 1996; 2038; 2045; 2112; 2122; | 100 | 100 | 100 | 100 | 100 | 7.77E−11 |
| | 2245; 2404; 2649; 2883; 2957; 2967; 3074; 3388; 3399; 3586; 3874; 4044; 4096; 4146; 4388; 4469; 4673; 4701; 4893; 5118 | | | | | | |
| 866 | 122; 729; 848; 928; 1036; 1481; 1789; 1916; 2228; 2391; 2394; 2662; 2767; 2848; 2936; 3126; 3746; | 100 | 100 | 100 | 100 | 100 | 7.77E−11 |
| | 3827; 3906; 3993; 3995; 4173; 4341; 4618; 4626; 4645; 4751; 4823; 5276 | | | | | | |
| 867 | 3; 127; 267; 379; 487; 554; 691; 776; 1159; 1548; 1591; 1641; 1717; 1759; 1896; 2354; 2400; 2462; | 100 | 100 | 100 | 100 | 100 | 7.77E−11 |
| | 2804; 3204; 3459; 3873; 3916; 4112; 4198; 4374; 4619; 4891; 5091; 5123 | | | | | | |
| 868 | 139; 885; 2424; 2522; 2846; 2966; 3306; 4637 | 100 | 100 | 100 | 100 | 100 | 7.77E−11 |
| 869 | 173; 367; 691; 1357; 1550; 1658; 1995; 3182; 3298; 3394; 3796; 3901; 4186; 4316; 4432; 4444; 4977; 5144 | 100 | 100 | 100 | 100 | 100 | 7.77E−11 |
| 870 | 152; 796; 1059; 1236; 1405; 1576; 2111; 2641; 2688; 3447; 3974; 4391 | 100 | 100 | 100 | 100 | 100 | 7.77E−11 |
| 871 | 287; 1095; 2849; 3127; 3146; 3653; 4013; 4399; 4982; 4983 | 100 | 100 | 100 | 100 | 100 | 7.77E−11 |
| 872 | 15; 119; 442; 543; 792; 833; 881; 973; 1117; 1630; 1802; 1966; 2516; 2534; 2559; 2622; 3043; 3261; | 100 | 100 | 100 | 100 | 100 | 7.77E−11 |
| | 3262; 3339; 3388; 3663; 3669; 3771; 3916; 4037; 4095; 4106; 4184; 4266; 4449; 4552; 4743; 5004; 5014; 5024; 5167 | | | | | | |
| 873 | 164; 2333; 2442; 4155; 5048 | 100 | 100 | 100 | 100 | 100 | 7.77E−11 |
| 874 | 64; 884; 969; 1740; 1884; 2085; 2517; 2667; 2821; 2875; 3053; 3150; 3164; 3254; 3307; 4262; 4695 | 100 | 100 | 100 | 100 | 100 | 7.77E−11 |
| 875 | 159; 231; 508; 655; 703; 710; 737; 783; 1286; 1353; 1563; 1595; 1668; 2122; 2174; 2312; 2677; 2684; | 100 | 100 | 100 | 100 | 100 | 7.77E−11 |
| | 2700; 2719; 2748; 2850; 2852; 2862; 3116; 3387; 3425; 3478; 3586; 3752; 4915; 4989; 5277 | | | | | | |
| 876 | 101; 221; 292; 529; 1150; 1160; 1297; 2228; 2467; 2486; 3794; 3826; 3909; 4028; 4134; 4170; 4860; 4942; 4972 | 100 | 100 | 100 | 100 | 100 | 7.77E−11 |
| 877 | 184; 185; 268; 338; 900; 1319; 1373; 1375; 1541; 1649; 2153; 2432; 2746; 2889; 3022; 3214; 3294; 3426; 3714; 3830; | 100 | 100 | 100 | 100 | 100 | 7.77E−11 |
| | 5004; 5142; 5265 | | | | | | |
| 878 | 94; 122; 221; 290; 404; 515; 545; 730; 892; 920; 1028; 1174; 1225; 1276; 1304; 1550; 1669; 1757; 1939; | 100 | 100 | 100 | 100 | 100 | 7.77E−11 |
| | 1952; 2046; 2295; 2415; 2511; 2556; 2616; 2815; 2894; 2968; 3219; 3269; 3420; 3512; 3552; 3786; | | | | | | |
| | 3815; 3881; 3904; 4181; 4433; 4472; 4761; 4776; 5039; 5058; 5070 | | | | | | |
| 879 | 321; 1190; 1858; 2760; 3182; 3611 | 100 | 100 | 100 | 100 | 100 | 7.77E−11 |
| 880 | 138; 646; 794; 1127; 1279; 2009; 2725; 2948; 2971; 3059; 3283; 3352; 3584; 4480; 4894 | 100 | 100 | 100 | 100 | 100 | 7.77E−11 |
| 881 | 49; 897; 1543; 2444; 2903; 3593; 3916; 4338; 4476; 5099 | 100 | 100 | 100 | 100 | 100 | 7.77E−11 |
| 882 | 306; 1682; 2670; 2744; 3502 | 100 | 100 | 100 | 100 | 100 | 7.77E−11 |
| 883 | 1371; 1420; 1989; 2593; 2998; 3191; 3363; 3378; 3782; 3806; 4036; 4302; 4656; 4693; 4708; 4760; 5109 | 100 | 100 | 100 | 100 | 100 | 7.77E−11 |
| 884 | 288; 378; 670; 751; 771; 1303; 3313; 3611; 3953; 4465; 4629; 4823; 4867; 5115; 5125 | 100 | 100 | 100 | 100 | 100 | 7.77E−11 |
| 885 | 755; 1606; 2790; 2939; 3321; 3447; 3588; 5141; 5253 | 100 | 100 | 100 | 100 | 100 | 7.77E−11 |
| 886 | 191; 370; 379; 407; 544; 647; 668; 685; 960; 963; 1190; 1377; 1450; 1609; 1707; 1772; 1958; 2003; 2029; 2046; | 100 | 100 | 100 | 100 | 100 | 7.77E−11 |
| | 2117; 2393; 2937; 2961; 2976; 2979; 3379; 3464; 3505; 3560; 3562; 3612; 3728; 3778; 4021; 4546; 4836; 4931; 5179 | | | | | | |
| 887 | 182; 264; 332; 491; 716; 782; 916; 1099; 1117; 1225; 1345; 1670; 2014; 2036; 2304; 2422; 2691; | 100 | 100 | 100 | 100 | 100 | 7.77E−11 |
| | 2692; 2695; 2799; 2895; 2921; 2974; 3033; 3329; 3481; 3528; 3570; 3579; 3680; 3699; 3774; 3842; | | | | | | |
| | 3945; 4125; 4153; 4165; 4175; 4203; 4298; 4398; 4630; 4752; 5102; 5204; 5220; 5269 | | | | | | |
| 888 | 1816; 2843; 3317; 4889 | 100 | 100 | 100 | 100 | 100 | 7.77E−11 |
| 889 | 207; 276; 561; 737; 1185; 1583; 1880; 1899; 2395; 2648; 2804; 2869; 3412; 3460; 4110; 4488; 4559; 4667; 4676; 4764; 4999; 5194 | 100 | 100 | 100 | 100 | 100 | 7.77E−11 |
| 890 | 874; 1283; 1509; 1583; 2367; 2831; 2861; 4075; 4274; 5176 | 100 | 100 | 100 | 100 | 100 | 7.77E−11 |
| 891 | 25; 52; 318; 472; 669; 681; 753; 796; 797; 926; 1138; 1323; 1654; 1765; 1791; 2225; 2302; 2477; 2809; | 100 | 100 | 100 | 100 | 100 | 7.77E−11 |
| | 2837; 2947; 3106; 3165; 3209; 3593; 3625; 3646; 3786; 4219; 4306; 4380; 4396; 4470; 4724 | | | | | | |
| 892 | 52; 131; 295; 342; 412; 532; 606; 826; 985; 1070; 1589; 1763; 1845; 2026; 2106; 2147; 2220; 2469; 2593; 2948; | 100 | 100 | 100 | 100 | 100 | 7.77E−11 |
| | 2986; 3205; 3241; 3799; 3926; 3999; 4002; 4142; 4147; 4263; 4278; 4514; 4541; 4565; 4606; 4720; 4738; 4882; 5163 | | | | | | |
| 893 | 95; 118; 270; 336; 339; 469; 586; 735; 789; 854; 1490; 1782; 1910; 2065; 2173; 2425; 2874; 3072; | 100 | 100 | 100 | 100 | 100 | 7.77E−11 |
| | 3657; 3701; 3721; 3816; 3896; 4402; 4861; 4964; 5081; 5257 | | | | | | |
| 894 | 1424; 1752; 1785; 3357; 3859 | 100 | 100 | 100 | 100 | 100 | 7.77E−11 |
| 895 | 480; 636; 819; 1106; 1170; 1384; 1438; 1594; 2253; 2611; 2620; 2787; 3364; 3490; 3673; 4414; 4535; 4637; 4783; 4860; 4914 | 100 | 100 | 100 | 100 | 100 | 7.77E−11 |
| 896 | 382; 667; 687; 715; 837; 954; 1109; 1110; 1481; 1686; 1921; 2846; 2887; 3348; 3467; 3758; 3771; 3868; 4032; 4039; 4625; | 100 | 100 | 100 | 100 | 100 | 7.77E−11 |
| | 5028; 5167; 5215 | | | | | | |
| 897 | 53; 207; 271; 342; 346; 401; 576; 632; 874; 934; 991; 1342; 1451; 1492; 1741; 2194; 2236; 2239; | 100 | 100 | 100 | 100 | 100 | 7.77E−11 |
| | 2246; 2342; 2398; 2645; 2701; 2722; 2862; 2946; 3062; 3186; 3232; 3444; 3550; 4089; 4160; 4339; | | | | | | |
| | 4415; 4444; 4469; 4634; 4635; 4659; 4788; 4811; 5021; 5061; 5075; 5085; 5139; 5158; 5262; 5280 | | | | | | |
| 898 | 728; 1162; 1254; 1311; 1315; 1392; 1630; 2405; 2444; 2922; 3121; 3275; 3899; 4146; 4207; 4692 | 100 | 100 | 100 | 100 | 100 | 7.77E−11 |
| 899 | 6; 108; 378; 510; 948; 1185; 1268; 1607; 1699; 1854; 1863; 2344; 2570; 2658; 2836; 3021; 3270; 3278; | 100 | 100 | 100 | 100 | 100 | 7.77E−11 |
| | 3443; 3507; 3886; 3908; 3918; 4218; 4230; 4293; 4313; 4331; 4532; 5100; 5144; 5271 | | | | | | |
| 900 | 160; 323; 859; 1148; 1891; 1933; 3388; 3605; 4088 | 100 | 100 | 100 | 100 | 100 | 7.77E−11 |
| 901 | 6; 466; 479; 640; 2354; 2837; 3319; 3628; 3847; 4144 | 100 | 100 | 100 | 100 | 100 | 7.77E−11 |
| 902 | 691; 1084; 3229; 3934; 3978; 4073; 4108; 4144; 4255; 4665 | 100 | 100 | 100 | 100 | 100 | 7.77E−11 |
| 903 | 2642; 3214; 4028 | 100 | 100 | 100 | 100 | 100 | 7.77E−11 |
| 904 | 40; 595; 690; 709; 1653; 1735; 2220; 3079; 3112; 3330; 3358; 3457; 3586; 3607; 4015; 4081; 4384; 4429; 4802; 4948; 4955 | 100 | 100 | 100 | 100 | 100 | 7.77E−11 |
| 905 | 641; 804; 1145; 1172; 1308; 1661; 2183; 3453; 3653; 4098 | 100 | 100 | 100 | 100 | 100 | 7.77E−11 |
| 906 | 326; 1400; 1997; 2172; 5207; 5233 | 100 | 100 | 100 | 100 | 100 | 7.77E−11 |
| 907 | 376; 509; 2239; 2639; 3379; 4001; 4616; 5022; 5180 | 100 | 100 | 100 | 100 | 100 | 7.77E−11 |
| 908 | 548; 932; 2022; 2124; 3025; 3269; 3377; 4618 | 100 | 100 | 100 | 100 | 100 | 7.77E−11 |
| 909 | 151; 200; 202; 266; 379; 459; 519; 600; 622; 683; 950; 989; 1081; 1088; 1135; 1158; 1173; 1348; 1657; 1725; | 100 | 100 | 100 | 100 | 100 | 7.77E−11 |
| | 1746; 1827; 2111; 2213; 2234; 2413; 2575; 3014; 3642; 3967; 4107; 4180; 4188; 4275; 4343; 4464; 4515; 4618; 4625; 5025 | | | | | | |
| 910 | 123; 146; 284; 399; 806; 834; 1050; 1153; 1235; 1351; 1435; 1436; 1822; 1875; 1921; 1953; 2259; | 100 | 100 | 100 | 100 | 100 | 7.77E−11 |
| | 2349; 2351; 2460; 2573; 2624; 2675; 2746; 2760; 2917; 2937; 3391; 3431; 3542; 3726; 4041; 4085; | | | | | | |
| | 4114; 4637; 4738; 4760; 4913; 4928; 5006; 5182; 5220; 5259 | | | | | | |
| 911 | 758; 945; 1886; 3356; 3561; 3669; 4977; 5009 | 100 | 100 | 100 | 100 | 100 | 7.77E−11 |
| 912 | 941; 1072; 1245; 2667; 2727; 3270; 4588; 4833; 4960 | 100 | 100 | 100 | 100 | 100 | 7.77E−11 |
| 913 | 16; 203; 293; 326; 661; 917; 963; 1233; 1441; 1442; 1561; 2141; 2184; 2257; 2581; 2870; 2900; 3011; | 100 | 100 | 100 | 100 | 100 | 7.77E−11 |
| | 3297; 3339; 3421; 3737; 3911; 4062; 4089; 4604; 4794; 4844; 5042; 5043; 5081; 5164 | | | | | | |
| 914 | 247; 419; 670; 698; 1049; 1077; 1186; 1388; 1812; 2582; 2805; 3268; 3582; 3740; 4165; 4283; 4397; 4456; 4513; 4634; 4692 | 100 | 100 | 100 | 100 | 100 | 7.77E−11 |
| 915 | 134; 137; 218; 227; 498; 503; 606; 707; 801; 905; 1176; 1184; 1259; 1349; 1519; 1551; 1596; 1600; | 100 | 100 | 100 | 100 | 100 | 7.77E−11 |
| | 1727; 2660; 2682; 2830; 3010; 3093; 3209; 3481; 3569; 3866; 4045; 4170; 4207; 4308; 4431; 4519; 4576; 4957; 5039 | | | | | | |
| 916 | 530; 761; 806; 1354; 2465; 2845; 2918; 3217; 3924; 4129; 4976; 5090; 5104 | 100 | 100 | 100 | 100 | 100 | 7.77E−11 |
| 917 | 837; 1112; 2336; 2739; 2757; 2841; 3068 | 100 | 100 | 100 | 100 | 100 | 7.77E−11 |
| 918 | 982; 1149; 1945; 2618; 2875 | 100 | 100 | 100 | 100 | 100 | 7.77E−11 |
| 919 | 136; 538; 742; 963; 1071; 1550; 1615; 1745; 1813; 1965; 2041; 2064; 2248; 2429; 2868; 2969; 3289; | 100 | 100 | 100 | 100 | 100 | 7.77E−11 |
| | 3391; 3588; 3660; 3736; 3747; 3901; 4015; 4274; 4507; 4520; 4637; 4897; 5059; 5150; 5253 | | | | | | |
| 920 | 301; 1702; 2311; 2335; 2636; 2949; 4752; 5201 | 100 | 100 | 100 | 100 | 100 | 7.77E−11 |
| 921 | 269; 379; 441; 730; 1160; 1381; 1426; 1603; 1664; 2409; 2616; 2997; 3015; 3528; 3556; 3610; 3691; | 100 | 100 | 100 | 100 | 100 | 7.77E−11 |
| | 3954; 4127; 4378; 4600; 4630; 4665; 4777; 4875; 5017; 5091; 5111; 5129 | | | | | | |
| 922 | 529; 790; 1966; 3890 | 100 | 100 | 100 | 100 | 100 | 7.77E−11 |
| 923 | 132; 188; 412; 545; 673; 1098; 1185; 1569; 1651; 1701; 1730; 1745; 1746; 1802; 1846; 1849; 1859; 2007; 2052; | 100 | 100 | 100 | 100 | 100 | 7.77E−11 |
| | 2091; 2134; 2181; 2182; 2430; 2460; 2517; 2673; 2713; 3041; 3110; 3400; 3562; 3922; 3923; 4175; 4176; 4259; 4316; 4346; 4813; 5227 | | | | | | |
| 924 | 105; 1042; 1218; 1420; 1564; 1569; 2073; 2265; 2578; 2765; 3215; 3312; 3702; 3903; 4351; 4371; | 100 | 100 | 100 | 100 | 100 | 7.77E−11 |
| | 4517; 4529; 4544; 4714; 4966; 5046 | | | | | | |
| 925 | 339; 1314; 1340; 2040; 2678; 2755; 2816; 2847; 3272; 4834; 4917 | 100 | 100 | 100 | 100 | 100 | 7.77E−11 |
| 926 | 687; 1178; 1931; 2334; 2502; 2701; 2792; 2962; 3280; 3820; 4166; 4178; 4229; 4319; 4970 | 100 | 100 | 100 | 100 | 100 | 7.77E−11 |
| 927 | 198; 590; 657; 779; 1095; 1517; 1905; 2070; 3256; 4750; 4830; 4944 | 100 | 100 | 100 | 100 | 100 | 7.77E−11 |
| 928 | 396; 812; 1090; 1138; 1300; 2836; 2912; 2918; 3177; 3212; 3652; 4408; 4684; 5075; 5078 | 100 | 100 | 100 | 100 | 100 | 7.77E−11 |
| 929 | 83; 432; 822; 1294; 2052; 2699; 3879; 4265 | 100 | 100 | 100 | 100 | 100 | 7.77E−11 |
| 930 | 91; 117; 500; 588; 814; 828; 837; 879; 1043; 1117; 1127; 1223; 1243; 1257; 1442; 1501; 1600; 1723; | 100 | 100 | 100 | 100 | 100 | 7.77E−11 |
| | 1807; 1840; 1841; 2052; 2080; 2335; 2523; 2688; 2702; 2706; 2905; 3250; 3374; 3421; 3522; 3571; | | | | | | |
| | 3667; 3853; 3939; 3989; 4184; 4208; 4216; 4276; 4387; 4420; 4676; 4938; 4963; 5020 | | | | | | |
| 931 | 0; 7; 179; 218; 474; 805; 835; 962; 1087; 1128; 1132; 1153; 1176; 1218; 1339; 1464; 1528; 1669; 1679; 1842; 1895; 2074; 2439; | 100 | 100 | 100 | 100 | 100 | 7.77E−11 |
| | 2545; 2677; 2727; 2861; 2864; 3037; 3066; 3107; 3518; 3649; 3877; 4069; 4175; 4221; 4404; 4563; 4645; 4807; 4823; 4838; 5096 | | | | | | |
| 932 | 58; 102; 404; 539; 1126; 1144; 1215; 1366; 1450; 1458; 1584; 1689; 2155; 2717; 2779; 2963; 3155; | 100 | 100 | 100 | 100 | 100 | 7.77E−11 |
| | 3209; 3291; 3359; 3435; 3499; 3761; 3805; 4459; 4650; 4879; 4975 | | | | | | |
| 933 | 2463; 2632; 2842; 4528; 5264 | 100 | 100 | 100 | 100 | 100 | 7.77E−11 |
| 934 | 46; 55; 129; 398; 443; 462; 529; 731; 820; 862; 1335; 1388; 1523; 1598; 1971; 2225; 2289; 2617; 3040; 3155; 3269; 3314; | 100 | 100 | 100 | 100 | 100 | 7.77E−11 |
| | 3357; 3464; 3480; 3970; 4002; 4051; 4097; 4147; 4152; 4263; 4313; 4368; 4457; 4594; 4705; 4801; 4887; 4997; 5045; 5071; 5173 | | | | | | |
| 935 | 304; 368; 1181; 1252; 1606; 1648; 2180; 3662; 4401; 4997 | 100 | 100 | 100 | 100 | 100 | 7.77E−11 |
| 936 | 877; 2398; 2592; 2767; 2801; 4136 | 100 | 100 | 100 | 100 | 100 | 7.77E−11 |
| 937 | 89; 508; 772; 1160; 1247; 2241; 2311; 2677; 2725; 2877; 3161; 3946; 4083; 4201; 4819 | 100 | 100 | 100 | 100 | 100 | 7.77E−11 |
| 938 | 272; 299; 917; 2143; 2446; 2686; 3650; 4098; 5173 | 100 | 100 | 100 | 100 | 100 | 7.77E−11 |
| 939 | 617; 1057; 1578; 1837; 1901; 2106; 2148; 2666; 3247; 3576; 3727; 3755; 4106; 4882; 4974 | 100 | 100 | 100 | 100 | 100 | 7.77E−11 |
| 940 | 606; 1080; 2338; 3214; 4778 | 100 | 100 | 100 | 100 | 100 | 7.77E−11 |
| 941 | 377; 482; 976; 1081; 1111; 1999; 2322; 2994; 3750; 3961; 4707 | 100 | 100 | 100 | 100 | 100 | 7.77E−11 |
| 942 | 389; 933; 1078; 1471; 1664; 1668; 1968; 3019; 3254; 3388; 4508; 4566; 4569; 4895 | 100 | 100 | 100 | 100 | 100 | 7.77E−11 |
| 943 | 586; 2352; 2676; 2807; 3001; 3761; 3986; 4033; 4498 | 100 | 100 | 100 | 100 | 100 | 7.77E−11 |
| 944 | 627; 1127; 1375; 2781; 4422; 4856; 5093 | 100 | 100 | 100 | 100 | 100 | 7.77E−11 |
| 945 | 135; 384; 1213; 1635; 2842; 3084; 3432; 3445; 3864; 4394 | 100 | 100 | 100 | 100 | 100 | 7.77E−11 |
| 946 | 246; 1563; 2105; 2482; 2597; 3456; 4220 | 100 | 100 | 100 | 100 | 100 | 7.77E−11 |
| 947 | 1631; 1954; 2257; 2395; 2450; 3628; 4183; 4399 | 100 | 100 | 100 | 100 | 100 | 7.77E−11 |
| 948 | 138; 2675; 3042; 4933 | 100 | 100 | 100 | 100 | 100 | 7.77E−11 |
| 949 | 512; 529; 620; 644; 1271; 1591; 1659; 2116; 2148; 2604; 3071; 3263; 3437; 3454; 3940; 4417; 4437; 4999 | 100 | 100 | 100 | 100 | 100 | 7.77E−11 |
| 950 | 46; 70; 1145; 1149; 1376; 1745; 1959; 2042; 3377; 3722; 4059; 4182; 4595; 4656; 4774; 4879; 4976 | 100 | 100 | 100 | 100 | 100 | 7.77E−11 |
| 951 | 57; 81; 198; 278; 498; 671; 925; 1066; 1156; 1266; 1392; 1481; 1501; 1578; 1592; 1693; 1855; 1925; | 100 | 100 | 100 | 100 | 100 | 7.77E−11 |
| | 1944; 2036; 2060; 2122; 2403; 2444; 2558; 2702; 3016; 3311; 3342; 3527; 3552; 3672; 3701; 3876; | | | | | | |
| | 3982; 4107; 4224; 4234; 4336; 4456; 4662; 4697; 4792; 4879; 5070; 5090 | | | | | | |
| 952 | 181; 1086; 1332; 1606; 2867; 3188; 3258; 3497; 3584; 3696; 4375; 4434; 4730; 5199 | 100 | 100 | 100 | 100 | 100 | 7.77E−11 |
| 953 | 45; 110; 162; 350; 355; 377; 445; 646; 976; 1297; 1340; 1400; 1450; 1611; 1672; 1708; 2011; 2077; | 100 | 100 | 100 | 100 | 100 | 7.77E−11 |
| | 2216; 2240; 2516; 2573; 2668; 2814; 2845; 3166; 3235; 3401; 3547; 3791; 3986; 4265; 4369; 4412; | | | | | | |
| | 4509; 4511; 4571; 4603; 4682; 4694; 4788; 4790; 4918; 4953; 5070 | | | | | | |
| 954 | 163; 370; 877; 962; 1051; 1337; 1356; 1555; 1587; 1660; 1690; 1717; 1747; 2141; 2342; 3091; 3120; | 100 | 100 | 100 | 100 | 100 | 7.77E−11 |
| | 3257; 3386; 3478; 3577; 3718; 4100; 4346; 4618; 4678; 4679; 5179 | | | | | | |
| 955 | 1582; 1883; 2783; 2891; 3151; 4347; 4994; 5239 | 100 | 100 | 100 | 100 | 100 | 7.77E−11 |
| 956 | 51; 1110; 2676; 4707; 5152 | 100 | 100 | 100 | 100 | 100 | 7.77E−11 |
| 957 | 49; 67; 365; 639; 711; 955; 1026; 1057; 1287; 1403; 1451; 1611; 1639; 1723; 1864; 2423; 2458; 2513; | 100 | 100 | 100 | 100 | 100 | 7.77E−11 |
| | 2606; 2991; 3188; 3496; 4433; 4618; 4693; 4965 | | | | | | |
| 958 | 30; 87; 458; 528; 531; 813; 1399; 1538; 1957; 3102; 3209; 3221; 4442; 4964; 5070 | 100 | 100 | 100 | 100 | 100 | 7.77E−11 |
| 959 | 213; 343; 458; 798; 1036; 1387; 1738; 1805; 2182; 2847; 3188; 3215; 3222; 3282; 3353; 3396; 3440; | 100 | 100 | 100 | 100 | 100 | 7.77E−11 |
| | 3884; 3917; 3940; 4053; 4548; 4666; 4756; 4824; 5001; 5101 | | | | | | |
| 960 | 418; 466; 519; 855; 926; 1127; 1335; 1561; 1606; 1658; 2046; 2049; 2203; 2646; 2658; 2719; 2843; 3027; 3088; 3121; | 100 | 100 | 100 | 100 | 100 | 7.77E−11 |
| | 3202; 3338; 3445; 3552; 3581; 3652; 3658; 3684; 3769; 3852; 4038; 4058; 4134; 4144; 4519; 4687; 4994; 5053; 5068; 5132; 5239 | | | | | | |
| 961 | 564; 2091 | 100 | 100 | 100 | 100 | 100 | 7.77E−11 |
| 962 | 1687; 1933; 1943; 2268; 2654; 3729; 3876; 3920; 4035; 4495; 4554; 4861 | 100 | 100 | 100 | 100 | 100 | 7.77E−11 |
| 963 | 74; 3042; 3216 | 100 | 100 | 100 | 100 | 100 | 7.77E−11 |
| 964 | 19; 310; 379; 545; 679; 887; 905; 1003; 1389; 1800; 1865; 1982; 2739; 3025; 3326; 3376; 3628; 3691; 4361; 4424; 4723; 4813; 4918 | 100 | 100 | 100 | 100 | 100 | 7.77E−11 |
| 965 | 32; 537; 581; 666; 973; 1689; 1854; 2946; 2989; 3080; 3209; 3920; 4092; 4447; 4871 | 100 | 100 | 100 | 100 | 100 | 7.77E−11 |
| 966 | 80; 85; 168; 275; 424; 508; 653; 824; 871; 933; 1015; 1106; 1275; 1325; 1359; 1380; 1389; 1507; 1716; | 100 | 100 | 100 | 100 | 100 | 7.77E−11 |
| | 1919; 2035; 2071; 2073; 2154; 2264; 2598; 2610; 2690; 2704; 2846; 3000; 3100; 3170; 3171; 3366; | | | | | | |
| | 3453; 3586; 3756; 3813; 3906; 3961; 4041; 4235; 4300; 4312; 4364; 4489; 4523; 4681; 4788 | | | | | | |
| 967 | 8; 42; 50; 107; 292; 402; 440; 552; 610; 713; 787; 899; 1138; 1212; 1245; 1261; 1402; 1659; 1720; 1991; | 100 | 100 | 100 | 100 | 100 | 7.77E−11 |
| | 2183; 2538; 2942; 3093; 3137; 3253; 3432; 3474; 3654; 3702; 3882; 3982; 4102; 4221; 4297; 4303; | | | | | | |
| | 4327; 4336; 4471; 4507; 4684; 4768; 4853; 4928; 5006; 5094; 5229; 5252 | | | | | | |
| 968 | 129; 137; 263; 583; 599; 1150; 1210; 1431; 2204; 2297; 2404; 2642; 2665; 2913; 3452; 3489; 3532; | 100 | 100 | 100 | 100 | 100 | 7.77E−11 |
| | 3819; 3840; 3917; 4243; 4441; 4456; 4694; 5260 | | | | | | |
| 969 | 624; 1120; 1370; 2532; 3364; 3516; 3677; 4100; 4442 | 100 | 100 | 100 | 100 | 100 | 7.77E−11 |
| 970 | 4; 230; 612; 628; 668; 788; 805; 1089; 1163; 1192; 1710; 1922; 1992; 2054; 2289; 2514; 2832; 2870; | 100 | 100 | 100 | 100 | 100 | 7.77E−11 |
| | 3911; 3992; 4174; 4275; 4660; 5241; 5256 | | | | | | |
| 971 | 210; 2158; 2340; 2765; 4681; 4948 | 100 | 100 | 100 | 100 | 100 | 7.77E−11 |
| 972 | 64; 443; 1098; 1326; 1497; 2079; 2101; 2525; 2537; 4031; 4447; 4844; 4917; 5000; 5065 | 100 | 100 | 100 | 100 | 100 | 7.77E−11 |
| 973 | 52; 936; 2415; 2494; 2675; 3302; 3753; 4508; 4984 | 100 | 100 | 100 | 100 | 100 | 7.77E−11 |
| 974 | 1638; 1800; 1869; 2343; 2443; 3225; 3428; 4560 | 100 | 100 | 100 | 100 | 100 | 7.77E−11 |
| 975 | 10; 535; 605; 616; 1173; 1254; 1277; 1281; 1305; 1311; 1575; 1750; 1782; 1845; 1897; 1903; 2069; | 100 | 100 | 100 | 100 | 100 | 7.77E−11 |
| | 2119; 2441; 2450; 2547; 2642; 2753; 2798; 2936; 2976; 3155; 3171; 3242; 3373; 3505; 3605; 3858; | | | | | | |
| | 3859; 3860; 4153; 4248; 4263; 4289; 4608; 4851; 4877; 4917 | | | | | | |
| 976 | 202; 286; 292; 293; 294; 333; 477; 554; 716; 1335; 1391; 2413; 2563; 2691; 2694; 3011; 3285; 3444; | 100 | 100 | 100 | 100 | 100 | 7.77E−11 |
| | 3578; 3630; 3690; 3867; 3931; 3977; 4323; 4372; 4478; 4618; 4914; 5159; 5263 | | | | | | |
| 977 | 233; 487; 712; 934; 937; 950; 1001; 1035; 1082; 1138; 1254; 1564; 1648; 1689; 2365; 2577; 2898; | 100 | 100 | 100 | 100 | 100 | 7.77E−11 |
| | 2916; 2949; 3048; 3207; 3322; 3608; 3663; 3843; 4094; 4155; 4260; 4262; 4323; 4395; 4529; 4827; 4895 | | | | | | |
| 978 | 44; 919; 1163; 1242; 1455; 1633; 1715; 1767; 3306; 3496; 3586; 4297; 4337 | 100 | 100 | 100 | 100 | 100 | 7.77E−11 |
| 979 | 85; 250; 308; 424; 648; 733; 774; 783; 826; 889; 903; 950; 1067; 1214; 1228; 1281; 1288; 1308; 1323; | 100 | 100 | 100 | 100 | 100 | 7.77E−11 |
| | 1461; 1464; 1558; 1900; 2326; 2390; 2409; 2520; 2552; 2683; 2690; 2726; 2944; 3013; 3024; 3208; | | | | | | |
| | 3303; 3457; 3693; 3975; 4008; 4134; 4241; 4259; 4295; 4395; 4567; 4781; 4814; 4886; 5194 | | | | | | |
| 980 | 582; 1059; 1153; 1396; 2050; 2642; 2945; 3342; 3746; 4097; 4802; 4844; 5020 | 100 | 100 | 100 | 100 | 100 | 7.77E−11 |
| 981 | 610; 1800; 2919; 3716; 3790; 3978; 4250 | 100 | 100 | 100 | 100 | 100 | 7.77E−11 |
| 982 | 323; 345; 514; 1574; 1706; 2153; 2403; 2635; 2812; 2834; 2874; 3110; 3401; 3819; 4040; 4344; 4609; 4633; 5237 | 100 | 100 | 100 | 100 | 100 | 7.77E−11 |
| 983 | 75; 318; 534; 636; 1321; 1377; 2026; 2128; 2193; 2370; 2651; 2731; 2959; 2976; 3097; 3392; 3867; | 100 | 100 | 100 | 100 | 100 | 7.77E−11 |
| | 4127; 4207; 4671; 4685; 4719; 4820; 5003; 5173; 5254 | | | | | | |
| 984 | 734; 1098; 1310; 1552; 2574 | 100 | 100 | 100 | 100 | 100 | 7.77E−11 |
| 985 | 492; 799; 1523; 1903; 2144; 2430; 3580; 4477; 4515; 4537 | 100 | 100 | 100 | 100 | 100 | 7.77E−11 |
| 986 | 143; 590; 1505; 1851; 2123; 2530; 3058; 3300; 3610; 3904; 4188; 4547; 4843 | 100 | 100 | 100 | 100 | 100 | 7.77E−11 |
| 987 | 264; 417; 517; 643; 650; 939; 1034; 1286; 1551; 1607; 1707; 1740; 1755; 1785; 1800; 1877; 2084; | 100 | 100 | 100 | 100 | 100 | 7.77E−11 |
| | 2136; 2454; 2564; 2602; 2620; 2843; 2845; 2883; 2927; 3000; 3172; 3177; 3346; 3485; 3594; 3683; | | | | | | |
| | 3748; 3803; 4043; 4218; 4231; 4476; 4590; 4690; 4853; 4856; 4948; 5104; 5149; 5233 | | | | | | |
| 988 | 219; 744; 1118; 4505; 4703; 4843 | 100 | 100 | 100 | 100 | 100 | 7.77E−11 |
| 989 | 725; 823; 1393; 1867; 3629; 4595; 4630; 4644; 5110 | 100 | 100 | 100 | 100 | 100 | 7.77E−11 |
| 990 | 212; 407; 949; 1214; 1581; 1665; 1720; 2789; 2924; 3511; 3928; 4074; 4890; 5242 | 100 | 100 | 100 | 100 | 100 | 7.77E−11 |
| 991 | 8; 31; 159; 316; 342; 499; 770; 804; 988; 1049; 1061; 1164; 1206; 1286; 1616; 1676; 1882; 2055; 2068; | 100 | 100 | 100 | 100 | 100 | 7.77E−11 |
| | 2083; 2187; 2220; 2246; 2351; 2576; 2759; 2951; 3042; 3145; 3250; 3271; 3446; 3535; 3615; 3790; | | | | | | |
| | 3941; 4134; 4199; 4269; 4493; 4515; 4672; 4828; 4833; 5083; 5169; 5234; 5278 | | | | | | |
| 992 | 1581; 2358; 3218; 3941; 4230; 4804; 4816; 4952; 5200 | 100 | 100 | 100 | 100 | 100 | 7.77E−11 |
| 993 | 668; 1720; 1887; 3636 | 100 | 100 | 100 | 100 | 100 | 7.77E−11 |
| 994 | 540; 3395; 3710; 4542 | 100 | 100 | 100 | 100 | 100 | 7.77E−11 |
| 995 | 992; 1208; 2903; 3173; 3423; 5097 | 100 | 100 | 100 | 100 | 100 | 7.77E−11 |
| 996 | 43; 428; 493; 827; 910; 919; 1017; 1350; 1415; 1455; 1678; 1752; 1865; 2376; 2428; 2468; 2490; 2576; | 100 | 100 | 100 | 100 | 100 | 7.77E−11 |
| | 2585; 2779; 3005; 3466; 3496; 3704; 3877; 3934; 3978; 4353; 4477; 4683; 4704; 4841; 4901; 4942; 4999; 5133; 5252 | | | | | | |
| 997 | 41; 74; 137; 202; 327; 903; 1090; 1138; 1167; 1405; 1468; 1657; 1701; 1741; 2001; 2222; 2274; 2488; | 100 | 100 | 100 | 100 | 100 | 7.77E−11 |
| | 2586; 2639; 2706; 2974; 3012; 3091; 3501; 3607; 3616; 3677; 3996; 4065; 4068; 4130; 4141; 4184; | | | | | | |
| | 4339; 4416; 4542; 4591; 4620; 4653; 4682; 4832; 4847; 5012; 5156; 5231 | | | | | | |
| 998 | 856; 1243; 1654; 1665; 2012; 2120; 2326; 3096; 3169; 3197; 3593; 3860; 3869; 4028; 4199; 4416; 4435; 4438; 4603; 4949 | 100 | 100 | 100 | 100 | 100 | 7.77E−11 |
| 999 | 226; 314; 702; 1143; 1155; 1186; 1240; 1508; 1550; 1719; 1753; 1975; 2005; 2206; 2490; 2760; 2960; | 100 | 100 | 100 | 100 | 100 | 7.77E−11 |
| | 2981; 3037; 3316; 3639; 3670; 3816; 3939; 4188; 4219; 4405; 4562; 4808; 4879; 5075 | | | | | | |
| 1000 | 794; 1127; 3358; 4569 | 100 | 100 | 100 | 100 | 100 | 7.77E−11 |
| 1001 | 64; 109; 326; 606; 2737; 3478; 3550; 4157; 4520; 5063 | 100 | 100 | 100 | 100 | 100 | 7.77E−11 |
| 1002 | 44; 241; 1142; 1332; 1541; 2144; 2192; 2281; 2372; 2665; 3218; 4320; 4487; 5231; 5285 | 100 | 100 | 100 | 100 | 100 | 7.77E−11 |
| 1003 | 165; 427; 535; 574; 1095; 1122; 1584; 2322; 2415; 2623; 2654; 2685; 3193; 3206; 3396; 3648; 3722; 3861; 4136; 4860; 4906 | 100 | 100 | 100 | 100 | 100 | 7.77E−11 |
| 1004 | 219; 545; 1536; 1773; 2736; 3919; 4684; 4758; 4860 | 100 | 100 | 100 | 100 | 100 | 7.77E−11 |
| 1005 | 270; 492; 830; 1114; 1121; 1157; 1231; 1435; 1458; 1646; 1902; 2012; 2127; 2201; 2522; 2634; 2642; | 100 | 100 | 100 | 100 | 100 | 7.77E−11 |
| | 2732; 3383; 3721; 4782; 4806; 5195 | | | | | | |
| 1006 | 504; 685; 1309; 1816; 2714; 3299; 4497; 4511; 4978; 5271 | 100 | 100 | 100 | 100 | 100 | 7.77E−11 |
| 1007 | 539; 932; 1369; 1888; 2355; 2608; 2632; 2789; 3180; 3325; 3425; 3718; 3834; 4022; 4371; 4763; 4870; 5161; 5201 | 100 | 100 | 100 | 100 | 100 | 7.77E−11 |
| 1008 | 616; 1226; 2925 | 100 | 100 | 100 | 100 | 100 | 7.77E−11 |
| 1009 | 309; 707; 909; 961; 1261; 1313; 1375; 1761; 2009; 3055; 4725; 5100; 5129 | 100 | 100 | 100 | 100 | 100 | 7.77E−11 |
| 1010 | 1182; 1854; 1998; 3512; 4433 | 100 | 100 | 100 | 100 | 100 | 7.77E−11 |
| 1011 | 146; 218; 2324; 2389; 2537; 2576; 4379; 4649; 5149 | 100 | 100 | 100 | 100 | 100 | 7.77E−11 |
| 1012 | 471; 592; 616; 675; 782; 1553; 1671; 1715; 1764; 1928; 2500; 3855; 3941; 4171; 4224; 4568; 4778; 4854; 5008 | 100 | 100 | 100 | 100 | 100 | 7.77E−11 |
| 1013 | 219; 255; 671; 730; 779; 852; 899; 938; 1153; 1588; 1880; 1930; 2075; 2192; 2335; 2337; 2507; 2559; | 100 | 100 | 100 | 100 | 100 | 7.77E−11 |
| | 2577; 2610; 2779; 3055; 3078; 3091; 3145; 3154; 3166; 3511; 3530; 3543; 3561; 3565; 3695; 3790; | | | | | | |
| | 3904; 4233; 4265; 4280; 4341; 4483; 4486; 4625; 4705; 4720; 5136; 5137 | | | | | | |
| 1014 | 1601; 1909; 3688; 3990; 4685; 5030 | 100 | 100 | 100 | 100 | 100 | 7.77E−11 |
| 1015 | 110; 219; 907; 1038; 1191; 1428; 2670; 4619; 5195 | 100 | 100 | 100 | 100 | 100 | 7.77E−11 |
| 1016 | 4; 63; 91; 152; 357; 578; 836; 872; 1297; 1623; 1659; 1664; 1938; 2040; 2141; 2457; 2568; 2652; 2749; | 100 | 100 | 100 | 100 | 100 | 7.77E−11 |
| | 3128; 3586; 3850; 4100; 4457; 4637; 4710; 4836; 5130; 5260; 5281 | | | | | | |
| 1017 | 58; 115; 533; 596; 605; 639; 775; 992; 1012; 1199; 1242; 1330; 1408; 1447; 1532; 1565; 1607; 1618; | 100 | 100 | 100 | 100 | 100 | 7.77E−11 |
| | 1869; 1954; 2039; 2410; 2542; 2640; 2760; 2762; 2870; 3218; 3245; 3365; 3368; 3422; 3428; 3729; | | | | | | |
| | 3753; 3863; 4382; 4528; 4558; 4633; 4646; 4716; 4881; 4946; 4998; 5131; 5190; 5231; 5260 | | | | | | |
| 1018 | 238; 353; 501; 688; 745; 771; 1166; 1467; 1529; 2024; 2081; 2382; 2488; 2579; 2908; 3049; 3281; | 100 | 100 | 100 | 100 | 100 | 7.77E−11 |
| | 3580; 4130; 4867; 5108; 5156; 5225 | | | | | | |
| 1019 | 309; 1359; 2891; 3921; 4086 | 100 | 100 | 100 | 100 | 100 | 7.77E−11 |
| 1020 | 3; 30; 553; 668; 701; 1146; 1328; 1370; 1929; 2024; 2153; 2434; 2642; 2902; 3160; 3908; 4043; 4315; 4630; 4675; 4704; 5057 | 100 | 100 | 100 | 100 | 100 | 7.77E−11 |
| 1021 | 200; 377; 520; 529; 598; 720; 1321; 1539; 1719; 1950; 1965; 2219; 2234; 2317; 2344; 2626; 2797; | 100 | 100 | 100 | 100 | 100 | 7.77E−11 |
| | 2831; 2836; 2965; 3041; 3300; 3376; 3465; 3522; 3665; 4000; 4193; 4313; 4314; 4703; 4769; 5147 | | | | | | |
| 1022 | 174; 363; 536; 558; 562; 641; 716; 964; 1004; 1481; 1500; 1805; 1987; 2209; 2240; 2275; 2401; 2494; | 100 | 100 | 100 | 100 | 100 | 7.77E−11 |
| | 2826; 2914; 3180; 3222; 3303; 3402; 3444; 3543; 3685; 3985; 4034; 4184; 4291; 4471; 4546; 4656; 4704; 5254; 5274 | | | | | | |
| 1023 | 483; 1193; 1644; 1679; 1870; 2129; 2235; 3127; 3529; 3856; 3860; 4103; 4206; 4387; 4630 | 100 | 100 | 100 | 100 | 100 | 7.77E−11 |
| 1024 | 432; 584; 825; 977; 1072; 1252; 1276; 1571; 2273; 2349; 2545; 2615; 2628; 2760; 3099; 3160; 3215; | 100 | 100 | 100 | 100 | 100 | 7.77E−11 |
| | 3249; 3327; 3391; 4021; 4131; 4224; 4281; 4349; 4371; 4662; 4685; 4798 | | | | | | |
| 1025 | 68; 465; 1163; 1238; 1247; 1701; 1738; 2266; 2439; 2846; 3147; 3555; 3585; 3594; 4564; 4657 | 100 | 100 | 100 | 100 | 100 | 7.77E−11 |
| 1026 | 444; 536; 603; 1497; 1507; 2516; 2795; 4302; 4716 | 100 | 100 | 100 | 100 | 100 | 7.77E−11 |
| 1027 | 1135; 3975 | 100 | 100 | 100 | 100 | 100 | 7.77E−11 |
| 1028 | 48; 72; 219; 222; 368; 535; 647; 787; 1161; 1595; 1691; 2257; 2391; 2437; 2477; 2869; 3103; 3379; | 100 | 100 | 100 | 100 | 100 | 7.77E−11 |
| | 3420; 3521; 3653; 3889; 3986; 4109; 4113; 4193; 4303; 4675; 4820; 5083 | | | | | | |
| 1029 | 101; 234; 374; 437; 1870; 2431; 3168; 3898; 4024; 4029; 4048; 4142; 4300; 4373 | 100 | 100 | 100 | 100 | 100 | 7.77E−11 |
| 1030 | 10; 508; 1075; 1110; 1239; 1271; 1881; 2164; 2708; 3011; 3091; 4438; 4670; 4942; 5257 | 100 | 100 | 100 | 100 | 100 | 7.77E−11 |
| 1031 | 54; 185; 1005; 1539; 4592; 5242; 5286 | 100 | 100 | 100 | 100 | 100 | 7.77E−11 |
| 1032 | 123; 188; 2876; 3586; 4411; 4518; 5066 | 100 | 100 | 100 | 100 | 100 | 7.77E−11 |
| 1033 | 190; 365; 400; 414; 554; 556; 639; 777; 878; 914; 1001; 1415; 1569; 1615; 1640; 1684; 1830; 1858; | 100 | 100 | 100 | 100 | 100 | 7.77E−11 |
| | 1908; 2275; 2529; 2917; 2935; 2954; 3233; 3351; 3631; 3670; 3698; 3778; 3783; 3854; 3959; 4032; | | | | | | |
| | 4231; 4372; 4550; 4654; 4700; 4892; 4897; 4943; 4944; 5106; 5215 | | | | | | |
| 1034 | 356; 498; 1581; 1786; 1927; 2595; 3118; 3143; 4283; 4735; 5241 | 100 | 100 | 100 | 100 | 100 | 7.77E−11 |
| 1035 | 346; 522; 711; 756; 1192; 2622; 3319; 3988; 4004; 4021; 4417; 4748; 4979 | 100 | 100 | 100 | 100 | 100 | 7.77E−11 |
| 1036 | 455; 980; 1328; 2400; 4719; 4908; 5181 | 100 | 100 | 100 | 100 | 100 | 7.77E−11 |
| 1037 | 821; 2394; 2887; 3978; 4693; 4948; 5111 | 100 | 100 | 100 | 100 | 100 | 7.77E−11 |
| 1038 | 153; 761; 1342; 2612; 3304; 3388; 3628; 3946; 4536; 5024 | 100 | 100 | 100 | 100 | 100 | 7.77E−11 |
| 1039 | 917; 1489; 1582; 1705; 2053; 2114; 2388; 2429; 2488; 2670; 3619; 3877; 4117; 4219; 4280; 4379; 4542; 4556 | 100 | 100 | 100 | 100 | 100 | 7.77E−11 |
| 1040 | 208; 599; 969; 1260; 1783; 1993; 2184; 2221; 2660; 3045; 3235; 3278; 3372; 3567; 3586; 3587; 3632; | 100 | 100 | 100 | 100 | 100 | 7.77E−11 |
| | 3892; 3998; 4191; 4388; 4437; 4656; 4964; 5030; 5105; 5171; 5205 | | | | | | |
| 1041 | 47; 54; 110; 269; 484; 972; 1132; 1186; 1198; 1252; 1258; 1341; 1382; 1606; 1747; 1936; 2154; 2576; | 100 | 100 | 100 | 100 | 100 | 7.77E−11 |
| | 2757; 2810; 2863; 3007; 3362; 3403; 3548; 3761; 3776; 3777; 3852; 3952; 4012; 4018; 4307; 4692; 4698; 5150 | | | | | | |
| 1042 | 3; 377; 530; 627; 711; 748; 815; 841; 1190; 1196; 1346; 1520; 2167; 2457; 2508; 2634; 2720; 2792; | 100 | 100 | 100 | 100 | 100 | 7.77E−11 |
| | 3709; 4105; 4176; 4377; 4638; 4989; 5114; 5169 | | | | | | |
| 1043 | 193; 369; 607; 1191; 3047; 3421; 3919; 4528; 4562; 4652; 4861; 5247 | 100 | 100 | 100 | 100 | 100 | 7.77E−11 |
| 1044 | 301; 325; 329; 380; 407; 621; 677; 917; 1135; 1254; 1268; 1411; 1549; 1551; 1591; 1710; 1734; 1800; | 100 | 100 | 100 | 100 | 100 | 7.77E−11 |
| | 2012; 2101; 2242; 2636; 2783; 2843; 2857; 2930; 3245; 3335; 3715; 3739; 3998; 4008; 4252; 4308; | | | | | | |
| | 4430; 4500; 4532; 4607; 4766; 4773; 4779; 4859; 4995 | | | | | | |
| 1045 | 42; 223; 294; 350; 419; 816; 963; 1169; 1282; 1428; 1489; 1519; 1646; 1732; 2647; 2746; 2793; 2820; | 100 | 100 | 100 | 100 | 100 | 7.77E−11 |
| | 2883; 3328; 3363; 3702; 3841; 3862; 3919; 4011; 4029; 4118; 4224; 4241; 4416; 4493; 4604; 4669; 4752; 4802; 4822; 4863 | | | | | | |
| 1046 | 1140; 2665; 2716; 3174; 3303; 3558; 3961; 3980; 4007 | 100 | 100 | 100 | 100 | 100 | 7.77E−11 |
| 1047 | 385; 594; 3498; 3639; 4238; 4741 | 100 | 100 | 100 | 100 | 100 | 7.77E−11 |
| 1048 | 941; 1257; 1437; 1870; 2151; 2469; 2664; 2749; 2930; 3464 | 100 | 100 | 100 | 100 | 100 | 7.77E−11 |
| 1049 | 343; 367; 554; 1004; 1171; 1192; 2508; 2783; 3467; 3555; 3912; 3985; 4282; 4829 | 100 | 100 | 100 | 100 | 100 | 7.77E−11 |
| 1050 | 393; 780; 1599; 4186; 4376; 4659; 4897; 5181 | 100 | 100 | 100 | 100 | 100 | 7.77E−11 |
| 1051 | 1459; 2076; 2428; 2675; 3031; 3777; 4488 | 100 | 100 | 100 | 100 | 100 | 7.77E−11 |
| 1052 | 460; 1367; 2755; 3271 | 100 | 100 | 100 | 100 | 100 | 7.77E−11 |
| 1053 | 50; 189; 191; 463; 500; 757; 958; 971; 1063; 1066; 1331; 1561; 2057; 2651; 3112; 3220; 3446; 3567; | 100 | 100 | 100 | 100 | 100 | 7.77E−11 |
| | 3727; 3771; 3832; 3858; 4417; 4458; 4765; 4860; 4872; 4953; 5206 | | | | | | |
| 1054 | 40; 162; 353; 988; 1481; 1497; 1614; 1785; 1857; 2010; 2161; 2180; 2385; 2521; 2529; 2758; 2847; | 100 | 100 | 100 | 100 | 100 | 7.77E−11 |
| | 2875; 2891; 3441; 3472; 3525; 3731; 4338; 4346; 4600; 4768; 4894; 4956; 5169; 5177; 5253 | | | | | | |
| 1055 | 792; 1740; 2223; 3375; 4597; 5257 | 100 | 100 | 100 | 100 | 100 | 7.77E−11 |
| 1056 | 79; 148; 306; 375; 636; 639; 651; 789; 804; 941; 972; 1026; 1187; 1215; 1390; 1404; 1405; 1542; 1853; 1910; 1949; | 100 | 100 | 100 | 100 | 100 | 7.77E−11 |
| | 2078; 2140; 2234; 2317; 2335; 2425; 2511; 2543; 3147; 3175; 3351; 3433; 3900; 3986; 4100; 4162; 4173; 4452; 4610 | | | | | | |
| 1057 | 121; 190; 782; 1340; 2016; 2022; 3032; 3626; 4361; 4656 | 100 | 100 | 100 | 100 | 100 | 7.77E−11 |
| 1058 | 41; 694; 2507; 2521; 2831; 3514; 3630; 5153 | 100 | 100 | 100 | 100 | 100 | 7.77E−11 |
| 1059 | 34; 171; 1719; 2122; 4530; 5021; 5239 | 100 | 100 | 100 | 100 | 100 | 7.77E−11 |
| 1060 | 108; 235; 1078; 1099; 1102; 1172; 1417; 1425; 1736; 2251; 2413; 2609; 2624; 3507; 3582; 3652; 3682; 4285; 4618; 5237 | 100 | 100 | 100 | 100 | 100 | 7.77E−11 |
| 1061 | 2022; 3586; 4545; 4698; 4745; 5029 | 100 | 100 | 100 | 100 | 100 | 7.77E−11 |
| 1062 | 85; 108; 718; 1000; 2675; 3077; 3243; 3960; 4068; 4400 | 100 | 100 | 100 | 100 | 100 | 7.77E−11 |
| 1063 | 1452; 1457; 2022; 2643; 4643; 5001; 5011; 5093; 5173; 5190 | 100 | 100 | 100 | 100 | 100 | 7.77E−11 |
| 1064 | 63; 528; 994; 1579; 1758; 2239; 2329; 2613; 2649; 2721; 2986; 3248; 3549; 3645; 4146; 4702; 5112 | 100 | 100 | 100 | 100 | 100 | 7.77E−11 |
| 1065 | 54; 174; 296; 459; 1355; 1910; 4365; 4839 | 100 | 100 | 100 | 100 | 100 | 7.77E−11 |
| 1066 | 178; 605; 2555; 4064; 4120; 4364; 5070; 5201 | 100 | 100 | 100 | 100 | 100 | 7.77E−11 |
| 1067 | 148; 152; 232; 243; 348; 450; 482; 565; 696; 1105; 1329; 1389; 1616; 1683; 2190; 2399; 2801; 3027; 3168; 3331; 4759; 4822; 4968; 5232; 5274 | 100 | 100 | 100 | 100 | 100 | 7.77E−11 |
| 1068 | 114; 526; 528; 681; 795; 838; 1081; 1162; 1228; 1279; 1355; 1384; 1404; 1432; 1456; 1549; 1583; 1770; | 100 | 100 | 100 | 100 | 100 | 7.77E−11 |
| | 1839; 1852; 2058; 2206; 2591; 2654; 2713; 2784; 2864; 2873; 3077; 3157; 3426; 3613; 3688; 3763; | | | | | | |
| | 3878; 3887; 3899; 4081; 4126; 4129; 4276; 4380; 4417; 4653; 4811; 4854 | | | | | | |
| 1069 | 20; 299; 530; 676; 776; 883; 1060; 1814; 1943; 1981; 2010; 2079; 2180; 2190; 2690; 2731; 2976; 3353; | 100 | 100 | 100 | 100 | 100 | 7.77E−11 |
| | 3384; 3572; 3586; 4296; 4964; 5274 | | | | | | |
| 1070 | 269; 632; 778; 818; 1011; 1274; 1337; 2702; 2837; 3054; 3302; 3740; 4618; 4639; 4793 | 100 | 100 | 100 | 100 | 100 | 7.77E−11 |
| 1071 | 759; 1262; 1377; 3975 | 100 | 100 | 100 | 100 | 100 | 7.77E−11 |
| 1072 | 29; 137; 321; 379; 588; 617; 779; 809; 934; 963; 1011; 1044; 2051; 2114; 2192; 2418; 2536; 2549; 2836; | 100 | 100 | 100 | 100 | 100 | 7.77E−11 |
| | 973; 2989; 3054; 3172; 3350; 3362; 3890; 3915; 3959; 4222; 4354; 4756; 4922; 4933; 4997; 5208; 5242; 5285 | | | | | | |
| 1073 | 379; 1516; 2039; 2268; 2314; 2642; 3344; 3808; 3817 | 100 | 100 | 100 | 100 | 100 | 7.77E−11 |
| 1074 | 74; 194; 412; 423; 1453; 1688; 1863; 1936; 2857; 2888; 3209; 3493; 4000; 4325; 4363; 4394; 4583; 4664; 4825; 5193 | 100 | 100 | 100 | 100 | 100 | 7.77E−11 |
| 1075 | 420; 1046; 1606; 2815; 3271; 3428; 4408; 4740; 4892 | 100 | 100 | 100 | 100 | 100 | 7.77E−11 |
| 1076 | 202; 572; 633; 678; 719; 1049; 1458; 1598; 2412; 2479; 4032; 4291 | 100 | 100 | 100 | 100 | 100 | 7.77E−11 |
| 1077 | 128; 340; 747; 823; 1565; 2912; 3751; 4433; 4887; 5023 | 100 | 100 | 100 | 100 | 100 | 7.77E−11 |
| 1078 | 227; 679; 980; 1149; 2065; 4099; 4582; 4896 | 100 | 100 | 100 | 100 | 100 | 7.77E−11 |
| 1079 | 207; 280; 1004; 3216; 3307; 3359; 3523; 3657; 4049; 4099; 4244; 4558; 4560; 5077 | 100 | 100 | 100 | 100 | 100 | 7.77E−11 |
| 1080 | 219; 1495; 3608 | 100 | 100 | 100 | 100 | 100 | 7.77E−11 |
| 1081 | 596; 933; 1336; 2020; 3284; 3416; 3764; 4005; 4067; 4149 | 100 | 100 | 100 | 100 | 100 | 7.77E−11 |
| 1082 | 572; 622; 750; 1172; 1197; 1248; 1312; 1358; 1425; 1501; 1552; 1834; 1913; 2148; 2182; 2309; 2500; | 100 | 100 | 100 | 100 | 100 | 7.77E−11 |
| | 2707; 2715; 2841; 3476; 3674; 3931; 3970; 4094; 4102; 4394; 4413; 4919; 4951 | | | | | | |
| 1083 | 750; 1210; 1941; 2616; 2666; 4025; 4174; 4365; 4464 | 100 | 100 | 100 | 100 | 100 | 7.77E−11 |
| 1084 | 603; 1017; 1341; 1573; 1592; 1610; 1802; 1827; 1907; 2106; 2188; 2215; 2787; 2796; 2923; 2951; | 100 | 100 | 100 | 100 | 100 | 7.77E−11 |
| | 3039; 3419; 3697; 3707; 4125; 4238; 4835; 4838; 4926; 5192; 5243 | | | | | | |
| 1085 | 322; 416; 458; 615; 689; 755; 793; 1583; 1783; 1787; 1971; 2124; 2606; 2640; 3038; 3039; 3067; 3109; | 100 | 100 | 100 | 100 | 100 | 7.77E−11 |
| | 3123; 3152; 3210; 3477; 3702; 4376; 4558; 4684; 4880; 4897; 5098; 5172; 5177 | | | | | | |
| 1086 | 34; 95; 237; 292; 296; 321; 890; 1324; 1506; 1700; 1759; 1788; 2471; 2723; 2857; 3420; 3818; 4402; 4974 | 100 | 100 | 100 | 100 | 100 | 7.77E−11 |
| 1087 | 1293; 1661; 2670 | 100 | 100 | 100 | 100 | 100 | 7.77E−11 |

TABLE 5

| | | | | | | (min/ |
|---|---|---|---|---|---|---|
| | | (min/ | (min/ | (min/ | (min/ | max/ |
| | | max/ | max/ | max/ | max/ | mean/ |
| | | mean/ | mean/ | mean/ | mean/ | stderr) |
| | | stderr) | stderr) | stderr) | stderr) | Fisher's |
| | kmers | SN | SP | PPV | NPV | P-value |
| 1 | 458; 1592; 1767; 2782; 2846; 3653; | 100.000/ | 100.000/ | 100.000/ | 100.000/ | 0.0178571/ |
| | 3751; 3930; 4440; 5098 | 100.000/ | 100.000/ | 100.000/ | 100.000/ | 0.125/ |
| | | 100.000/ | 100.000/ | 100.000/ | 100.000/ | 0.0323571/ |
| | | 0.000 | 0.000 | 0.000 | 0.000 | 0.00265463 |
| 2 | 449; 876; 1830; 2704; 3139; 3205; | 100.000/ | 100.000/ | 100.000/ | 100.000/ | 0.0178571/ |
| | 3829; 4149; 4571 | 100.000/ | 100.000/ | 100.000/ | 100.000/ | 0.125/ |
| | | 100.000/ | 100.000/ | 100.000/ | 100.000/ | 0.0333571/ |
| | | 0.000 | 0.000 | 0.000 | 0.000 | 0.00281454 |
| 3 | 188; 1165; 1876; 2272; 2677; 2886; | 100.000/ | 100.000/ | 100.000/ | 100.000/ | 0.0178571/ |
| | 3172; 3341; 3588; 4179; 4300; 5104; | 100.000/ | 100.000/ | 100.000/ | 100.000/ | 0.125/ |
| | 5156 | 100.000/ | 100.000/ | 100.000/ | 100.000/ | 0.0308571/ |
| | | 0.000 | 0.000 | 0.000 | 0.000 | 0.00268056 |
| 4 | 769; 824; 941; 1472; 1817; 4139; | 100.000/ | 100.000/ | 100.000/ | 100.000/ | 0.0178571/ |
| | 5068 | 100.000/ | 100.000/ | 100.000/ | 100.000/ | 0.125/ |
| | | 100.000/ | 100.000/ | 100.000/ | 100.000/ | 0.0334643/ |
| | | 0.000 | 0.000 | 0.000 | 0.000 | 0.00279683 |
| 5 | 442; 1448; 1590; 1870; 4026; 4207 | 100.000/ | 100.000/ | 100.000/ | 100.000/ | 0.0178571/ |
| | | 100.000/ | 100.000/ | 100.000/ | 100.000/ | 0.125/ |
| | | 100.000/ | 100.000/ | 100.000/ | 100.000/ | 0.0325714/ |
| | | 0.000 | 0.000 | 0.000 | 0.000 | 0.00263976 |
| 6 | 152; 1167; 4476 | 100.000/ | 100.000/ | 100.000/ | 100.000/ | 0.0178571/ |
| | | 100.000/ | 100.000/ | 100.000/ | 100.000/ | 0.125/ |
| | | 100.000/ | 100.000/ | 100.000/ | 100.000/ | 0.0290714/ |
| | | 0.000 | 0.000 | 0.000 | 0.000 | 0.00231679 |
| 7 | 160; 506; 1401; 2810; 2960; 4435; | 100.000/ | 100.000/ | 100.000/ | 100.000/ | 0.0178571/ |
| | 4514; 4646; 4982 | 100.000/ | 100.000/ | 100.000/ | 100.000/ | 0.125/ |
| | | 100.000/ | 100.000/ | 100.000/ | 100.000/ | 0.0268214/ |
| | | 0.000 | 0.000 | 0.000 | 0.000 | 0.00156582 |
| 8 | 219; 1178; 1347; 1818; 2904; 5070 | 100.000/ | 100.000/ | 100.000/ | 100.000/ | 0.0178571/ |
| | | 100.000/ | 100.000/ | 100.000/ | 100.000/ | 0.125/ |
| | | 100.000/ | 100.000/ | 100.000/ | 100.000/ | 0.0300357/ |
| | | 0.000 | 0.000 | 0.000 | 0.000 | 0.00206826 |
| 9 | 4952 | 100.000/ | 100.000/ | 100.000/ | 100.000/ | 0.0178571/ |
| | | 100.000/ | 100.000/ | 100.000/ | 100.000/ | 0.125/ |
| | | 100.000/ | 100.000/ | 100.000/ | 100.000/ | 0.0270357/ |
| | | 0.000 | 0.000 | 0.000 | 0.000 | 0.00154825 |
| 10 | 784; 1033; 3807; 4589 | 100.000/ | 100.000/ | 100.000/ | 100.000/ | 0.0178571/ |
| | | 100.000/ | 100.000/ | 100.000/ | 100.000/ | 0.125/ |
| | | 100.000/ | 100.000/ | 100.000/ | 100.000/ | 0.0332143/ |
| | | 0.000 | 0.000 | 0.000 | 0.000 | 0.00263058 |
| 11 | 404; 1149; 2670; 2909; 3552; 3561; | 100.000/ | 100.000/ | 100.000/ | 100.000/ | 0.0178571/ |
| | 4300; 4633 | 100.000/ | 100.000/ | 100.000/ | 100.000/ | 0.125/ |
| | | 100.000/ | 100.000/ | 100.000/ | 100.000/ | 0.0328929/ |
| | | 0.000 | 0.000 | 0.000 | 0.000 | 0.00281274 |
| 12 | 171; 319; 1046; 2036; 3253; 3362; | 100.000/ | 100.000/ | 100.000/ | 100.000/ | 0.0178571/ |
| | 4102; 5208 | 100.000/ | 100.000/ | 100.000/ | 100.000/ | 0.125/ |
| | | 100.000/ | 100.000/ | 100.000/ | 100.000/ | 0.0292857/ |
| | | 0.000 | 0.000 | 0.000 | 0.000 | 0.00210096 |
| 13 | 346; 967; 1344; 2082; 2837; 3122; | 100.000/ | 100.000/ | 100.000/ | 100.000/ | 0.0178571/ |
| | 3558 | 100.000/ | 100.000/ | 100.000/ | 100.000/ | 0.125/ |
| | | 100.000/ | 100.000/ | 100.000/ | 100.000/ | 0.0323929/ |
| | | 0.000 | 0.000 | 0.000 | 0.000 | 0.00282734 |
| 14 | 207; 621; 961; 2137; 3367; 3576; | 100.000/ | 100.000/ | 100.000/ | 100.000/ | 0.0178571/ |
| | 4702 | 100.000/ | 100.000/ | 100.000/ | 100.000/ | 0.125/ |
| | | 100.000/ | 100.000/ | 100.000/ | 100.000/ | 0.0299643/ |
| | | 0.000 | 0.000 | 0.000 | 0.000 | 0.00251459 |
| 15 | 19; 62; 1315; 1372; 2287; 3227; | 100.000/ | 100.000/ | 100.000/ | 100.000/ | 0.0178571/ |
| | 3866; 3954; 4363; 4630 | 100.000/ | 100.000/ | 100.000/ | 100.000/ | 0.125/ |
| | | 100.000/ | 100.000/ | 100.000/ | 100.000/ | 0.0288571/ |
| | | 0.000 | 0.000 | 0.000 | 0.000 | 0.00233046 |
| 16 | 2082; 3611; 4956; 5207 | 100.000/ | 100.000/ | 100.000/ | 100.000/ | 0.0178571/ |
| | | 100.000/ | 100.000/ | 100.000/ | 100.000/ | 0.125/ |
| | | 100.000/ | 100.000/ | 100.000/ | 100.000/ | 0.0284286/ |
| | | 0.000 | 0.000 | 0.000 | 0.000 | 0.00211497 |
| 17 | 1371; 1420; 1989; 2593; 2998; 3191; | 100.000/ | 100.000/ | 100.000/ | 100.000/ | 0.0178571/ |
| | 3363; 3378; 3782; 3806; 4036; 4302; | 100.000/ | 100.000/ | 100.000/ | 100.000/ | 0.125/ |
| | 4656; 4693; 4708; 4760; 5109 | 100.000/ | 100.000/ | 100.000/ | 100.000/ | 0.0305714/ |
| | | 0.000 | 0.000 | 0.000 | 0.000 | 0.00250763 |

TABLE 6

| | | | | | | | Top | | | | | |
|---|---|---|---|---|---|---|---|---|---|---|---|---|
| | | | | | | | signatures, | | | | | |
| | | | | | | Total | selected | | | | | |
| | | | | | Significant | Frigate | for cross | | | | | |
| | | | | Total | features, | explore | validation | | | | | |
| | | | | number | false | iterations | (if >100, | | | | | Top mean |
| | | | | of total | discovery | including | then all | Top mean | Top mean | Top mean | Top mean | P-value, |
| | Samples, | Samples, | | distinct | rate | single | had 100% | SN after | SP after | PPV after | NPV after | after |
| | Population | Population | Kmer | features | [FDR] | feature | initial | cross- | cross- | cross- | cross- | cross- |
| Classification | 1 | 2 | size | (kmers) | <5% | testing | accuracy) | validation | validation | validation | validation | validation |
| HMP Stool | 139 | 491 | 2 | 10 | 6 | 64 | 63 | 94.48% | 98.14% | 93.53% | 98.44% | 0.0000000 |
| vs. HMP | | | | | | | | | | | | |
| Other | | | | | | | | | | | | |
| HMP Stool | 139 | 491 | 3 | 32 | 23 | 50,049 | 1,205 | 100.00% | 99.99% | 99.97% | 100.00% | 0.0000000 |
| vs. HMP | | | | | | | | | | | | |
| Other | | | | | | | | | | | | |
| HMP Stool | 139 | 491 | 4 | 136 | 116 | 100,116 | 12,753 | 100.00% | 100.00% | 100.00% | 100.00% | 0.0000000 |
| vs. HMP | | | | | | | | | | | | |
| Other | | | | | | | | | | | | |
| HMP Stool | 139 | 491 | 8 | 32,896 | 29,358 | 106,822 | 8556 | 100.00% | 100.00% | 100.00% | 100.00% | 0.0000000 |
| vs. HMP | | | | | | | | | | | | |
| Other | | | | | | | | | | | | |
| CD vs. | 16 | 128 | 8 | 32,896 | 3,295 | 103,295 | 100 | 84.47% | 98.14% | 86.91% | 97.76% | 0.0170000 |
| Healthy | | | | | | | | | | | | |
| CD vs. | 16 | 128 | 8 | 32,896 | 3,295 | 103,295 | 100 | 85.01% | 96.86% | 78.90% | 97.78% | 0.0370000 |
| Healthy | | | | | | | | | | | | |
| CD vs. | 16 | 128 | 8 | 32,896 | 3,295 | 103,295 | 100 | 80.92% | 98.03% | 84.31% | 97.41% | 0.0260000 |
| Healthy | | | | | | | | | | | | |
| CD vs. | 16 | 128 | 8 | 32,896 | 3,295 | 103,295 | 100 | 82.73% | 97.32% | 81.88% | 97.26% | 0.0260000 |
| Healthy | | | | | | | | | | | | |
| UC vs. | 21 | 121 | 2 | 10 | 0 (all | 1,024 | 100 | 45.28% | 90.45% | 46.34% | 89.83% | 0.2720000 |
| Healthy | | | | | features | | | | | | | |
| | | | | | kept) | | | | | | | |
| UC vs. | 21 | 121 | 3 | 32 | 1 | 3 | 2 | 26.20% | 88.07% | 27.26% | 87.80% | 0.5600000 |
| Healthy | | | | | | | | | | | | |
| UC vs. | 21 | 121 | 4 | 136 | 0 (all | 100,136 | 100 | 60.17% | 92.92% | 62.75% | 92.16% | 0.0770000 |
| Healthy | | | | | features | | | | | | | |
| | | | | | kept) | | | | | | | |
| UC vs. | 21 | 121 | 8 | 32,896 | 138 (FDR | 100,138 | 100 | 81.53% | 96.18% | 80.69% | 96.44% | 0.0157000 |
| Healthy | | | | | < 10%) | | | | | | | |
| CD vs. UC | 16 | 21 | 8 | 32,896 | 5,287 | 105,287 | 1,087 | 100.00% | 100.00% | 100.00% | 100.00% | 0.0268200 |
| CD vs. UC | 16 | 21 | 8 | 32,896 | 5,287 | 105,287 | 1,087 | 100.00% | 100.00% | 100.00% | 100.00% | 0.0284286 |
| CD vs. UC | 16 | 21 | 8 | 32,896 | 5,287 | 105,287 | 1,087 | 100.00% | 100.00% | 100.00% | 100.00% | 0.0299643 |
| CD vs. UC | 16 | 21 | 8 | 32,896 | 5,287 | 105,287 | 1,087 | 100.00% | 100.00% | 100.00% | 100.00% | 0.0320000 |
| Random | 100 | 100 | 4 | 136 | 0 (all | 100,136 | 100 | 73.23% | 52.04% | 59.10% | 67.20% | 0.2540000 |
| DNA | | | | | features | | | | | | | |
| sequences | | | | | kept) | | | | | | | |

TABLE 7
IBD vs. Non-IBD Kmer Signature

| SIGNATURE | SIGNATURE |
|---|---|
| NUMBER | SEQUENCE |
| IBD KMR. NO. 1 | AAGTGGTC |
| IBD KMR. NO. 2 | ACACCGGT |
| IBD KMR. NO. 3 | ACATCTCA |
| IBD KMR. NO. 4 | AGAGTAGC |
| IBD KMR. NO. 5 | AGCCTTTG |
| IBD KMR. NO. 6 | AGTATGGA |
| IBD KMR. NO. 7 | CAAGTTCC |
| IBD KMR. NO. 8 | CAATCTGC |
| IBD KMR. NO. 9 | CACGAGCA |
| IBD KMR. NO. 10 | CAGTGCCG |
| IBD KMR. NO. 11 | CCGCTCCG |
| IBD KMR. NO. 12 | CGAGGAGC |
| IBD KMR. NO. 13 | CTGACGCA |
| IBD KMR. NO. 14 | GACATTGC |
| IBD KMR. NO. 15 | GCAGCCAA |
| IBD KMR. NO. 16 | GTATCACA |
| IBD KMR. NO. 17 | ACTATGCC |
| IBD KMR. NO. 18 | CCGCAAAG |
| IBD KMR. NO. 19 | GATATCCA |
| IBD KMR. NO. 20 | GGTTATGA |
| IBD KMR. NO. 21 | GTCAAAAC |

TABLE 8
Results of IBD vs. Non-IBD Kmer Signature Set

| | TRUE | TRUE | |
|---|---|---|---|
| | IBD | NON-IBD | TOTAL |
| PREDICTED IBD | 322 | 17 | 339 |
| PREDICTED | 67 | 291 | 358 |
| NON-IBD | | | |
| TOTAL | 389 | 308 | 697 |

TABLE 9
Results Summary for IBD v. Non-IBD Kmer Signature Set
RESULTS SUMMARY

| | |
|---|---|
| SENSITIVITY | 82.8% |
| SPECIFICITY | 94.5% |
| POSITIVE | 95.0% |
| PREDICTIVE VALUE | |
| NEGATIVE | 81.3% |
| PREDICTIVE VALUE | |
| FISHER'S EXACT | 6.16E−105 |
| TEST | |

TABLE 10
Kmers

| SIGNATURE | SIGNATURE |
|---|---|
| NUMBER | SEQUENCE |
| Kmr No. 5288. | AAGTGGTC |
| Kmr No. 5289. | ACACCGGT |
| Kmr No. 5290. | ACATCTCA |
| Kmr No. 5291. | AGAGTAGC |
| Kmr No. 5292. | AGCCTTTG |
| Kmr No. 5293. | AGTATGGA |
| Kmr No. 5294. | CAAGTTCC |
| Kmr No. 5295. | CAATCTGC |
| Kmr No. 5296. | CACGAGCA |
| Kmr No. 5297. | CAGTGCCG |
| Kmr No. 5298. | CCGCTCCG |
| Kmr No. 5299. | CGAGGAGC |
| Kmr No. 5300. | CTGACGCA |
| Kmr No. 5301. | GACATTGC |
| Kmr No. 5302. | GCAGCCAA |
| Kmr No. 5303. | GTATCACA |
| Kmr No. 5304. | AAAGGATT |
| Kmr No. 5305. | AGTATTTA |
| Kmr No. 5306. | ATCCACCC |
| Kmr No. 5307. | ATGAAGTA |
| Kmr No. 5308. | CGCCAGAC |
| Kmr No. 5309. | AAAAGCGA |
| Kmr No. 5310. | AACCCTAA |
| Kmr No. 5311. | AAGATGGT |
| Kmr No. 5312. | AAGGTTAG |
| Kmr No. 5313. | AAGTGTCG |
| Kmr No. 5314. | AATATTAT |
| Kmr No. 5315. | AATTGTAT |
| Kmr No. 5316. | ACAACCGC |
| Kmr No. 5317. | ACCTTCCC |
| Kmr No. 5318. | ACTAAATC |
| Kmr No. 5319. | ACTTCACT |
| Kmr No. 5320. | AGAACGAC |
| Kmr No. 5321. | AGATACAA |
| Kmr No. 5322. | AGCACCGA |
| Kmr No. 5323. | AGGAAAGC |
| Kmr No. 5324. | ATGACGAC |
| Kmr No. 5325. | ATTTGCCG |
| Kmr No. 5326. | CACGATGA |
| Kmr No. 5327. | CACTCCAG |
| Kmr No. 5328. | CACTCGAA |
| Kmr No. 5329. | CATCTGTA |
| Kmr No. 5330. | CCATATCG |
| Kmr No. 5331. | CGATCTTA |
| Kmr No. 5332. | CGGATGAC |
| Kmr No. 5333. | CGTGCAGA |
| Kmr No. 5334. | CTAAAAAG |
| Kmr No. 5335. | GAAAGGAC |
| Kmr No. 5336. | GAACCCTC |
| Kmr No. 5337. | GATATTGA |
| Kmr No. 5338. | GATGCGAA |
| Kmr No. 5339. | GCAAGCGC |
| Kmr No. 5340. | GCACGAGC |
| Kmr No. 5341. | GCACTGTA |
| Kmr No. 5342. | GCAGACGA |
| Kmr No. 5343. | GCATTGCC |
| Kmr No. 5344. | GCGAGTAC |
| Kmr No. 5345. | GCGTCAAA |
| Kmr No. 5346. | GGTCATAC |
| Kmr No. 5347. | GTCTTCAC |
| Kmr No. 5348. | TCAAAAGA |
| Kmr No. 5349. | TCAGGACA |
| Kmr No. 5350. | TCATCGCA |
| Kmr No. 5351. | TGGTATAA |
| Kmr No. 5352. | TGGTGAAA |
| Kmr No. 5353. | AAAAAGTT |
| Kmr No. 5354. | AAAAGTTA |
| Kmr No. 5355. | AAATGAGT |
| Kmr No. 5356. | AAATGCTA |
| Kmr No. 5357. | AACGGCGG |
| Kmr No. 5358. | AACTAAAC |
| Kmr No. 5359. | AAGCGCCT |
| Kmr No. 5360. | AAGGCGCG |
| Kmr No. 5361. | AAGTGCTA |
| Kmr No. 5362. | AATAAAAA |
| Kmr No. 5363. | AATAAAAT |
| Kmr No. 5364. | AATTAGTC |
| Kmr No. 5365. | AATTTAAT |
| Kmr No. 5366. | ACAAACAA |
| Kmr No. 5367. | ACGCTGAT |
| Kmr No. 5368. | ACTAGAGG |
| Kmr No. 5369. | AGGAAAGA |
| Kmr No. 5370. | AGGAGGCG |
| Kmr No. 5371. | AGGATCGG |
| Kmr No. 5372. | AGGCGCAT |
| Kmr No. 5373. | AGTATCGC |
| Kmr No. 5374. | ATCACTTA |
| Kmr No. 5375. | ATCAGCTC |
| Kmr No. 5376. | ATCCACGC |
| Kmr No. 5377. | ATCGAGGA |
| Kmr No. 5378. | ATCTAAAT |
| Kmr No. 5379. | CCATACCG |
| Kmr No. 5380. | CCGTGAGA |
| Kmr No. 5381. | CGCCCATA |
| Kmr No. 5382. | CGCCGCTA |
| Kmr No. 5383. | CGGTATGC |
| Kmr No. 5384. | CTAAAGCA |
| Kmr No. 5385. | CTACTGAA |
| Kmr No. 5386. | CTATGCGA |
| Kmr No. 5387. | CTATTTAA |
| Kmr No. 5388. | CTGCGGTA |
| Kmr No. 5389. | GAAGGCGC |
| Kmr No. 5390. | GAGTCCGA |
| Kmr No. 5391. | GATTGACC |
| Kmr No. 5392. | GATTGAGC |
| Kmr No. 5393. | GCCTATGA |

What is claimed is:

1. A method of diagnosing inflammatory bowel disease in a subject in need thereof, comprising the steps of:
(a) in a microbiome sample obtained from said subject, determining the presence of a nucleic acid oligomer that distinguishes subjects having inflammatory bowel disease from subjects not having inflammatory bowel disease;
(b) recording the presence and identity of said nucleic acid oligomer in an electronic format; and
(c) making and recording a diagnosis of inflammatory bowel disease based on the presence of said nucleic acid oligomer.

2. The method of claim 1, wherein said microbiome sample is isolated from a stool sample.

3. The method of claim 1, wherein the presence of said nucleic acid oligomer is determined by whole-genome shotgun sequencing of total DNA isolated from said microbiome sample.

4. The method of claim 1, wherein said nucleic acid oligomer comprises between 2 and 10 nucleotides.

5. The method of claim 4, wherein said oligomer comprises an octamer.

6. The method of claim 5, wherein said octamer is selected from the group consisting of the octamers listed in Table 3 and 10.

7. The method of claim 6, wherein said octamer is selected from the group consisting of the octamers listed in Table 7.

8. The method of claim 1, further comprising the step of reporting said diagnosis to a medical professional, to the subject or a representative of the subject, or to a combination thereof.

9. A method of differentially diagnosing Crohn's disease and ulcerative colitis in a subject in need thereof, comprising the steps of:
(a) in a microbiome sample obtained from said subject, determining the presence of a signature comprising a plurality of nucleic acid oligomers that distinguishes subjects having Crohn's Disease and subjects having ulcerative colitis;
(b) recording the presence and identity of said signature in an electronic format; and
(c) making a diagnosis of Crohn's disease or ulcerative colitis based on the presence of said signature.

10. The method of claim 9, wherein said microbiome sample is isolated from a stool sample.

11. The method of claim 9, wherein the presence of each said nucleic acid oligomer is determined by whole-genome shotgun sequencing of total DNA isolated from said microbiome sample.

12. The method of claim 9, wherein each said nucleic acid oligomer comprises between 2 and 10 nucleotides.

13. The method of claim 12, wherein each said nucleic acid oligomer comprises an octamer.

14. The method of claim 9, wherein said signature is selected from the group consisting of the signatures listed in Table 4.

15. The method of claim 14, wherein said signature is selected from the group consisting of the signatures listed in Table 5.

16. The method of claim 9, further comprising the step of reporting said diagnosis to a medical professional, to the subject or a representative of the subject, or to a combination thereof.
